SEQUENCE LISTING (129 sequences)

SEQ ID NO: 1 - protein, 118 aa, Artificial Sequence
  source: /note=“Description of Artificial Sequence Synthetic polypeptide”
EVQLLEQSGAELKKPGASVKVSCKASGYTFTNHYMHWVRQAPGQGLEWMGIINPISGSTSYAQKFQGRVTMTRDTSSSTAYMDLSRLTSDDTAVYYCARDIVDAFDFWGQGTTVTVSS

SEQ ID NO: 2 - protein, 118 aa, Artificial Sequence
  source: /note=“Description of Artificial Sequence Synthetic polypeptide”
EVQLLEQSGAELKKPGASVKVSCKASGYTFTSHYMHWVRQAPGQGLEWMGIINPISGSTTYAQKFQGRVTMTRDTSSSTAYMDLSRLTSDDTAVYYCARDIVDAFDYWGQGTTVTVSS

SEQ ID NO: 3 - protein, 118 aa, Artificial Sequence
  source: /note=“Description of Artificial Sequence Synthetic polypeptide”
EVQLLEQSGAELKKPGASVKVSCKASGYTFTNHFMHWVRQAPGQGLEWMGTINPVSGSTSYAQKFQGRVTMTRDTSSSTAYMDLSRLTSDDTAVYYCAREILDAFDYWGQGTTVTVSS

SEQ ID NO: 4 - protein, 118 aa, Artificial Sequence
  source: /note=“Description of Artificial Sequence Synthetic polypeptide”
EVQLLEQSGAELKKPGASVKVSCKASGYTFTNHFIHWVRQAPGQGLEWMGTINPVSGSTNYAQKFQGRVTMTRDTSSSTAYMDLSRLTSDDTAVYYCAREILDAFDYWGQGTTVTVSS

SEQ ID NO: 5 - protein, 117 aa, Artificial Sequence
  source: /note=“Description of Artificial Sequence Synthetic polypeptide”
QVQLVQSGAELKKPGASVKVSCKASGYTFTNHYMHWVRQAPGQGLEWMGIINPISGSTTYAQKFQGRVTMTRDTSSSTAYMDLSRLTSDDTAVYYCARDIVDAFDFWGQGTTVTVSS

SEQ ID NO: 6 - protein, 117 aa, Artificial Sequence
  source: /note=“Description of Artificial Sequence Synthetic polypeptide”
QVQLLESGAVLARPGTSVKISCKASGYTFTNHYMHWVKQRPGQGLEWIGIINPISGSSSNAQKFQGRVTITRDTSTSTVFMELSSLRSEDTAVYYCARDIVDAFDFWGQGTLVTVSS

SEQ ID NO: 7 - protein, 117 aa, Artificial Sequence
  source: /note=“Description of Artificial Sequence Synthetic polypeptide”
EVQLVQSGAEVRKPGASVKVSCKASGYTFTNHYMHWVRQAPGQGLEWMGIINPISGSTSNAQKLQGRVTFTRDTSANTAYMELSGLIPEDTAIYYCARDIVDAFDFWGQGTLVTVSS

SEQ ID NO: 8 - protein, 117 aa, Artificial Sequence
  source: /note=“Description of Artificial Sequence Synthetic polypeptide”
QVQLLESGAVLARPGTSVKISCKASGYTFTNHYMHWVKQRPGQGLEWIGIINPITGSSSNAQKFQGRVTITRDTSTSTVFMELSSLRSEDTAVYYCARDIVDAFDFWGQGTLVTVSS

SEQ ID NO: 9 - protein, 117 aa, Artificial Sequence
  source: /note=“Description of Artificial Sequence Synthetic polypeptide”
EVQLVQSGAEVRKPGASVKVSCKASGYTFTNHYMHWVRQAPGQGLEWMGIINPISGSTSNAQKLQGRVTFTRDTSANTAYMELSGLIPEDTAIYYCAREIVDAFDFWGQGTLVTVSS

SEQ ID NO: 10 - protein, 117 aa, Artificial Sequence
  source: /note=“Description of Artificial Sequence Synthetic polypeptide”
QVQLLESGAVLARPGTSVKISCKASGYTFTNYYMHWVKQRPGQGLEWIGIINPITGSSSNAQKFQGRVTITRDTSTSTVFMELSSLRSEDTAVYYCAREIVDAFDFWGQGTLVTVSS

SEQ ID NO: 11 - protein, 117 aa, Artificial Sequence
  source: /note=“Description of Artificial Sequence Synthetic polypeptide”
EVQLVQSGAEVRKPGASVKVSCKASGYSFTNHYMHWVRQAPGQGLEWMGIINPNSGSTSNAQKLQGRVTFTRDTSANTAYMELSGLIPEDTAIYYCARDIVDAFDYWGQGTLVTVSS

SEQ ID NO: 12 - protein, 117 aa, Artificial Sequence
  source: /note=“Description of Artificial Sequence Synthetic polypeptide”
QVQLLESGAVLARPGTSVKISCKASGYSFTNHYMHWVKQRPGQGLEWIGIINPITGSSSNAQKFQGRVTITRDTSTSTVFMELSSLRSEDTAVYYCARDIVDAFDFWGQGTLVTVSS

SEQ ID NO: 13 - protein, 117 aa, Artificial Sequence
  source: /note=“Description of Artificial Sequence Synthetic polypeptide”
QVQLLESGAVLARPGTSVKISCKASGYSFTNHYMHWVKQRPGQGLEWIGIINPITGSSSNAQKFQGRVTITRDTSTSTVFMELSSLRSEDTAVYYCARDLVDAFDFWGQGTLVTVSS

SEQ ID NO: 14 - protein, 106 aa, Artificial Sequence
  source: /note=“Description of Artificial Sequence Synthetic polypeptide”
EIQLTQSPATLSLSPGERATLSCRASQGISSALVWYQQKPGQAPRLLIYDASSLESGIPARFSGSGSGTDFTLTISSLEPEDFAVYYCQQFNDYFTFGQGTKLEIK

SEQ ID NO: 15 - protein, 106 aa, Artificial Sequence
  source: /note=“Description of Artificial Sequence Synthetic polypeptide”
EIQLTQSPATLSLSPGERATLSCRASQGVSSALVWYQQKPGQAPRLLIYAASSLESGIPARFSGSGSGTDFTLTISSLEPEDFAVYYCQQFNSYFTFGQGTKLEIK

SEQ ID NO: 16 - protein, 106 aa, Artificial Sequence
  source: /note=“Description of Artificial Sequence Synthetic polypeptide”
EIQLTQSPATLSLSPGERATLSCRASQGISSALAWYQQKPGQAPRLLIYAASNLESGIPARFSGSGSGTDFTLTISSLEPEDFAVYYCQQYNSYYTFGQGTKLEIK

SEQ ID NO: 17 - protein, 106 aa, Artificial Sequence
  source: /note=“Description of Artificial Sequence Synthetic polypeptide”
EIQLTQSPATLSLSPGERATLSCKASQGISSALVWYQQKPGQAPRLLIYDASSRATGIPARFSGSGSGTDFTLTISSLEPEDFAVYYCQQFNNYFTFGQGTKLEIK

SEQ ID NO: 18 - protein, 106 aa, Artificial Sequence
  source: /note=“Description of Artificial Sequence Synthetic polypeptide”
AIQLTQSPATLSLSPGERATLSCRASQGISSALVWYQQKPGQAPRLLIYDASSLESGIPARFSGSGSGTDFTLTISSLEPEDFAVYYCQQFNDYFTFGQGTKLEIK

SEQ ID NO: 19 - protein, 106 aa, Artificial Sequence
  source: /note=“Description of Artificial Sequence Synthetic polypeptide”
AIQLTQSPSSLSASVGDRVTITCRASQGISSALVWYQQKPGQAPRLLIYDASSLESGIPARFSGSGSGTDFTLTISSLEPEDFAVYYCQQFNDYFTFGQGTKLEIK

SEQ ID NO: 20 - protein, 106 aa, Artificial Sequence
  source: /note=“Description of Artificial Sequence Synthetic polypeptide”
AIQMTQSPSSLSASVGDRVTITCRASQGISSALVWYQQKPGKAPKLLIYDASSLESGVPSRFSGSGSGTDFTFTISSLQPEDIATYYCQQFNDYFTFGPGTKLEIK

SEQ ID NO: 21 - protein, 106 aa, Artificial Sequence
  source: /note=“Description of Artificial Sequence Synthetic polypeptide”
AIQLTQSPATLSVSPGERATLSCRASQGISSALVWYQQKPGQAPRLLIYDASSLESGIPARFSGSGSGTEFTLTISSLQSEDFAVYYCQQFNDYFTFGPGTKVEIK

SEQ ID NO: 22 - protein, 106 aa, Artificial Sequence
  source: /note=“Description of Artificial Sequence Synthetic polypeptide”
AIQLTQSPATLSVSPGERATLSCRASQGISSALVWYQQKPGQAPRLLIYKASSLESGIPARFSGSGSGTEFTLTISSLQSEDFAVYYCQQFNDYFTFGPGTKVEIK

SEQ ID NO: 23 - protein, 106 aa, Artificial Sequence
  source: /note=“Description of Artificial Sequence Synthetic polypeptide”
AIQMTQSPSSLSASVGDRVTITCKASQGISSALAWYQQKPGKAPKLLIYDASNLESGVPSRFSGSGSGTDFTFTISSLQPEDIATYYCQQYNDYFTFGPGTKLEIK

SEQ ID NO: 24 - protein, 106 aa, Artificial Sequence
  source: /note=“Description of Artificial Sequence Synthetic polypeptide”
AIQLTQSPATLSVSPGERATLSCRASQGISSYLVWYQQKPGQAPRLLIYDASNLESGIPARFSGSGSGTEFTLTISSLQSEDFAVYYCQQFSDYFTFGPGTKVEIK

SEQ ID NO: 25 - protein, 106 aa, Artificial Sequence
  source: /note=“Description of Artificial Sequence Synthetic polypeptide”
DIQLTQSPSSLSASVGDRVTITCRASQGISSALVWYQQKPGQAPRLLIYDASSLQSGIPARFSGSGSGTDFTLTISSLEPEDFAVYYCQQFNDYFTFGQGTKLEIK

SEQ ID NO: 26 - protein, 106 aa, Artificial Sequence
  source: /note=“Description of Artificial Sequence Synthetic polypeptide”
DIQLTQSPSSLSASVGDRVTITCRASQGISSALVWYQQKPGQAPRLLIYDASSLQSGIPARFSGSGSGTDFTLTISSLEPEDFAVYYCQQFNDYFSFGQGTKLEIK

SEQ ID NO: 27 - protein, 106 aa, Artificial Sequence
  source: /note=“Description of Artificial Sequence Synthetic polypeptide”
DIQLTQSPSSLSASVGDRVTITCKASQGISSALVWYQQKPGQAPRLLIYDASSLQSGIPARFSGSGSGTDFTLTISSLEPEDFAVYYCQQFNDYFSFGQGTKLEIK

SEQ ID NO: 28 - protein, 106 aa, Artificial Sequence
  source: /note=“Description of Artificial Sequence Synthetic polypeptide”
DIQLTQSPSSLSASVGDRVTITCRASQGISSALVWYQQKPGQAPRLLIYDASSLQSGIPARFSGSGSGTDFTLTISSLEPEDFAVYYCQQFNDLFTFGQGTKLEIK

SEQ ID NO: 29 - protein, 106 aa, Artificial Sequence
  source: /note=“Description of Artificial Sequence Synthetic polypeptide”
DIQLTQSPSSLSASVGDRVTITCKASQGISSALVWYQQKPGQAPRLLIYDASSLETGIPARFSGSGSGTDFTLTISSLEPEDFAVYYCQQFNDYFSFGQGTKLEIK

SEQ ID NO: 30 - protein, 106 aa, Artificial Sequence
  source: /note=“Description of Artificial Sequence Synthetic polypeptide”
DIQLTQSPSSLSASVGDRVTITCKASQGISSALVWYQQKPGQAPRLLIYDASSLQTGIPARFSGSGSGTDFTLTISSLEPEDFAVYYCQQFNDYFSFGQGTKLEIK

SEQ ID NO: 31 - protein, 106 aa, Artificial Sequence
  source: /note=“Description of Artificial Sequence Synthetic polypeptide”
DIQLTQSPSSLSASVGDRVTITCKASQGISSALVWYQQKPGQAPRLLIYDASSLQTGIPARFSGSGSGTDFTLTISSLEPEDFAVYYCQQFNDLFTFGQGTKLEIK

SEQ ID NO: 32 - protein, 106 aa, Artificial Sequence
  source: /note=“Description of Artificial Sequence Synthetic polypeptide”
DIQMTQSPSSLSASVGDRVTITCRASQGISSALVWYQQKPGKAPKLLIYDASSLETGVPSRFSGSGSGTDFTFTISSLQPEDIATYYCQQFNDYFTFGPGTKLEIK

SEQ ID NO: 33 - protein, 106 aa, Artificial Sequence
  source: /note=“Description of Artificial Sequence Synthetic polypeptide”
DIQMTQSPSSLSASVGDRVTITCRASQGISSALVWYQQKPGKAPKLLIYDASSLETGVPSRFSGSGSGTDFTFTISSLQPEDIATYYCQQFNDYFSFGPGTKLEIK

SEQ ID NO: 34 - protein, 106 aa, Artificial Sequence
  source: /note=“Description of Artificial Sequence Synthetic polypeptide”
DIQMTQSPSSLSASVGDRVTITCKASQGISSALVWYQQKPGKAPKLLIYDASSLETGVPSRFSGSGSGTDFTFTISSLQPEDIATYYCQQFNDYFSFGPGTKLEIK

SEQ ID NO: 35 - protein, 106 aa, Artificial Sequence
  source: /note=“Description of Artificial Sequence Synthetic polypeptide”
DIQMTQSPSSLSASVGDRVTITCRASQGISSALVWYQQKPGKAPKLLIYDASSLETGVPSRFSGSGSGTDFTFTISSLQPEDIATYYCQQFNDLFTFGPGTKLEIK

SEQ ID NO: 36 - protein, 106 aa, Artificial Sequence
  source: /note=“Description of Artificial Sequence Synthetic polypeptide”
DIQMTQSPSSLSASVGDRVTITCKASQGISSALVWYQQKPGKAPKLLIYDASSLQSGVPSRFSGSGSGTDFTFTISSLQPEDIATYYCQQFNDYFSFGPGTKLEIK

SEQ ID NO: 37 - protein, 106 aa, Artificial Sequence
  source: /note=“Description of Artificial Sequence Synthetic polypeptide”
DIQMTQSPSSLSASVGDRVTITCKASQGISSALVWYQQKPGKAPKLLIYDASSLQTGVPSRFSGSGSGTDFTFTISSLQPEDIATYYCQQFNDYFSFGPGTKLEIK

SEQ ID NO: 38 - protein, 106 aa, Artificial Sequence
  source: /note=“Description of Artificial Sequence Synthetic polypeptide”
DIQMTQSPSSLSASVGDRVTITCKASQGISSALVWYQQKPGKAPKLLIYDASSLQTGVPSRFSGSGSGTDFTFTISSLQPEDIATYYCQQFNDLFTFGPGTKLEIK

SEQ ID NO: 39 - protein, 5 aa, Artificial Sequence
  source: /note=“Description of Artificial Sequence Synthetic peptide”
NHFIH

SEQ ID NO: 40 - protein, 5 aa, Artificial Sequence
  source: /note=“Description of Artificial Sequence Synthetic peptide”
NHFMH

SEQ ID NO: 41 - protein, 5 aa, Artificial Sequence
  source: /note=“Description of Artificial Sequence Synthetic peptide”
NHYMH

SEQ ID NO: 42 - protein, 5 aa, Artificial Sequence
  source: /note=“Description of Artificial Sequence Synthetic peptide”
NYYMH

SEQ ID NO: 43 - protein, 5 aa, Artificial Sequence
  source: /note=“Description of Artificial Sequence Synthetic peptide”
SHYMH

SEQ ID NO: 44 - protein, 17 aa, Artificial Sequence
  source: /note=“Description of Artificial Sequence Synthetic peptide”
IINPISGSSSNAQKFQG

SEQ ID NO: 45 - protein, 17 aa, Artificial Sequence
  source: /note=“Description of Artificial Sequence Synthetic peptide”
IINPISGSTSNAQKFQG

SEQ ID NO: 46 - protein, 17 aa, Artificial Sequence
  source: /note=“Description of Artificial Sequence Synthetic peptide”
IINPISGSTSNAQKLQG

SEQ ID NO: 47 - protein, 17 aa, Artificial Sequence
  source: /note=“Description of Artificial Sequence Synthetic peptide”
IINPISGSTSYAQKFQG

SEQ ID NO: 48 - protein, 17 aa, Artificial Sequence
  source: /note=“Description of Artificial Sequence Synthetic peptide”
IINPISGSTTYAQKFQG

SEQ ID NO: 49 - protein, 17 aa, Artificial Sequence
  source: /note=“Description of Artificial Sequence Synthetic peptide”
IINPITGSSSNAQKFQG

SEQ ID NO: 50 - protein, 17 aa, Artificial Sequence
  source: /note=“Description of Artificial Sequence Synthetic peptide”
IINPNSGSTSNAQKLQG

SEQ ID NO: 51 - protein, 17 aa, Artificial Sequence
  source: /note=“Description of Artificial Sequence Synthetic peptide”
TINPVSGSTNYAQKFQG

SEQ ID NO: 52 - protein, 17 aa, Artificial Sequence
  source: /note=“Description of Artificial Sequence Synthetic peptide”
TINPVSGSTSYAQKFQG

SEQ ID NO: 53 - protein, 8 aa, Artificial Sequence
  source: /note=“Description of Artificial Sequence Synthetic peptide”
DIVDAFDF

SEQ ID NO: 54 - protein, 8 aa, Artificial Sequence
  source: /note=“Description of Artificial Sequence Synthetic peptide”
DIVDAFDY

SEQ ID NO: 55 - protein, 8 aa, Artificial Sequence
  source: /note=“Description of Artificial Sequence Synthetic peptide”
DLVDAFDF

SEQ ID NO: 56 - protein, 8 aa, Artificial Sequence
  source: /note=“Description of Artificial Sequence Synthetic peptide”
EILDAFDY

SEQ ID NO: 57 - protein, 8 aa, Artificial Sequence
  source: /note=“Description of Artificial Sequence Synthetic peptide”
EIVDAFDF

SEQ ID NO: 58 - protein, 11 aa, Artificial Sequence
  source: /note=“Description of Artificial Sequence Synthetic peptide”
KASQGISSALA

SEQ ID NO: 59 - protein, 11 aa, Artificial Sequence
  source: /note=“Description of Artificial Sequence Synthetic peptide”
KASQGISSALV

SEQ ID NO: 60 - protein, 11 aa, Artificial Sequence
  source: /note=“Description of Artificial Sequence Synthetic peptide”
RASQGISSALA

SEQ ID NO: 61 - protein, 11 aa, Artificial Sequence
  source: /note=“Description of Artificial Sequence Synthetic peptide”
RASQGISSALV

SEQ ID NO: 62 - protein, 11 aa, Artificial Sequence
  source: /note=“Description of Artificial Sequence Synthetic peptide”
RASQGISSYLV

SEQ ID NO: 63 - protein, 11 aa, Artificial Sequence
  source: /note=“Description of Artificial Sequence Synthetic peptide”
RASQGVSSALV

SEQ ID NO: 64 - protein, 7 aa, Artificial Sequence
  source: /note=“Description of Artificial Sequence Synthetic peptide”
AASNLES

SEQ ID NO: 65 - protein, 7 aa, Artificial Sequence
  source: /note=“Description of Artificial Sequence Synthetic peptide”
AASSLES

SEQ ID NO: 66 - protein, 7 aa, Artificial Sequence
  source: /note=“Description of Artificial Sequence Synthetic peptide”
DASNLES

SEQ ID NO: 67 - protein, 7 aa, Artificial Sequence
  source: /note=“Description of Artificial Sequence Synthetic peptide”
DASSLES

SEQ ID NO: 68 - protein, 7 aa, Artificial Sequence
  source: /note=“Description of Artificial Sequence Synthetic peptide”
DASSLET

SEQ ID NO: 69 - protein, 7 aa, Artificial Sequence
  source: /note=“Description of Artificial Sequence Synthetic peptide”
DASSLQS

SEQ ID NO: 70 - protein, 7 aa, Artificial Sequence
  source: /note=“Description of Artificial Sequence Synthetic peptide”
DASSLQT

SEQ ID NO: 71 - protein, 7 aa, Artificial Sequence
  source: /note=“Description of Artificial Sequence Synthetic peptide”
DASSRAT

SEQ ID NO: 72 - protein, 7 aa, Artificial Sequence
  source: /note=“Description of Artificial Sequence Synthetic peptide”
KASSLES

SEQ ID NO: 73 - protein, 8 aa, Artificial Sequence
  source: /note=“Description of Artificial Sequence Synthetic peptide”
QQFNDYFS

SEQ ID NO: 74 - protein, 8 aa, Artificial Sequence
  source: /note=“Description of Artificial Sequence Synthetic peptide”
QQFNDYFT

SEQ ID NO: 75 - protein, 8 aa, Artificial Sequence
  source: /note=“Description of Artificial Sequence Synthetic peptide”
QQFNNYFT

SEQ ID NO: 76 - protein, 8 aa, Artificial Sequence
  source: /note=“Description of Artificial Sequence Synthetic peptide”
QQFNSYFT

SEQ ID NO: 77 - protein, 8 aa, Artificial Sequence
  source: /note=“Description of Artificial Sequence Synthetic peptide”
QQFSDYFT

SEQ ID NO: 78 - protein, 8 aa, Artificial Sequence
  source: /note=“Description of Artificial Sequence Synthetic peptide”
QQYNDYFT

SEQ ID NO: 79 - protein, 8 aa, Artificial Sequence
  source: /note=“Description of Artificial Sequence Synthetic peptide”
QQYNSYYT

SEQ ID NO: 80 - protein, 330 aa, Unknown
  source: /note=“Description of Unknown IgG1 sequence”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 107 aa, Artificial Sequence [sequence omitted]

SEQ ID NO: 82 - protein, 251 aa, Homo sapiens
MLGARLRLWVCALCSVCSMSVLRAYPNASPLLGSSWGGLIHLYTATARNSYHLQIHKNGHVDGAPHQTIYSALMIRSEDAGFVVITGVMSRRYLCMDFRGNIFGSHYFDPENCRFQHQTLENGYDVYHSPQYHFLVSLGRAKRAFLPGMNPPPYSQFLSRRNEIPLIHFNTPIPRRHTRSAEDDSERDPLNVLKPRARMTPAPASCSQELPSAEDNSPMASDPLGVVRGGRVNTHAGGTGPEGCRPFAKFI

SEQ ID NO: 83 - protein, 5 aa, Artificial Sequence
  source: /note=“Description of Artificial Sequence Synthetic peptide”
  VARIANT (1)..(1): /replace=“S” or “A”
  VARIANT (2)..(2): /replace=“Y”
  VARIANT (3)..(3): /replace=“Y”
  VARIANT (4)..(4): /replace=“M”
  SITE (1)..(5): /note=“Variant residues given in the sequence have no preference with respect to those in the annotations for variant positions”
NHFIH

SEQ ID NO: 84 - protein, 17 aa, Artificial Sequence
  source: /note=“Description of Artificial Sequence Synthetic peptide”
  VARIANT (1)..(1): /replace=“T”
  VARIANT (5)..(5): /replace=“N” or “V”
  VARIANT (6)..(6): /replace=“T”
  VARIANT (9)..(9): /replace=“T”
  VARIANT (10)..(10): /replace=“T” or “N”
  VARIANT (11)..(11): /replace=“Y”
  VARIANT (15)..(15): /replace=“L”
  SITE (1)..(17): /note=“Variant residues given in the sequence have no preference with respect to those in the annotations for variant positions”
IINPISGSSSNAQKFQG

SEQ ID NO: 85 - protein, 8 aa, Artificial Sequence
  source: /note=“Description of Artificial Sequence Synthetic peptide”
  VARIANT (1)..(1): /replace=“E”
  VARIANT (2)..(2): /replace=“I”
  VARIANT (3)..(3): /replace=“L”
  VARIANT (8)..(8): /replace=“Y”
  SITE (1)..(8): /note=“Variant residues given in the sequence have no preference with respect to those in the annotations for variant positions”
DLVDAFDF

SEQ ID NO: 86 - protein, 11 aa, Artificial Sequence
  source: /note=“Description of Artificial Sequence Synthetic peptide”
  VARIANT (1)..(1): /replace=“R”
  VARIANT (4)..(4): /replace=“A”
  VARIANT (6)..(6): /replace=“V”
  VARIANT (9)..(9): /replace=“Y”
  VARIANT (11)..(11): /replace=“V”
  SITE (1)..(11): /note=“Variant residues given in the sequence have no preference with respect to those in the annotations for variant positions”
KASQGISSALA

SEQ ID NO: 87 - protein, 7 aa, Artificial Sequence
  source: /note=“Description of Artificial Sequence Synthetic peptide”
  VARIANT (1)..(1): /replace=“D” or “K”
  VARIANT (4)..(4): /replace=“S”
  VARIANT (5)..(5): /replace=“R”
  VARIANT (6)..(6): /replace=“Q” or “A”
  VARIANT (7)..(7): /replace=“T”
  SITE (1)..(7): /note=“Variant residues given in the sequence have no preference with respect to those in the annotations for variant positions”
AASNLES

SEQ ID NO: 88 - protein, 8 aa, Artificial Sequence
  source: /note=“Description of Artificial Sequence Synthetic peptide”
  VARIANT (3)..(3): /replace=“Y”
  VARIANT (4)..(4): /replace=“S”
  VARIANT (5)..(5): /replace=“N” or “S”
  VARIANT (6)..(6): /replace=“L”
  VARIANT (7)..(7): /replace=“Y”
  VARIANT (8)..(8): /replace=“T”
  SITE (1)..(8): /note=“Variant residues given in the sequence have no preference with respect to those in the annotations for variant positions”
QQFNDYFS

SEQ ID NO: 89 - protein, 8 aa, Artificial Sequence
  source: /note=“Description of Artificial Sequence Synthetic peptide”
QQFNDLFT

SEQ ID NO: 90 - protein, 117 aa, Artificial Sequence
  source: /note=“Description of Artificial Sequence Synthetic polypeptide”
QVQLLESGAVLARPGTSVKISCKASGYSFTAHYMHWVKQRPGQGLEWIGIINPITGSSSNAQKFQGRVTITRDTSTSTVFMELSSLRSEDTAVYYCARDLVDAFDFWGQGTLVTVSS

SEQ ID NO: 91 - protein, 117 aa, Artificial Sequence
  source: /note=“Description of Artificial Sequence Synthetic polypeptide”
EVQLVQSGAEVRKPGASVKVSCKASGYTFTAHYMHWVRQAPGQGLEWMGIINPISGSTSNAQKLQGRVTFTRDTSANTAYMELSGLIPEDTAIYYCARDIVDAFDFWGQGTLVTVSS

SEQ ID NO: 92 - protein, 106 aa, Artificial Sequence
  source: /note=“Description of Artificial Sequence Synthetic polypeptide”
DIQLTQSPSSLSASVGDRVTITCRASAGISSALVWYQQKPGQAPRLLIYDASSLQSGIPARFSGSGSGTDFTLTISSLEPEDFAVYYCQQFNDLFTFGQGTKLEIK

SEQ ID NO: 93 - protein, 106 aa, Artificial Sequence
  source: /note=“Description of Artificial Sequence Synthetic polypeptide”
AIQLTQSPSSLSASVGDRVTITCRASAGISSALVWYQQKPGQAPRLLIYDASSLQSGIPARFSGSGSGTDFTLTISSLEPEDFAVYYCQQFNDLFTFGQGTKLEIK

SEQ ID NO: 94 - protein, 106 aa, Artificial Sequence
  source: /note=“Description of Artificial Sequence Synthetic polypeptide”
DIQMTQSPSSLSASVGDRVTITCKASQGISSALVWYQQKPGKAPKLLIYDASSLQTGVPSRFSGSGSGTDFTFTISSLQPEDIATYYCQQFNDYFTFGPGTKLEIK

SEQ ID NO: 95 - protein, 106 aa, Artificial Sequence
  source: /note=“Description of Artificial Sequence Synthetic polypeptide”
DIQMTQSPSSLSASVGDRVTITCRASQGISSALVWYQQKPGKAPKLLIYDASSLQTGVPSRFSGSGSGTDFTFTISSLQPEDIATYYCQQFNDYFSFGPGTKLEIK

SEQ ID NO: 96 - protein, 106 aa, Artificial Sequence
  source: /note=“Description of Artificial Sequence Synthetic polypeptide”
AIQMTQSPSSLSASVGDRVTITCKASQGISSALVWYQQKPGKAPKLLIYDASSLQTGVPSRFSGSGSGTDFTFTISSLQPEDIATYYCQQFNDYFSFGPGTKLEIK

SEQ ID NO: 97 - DNA, 452 bp, Artificial Sequence
  source: /note=“Description of Artificial Sequence Synthetic polynucleotide”
tctagaggatcgaacccttcaccatggaaactgacactctcctgctttgggtcttgctgctctgggtgcctggttcaaccggcgaagtgcaactggtgcagtccggtgccgaggtccgcaagcccggagccagcgtgaaagtgtcctgcaaggcctccgggtacaccttcaccaatcactacatgcattgggtcagacaggccccgggccagggactggagtggatgggcatcatcaacccaatttcggggtcaacctcgaacgctcaaaagctgcagggccgcgtgacctttactcgggacacctccgcaaacacagcgtacatggaactgtccggactgattcccgaggataccgccatctactattgtgcccgggatatcgtggacgccttcgacttctggggacagggaactctcgtgacggtgtccagcgcgagcaccaaggggccc

SEQ ID NO: 98 - DNA, 407 bp, Artificial Sequence
  source: /note=“Description of Artificial Sequence Synthetic polynucleotide”
tctagaggatcgaacccttcaccatggaaactgatactctgcttctctgggtcttgctgctctgggtccctggttcaactggcgctatccagatgacccagtccccgtcgtcactgtccgcctccgtgggtgatcgcgtgaccatcacgtgtcgggccagccagggaatctcctccgcactcgtgtggtatcagcagaagcctggaaaagccccgaagctgctgatctacgacgcctcctccctggaatcgggagtgccatcgagattctccggctctgggagcgggaccgacttcaccttcacaattagcagcctgcagcccgaggacatcgcgacctactactgccaacaattcaacgactactttaccttcggacccggcaccaagctcgagattaagggatcc

SEQ ID NO: 99 - DNA, 452 bp, Artificial Sequence
  source: /note=“Description of Artificial Sequence Synthetic polynucleotide”
tctagaggatcgaacccttcaccatggaaactgacactctcttgctctgggtacttctcctctgggtccctggttccaccggccaagtgcagctgctggaatcgggagcggtgctggcccggccgggaacttccgtgaagatcagctgcaaagcatcagggtacaccttcaccaaccactacatgcattgggtcaagcagaggcccggccagggactggagtggatcggcatcatcaacccaattaccggcagctcctccaacgctcaaaagttccagggtcgcgtgaccattaccagagatacctccacctcgaccgtgttcatggaactgtcatccctgcggtccgaggacactgccgtgtattactgcgcccgcgatatcgtggacgcctttgacttctggggacaggggacgctggtcacagtgtcgagcgccagcaccaaggggccc

SEQ ID NO: 100 - DNA, 407 bp, Artificial Sequence
  source: /note=“Description of Artificial Sequence Synthetic polynucleotide”
tctagaggatcgaacccttcaccatggaaactgacacattgctgctttgggtgttgctcctttgggtccccggttcaactggcgctatccagctgacccagagcccgtcatccctgtccgcctccgtgggagacagagtcaccattacttgccgggcctcccaagggatttccagcgcgctcgtgtggtatcagcagaaaccgggacaggcacccaggctgctgatctacgacgccagctcgctggagagcggaatccctgcccgcttttcggggtcgggttctggcaccgatttcaccctgaccatctcctccctggaaccagaggatttcgccgtgtactactgccaacagttcaacgactacttcacgttcggccagggaaccaagctcgaaatcaagggatcc

SEQ ID NO: 101 - DNA, 452 bp, Artificial Sequence
  source: /note=“Description of Artificial Sequence Synthetic polynucleotide”
tctagaggatcgaacccttcaccatggaaactgatactttgctcctttgggtcctcctgctttgggtgcctggctcgaccggagaagtgcaactggtgcagtccggagccgaagtccggaagcccggagcctccgtgaaagtgtcctgcaaggcctcgggctataccttcaccaaccactacatgcattgggtccgccaagcaccaggacagggcctggagtggatgggtatcattaacccgatcagcgggagcacttcaaacgctcagaagctgcagggcagagtgacctttacccgcgacaccagcgccaatacggcgtacatggaactgtccgggctgatccccgaggacacagcgatctactactgtgcccgggagattgtggatgccttcgacttctggggacagggtaccctcgtgaccgtgtcatccgcctccactaaggggccc

SEQ ID NO: 102 - DNA, 407 bp, Artificial Sequence; identical to SEQ ID NO: 100
  source: /note=“Description of Artificial Sequence Synthetic polynucleotide”

SEQ ID NO: 103 - DNA, 452 bp, Artificial Sequence
  source: /note=“Description of Artificial Sequence Synthetic polynucleotide”
tctagaggatcgaacccttcaccatggaaaccgacacgctgctcctctgggtccttcttctctgggtgcctggttcaactggagaggtgcagctggtgcagtccggcgcagaagtccggaagcccggcgccagcgtgaaagtgtcctgcaaggcctccgggtactcattcaccaaccattacatgcactgggtccgccaagcgccgggacaagggctggagtggatgggaatcattaacccaaacagcggttccacctcgaacgcccagaagctgcagggcagagtgaccttcactcgcgacacctccgctaataccgcctacatggaattgtcgggcctgattcccgaggataccgcgatctactactgtgcccgggacatcgtggatgcctttgactattggggacagggaactctggtcaccgtgtcgagcgcctccactaaggggccc

SEQ ID NO: 104 - DNA, 407 bp, Artificial Sequence; identical to SEQ ID NO: 98
  source: /note=“Description of Artificial Sequence Synthetic polynucleotide”

SEQ ID NO: 105 - DNA, 452 bp, Artificial Sequence; identical to SEQ ID NO: 97
  source: /note=“Description of Artificial Sequence Synthetic polynucleotide”

SEQ ID NO: 106 - DNA, 407 bp, Artificial Sequence
  source: /note=“Description of Artificial Sequence Synthetic polynucleotide”
tctagaggatcgaacccttcaccatggaaactgatactttgctgctctgggtgcttctgctttgggtccctggctcgaccggcgacatccagctgacccagtcgccgtcatctctgtccgcctccgtgggcgatagagtcactatcacgtgtcgcgcaagccagggaattagctccgccctcgtgtggtatcagcagaagcccgggcaggcccctaggctgctgatctacgacgcgtccagcctgcaatccggaatcccagcccggttctccggttccggatcgggaaccgactttaccctgaccatctcaagcctggaacccgaggacttcgctgtgtactactgccagcaattcaacgacctcttcaccttcgggcagggtactaagctcgagattaagggatcc

SEQ ID NO: 107 - DNA, 452 bp, Artificial Sequence
  source: /note=“Description of Artificial Sequence Synthetic polynucleotide”
tctagaggatcgaacccttcaccatggaaaccgatactctgctcctctgggtcttgctgctctgggtccccggttcgactggccaagtgcagctgctggagtcaggggccgtgctggcccgccctgggaccagcgtgaagatcagctgcaaggcctctggatactcctttaccaaccactacatgcattgggtcaagcagaggccgggccagggtcttgagtggatcggaattatcaacccaatcaccggctcgtccagcaacgcacaaaagttccagggcagagtgacgatcacccgggacacctcgacctccaccgtgttcatggaactgagctccctgcggtccgaggatactgccgtgtattactgtgcccgcgacctggtggacgctttcgacttctggggacagggaacactcgtgactgtgtcctccgcgtcaaccaaagggccc

SEQ ID NO: 108 - DNA, 407 bp, Artificial Sequence
  source: /note=“Description of Artificial Sequence Synthetic polynucleotide”
tctagaggatcgaacccttcaccatggaaactgatactttgctgctctgggtgcttctgctttgggtccctggctcgaccggcgacatccagctgacccagtcgccgtcatctctgtccgcctccgtgggcgatagagtcactatcacgtgtcgcgcaagccagggaattagctccgccctcgtgtggtatcagcagaagcccgggcaggcccctaggctgctgatctacgacgcgtccagcctgcaatccggaatcccagcccggttctccggttccggatcgggaaccgactttaccctgaccatctcaagcctggaacccgaggacttcgctgtgtactactgccagcaattcaacgactacttcaccttcgggcagggtactaagctcgagattaagggatcc

SEQ ID NO: 109 - protein, 11 aa, Artificial Sequence
  source: /note=“Description of Artificial Sequence Synthetic peptide”
RASAGISSALV

SEQ ID NO: 110 - protein, 5 aa, Artificial Sequence
  source: /note=“Description of Artificial Sequence Synthetic peptide”
AHYMH

SEQ ID NO: 111 - DNA, 452 bp, Artificial Sequence
  source: /note=“Description of Artificial Sequence Synthetic polynucleotide”
tctagaggatcgaacccttcaccatggaaaccgacactttgctcctctgggtcctcctgctttgggtgcctggctcgactggacaagtgcagctggtgcagtccggggccgaactgaagaagccaggcgcctcggtcaaagtgtcctgcaaagccagcggatacaccttcaccaaccattacatgcactgggtcagacaggccccgggacagggtctggagtggatgggtatcatcaaccccatttccggctccaccacctacgcgcaaaagtttcagggccgcgtgacaatgactcgggatacctccagctccaccgcttatatggacctgtcgaggctgacgagcgacgatactgccgtgtactactgtgcccgggacatcgtggacgcgttcgatttctggggacaggggactaccgtgaccgtgtcatcagcatccaccaaggggccc

SEQ ID NO: 112 - DNA, 407 bp, Artificial Sequence; identical to SEQ ID NO: 100
  source: /note=“Description of Artificial Sequence Synthetic polynucleotide”

SEQ ID NO: 113 - DNA, 351 bp, Artificial Sequence
  source: /note=“Description of Artificial Sequence Synthetic polynucleotide”
gaagtgcagctggtgcagagcggcgcggaagtgcgcaaaccgggcgcgagcgtgaaagtgagctgcaaagcgagcggctatacctttaccaaccattatatgcattgggtgcgccaggcgccgggccagggcctggaatggatgggcattattaacccgattagcggcagcaccagcaacgcgcagaaactgcagggccgcgtgacctttacccgcgataccagcgcgaacaccgcgtatatggaactgagcggcctgattccggaagataccgcgatttattattgcgcgcgcgatattgtggatgcgtttgatttttggggccagggcaccctggtgaccgtgagcagc

SEQ ID NO: 114 - DNA, 318 bp, Artificial Sequence
  source: /note=“Description of Artificial Sequence Synthetic polynucleotide”
gatattcagatgacccagagcccgagcagcctgagcgcgagcgtgggcgatcgcgtgaccattacctgcaaagcgagccagggcattagcagcgcgctggtgtggtatcagcagaaaccgggcaaagcgccgaaactgctgatttatgatgcgagcagcctgcagaccggcgtgccgagccgctttagcggcagcggcagcggcaccgattttacctttaccattagcagcctgcagccggaagatattgcgacctattattgccagcagtttaacgattattttagctttggcccgggcaccaaactggaaattaaa

SEQ ID NO: 115 - DNA, 351 bp, Artificial Sequence; identical to SEQ ID NO: 113
  source: /note=“Description of Artificial Sequence Synthetic polynucleotide”

SEQ ID NO: 116 - DNA, 318 bp, Artificial Sequence
  source: /note=“Description of Artificial Sequence Synthetic polynucleotide”
gatattcagatgacccagagcccgagcagcctgagcgcgagcgtgggcgatcgcgtgaccattacctgcaaagcgagccagggcattagcagcgcgctggtgtggtatcagcagaaaccgggcaaagcgccgaaactgctgatttatgatgcgagcagcctggaaaccggcgtgccgagccgctttagcggcagcggcagcggcaccgattttacctttaccattagcagcctgcagccggaagatattgcgacctattattgccagcagtttaacgattattttagctttggcccgggcaccaaactggaaattaaa

SEQ ID NO: 117 - DNA, 351 bp, Artificial Sequence; identical to SEQ ID NO: 113
  source: /note=“Description of Artificial Sequence Synthetic polynucleotide”

SEQ ID NO: 118 - DNA, 318 bp, Artificial Sequence
  source: /note=“Description of Artificial Sequence Synthetic polynucleotide”
gatattcagatgacccagagcccgagcagcctgagcgcgagcgtgggcgatcgcgtgaccattacctgcaaagcgagccagggcattagcagcgcgctggtgtggtatcagcagaaaccgggcaaagcgccgaaactgctgatttatgatgcgagcagcctgcagagcggcgtgccgagccgctttagcggcagcggcagcggcaccgattttacctttaccattagcagcctgcagccggaagatattgcgacctattattgccagcagtttaacgattattttagctttggcccgggcaccaaactggaaattaaa

SEQ ID NO: 119 - DNA, 351 bp, Artificial Sequence; identical to SEQ ID NO: 113
  source: /note=“Description of Artificial Sequence Synthetic polynucleotide”

SEQ ID NO: 120 - DNA, 318 bp, Artificial Sequence
  source: /note=“Description of Artificial Sequence Synthetic polynucleotide”
gatattcagatgacccagagcccgagcagcctgagcgcgagcgtgggcgatcgcgtgaccattacctgcaaagcgagccagggcattagcagcgcgctggtgtggtatcagcagaaaccgggcaaagcgccgaaactgctgatttatgatgcgagcagcctgcagaccggcgtgccgagccgctttagcggcagcggcagcggcaccgattttacctttaccattagcagcctgcagccggaagatattgcgacctattattgccagcagtttaacgattattttacctttggcccgggcaccaaactggaaattaaa

SEQ ID NO: 121 - DNA, 351 bp, Artificial Sequence; identical to SEQ ID NO: 113
  source: /note=“Description of Artificial Sequence Synthetic polynucleotide”

SEQ ID NO: 122 - DNA, 318 bp, Artificial Sequence
  source: /note=“Description of Artificial Sequence Synthetic polynucleotide”
gatattcagatgacccagagcccgagcagcctgagcgcgagcgtgggcgatcgcgtgaccattacctgccgcgcgagccagggcattagcagcgcgctggtgtggtatcagcagaaaccgggcaaagcgccgaaactgctgatttatgatgcgagcagcctgcagaccggcgtgccgagccgctttagcggcagcggcagcggcaccgattttacctttaccattagcagcctgcagccggaagatattgcgacctattattgccagcagtttaacgattattttagctttggcccgggcaccaaactggaaattaaa

SEQ ID NO: 123 - DNA, 351 bp, Artificial Sequence; identical to SEQ ID NO: 113
  source: /note=“Description of Artificial Sequence Synthetic polynucleotide”

SEQ ID NO: 124 - DNA, 318 bp, Artificial Sequence
  source: /note=“Description of Artificial Sequence Synthetic polynucleotide”
gcgattcagatgacccagagcccgagcagcctgagcgcgagcgtgggcgatcgcgtgaccattacctgcaaagcgagccagggcattagcagcgcgctggtgtggtatcagcagaaaccgggcaaagcgccgaaactgctgatttatgatgcgagcagcctgcagaccggcgtgccgagccgctttagcggcagcggcagcggcaccgattttacctttaccattagcagcctgcagccggaagatattgcgacctattattgccagcagtttaacgattattttagctttggcccgggcaccaaactggaaattaaa

SEQ ID NO: 125 - protein, 330 aa, Artificial Sequence
  source: /note=“Description of Artificial Sequence Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - protein, 326 aa, Unknown
  source: /note=“Description of Unknown IgG4 sequence”
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 127 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 128 - protein, 326 aa, Unknown
  source: /note=“Description of Unknown IgG2/4 sequence”
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 129 - protein, 326 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2022/0185876 A1
(43) Pub. Date: Jun. 16, 2022

(54) ANTI FGF23 ANTIBODY

(71) Applicant: Atarga, LLC, Winchester, MA (US)

(72) Inventors: Akila JAYARAMAN, Winchester, MA (US); Akshay PAINTAL, Winchester, MA (US)

(21) Appl. No.: 17/600,070

(22) PCT Filed: Mar. 27, 2020
(86) PCT No.: PCT/US2020/025235

§ 371 Date: Sep. 29, 2021

Related U.S. Application Data
(60) Provisional application No. 62/826,199, filed on Mar. 29, 2019.

(51) Int. Cl. C07K 16/22; A61K 39/395

(52) U.S. Cl. CPC C07K 16/22; A61K 39/3955; C07K 2317/565; A61K 2039/545; C07K 2317/52; C07K 2317/92; C07K 2317/73; C07K 2317/24

(57) ABSTRACT

Antibody molecules that specifically bind to FGF23 are disclosed. The antibody molecules can be used to treat, prevent, and/or diagnose disorders, such as FGF23-associated disorders.

### CROSS REFERENCE TO RELATED APPLICATIONS

This application claims the benefit of U.S. Provisional Application No. 62/826,199, filed Mar. 29, 2019. The contents of the aforementioned application are hereby incorporated by reference in its entirety.

### SEQUENCE LISTING

The instant application contains a Sequence Listing which has been submitted electronically in ASCII format and is hereby incorporated by reference in its entirety. Said ASCII copy, created on Mar. 26, 2020, is named A2176-7002WO_SL.txt and is 105,507 bytes in size.

### BACKGROUND

Fibroblast growth factor (FGF) is a representative growth factor which has shown the potential effects on the repair and regeneration of tissues. It was originally identified as a protein capable of promoting fibroblast proliferation and is now known to comprise 22 members. FGFs exert multiple functions through the binding into and activation of fibroblast growth factor receptors (FGFRs), and the main signaling through the stimulation of FGFRs is the RAS/MAP kinase pathway. Fibroblast growth factor 23 (FGF23) is a circulating factor secreted by osteocytes that is essential for phosphate homeostasis. In kidney proximal tubular cells FGF23 inhibits phosphate reabsorption and leads to decreased synthesis and enhanced catabolism of 1,25-dihydroxyvitamin D3 (1,25[OH]2 D3).

Excess levels of FGF23 cause renal phosphate wasting and suppression of circulating 1,25(OH)2 D3 levels and are associated with several hereditary hypophosphatemic disorders with skeletal abnormalities, including X-linked hypophosphatemic rickets (XLH) and autosomal recessive hypophosphatemic rickets (ARHR). Currently, therapeutic approaches to these diseases are limited to treatment with activated vitamin D analogues and phosphate supplementation, often merely resulting in partial correction of the skeletal aberrations. X-linked hypophosphatemia (XLH) is caused by inactivating mutations in the PHEX gene (phosphate regulating gene with homology to endopeptidases on the X chromosome). The PHEX loss of function causes osteocytes to secrete excess fibroblast growth factor 23 (FGF23), which is a key physiologic regulator of phosphate and vitamin D metabolism. FGF23 binds to the FGFR1c receptor and α-klotho co-receptor on proximal renal tubule cells, and can cause at least two effects: (1) reduced expression of sodium phosphate co-transporters (NaPi-IIa and NaPi-IIc), causing urinary phosphate wasting; and (2) downregulation of 1α-hydroxylase with reduced secretion of 1,25 dihydroxyvitamin D, causing reduced calcium and phosphate absorption in the gut. In XLH, excess FGF23 leads to hypophosphatemia, resulting in inadequate skeletal mineralization, i.e., rickets and osteomalacia.

As such, there exists a need for developing new approaches, including improved agents, for treating, preventing and diagnosing FGF23-associated disorders and other disorders that share similar disease mechanisms.

### SUMMARY

This disclosure provides, at least in part, antibody molecules that bind to FGF23, e.g., human FGF23 (e.g., human FGF23 comprising the amino acid sequence of SEQ ID NO: 82), and that comprise one or more functional and structural properties disclosed herein. In an embodiment, the antibody molecule binds to and/or reduces (e.g., inhibits, blocks, or neutralizes) one or more activities of FGF23. In an embodiment, the antibody molecule is selected from Table 1, or competes for binding to FGF23 with an antibody molecule selected from Table 1. In an embodiment, the antibody molecule binds to the same or overlapping epitope as the epitope recognized by an antibody molecule selected from Table 1. In an embodiment, the antibody molecule comprises one or more heavy chain variable regions and/or one or more light chain variable regions described in Table 1. In an embodiment, the antibody molecule comprises one or more heavy chain CDRs and/or one or more light chain CDRs described in Table 1. In an embodiment, nucleic acid molecules encoding the antibody molecules, expression vectors, host cells, compositions (e.g., pharmaceutical compositions), kits, containers, and methods for making the antibody molecules, are also provided. The antibody molecules disclosed herein are suitable for use in reducing or inhibiting undesired activation of the FGF pathway (e.g., by abnormally increasing the level of FGF23) or a component thereof. The antibody molecules disclosed herein can be used (alone or in combination with other agents or therapeutic modalities) to treat, prevent and/or diagnose FGF23-associated disorders.

Accordingly, in an aspect, this disclosure provides an antibody molecule, e.g., an antibody molecule described herein, having one or more (e.g., 1, 2, 3, 4, 5, 6, 7, 8, 9, 10, 11, 12, 13, 14, or all) of the following properties:
a) Binds to FGF23 (e.g., human FGF23) with high affinity, e.g., with a dissociation constant (K_{D}′) of about 50 nM or less, e.g., about 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.9 nM or less, about 0.8 nM or less, about 0.7 nM or less, about 0.6 nM or less, about 0.5 nM or less, about 0.4 nM or less, about 0.3 nM or less, about 0.2 nM or less, about 0.1 nM or less, about 0.05 nM or less, about 0.04 nM or less, about 0.03 nM or less, about 0.02 nM or less, about 0.01 nM or less, 0.005 nM or less, 0.002 nM or less, or 0.001 nM or less, e.g., between 0.001 nM and 1 nM, between 0.001 nM and 0.9 nM, between 0.001 nM and 0.8 nM, between 0.001 nM and 0.7 nM, between 0.001 nM and 0.6 nM, between 0.001 nM and 0.5 nM, between 0.001 nM and 0.4 nM, between 0.001 nM and 0.3 nM, between 0.001 nM and 0.2 nM, between 0.001 nM and 0.1 nM, between 0.01 nM and 1 nM, between 0.01 nM and 0.9 nM, between 0.01 nM and 0.8 nM, between 0.01 nM and 0.7 nM, between 0.01 nM and 0.6 nM, between 0.01 nM and 0.5 nM, between 0.01 nM and 0.4 nM, between 0.01 nM and 0.3 nM, between 0.01 nM and 0.2 nM, between 0.01 nM and 0.1 nM, between 0.001 nM and 10 nM, between 0.001 nM and 5 nM, between 0.001 nM and 2 nM, between 0.001 nM and 1 nM, between 0.001 nM and 0.5 nM, between 0.001 nM and 0.2 nM, between 0.001 nM and 0.1 nM, between 0.001 and 0.05 nM, between 0.001 and 0.02 nM, between 0.001 and 0.005 nM, between 5 nM and 10 nM, between 2 nM and 10 nM, between 1 nM and 10 nM, between 0.5 nM and 10 nM, between 0.2 nM and 10 nM, between 0.1 nM and 10 nM, between 0.05 nM and 10 nM, between 0.02 nM and 10 nM, between 0.01 nM and 10 nM, between 0.005 nM and 10 nM, between 0.002 and 10 nM, between 0.002 nM and 5 nM, between 0.005 nM and 2 nM, between 0.01 nM and 1 nM, between 0.02 nM and 0.5 nM, between 0.05 nM and 0.2 nM, between 0.001 nM and 0.002 nM, between 0.002 nM and 0.005 nM, between 0.005 nM and 0.01 nM, between 0.01 nM and 0.02 nM, between 0.02 nM and 0.05 nM, between 0.05 nM and 0.1 nM, between 0.1 nM and 0.2 nM, between 0.2 nM and 0.5 nM, between 0.5 nM and 1 nM, between 1 nM and 2 nM, between 2 nM and 5 nM, or between 5 nM and 10 nM, e.g., between 0.1 nM and 0.6 nM or between 0.2 nM and 0.53 nM, e.g., as determined by a method described herein;
b) Binds to FGF23 (e.g., human FGF23) with high affinity, e.g., with a half maximal effective concentration (EC₅₀) of about 1 μg/ml or less, e.g., about 0.9 μg/ml or less, 0.8 μg/ml or less, 0.7 μg/ml or less, 0.6 μg/ml or less, 0.5 μg/ml or less, 0.4 μg/ml or less, 0.3 μg/ml or less, 0.2 μg/ml or less, 0.1 μg/ml or less, 0.09 μg/ml or less, 0.08 μg/ml or less, 0.07 μg/ml or less, 0.06 μg/ml or less, 0.05 μg/ml or less, 0.04 μg/ml or less, 0.03 μg/ml or less, 0.02 μg/ml or less, 0.01 μg/ml or less, 0.005 μg/ml or less, 0.002 μg/ml or less, 0.001 μg/ml or less, e.g., between 0.001 μg/ml and 1 μg/ml, e.g., between 0.001 μg/ml and 1 μg/ml, between 0.001 μg/ml and 0.5 μg/ml, between 0.001 μg/ml and 0.2 μg/ml, between 0.001 μg/ml and 0.1 μg/ml, between 0.001 μg/ml and 0.05 μg/ml, between 0.001 μg/ml and 0.02 μg/ml, between 0.001 μg/ml and 0.01 μg/ml, between 0.001 μg/ml and 0.005 μg/ml, between 0.002 μg/ml and 1 μg/ml, between 0.005 μg/ml and 1 μg/ml, between 0.01 μg/ml and 1 μg/ml, between 0.02 μg/ml and 1 μg/ml, between 0.05 μg/ml and 1 μg/ml, between 0.1 μg/ml and 1 μg/ml, between 0.2 μg/ml and 1 μg/ml, between 0.5 μg/ml and 1 μg/ml, between 0.001 μg/ml and 1 μg/ml, between 0.002 μg/ml and 0.5 μg/ml, between 0.005 μg/ml and 0.2 μg/ml, between 0.01 μg/ml and 0.1 μg/ml, between 0.1 μg/ml and 0.6 μg/ml, between 0.2 μg/ml and 0.6 μg/ml, between 0.3 μg/ml and 0.6 μg/ml, between 0.3 μg/ml and 0.7 μg/ml, between 0.3 μg/ml and 0.8 μg/ml, between 0.3 μg/ml and 0.9 μg/ml, between 0.3 μg/ml and 1 μg/ml, or between 0.02 μg/ml and 0.05 μg/ml, e.g., as determined by a method described herein;
c) Binds specifically to an epitope on FGF23 (e.g., human FGF23), e.g., the same, similar, or overlapping epitope as the epitope recognized by a monoclonal antibody described herein (e.g., an antibody as listed in Table 1, e.g., ExA11, ExA28, ExA35, ExA43, ExA60, ExC17, ExC50, Exc23, Exc23.1, Exc23.2, Exc23.3, Exc23.4, or Exc23.5);
d) Reduces (e.g., inhibits, blocks, or neutralizes) one or more biological activities of FGF23 (e.g., human FGF23), in vitro, ex vivo, or in vivo;
e) Reduces (e.g., inhibits, blocks, or neutralizes) one or more biological activities of FGF23 (e.g., human FGF23), e.g., at a half maximal inhibitory concentration (IC₅₀) of about 1 μg/ml or less, 0.9 μg/ml or less, 0.8 μg/ml or less, 0.7 μg/ml or less, 0.6 μg/ml or less, 0.5 μg/ml or less, 0.4 μg/ml or less, 0.3 μg/ml or less, 0.2 μg/ml or less, 0.1 μg/ml or less, 0.05 μg/ml or less, 0.02 μg/ml or less, 0.01 μg/ml or less, 0.005 μg/ml or less, 0.002 μg/ml or less, or 0.001 μg/ml or less, e.g., between 0.001 μg/ml and 1 μg/ml, between 0.001 μg/ml and 0.9 μg/ml, between 0.001 μg/ml and 0.8 μg/ml, between 0.001 μg/ml and 0.7 μg/ml, between 0.001 μg/ml and 0.6 μg/ml, between 0.001 μg/ml and 0.5 μg/ml, between 0.001 μg/ml and 0.2 μg/ml, between 0.001 μg/ml and 0.1 μg/ml, between 0.001 and 0.05 μg/ml, between 0.001 and 0.02 μg/ml, between 0.001 and 0.005 μg/ml, between 0.01 μg/ml and 1 μg/ml, between 0.01 μg/ml and 0.9 μg/ml, between 0.01 μg/ml and 0.8 μg/ml, between 0.01 μg/ml and 0.7 μg/ml, between 0.01 μg/ml and 0.6 μg/ml, between 0.01 μg/ml and 0.5 μg/ml, between 0.02 μg/ml and 0.7 μg/ml, between 0.02 μg/ml and 0.6 μg/ml, between 0.02 μg/ml and 0.5 μg/ml, between 0.05 μg/ml and 0.2 μg/ml, between 0.001 μg/ml and 0.002 μg/ml, between 0.002 μg/ml and 0.005 μg/ml, between 0.005 μg/ml and 0.01 μg/ml, between 0.01 μg/ml and 0.02 μg/ml, between 0.02 μg/ml and 0.05 μg/ml, between 0.05 μg/ml and 0.1 μg/ml, between 0.1 μg/ml and 0.2 μg/ml, between 0.1 μg/ml and 0.3 μg/ml, between 0.1 μg/ml and 0.4 μg/ml, between 0.1 μg/ml and 0.5 μg/ml, between 0.1 μg/ml and 0.6 μg/ml, between 0.1 μg/ml and 0.7 μg/ml, between 0.1 μg/ml and 0.8 μg/ml, between 0.1 μg/ml and 0.9 μg/ml, between 0.2 μg/ml and 0.3 μg/ml, between 0.2 μg/ml and 0.4 μg/ml, between 0.2 μg/ml and 0.5 μg/ml, between 0.2 μg/ml and 0.6 μg/ml, between 0.2 μg/ml and 0.7 μg/ml, between 0.2 μg/ml and 0.8 μg/ml, between 0.2 μg/ml and 0.9 μg/ml, between 0.3 μg/ml and 0.4 μg/ml, between 0.3 μg/ml and 0.5 μg/ml, between 0.3 μg/ml and 0.6 μg/ml, between 0.3 μg/ml and 0.7 μg/ml, between 0.3 μg/ml and 0.8 μg/ml, between 0.3 μg/ml and 0.9 μg/ml, between 0.4 μg/ml and 0.5 μg/ml, between 0.4 μg/ml and 0.6 μg/ml, between 0.4 μg/ml and 0.7 μg/ml, between 0.4 μg/ml and 0.8 μg/ml, between 0.4 μg/ml and 0.9 μg/ml, between 0.5 μg/ml and 1 μg/ml, between 1 μg/ml and 2 μg/ml, between 2 μg/ml and 5 μg/ml, or between 5 μg/ml and 10 μg/ml, e.g., between 1 μg/ml and 8 μg/ml or between 2 μg/ml and 6 μg/ml, e.g., as determined by a method described herein;
f) Shows the same or similar binding affinity or specificity, or both, as a monoclonal antibody described in Table 1, e.g., any of monoclonal antibodies ExA11, ExA28, ExA35, ExA43, ExA60, ExC17, ExC50, Exc23, Exc23.1, Exc23.2, Exc23.3, Exc23.4, or Exc23.5;
g) Shows the same or similar binding affinity or specificity, or both, as an antibody molecule comprising a heavy chain variable region and/or light chain variable region described in Table 1, e.g., a heavy chain variable region and/or light chain variable region of any of monoclonal antibodies ExA11, ExA28, ExA35, ExA43, ExA60, ExC17, ExC50, Exc23, Exc23.1, Exc23.2, Exc23.3, Exc23.4, or Exc23.5;
h) Shows the same or similar binding affinity or specificity, or both, as an antibody molecule comprising one or more (e.g., two or three) heavy chain CDRs and/or one or more (e.g., two or three) light chain CDRs described in Table 1, e.g., one or more (e.g., two or three) heavy chain CDRs and/or one or more (two or three) light chain CDRs of any of monoclonal antibodies ExA11, ExA28, ExA35, ExA43, ExA60, ExC17, ExC50, Exc23, Exc23.1, Exc23.2, Exc23.3, Exc23.4, or Exc23.5;
i) Shows the same or similar binding affinity or specificity, or both, as an antibody molecule comprising an amino acid sequence shown in Table 1;
j) Shows the same or similar binding affinity or specificity, or both, as an antibody molecule comprising an amino acid sequence encoded by a nucleotide sequence shown in Table 5;
k) Inhibits, e.g., competitively inhibits, the binding of a second antibody molecule to FGF23 (e.g., human FGF23), e.g., human FGF23, wherein the second antibody molecule is an antibody molecule chosen from Table 1, e.g., any of monoclonal antibodies ExA11, ExA28, ExA35, ExA43, ExA60, ExC17, ExC50, Exc23, Exc23.1, Exc23.2, Exc23.3, Exc23.4, or Exc23.5;
l) Competes for binding with a second antibody molecule to FGF23 (e.g., human FGF23), wherein the second antibody molecule is a monoclonal antibody chosen from Table 1, e.g., any of monoclonal antibodies ExA11, ExA28, ExA35, ExA43, ExA60, ExC17, ExC50, Exc23, Exc23.1, Exc23.2, Exc23.3, Exc23.4, or Exc23.5;
m) Has one or more biological properties of a monoclonal antibody chosen from Table 1, e.g., any of monoclonal antibodies ExA11, ExA28, ExA35, ExA43, ExA60, ExC17, ExC50, Exc23, Exc23.1, Exc23.2, Exc23.3, Exc23.4, or Exc23.5;
n) Has one or more structural properties of a monoclonal antibody chosen from Table 1, e.g., any of monoclonal antibodies ExA11, ExA28, ExA35, ExA43, ExA60, ExC17, ExC50, Exc23, Exc23.1, Exc23.2, Exc23.3, Exc23.4, or Exc23.5; or
o) Has one or more pharmacokinetic properties of a monoclonal antibody chosen from Table 1, e.g., any of monoclonal antibodies ExA11, ExA28, ExA35, ExA43, ExA60, ExC17, ExC50, Exc23, Exc23.1, Exc23.2, Exc23.3, Exc23.4, or Exc23.5.

In an aspect, this disclosure provides an isolated antibody molecule capable of binding to FGF23, comprising:

(a) a heavy chain variable region (VH) comprising an HCDR1 amino acid sequence of SEQ ID NO: 41, an HCDR2 amino acid sequence of SEQ ID NO: 48, and an HCDR3 amino acid sequence of SEQ ID NO: 53; and a light chain variable region (VL) comprising an LCDR1 amino acid sequence of SEQ ID NO: 61, an LCDR2 amino acid sequence of SEQ ID NO: 67, and an LCDR3 amino acid sequence of SEQ ID NO: 74;

(b) a VH comprising an HCDR1 amino acid sequence of SEQ ID NO: 41, an HCDR2 amino acid sequence of SEQ ID NO: 46, and an HCDR3 amino acid sequence of SEQ ID NO: 53; and a light chain variable region (VL) comprising an LCDR1 amino acid sequence of SEQ ID NO: 61, an LCDR2 amino acid sequence of SEQ ID NO: 67, and an LCDR3 amino acid sequence of SEQ ID NO: 74;

(c) a VH comprising an HCDR1 amino acid sequence of SEQ ID NO: 41, an HCDR2 amino acid sequence of SEQ ID NO: 49, and an HCDR3 amino acid sequence of SEQ ID NO: 53; and a light chain variable region (VL) comprising an LCDR1 amino acid sequence of SEQ ID NO: 61, an LCDR2 amino acid sequence of SEQ ID NO: 67, and an LCDR3 amino acid sequence of SEQ ID NO: 74;

(d) a VH comprising an HCDR1 amino acid sequence of SEQ ID NO: 41, an HCDR2 amino acid sequence of SEQ ID NO: 46, and an HCDR3 amino acid sequence of SEQ ID NO: 57; and a light chain variable region (VL) comprising an LCDR1 amino acid sequence of SEQ ID NO: 61, an LCDR2 amino acid sequence of SEQ ID NO: 67, and an LCDR3 amino acid sequence of SEQ ID NO: 74;

(e) a VH comprising an HCDR1 amino acid sequence of SEQ ID NO: 41, an HCDR2 amino acid sequence of SEQ ID NO: 50, and an HCDR3 amino acid sequence of SEQ ID NO: 54; and a light chain variable region (VL) comprising an LCDR1 amino acid sequence of SEQ ID NO: 61, an LCDR2 amino acid sequence of SEQ ID NO: 67, and an LCDR3 amino acid sequence of SEQ ID NO: 74;

(f) a VH comprising an HCDR1 amino acid sequence of SEQ ID NO: 41, an HCDR2 amino acid sequence of SEQ ID NO: 46, and an HCDR3 amino acid sequence of SEQ ID NO: 53; and a light chain variable region (VL) comprising an LCDR1 amino acid sequence of SEQ ID NO: 61, an LCDR2 amino acid sequence of SEQ ID NO: 69, and an LCDR3 amino acid sequence of SEQ ID NO: 89;

(g) a VH comprising an HCDR1 amino acid sequence of SEQ ID NO: 41, an HCDR2 amino acid sequence of SEQ ID NO: 49, and an HCDR3 amino acid sequence of SEQ ID NO: 55; and a light chain variable region (VL) comprising an LCDR1 amino acid sequence of SEQ ID NO: 61, an LCDR2 amino acid sequence of SEQ ID NO: 69, and an LCDR3 amino acid sequence of SEQ ID NO: 74;

(h) a VH comprising an HCDR1 amino acid sequence of SEQ ID NO: 41, an HCDR2 amino acid sequence of SEQ ID NO: 46, and an HCDR3 amino acid sequence of SEQ ID NO: 53; and a light chain variable region (VL) comprising an LCDR1 amino acid sequence of SEQ ID NO: 59, an LCDR2 amino acid sequence of SEQ ID NO: 70, and an LCDR3 amino acid sequence of SEQ ID NO: 73;

(i) a VH comprising an HCDR1 amino acid sequence of SEQ ID NO: 41, an HCDR2 amino acid sequence of SEQ ID NO: 46, and an HCDR3 amino acid sequence of SEQ ID NO: 53; and a light chain variable region (VL) comprising an LCDR1 amino acid sequence of SEQ ID NO: 59, an LCDR2 amino acid sequence of SEQ ID NO: 68, and an LCDR3 amino acid sequence of SEQ ID NO: 73;

(j) a VH comprising an HCDR1 amino acid sequence of SEQ ID NO: 41, an HCDR2 amino acid sequence of SEQ ID NO: 46, and an HCDR3 amino acid sequence of SEQ ID NO: 53; and a light chain variable region (VL) comprising an LCDR1 amino acid sequence of SEQ ID NO: 59, an LCDR2 amino acid sequence of SEQ ID NO: 69, and an LCDR3 amino acid sequence of SEQ ID NO: 73;

(k) a VH comprising an HCDR1 amino acid sequence of SEQ ID NO: 41, an HCDR2 amino acid sequence of SEQ ID NO: 46, and an HCDR3 amino acid sequence of SEQ ID NO: 53; and a light chain variable region (VL) comprising an LCDR1 amino acid sequence of SEQ ID NO: 59, an LCDR2 amino acid sequence of SEQ ID NO: 70, and an LCDR3 amino acid sequence of SEQ ID NO: 74;

(l) a VH comprising an HCDR1 amino acid sequence of SEQ ID NO: 41, an HCDR2 amino acid sequence of SEQ ID NO: 46, and an HCDR3 amino acid sequence of SEQ ID NO: 53; and a light chain variable region (VL) comprising an LCDR1 amino acid sequence of SEQ ID NO: 61, an LCDR2 amino acid sequence of SEQ ID NO: 70, and an LCDR3 amino acid sequence of SEQ ID NO: 73; or

(m) a VH comprising an HCDR1 amino acid sequence of SEQ ID NO: 41, an HCDR2 amino acid sequence of SEQ ID NO: 46, and an HCDR3 amino acid sequence of SEQ ID NO: 53; and a light chain variable region (VL) comprising an LCDR1 amino acid sequence of SEQ ID NO: 59, an LCDR2 amino acid sequence of SEQ ID NO: 70, and an LCDR3 amino acid sequence of SEQ ID NO: 73.

In an embodiment, the antibody molecule comprises a VH comprising an HCDR1 amino acid sequence of any of SEQ ID NOs: 39-43 or 110; an HCDR2 amino acid sequence of any of SEQ ID NOs: 44-52; and an HCDR3 amino acid sequence of any of SEQ ID NOs: 53-57; and a VL comprising an LCDR1 amino acid sequence of any of SEQ ID NOs: 58-63 and 109, an LCDR2 amino acid sequence of any of SEQ ID NOs: 64-72, and an LCDR3 amino acid sequence of any of SEQ ID NOs: 73-89.

In an embodiment, the antibody molecule comprises a VH comprising an amino acid sequence at least 85%, 90%, 95%, 96%, 97%, 98%, or 99% identical to, or differing by no more than 1, 2, 3, 4, 5, 6, 7, 8, 9, 10, 11, 12, 13, 14, or 15 amino acid residues from, any of SEQ ID NOs: 1-13, 90, or 91. In an embodiment, the antibody molecule comprises a VH comprising an amino acid sequence of any of SEQ ID NOs: 1-13, 90, or 91.

In an embodiment, the antibody molecule comprises a VL comprising an amino acid sequence at least 85%, 90%, 95%, 96%, 97%, 98%, or 99% identical to, or differing by no more than 1, 2, 3, 4, 5, 6, 7, 8, 9, 10, 11, 12, 13, 14, or 15 amino acid residues from, any of SEQ ID NOs: 14-38 or 92-96. In an embodiment, the antibody molecule comprises a VL comprising an amino acid sequence of any of SEQ ID NOs: 14-38 or 92-96.

In an embodiment, the antibody molecule comprises a VH comprising an amino acid sequence at least 85%, 90%, 95%, 96%, 97%, 98%, or 99% identical to, or differing by no more than 1, 2, 3, 4, 5, 6, 7, 8, 9, 10, 11, 12, 13, 14, or 15 amino acid residues from, any of SEQ ID NOs: 1-13, 90, or 91 and a VL comprising an amino acid sequence at least 85%, 90%, 95%, 96%, 97%, 98%, or 99% identical to, or differing by no more than 1, 2, 3, 4, 5, 6, 7, 8, 9, 10, 11, 12, 13, 14, or 15 amino acid residues from, any of SEQ ID NOs: 14-38 or 92-96. In an embodiment, the antibody molecule comprises a VH comprising an amino acid sequence of any of SEQ ID NOs: 1-13, 90, or 91 and a VL comprising an amino acid sequence of any of SEQ ID NOs: 14-38 or 92-96.

In an embodiment, the antibody molecule comprises a VH comprising an amino acid sequence of SEQ ID NO: 5 and/or a VL comprising an amino acid sequence of SEQ ID NO: 19. In an embodiment, the antibody molecule comprises a VH comprising an amino acid sequence of SEQ ID NO: 7 and/or a VL comprising an amino acid sequence of SEQ ID NO: 20. In an embodiment, the antibody molecule comprises a VH comprising an amino acid sequence of SEQ ID NO: 8 and/or a VL comprising an amino acid sequence of SEQ ID NO: 19. In an embodiment, the antibody molecule comprises a VH comprising an amino acid sequence of SEQ ID NO: 9 and/or a VL comprising an amino acid sequence of SEQ ID NO: 19. In an embodiment, the antibody molecule comprises a VH comprising an amino acid sequence of SEQ ID NO: 11 and/or a VL comprising an amino acid sequence of SEQ ID NO: 20. In an embodiment, the antibody molecule comprises a VH comprising an amino acid sequence of SEQ ID NO: 7 and/or a VL comprising an amino acid sequence of SEQ ID NO: 28. In an embodiment, the antibody molecule comprise a VH comprising an amino acid sequence of SEQ ID NO: 13 and/or a VL comprising an amino acid sequence of SEQ ID NO: 25. In an embodiment, the antibody molecule comprise a VH comprising an amino acid sequence of SEQ ID NO: 7 and/or a VL comprising an amino acid sequence of SEQ ID NO: 37. In an embodiment, the antibody molecule comprise a VH comprising an amino acid sequence of SEQ ID NO: 7 and/or a VL comprising an amino acid sequence of SEQ ID NO: 34. In an embodiment, the antibody molecule comprise a VH comprising an amino acid sequence of SEQ ID NO: 7 and/or a VL comprising an amino acid sequence of SEQ ID NO: 36. In an embodiment, the antibody molecule comprise a VH comprising an amino acid sequence of SEQ ID NO: 7 and/or a VL comprising an amino acid sequence of SEQ ID NO: 94. In an embodiment, the antibody molecule comprise a VH comprising an amino acid sequence of SEQ ID NO: 7 and/or a VL comprising an amino acid sequence of SEQ ID NO: 95. In an embodiment, the antibody molecule comprise a VH comprising an amino acid sequence of SEQ ID NO: 7 and/or a VL comprising an amino acid sequence of SEQ ID NO: 96.

In an embodiment, the antibody molecule comprises an antigen-binding fragment. In an embodiment, the antigen-binding fragment comprises a Fab, F(ab′)2, Fv, scFv, or sc(Fv)2.

In an embodiment, the antibody molecule comprises a heavy chain constant region chosen from the heavy chain constant regions of IgG1, IgG2, IgG3, or IgG4, or a chimera of two or more isotypes (e.g. IgG2 and IgG4), and optionally, wherein the heavy chain constant region comprises one or more amino acid modifications in the hinge, CH2 or CH3 region.

In an embodiment, the antibody molecule comprises a light chain constant region chosen from the light chain constant regions of kappa or lambda.

In an embodiment, the antibody molecule comprises a heavy chain constant region chosen from the heavy chain constant regions of IgG1, IgG2, IgG3, IgG4, or a chimera of two or more isotypes (e.g. IgG2 and IgG4), and a light chain constant region chosen from the light chain constant regions of kappa or lambda.

In an embodiment, the antibody molecule comprises an Fc region.

In an embodiment, the antibody molecule comprises two VH and two VL, e.g., two VH and two VL described herein.

In an embodiment, the antibody molecule is a humanized antibody molecule. In an embodiment, the antibody molecule is a monoclonal antibody molecule. In an embodiment, the antibody molecule is a synthetic antibody molecule. In an embodiment, the antibody molecule is a monospecific antibody molecule. In an embodiment, the antibody molecule is a multispecific antibody molecule (e.g., a bispecific antibody molecule).

In an embodiment, the FGF23 is a mammalian FGF23, e.g., a human FGF23.

In an embodiment, the antibody molecule binds to human FGF23 at an EC₅₀ of less than 0.04 μg/ml, e.g., as determined by ELISA.

In an embodiment, the antibody molecule binds to human FGF23 at an EC₅₀ of between 0.01 μg/ml and 0.04 μg/ml, e.g., as determined by ELISA.

In an embodiment, the antibody molecule inhibits cell proliferation at an IC₅₀ of less than 10 μg/ml, e.g., as determined by a cell-based assay, e.g., as described in Example 2.

In an embodiment, the antibody molecule inhibits cell proliferation at an IC₅₀ of between 0.1 μg/ml and 0.6 μg/ml as determined by a cell-based assay, e.g., as described in Example 2.

In an embodiment, the antibody molecule binds to human FGF23 comprising the amino acid sequence of SEQ ID NO: 82.

In an embodiment, the LCDR1, LCDR2, LCDR3, HCDR1 and HCDR2 belong to Chothia CDR canonical classes 2, 1, 3, 1 and 3, respectively.

In an aspect, this disclosure provides an antibody molecule capable of binding to FGF23, comprising a VH comprising an HCDR1, an HCDR2, and an HCDR3, and a VL comprising an LCDR1, an LCDR2, and an LCDR3, wherein LCDR1, LCDR2, LCDR3, HCDR1 and HCDR2 belong to Chothia CDR canonical classes 2, 1, 3, 1 and 3, respectively.

In an aspect, this disclosure provides an antibody molecule that competes for binding to FGF23 with an antibody molecule as described herein.

In an aspect, this disclosure provides an antibody molecule that binds to the same or overlapping epitope as the epitope recognized by an antibody molecule as described herein.

In an aspect, this disclosure provides a pharmaceutical composition comprising the isolated antibody molecule of any of the preceding claims and a pharmaceutically acceptable carrier, excipient or stabilizer.

In an aspect, this disclosure provides an isolated nucleic acid encoding the VH, VL, or both, of the antibody molecule described herein.

In an embodiment, the isolated nucleic acid comprises:

(a) the nucleic acid sequence of SEQ ID NO: 111 and/or the nucleic acid sequence of SEQ ID NO: 112;

(b) the nucleic acid sequence of SEQ ID NO: 97 and/or the nucleic acid sequence of SEQ ID NO: 98;

(c) the nucleic acid sequence of SEQ ID NO: 99 and/or the nucleic acid sequence of SEQ ID NO: 100;

(d) the nucleic acid sequence of SEQ ID NO: 101 and/or the nucleic acid sequence of SEQ ID NO: 102;

(e) the nucleic acid sequence of SEQ ID NO: 103 and/or the nucleic acid sequence of SEQ ID NO: 104;

(f) the nucleic acid sequence of SEQ ID NO: 105 and/or the nucleic acid sequence of SEQ ID NO: 106;

(g) the nucleic acid sequence of SEQ ID NO: 107 and/or the nucleic acid sequence of SEQ ID NO: 108;

(h) the nucleic acid sequence of SEQ ID NO: 113 and/or the nucleic acid sequence of SEQ ID NO: 114;

(i) the nucleic acid sequence of SEQ ID NO: 115 and/or the nucleic acid sequence of SEQ ID NO: 116;

(j) the nucleic acid sequence of SEQ ID NO: 117 and/or the nucleic acid sequence of SEQ ID NO: 118;

(k) the nucleic acid sequence of SEQ ID NO: 119 and/or the nucleic acid sequence of SEQ ID NO: 120;

(l) the nucleic acid sequence of SEQ ID NO: 121 and/or the nucleic acid sequence of SEQ ID NO: 122;

(m) the nucleic acid sequence of SEQ ID NO: 123 and/or the nucleic acid sequence of SEQ ID NO: 124.

In an aspect, this disclosure provides an expression vector comprising a nucleic acid as described herein.

In an aspect, this disclosure provides a host cell comprising a nucleic acid as described herein or a vector as described herein.

In an aspect, this disclosure provides a method of producing an antibody molecule, comprising culturing a host cell as described herein under conditions suitable for gene expression.

In an aspect, this disclosure provides a method of inhibiting FGF23, comprising contacting FGF23 with an antibody molecule as described herein, or a pharmaceutical composition as described herein.

In an embodiment, the contacting step occurs in vitro, ex vivo, or in vivo.

In an aspect, this disclosure provides a method of treating a disorder, comprising administering to a subject in need thereof an antibody molecule as described herein, or a pharmaceutical composition as described herein, in an amount effective to treat the disorder. In an embodiment, the disorder is a FGF23-associated disorder, optionally, wherein the FGF23-associated disorder is chosen from X-linked hypophosphatemic rickets (XLH), autosomal recessive hypophosphatemic rickets (ARHR) (e.g., ARHR 1 or ARHR2), autosomal dominant hypophosphatemic rickets (ADHR), osteoglophonic dysplasia, Jansen-type metaphyseal chondrodysplasia, hypophosphatemia with dental abnormality and ectopic calcification, McCune-Albright syndrome, epidermal nevus syndrome (ENS), or tumor-induced osteomalacia (TIO).

In an embodiment, the antibody molecule is administered to the subject at a dose between 0.1 mg/kg and 50 mg/kg.

In an embodiment, the method further comprises administering a second therapeutic agent or modality.

In an embodiment, the second therapeutic agent or modality is administered before, during, or after the antibody molecule is administered.

In an aspect, this disclosure provides a method of preventing a disorder, comprising administering to a subject in need thereof an antibody molecule as described herein, or a pharmaceutical composition as described herein, in an amount effective to treat the disorder. In an embodiment, the disorder is a FGF23-associated disorder, optionally, wherein the FGF23-associated disorder is chosen from X-linked hypophosphatemic rickets (XLH), autosomal recessive hypophosphatemic rickets (ARHR) (e.g., ARHR 1 or ARHR2), autosomal dominant hypophosphatemic rickets (ADHR), osteoglophonic dysplasia, Jansen-type metaphyseal chondrodysplasia, hypophosphatemia with dental abnormality and ectopic calcification, McCune-Albright syndrome, epidermal nevus syndrome (ENS), or tumor-induced osteomalacia (TIO).

In an aspect, this disclosure provides a method of detecting FGF23, comprising (i) contacting a sample or a subject with an antibody molecule as described herein under conditions that allow interaction of the antibody molecule and FGF23 to occur, and (ii) detecting formation of a complex between the antibody molecule and the sample or subject.

In an embodiment, the method further comprises contacting a reference sample or subject with an antibody molecule of any of claims 1-64 under conditions that allow interaction of the antibody molecule and FGF23 to occur, and (ii) detecting formation of a complex between the antibody molecule and the sample or subject.

In an aspect, this disclosure provides an antibody molecule or a pharmaceutical composition as described herein for use in treating a disorder in a subject. In an embodiment, the disorder is a FGF23-associated disorder, optionally, wherein the FGF23-associated disorder is chosen from X-linked hypophosphatemic rickets (XLH), autosomal recessive hypophosphatemic rickets (ARHR) (e.g., ARHR 1 or ARHR2), autosomal dominant hypophosphatemic rickets (ADHR), osteoglophonic dysplasia, Jansen-type metaphyseal chondrodysplasia, hypophosphatemia with dental abnormality and ectopic calcification, McCune-Albright syndrome, epidermal nevus syndrome (ENS), or tumor-induced osteomalacia (TIO).

In an aspect, this disclosure provides a use of an antibody molecule or pharmaceutical compositions as described herein in the manufacture of a medicament for treating a disorder in a subject. In an embodiment, the disorder is a FGF23-associated disorder, optionally, wherein the FGF23-associated disorder is chosen from X-linked hypophosphatemic rickets (XLH), autosomal recessive hypophosphatemic rickets (ARHR) (e.g., ARHR 1 or ARHR2), autosomal dominant hypophosphatemic rickets (ADHR), osteoglophonic dysplasia, Jansen-type metaphyseal chondrodysplasia, hypophosphatemia with dental abnormality and ectopic calcification, McCune-Albright syndrome, epidermal nevus syndrome (ENS), or tumor-induced osteomalacia (TIO).

In an embodiment, the antibody molecule binds to FGF23 (e.g., human FGF23) with high affinity, e.g., with a K_{D}′ of about 50 nM or less, e.g., about 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.7 nM or less, 0.5 nM or less, 0.2 nM or less, 0.1 nM or less, 0.05 nM or less, 0.02 nM or less, 0.01 nM or less, 0.005 nM or less, 0.002 nM or less, or 0.001 nM or less, e.g., between 0.001 nM and 10 nM, between 0.001 nM and 5 nM, between 0.001 nM and 2 nM, between 0.001 nM and 1 nM, between 0.001 nM and 0.7 nM, between 0.001 nM and 0.5 nM, between 0.001 nM and 0.2 nM, between 0.001 nM and 0.1 nM, between 0.001 and 0.05 nM, between 0.001 and 0.02 nM, between 0.001 and 0.005 nM, between 0.01 nM and 10 nM, between 0.01 nM and 5 nM, between 0.01 nM and 2 nM, between 0.01 nM and 1 nM, between 0.01 nM and 0.7 nM, between 0.01 nM and 0.5 nM, between 0.01 nM and 0.2 nM, between 5 nM and 10 nM, between 2 nM and 10 nM, between 1 nM and 10 nM, between 0.5 nM and 10 nM, between 0.2 nM and 10 nM, between 0.1 nM and 10 nM, between 0.05 nM and 10 nM, between 0.02 nM and 10 nM, between 0.01 nM and 10 nM, between 0.005 nM and 10 nM, between 0.002 and 10 nM, between 0.002 nM and 5 nM, between 0.005 nM and 2 nM, between 0.01 nM and 1 nM, between 0.02 nM and 0.5 nM, between 0.05 nM and 0.2 nM, between 0.001 nM and 0.002 nM, between 0.002 nM and 0.005 nM, between 0.005 nM and 0.01 nM, between 0.01 nM and 0.02 nM, between 0.02 nM and 0.05 nM, between 0.05 nM and 0.1 nM, between 0.1 nM and 0.2 nM, between 0.2 nM and 0.5 nM, between 0.5 nM and 1 nM, between 1 nM and 2 nM, between 2 nM and 5 nM, or between 5 nM and 10 nM, e.g., between 0.1 nM and 0.6 nM or between 0.2 nM and 0.53 nM, e.g., as determined by a method described herein.

In an embodiment, the antibody molecule binds to FGF23 (e.g., human FGF23) at the neutral pH with an affinity that is at least 1, 2, 3, 4, 5, 6, 7, 8, 9, 10-fold higher than the affinity at an acidic pH, e.g., a pH below 7, 6.5, 6, 5.5, 5, or lower.

In an embodiment, the antibody molecule binds to FGF23 (e.g., human FGF23) with high affinity, e.g., with an EC₅₀ of about 2 μg/ml or less, e.g., about 1 μg/ml or less, 0.9 μg/ml or less, 0.8 μg/ml or less, 0.7 μg/ml or less, 0.6 μg/ml or less, 0.5 μg/ml or less, 0.4 μg/ml or less, 0.3 μg/ml or less, 0.2 μg/ml or less, 0.1 μg/ml or less, 0.09 μg/ml or less, 0.08 μg/ml or less, 0.07 μg/ml or less, 0.06 μg/ml or less, 0.05 μg/ml or less, 0.04 μg/ml or less, 0.03 μg/ml or less, 0.02 μg/ml or less, 0.01 μg/ml or less, 0.005 μg/ml or less, 0.002 μg/ml or less, 0.001 μg/ml or less, e.g., between 0.001 μg/ml and 2 μg/ml, e.g., between 0.001 μg/ml and 1 μg/ml, between 0.001 μg/ml and 0.5 μg/ml, between 0.001 μg/ml and 0.2 μg/ml, between 0.001 μg/ml and 0.1 μg/ml, between 0.001 μg/ml and 0.05 μg/ml, between 0.001 μg/ml and 0.02 μg/ml, between 0.001 μg/ml and 0.01 μg/ml, between 0.001 μg/ml and 0.005 μg/ml, between 0.002 μg/ml and 1 μg/ml, between 0.005 μg/ml and 1 μg/ml, between 0.01 μg/ml and 1 μg/ml, between 0.02 μg/ml and 1 μg/ml, between 0.05 μg/ml and 1 μg/ml, between 0.1 μg/ml and 1 μg/ml, between 0.2 μg/ml and 1 μg/ml, between 0.5 μg/ml and 1 μg/ml, between 0.001 μg/ml and 1 μg/ml, between 0.002 μg/ml and 0.5 μg/ml, between 0.005 μg/ml and 0.2 μg/ml, between 0.01 μg/ml and 0.1 μg/ml, or between 0.02 μg/ml and 0.05 μg/ml, e.g., as determined by a method described herein.

In an embodiment, the antibody molecule binds specifically to an epitope on FGF23 (e.g., human FGF23), e.g., the same, similar, or overlapping epitope as the epitope recognized by a monoclonal antibody described in Table 1, e.g., any of monoclonal antibodies ExA11, ExA28, ExA35, ExA43, ExA60, ExC17, ExC50, Exc23, Exc23.1, Exc23.2, Exc23.3, Exc23.4, or Exc23.5.

In an embodiment, the antibody molecule reduces (e.g., inhibits, blocks, or neutralizes) one or more biological activities of FGF23 (e.g., human FGF23), in vitro, ex vivo, or in vivo.

In an embodiment, the antibody molecule reduces (e.g., inhibits, blocks, or neutralizes) one or more biological activities of FGF23 (e.g., human FGF23), e.g., at an IC₅₀ of about 50 μg/ml or less, e.g., about 20 μg/ml or less, 10 μg/ml or less, 9 μg/ml or less, 8 μg/ml or less, 7 μg/ml or less, 6 μg/ml or less, 5 μg/ml or less, 4 μg/ml or less, 3 μg/ml or less, 2 μg/ml or less, 1 μg/ml or less, 0.5 μg/ml or less, 0.2 μg/ml or less, 0.1 μg/ml or less, 0.05 μg/ml or less, 0.02 μg/ml or less, 0.01 μg/ml or less, 0.005 μg/ml or less, 0.002 μg/ml or less, or 0.001 μg/ml or less, e.g., between 0.001 μg/ml and 10 μg/ml, between 0.001 μg/ml and 5 μg/ml, between 0.001 μg/ml and 2 μg/ml, between 0.001 μg/ml and 1 μg/ml, between 0.001 μg/ml and 0.5 μg/ml, between 0.001 μg/ml and 0.2 μg/ml, between 0.001 μg/ml and 0.1 μg/ml, between 0.001 and 0.05 μg/ml, between 0.001 and 0.02 μg/ml, between 0.001 and 0.005 μg/ml, between 5 μg/ml and 10 μg/ml, between 2 μg/ml and 10 μg/ml, between 1 μg/ml and 10 μg/ml, between 0.5 μg/ml and 10 μg/ml, between 0.2 μg/ml and 10 μg/ml, between 0.1 μg/ml and 10 μg/ml, between 0.05 μg/ml and 10 μg/ml, between 0.02 μg/ml and 10 μg/ml, between 0.01 μg/ml and 10 μg/ml, between 0.005 μg/ml and 10 μg/ml, between 0.002 and 10 μg/ml, between 0.002 μg/ml and 5 μg/ml, between 0.005 μg/ml and 2 μg/ml, between 0.01 μg/ml and 1 μg/ml, between 0.02 μg/ml and 0.5 μg/ml, between 0.05 μg/ml and 0.2 μg/ml, between 0.001 μg/ml and 0.002 μg/ml, between 0.002 μg/ml and 0.005 μg/ml, between 0.005 μg/ml and 0.01 μg/ml, between 0.01 μg/ml and 0.02 μg/ml, between 0.02 μg/ml and 0.05 μg/ml, between 0.05 μg/ml and 0.1 μg/ml, between 0.1 μg/ml and 0.2 μg/ml, between 0.2 μg/ml and 0.5 μg/ml, between 0.5 μg/ml and 1 μg/ml, between 1 μg/ml and 2 μg/ml, between 2 μg/ml and 5 μg/ml, or between 5 μg/ml and 10 μg/ml, e.g., between 1 μg/ml and 8 μg/ml or between 2 μg/ml and 6 μg/ml, e.g., as determined by a method described herein.

In an embodiment, the antibody molecule shows the same or similar binding affinity or specificity, or both, as a monoclonal antibody described in Table 1, e.g., any of monoclonal antibodies ExA11, ExA28, ExA35, ExA43, ExA60, ExC17, ExC50, Exc23, Exc23.1, Exc23.2, Exc23.3, Exc23.4, or Exc23.5.

In an embodiment, the antibody molecule shows the same or similar binding affinity or specificity, or both, as an antibody molecule comprising a heavy chain variable region and/or light chain variable region described in Table 1, e.g., a heavy chain variable region and/or light chain variable region of any of monoclonal antibodies ExA11, ExA28, ExA35, ExA43, ExA60, ExC17, ExC50, Exc23, Exc23.1, Exc23.2, Exc23.3, Exc23.4, or Exc23.5.

In an embodiment, the antibody molecule shows the same or similar binding affinity or specificity, or both, as an antibody molecule comprising one or more (e.g., two or three) heavy chain CDRs and/or one or more (e.g., two or three) light chain CDRs described in Table 1, e.g., one or more (e.g., two or three) heavy chain CDRs and/or one or more (two or three) light chain CDRs of any of monoclonal antibodies ExA11, ExA28, ExA35, ExA43, ExA60, ExC17, ExC50, Exc23, Exc23.1, Exc23.2, Exc23.3, Exc23.4, or Exc23.5.

In an embodiment, the antibody molecule shows the same or similar binding affinity or specificity, or both, as an antibody molecule comprising an amino acid sequence shown in Table 1,

In an embodiment, the antibody molecule shows the same or similar binding affinity or specificity, or both, as an antibody molecule comprising an amino acid sequence encoded by a nucleotide sequence shown in Table 5.

In an embodiment, the antibody molecule inhibits, e.g., competitively inhibits, the binding of a second antibody molecule to FGF23 (e.g., human FGF23) wherein the second antibody molecule is an antibody molecule chosen from Table 1, e.g., any of monoclonal antibodies ExA11, ExA28, ExA35, ExA43, ExA60, ExC17, ExC50, Exc23, Exc23.1, Exc23.2, Exc23.3, Exc23.4, or Exc23.5.

In an embodiment, the antibody molecule competes for binding with a second antibody molecule to FGF23 (e.g., human FGF23), wherein the second antibody molecule is a monoclonal antibody chosen from Table 1, e.g., any of monoclonal antibodies ExA11, ExA28, ExA35, ExA43, ExA60, ExC17, ExC50, Exc23, Exc23.1, Exc23.2, Exc23.3, Exc23.4, or Exc23.5.

In an embodiment, the antibody molecule has one or more biological properties of a monoclonal antibody chosen from Table 1, e.g., any of monoclonal antibodies ExA11, ExA28, ExA35, ExA43, ExA60, ExC17, ExC50, Exc23, Exc23.1, Exc23.2, Exc23.3, Exc23.4, or Exc23.5.

In an embodiment, the antibody molecule has one or more structural properties of a monoclonal antibody chosen from Table 1, e.g., any of monoclonal antibodies ExA11, ExA28, ExA35, ExA43, ExA60, ExC17, ExC50, Exc23, Exc23.1, Exc23.2, Exc23.3, Exc23.4, or Exc23.5.

In an embodiment, the antibody molecule has one or more pharmacokinetic properties of a monoclonal antibody chosen from Table 1, e.g., any of monoclonal antibodies ExA11, ExA28, ExA35, ExA43, ExA60, ExC17, ExC50, Exc23, Exc23.1, Exc23.2, Exc23.3, Exc23.4, or Exc23.5.

In an embodiment, the antibody molecule is a synthetic antibody molecule. In an embodiment, the antibody molecule is an isolated antibody molecule. In an embodiment, the antibody molecule is a recombinant antibody molecule. In an embodiment, the antibody molecule is a humanized antibody. In an embodiment, the antibody molecule is a mono-specific antibody molecule. In an embodiment, the antibody molecule is a multi-specific antibody molecule.

In an embodiment, the antibody molecule comprises a heavy chain variable region (VH) and a light chain variable region (VL), wherein the VH comprises three heavy chain complementarity determining regions (HCDR1, HCDR2, and HCDR3), wherein the VL comprises three light chain complementarity determining regions (LCDR1, LCDR2, and LCDR3).

In an embodiment, the VH comprises an HCDR1 sequence having at least 85% (e.g., at least 85%, 90%, 95%, 96%, 97%, 98%, 99%, or 100%) sequence identity to an HCDR1 sequence listed in Table 3. In an embodiment, the VH comprises an HCDR2 sequence having at least 85% (e.g., at least 85%, 90%, 95%, 96%, 97%, 98%, 99%, or 100%) sequence identity to an HCDR2 sequence listed in Table 3. In an embodiment, the VH comprises an HCDR3 sequence having at least 85% (e.g., at least 85%, 90%, 95%, 96%, 97%, 98%, 99%, or 100%) sequence identity to an HCDR3 sequence listed in Table 3. In an embodiment, the VH comprises HCDR1, HCDR2, and HCDR3 sequences, each having at least 85% (e.g., at least 85%, 90%, 95%, 96%, 97%, 98%, 99%, or 100%) sequence identity to an HCDR1, HCDR2, and HCDR3 sequence, respectively, as listed in Table 3.

In an embodiment, the VL comprises an LCDR1 sequence having at least 85% (e.g., at least 85%, 90%, 95%, 96%, 97%, 98%, 99%, or 100%) sequence identity to an LCDR1 sequence listed in Table 3. In an embodiment, the VL comprises an LCDR2 sequence having at least 85% (e.g., at least 85%, 90%, 95%, 96%, 97%, 98%, 99%, or 100%) sequence identity to an LCDR2 sequence listed in Table 3. In an embodiment, the VL comprises an LCDR3 sequence having at least 85% (e.g., at least 85%, 90%, 95%, 96%, 97%, 98%, 99%, or 100%) sequence identity to an LCDR3 sequence listed in Table 3. In an embodiment, the VL comprises LCDR1, LCDR2, and LCDR3 sequences, each having at least 85% (e.g., at least 85%, 90%, 95%, 96%, 97%, 98%, 99%, or 100%) sequence identity to an LCDR1, LCDR2, and LCDR3 sequence, respectively, as listed in Table 3.

In an embodiment, the VH comprises HCDR1, HCDR2, and HCDR3 sequences, each having at least 85% (e.g., at least 85%, 90%, 95%, 96%, 97%, 98%, 99%, or 100%) sequence identity to an HCDR1, HCDR2, and HCDR3 sequence, respectively, as listed in Table 3, and the VL comprises LCDR1, LCDR2, and LCDR3 sequences, each having at least 85% (e.g., at least 85%, 90%, 95%, 96%, 97%, 98%, 99%, or 100%) sequence identity to an LCDR1, LCDR2, and LCDR3 sequence, respectively, as listed in Table 3.

In an embodiment, the VH comprises one, two, or all of the following:

(i) an HCDR1 comprising the amino acid sequence:
X₁X₂X₃X₄H,
wherein: X₁ is N, S, or A;
   X₂ is H or Y;
   X₃ is F or Y; and
   X₄ is I or M;
(SEQ ID NO: 83);

(ii) an HCDR2 comprising the amino acid sequence:
X₁INPX₂X₃GSX₄X₅X₆AQKX₇QG,
wherein: X₁ is I or T;
   X₂ is I, N, or V;
   X₃ is S or T;
   X₄ is S or T;
   X₅ is S, T, or N;
   X₆ is N or Y; and
   X₇ is F or L;
(SEQ ID NO: 84);

(iii) an HCDR3 comprising the amino acid sequence:
X₁X₂X₃DAFDX_{4,}
wherein: X₁ is D or E;
   X₂ is L or I;
   X₃ is V or L; and
   X₄ is F or Y;
(SEQ ID NO: 85); and/or

the VL comprises one, two, or all of the following:

(iv) an LCDR1 comprising the amino acid sequence:
X₁ASX₂GX₃SSX₄LX_{5,}
wherein: X₁ is K or R;
   X₂ is Q or A;
   X₃ is I or V;
   X₄ is A or Y; and
   X₅ is A or V;
(SEQ ID NO: 86);

(v) an LCDR2 comprising the amino acid sequence:
X₁ASX₂X₃X₄X_{5,}
wherein: X₁ is A, D, or K;
   X₂ is N or S;
   X₃ is L or R;
   X₄ is E, Q, or A; and
   X₅ is S or T;
(SEQ ID NO: 87); and

(vi) an LCDR3 comprising the amino acid sequence:
QQX₁X₂X₃X₄X₅X_{6,}
wherein: X₁ is F or Y;
   X₂ is N or S;
   X₃ is D, N, or S;
   X₄ is Y or L;
   X₅ is F or Y; and
   X₆ is S or T;
(SEQ ID NO: 88).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ExA11 (e.g., SEQ ID NO: 41); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ExA11 (e.g., SEQ ID NO: 48); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ExA11 (e.g., SEQ ID NO: 53).

In an embodiment, the antibody molecule comprises a VL, wherein the VL comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ExA11 (e.g., SEQ ID NO: 61); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ExA11 (e.g., SEQ ID NO: 67); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ExA11 (e.g., SEQ ID NO: 74).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ExA11 (e.g., SEQ ID NO: 41); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ExA11 (e.g., SEQ ID NO: 48); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ExA11 (e.g., SEQ ID NO: 53), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ExA11 (e.g., SEQ ID NO: 61); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ExA11 (e.g., SEQ ID NO: 67); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ExA11 (e.g., SEQ ID NO: 74).

In an embodiment, the antibody molecule comprises: (i) a VH comprising: an HCDR1 comprising the amino acid sequence of the HCDR1 of monoclonal antibody ExA11 (e.g., SEQ ID NO: 41); an HCDR2 comprising the amino acid sequence of the HCDR2 of monoclonal antibody ExA11 (e.g., SEQ ID NO: 48); and an HCDR3 comprising the amino acid sequence of the HCDR3 of monoclonal antibody ExA11 (e.g., SEQ ID NO: 53), and (ii) a VL comprising: an LCDR1 comprising the amino acid sequence of the LCDR1 of monoclonal antibody ExA11 (e.g., SEQ ID NO: 61); an LCDR2 comprising the amino acid sequence of the LCDR2 of monoclonal antibody ExA11 (e.g., SEQ ID NO: 67); and an LCDR3 comprising the amino acid sequence of the LCDR3 of monoclonal antibody ExA11 (e.g., SEQ ID NO: 74).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ExA28 (e.g., SEQ ID NO: 41); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ExA28 (e.g., SEQ ID NO: 46); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ExA28 (e.g., SEQ ID NO: 53).

In an embodiment, the antibody molecule comprises a VL, wherein the VL comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ExA28 (e.g., SEQ ID NO: 61); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ExA28 (e.g., SEQ ID NO: 67); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ExA28 (e.g., SEQ ID NO: 74).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody (e.g., SEQ ID NO: 41); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ExA28 (e.g., SEQ ID NO: 64); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ExA28 (e.g., SEQ ID NO: 53), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ExA28 (e.g., SEQ ID NO: 61); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ExA28 (e.g., SEQ ID NO: 67); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ExA28 (e.g., SEQ ID NO: 74).

In an embodiment, the antibody molecule comprises: (i) a VH comprising: an HCDR1 comprising the amino acid sequence of the HCDR1 of monoclonal antibody ExA28 (e.g., SEQ ID NO: 41); an HCDR2 comprising the amino acid sequence of the HCDR2 of monoclonal antibody ExA28 (e.g., SEQ ID NO: 46); and an HCDR3 comprising the amino acid sequence of the HCDR3 of monoclonal antibody ExA28 (e.g., SEQ ID NO: 53), and (ii) a VL comprising: an LCDR1 comprising the amino acid sequence of the LCDR1 of monoclonal antibody ExA28 (e.g., SEQ ID NO: 61); an LCDR2 comprising the amino acid sequence of the LCDR2 of monoclonal antibody ExA28 (e.g., SEQ ID NO: 67); and an LCDR3 comprising the amino acid sequence of the LCDR3 of monoclonal antibody ExA28 (e.g., SEQ ID NO: 74).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ExA35 (e.g., SEQ ID NO: 41); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ExA35 (e.g., SEQ ID NO: 49); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ExA35 (e.g., SEQ ID NO: 53).

In an embodiment, the antibody molecule comprises a VL, wherein the VL comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ExA35 (e.g., SEQ ID NO: 61); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ExA35 (e.g., SEQ ID NO: 67); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ExA35 (e.g., SEQ ID NO: 74).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody (e.g., SEQ ID NO: 41); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ExA35 (e.g., SEQ ID NO: 49); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ExA35 (e.g., SEQ ID NO: 53), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ExA35 (e.g., SEQ ID NO: 61); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ExA35 (e.g., SEQ ID NO: 67); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ExA35 (e.g., SEQ ID NO: 74).

In an embodiment, the antibody molecule comprises: (i) a VH comprising: an HCDR1 comprising the amino acid sequence of the HCDR1 of monoclonal antibody ExA35 (e.g., SEQ ID NO: 41); an HCDR2 comprising the amino acid sequence of the HCDR2 of monoclonal antibody ExA35 (e.g., SEQ ID NO: 49); and an HCDR3 comprising the amino acid sequence of the HCDR3 of monoclonal antibody ExA35 (e.g., SEQ ID NO: 53), and (ii) a VL comprising: an LCDR1 comprising the amino acid sequence of the LCDR1 of monoclonal antibody ExA35 (e.g., SEQ ID NO: 61); an LCDR2 comprising the amino acid sequence of the LCDR2 of monoclonal antibody ExA35 (e.g., SEQ ID NO: 67); and an LCDR3 comprising the amino acid sequence of the LCDR3 of monoclonal antibody ExA35 (e.g., SEQ ID NO: 74).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ExA43 (e.g., SEQ ID NO: 41); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ExA43 (e.g., SEQ ID NO: 46); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ExA43 (e.g., SEQ ID NO: 57).

In an embodiment, the antibody molecule comprises a VL, wherein the VL comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ExA43 (e.g., SEQ ID NO: 61); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ExA43 (e.g., SEQ ID NO: 67); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ExA43 (e.g., SEQ ID NO: 74).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody (e.g., SEQ ID NO: 41); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ExA43 (e.g., SEQ ID NO: 46); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ExA43 (e.g., SEQ ID NO: 57), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ExA43 (e.g., SEQ ID NO: 61); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ExA43 (e.g., SEQ ID NO: 67); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ExA43 (e.g., SEQ ID NO: 74).

In an embodiment, the antibody molecule comprises: (i) a VH comprising: an HCDR1 comprising the amino acid sequence of the HCDR1 of monoclonal antibody ExA43 (e.g., SEQ ID NO: 41); an HCDR2 comprising the amino acid sequence of the HCDR2 of monoclonal antibody ExA43 (e.g., SEQ ID NO: 46); and an HCDR3 comprising the amino acid sequence of the HCDR3 of monoclonal antibody ExA43 (e.g., SEQ ID NO: 57), and (ii) a VL comprising: an LCDR1 comprising the amino acid sequence of the LCDR1 of monoclonal antibody ExA43 (e.g., SEQ ID NO: 61); an LCDR2 comprising the amino acid sequence of the LCDR2 of monoclonal antibody ExA43 (e.g., SEQ ID NO: 67); and an LCDR3 comprising the amino acid sequence of the LCDR3 of monoclonal antibody ExA43 (e.g., SEQ ID NO: 74).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ExA60 (e.g., SEQ ID NO: 41); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ExA60 (e.g., SEQ ID NO: 50); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ExA60 (e.g., SEQ ID NO: 54).

In an embodiment, the antibody molecule comprises a VL, wherein the VL comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ExA60 (e.g., SEQ ID NO: 61); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ExA60 (e.g., SEQ ID NO: 67); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ExA60 (e.g., SEQ ID NO: 74).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody (e.g., SEQ ID NO: 41); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ExA60 (e.g., SEQ ID NO: 50); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ExA60 (e.g., SEQ ID NO: 54), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ExA60 (e.g., SEQ ID NO: 61); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ExA60 (e.g., SEQ ID NO: 67); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ExA60 (e.g., SEQ ID NO: 74).

In an embodiment, the antibody molecule comprises: (i) a VH comprising: an HCDR1 comprising the amino acid sequence of the HCDR1 of monoclonal antibody ExA60 (e.g., SEQ ID NO: 41); an HCDR2 comprising the amino acid sequence of the HCDR2 of monoclonal antibody ExA60 (e.g., SEQ ID NO: 50); and an HCDR3 comprising the amino acid sequence of the HCDR3 of monoclonal antibody ExA60 (e.g., SEQ ID NO: 54), and (ii) a VL comprising: an LCDR1 comprising the amino acid sequence of the LCDR1 of monoclonal antibody ExA60 (e.g., SEQ ID NO: 61); an LCDR2 comprising the amino acid sequence of the LCDR2 of monoclonal antibody ExA60 (e.g., SEQ ID NO: 67); and an LCDR3 comprising the amino acid sequence of the LCDR3 of monoclonal antibody ExA60 (e.g., SEQ ID NO: 74).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ExC17 (e.g., SEQ ID NO: 41); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ExC17 (e.g., SEQ ID NO: 46); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ExC17 (e.g., SEQ ID NO: 53).

In an embodiment, the antibody molecule comprises a VL, wherein the VL comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ExC17 (e.g., SEQ ID NO: 61); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ExC17 (e.g., SEQ ID NO: 69); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ExC17 (e.g., SEQ ID NO: 89).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody (e.g., SEQ ID NO: 41); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ExC17 (e.g., SEQ ID NO: 46); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ExC17 (e.g., SEQ ID NO: 53), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ExC17 (e.g., SEQ ID NO: 61); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ExC17 (e.g., SEQ ID NO: 69); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ExC17 (e.g., SEQ ID NO: 89).

In an embodiment, the antibody molecule comprises: (i) a VH comprising: an HCDR1 comprising the amino acid sequence of the HCDR1 of monoclonal antibody ExC17 (e.g., SEQ ID NO: 41); an HCDR2 comprising the amino acid sequence of the HCDR2 of monoclonal antibody ExC17 (e.g., SEQ ID NO: 46); and an HCDR3 comprising the amino acid sequence of the HCDR3 of monoclonal antibody ExC17 (e.g., SEQ ID NO: 53), and (ii) a VL comprising: an LCDR1 comprising the amino acid sequence of the LCDR1 of monoclonal antibody ExC17 (e.g., SEQ ID NO: 61); an LCDR2 comprising the amino acid sequence of the LCDR2 of monoclonal antibody ExC17 (e.g., SEQ ID NO: 69); and an LCDR3 comprising the amino acid sequence of the LCDR3 of monoclonal antibody ExC17 (e.g., SEQ ID NO: 89).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ExC50 (e.g., SEQ ID NO: 41); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ExC50 (e.g., SEQ ID NO: 49); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ExC50 (e.g., SEQ ID NO: 55).

In an embodiment, the antibody molecule comprises a VL, wherein the VL comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ExC50 (e.g., SEQ ID NO: 61); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ExC50 (e.g., SEQ ID NO: 69); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ExC50 (e.g., SEQ ID NO: 74).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody (e.g., SEQ ID NO: 41); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ExC50 (e.g., SEQ ID NO: 49); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ExC50 (e.g., SEQ ID NO: 55), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ExC50 (e.g., SEQ ID NO: 61); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ExC50 (e.g., SEQ ID NO: 69); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ExC50 (e.g., SEQ ID NO: 74).

In an embodiment, the antibody molecule comprises: (i) a VH comprising: an HCDR1 comprising the amino acid sequence of the HCDR1 of monoclonal antibody ExC50 (e.g., SEQ ID NO: 41); an HCDR2 comprising the amino acid sequence of the HCDR2 of monoclonal antibody ExC50 (e.g., SEQ ID NO: 49); and an HCDR3 comprising the amino acid sequence of the HCDR3 of monoclonal antibody ExC50 (e.g., SEQ ID NO: 55), and (ii) a VL comprising: an LCDR1 comprising the amino acid sequence of the LCDR1 of monoclonal antibody ExC50 (e.g., SEQ ID NO: 61); an LCDR2 comprising the amino acid sequence of the LCDR2 of monoclonal antibody ExC50 (e.g., SEQ ID NO: 69); and an LCDR3 comprising the amino acid sequence of the LCDR3 of monoclonal antibody ExC50 (e.g., SEQ ID NO: 74).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody Exc23 (e.g., SEQ ID NO: 41); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody Exc23 (e.g., SEQ ID NO: 46); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody Exc23 (e.g., SEQ ID NO: 53).

In an embodiment, the antibody molecule comprises a VL, wherein the VL comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody Exc23 (e.g., SEQ ID NO: 59); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody Exc23 (e.g., SEQ ID NO: 70); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody Exc23 (e.g., SEQ ID NO: 73).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody (e.g., SEQ ID NO: 41); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody Exc23 (e.g., SEQ ID NO: 46); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody Exc23 (e.g., SEQ ID NO: 53), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody Exc23 (e.g., SEQ ID NO: 59); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody Exc23 (e.g., SEQ ID NO: 70); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody Exc23 (e.g., SEQ ID NO: 73).

In an embodiment, the antibody molecule comprises: (i) a VH comprising: an HCDR1 comprising the amino acid sequence of the HCDR1 of monoclonal antibody Exc23 (e.g., SEQ ID NO: 41); an HCDR2 comprising the amino acid sequence of the HCDR2 of monoclonal antibody Exc23 (e.g., SEQ ID NO: 46); and an HCDR3 comprising the amino acid sequence of the HCDR3 of monoclonal antibody Exc23 (e.g., SEQ ID NO: 53), and (ii) a VL comprising: an LCDR1 comprising the amino acid sequence of the LCDR1 of monoclonal antibody Exc23 (e.g., SEQ ID NO: 59); an LCDR2 comprising the amino acid sequence of the LCDR2 of monoclonal antibody Exc23 (e.g., SEQ ID NO: 70); and an LCDR3 comprising the amino acid sequence of the LCDR3 of monoclonal antibody Exc23 (e.g., SEQ ID NO: 73).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody Exc23.1 (e.g., SEQ ID NO: 41); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody Exc23.1 (e.g., SEQ ID NO: 46); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody Exc23.1 (e.g., SEQ ID NO: 53).

In an embodiment, the antibody molecule comprises a VL, wherein the VL comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody Exc23.1 (e.g., SEQ ID NO: 59); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody Exc23.1 (e.g., SEQ ID NO: 68); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody Exc23.1 (e.g., SEQ ID NO: 73).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody (e.g., SEQ ID NO: 41); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody Exc23.1 (e.g., SEQ ID NO: 46); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody Exc23.1 (e.g., SEQ ID NO: 53), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody Exc23.1 (e.g., SEQ ID NO: 59); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody Exc23.1 (e.g., SEQ ID NO: 68); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody Exc23.1 (e.g., SEQ ID NO: 73).

In an embodiment, the antibody molecule comprises: (i) a VH comprising: an HCDR1 comprising the amino acid sequence of the HCDR1 of monoclonal antibody Exc23.1 (e.g., SEQ ID NO: 41); an HCDR2 comprising the amino acid sequence of the HCDR2 of monoclonal antibody Exc23.1 (e.g., SEQ ID NO: 46); and an HCDR3 comprising the amino acid sequence of the HCDR3 of monoclonal antibody Exc23.1 (e.g., SEQ ID NO: 53), and (ii) a VL comprising: an LCDR1 comprising the amino acid sequence of the LCDR1 of monoclonal antibody Exc23.1 (e.g., SEQ ID NO: 59); an LCDR2 comprising the amino acid sequence of the LCDR2 of monoclonal antibody Exc23.1 (e.g., SEQ ID NO: 68); and an LCDR3 comprising the amino acid sequence of the LCDR3 of monoclonal antibody Exc23.1 (e.g., SEQ ID NO: 73).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody Exc23.2 (e.g., SEQ ID NO: 41); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody Exc23.2 (e.g., SEQ ID NO: 46); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody Exc23.2 (e.g., SEQ ID NO: 53).

In an embodiment, the antibody molecule comprises a VL, wherein the VL comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody Exc23.2 (e.g., SEQ ID NO: 59); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody Exc23.2 (e.g., SEQ ID NO: 69); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody Exc23.2 (e.g., SEQ ID NO: 73).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody (e.g., SEQ ID NO: 41); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody Exc23.2 (e.g., SEQ ID NO: 46); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody Exc23.2 (e.g., SEQ ID NO: 53), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody Exc23.2 (e.g., SEQ ID NO: 59); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody Exc23.2 (e.g., SEQ ID NO: 69); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody Exc23.2 (e.g., SEQ ID NO: 73).

In an embodiment, the antibody molecule comprises: (i) a VH comprising: an HCDR1 comprising the amino acid sequence of the HCDR1 of monoclonal antibody Exc23.2 (e.g., SEQ ID NO: 41); an HCDR2 comprising the amino acid sequence of the HCDR2 of monoclonal antibody Exc23.2 (e.g., SEQ ID NO: 46); and an HCDR3 comprising the amino acid sequence of the HCDR3 of monoclonal antibody Exc23.2 (e.g., SEQ ID NO: 53), and (ii) a VL comprising: an LCDR1 comprising the amino acid sequence of the LCDR1 of monoclonal antibody Exc23.2 (e.g., SEQ ID NO: 59); an LCDR2 comprising the amino acid sequence of the LCDR2 of monoclonal antibody Exc23.2 (e.g., SEQ ID NO: 69); and an LCDR3 comprising the amino acid sequence of the LCDR3 of monoclonal antibody Exc23.2 (e.g., SEQ ID NO: 73).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody Exc23.3 (e.g., SEQ ID NO: 41); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody Exc23.3 (e.g., SEQ ID NO: 46); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody Exc23.3 (e.g., SEQ ID NO: 53).

In an embodiment, the antibody molecule comprises a VL, wherein the VL comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody Exc23.3 (e.g., SEQ ID NO: 59); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody Exc23.3 (e.g., SEQ ID NO: 70); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody Exc23.3 (e.g., SEQ ID NO: 74).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody (e.g., SEQ ID NO: 41); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody Exc23.3 (e.g., SEQ ID NO: 46); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody Exc23.3 (e.g., SEQ ID NO: 53), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody Exc23.3 (e.g., SEQ ID NO: 59); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody Exc23.3 (e.g., SEQ ID NO: 70); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody Exc23.3 (e.g., SEQ ID NO: 74).

In an embodiment, the antibody molecule comprises: (i) a VH comprising: an HCDR1 comprising the amino acid sequence of the HCDR1 of monoclonal antibody Exc23.3 (e.g., SEQ ID NO: 41); an HCDR2 comprising the amino acid sequence of the HCDR2 of monoclonal antibody Exc23.3 (e.g., SEQ ID NO: 46); and an HCDR3 comprising the amino acid sequence of the HCDR3 of monoclonal antibody Exc23.3 (e.g., SEQ ID NO: 53), and (ii) a VL comprising: an LCDR1 comprising the amino acid sequence of the LCDR1 of monoclonal antibody Exc23.3 (e.g., SEQ ID NO: 59); an LCDR2 comprising the amino acid sequence of the LCDR2 of monoclonal antibody Exc23.3 (e.g., SEQ ID NO: 70); and an LCDR3 comprising the amino acid sequence of the LCDR3 of monoclonal antibody Exc23.3 (e.g., SEQ ID NO: 74).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody Exc23.4 (e.g., SEQ ID NO: 41); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody Exc23.4 (e.g., SEQ ID NO: 46); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody Exc23.4 (e.g., SEQ ID NO: 53).

In an embodiment, the antibody molecule comprises a VL, wherein the VL comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody Exc23.4 (e.g., SEQ ID NO: 61); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody Exc23.4 (e.g., SEQ ID NO: 70); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody Exc23.4 (e.g., SEQ ID NO: 73).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody (e.g., SEQ ID NO: 41); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody Exc23.4 (e.g., SEQ ID NO: 46); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody Exc23.4 (e.g., SEQ ID NO: 53), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody Exc23.4 (e.g., SEQ ID NO: 61); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody Exc23.4 (e.g., SEQ ID NO: 70); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody Exc23.4 (e.g., SEQ ID NO: 73).

In an embodiment, the antibody molecule comprises: (i) a VH comprising: an HCDR1 comprising the amino acid sequence of the HCDR1 of monoclonal antibody Exc23.4 (e.g., SEQ ID NO: 41); an HCDR2 comprising the amino acid sequence of the HCDR2 of monoclonal antibody Exc23.4 (e.g., SEQ ID NO: 46); and an HCDR3 comprising the amino acid sequence of the HCDR3 of monoclonal antibody Exc23.4 (e.g., SEQ ID NO: 53), and (ii) a VL comprising: an LCDR1 comprising the amino acid sequence of the LCDR1 of monoclonal antibody Exc23.4 (e.g., SEQ ID NO: 61); an LCDR2 comprising the amino acid sequence of the LCDR2 of monoclonal antibody Exc23.4 (e.g., SEQ ID NO: 70); and an LCDR3 comprising the amino acid sequence of the LCDR3 of monoclonal antibody Exc23.4 (e.g., SEQ ID NO: 73).

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody Exc23.5 (e.g., SEQ ID NO: 41); (ii) an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody Exc23.5 (e.g., SEQ ID NO: 46); or (ii) an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody Exc23.5 (e.g., SEQ ID NO: 53).

In an embodiment, the antibody molecule comprises a VL, wherein the VL comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody Exc23.5 (e.g., SEQ ID NO: 59); (ii) an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody Exc23.5 (e.g., SEQ ID NO: 70); or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody Exc23.5 (e.g., SEQ ID NO: 73).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody (e.g., SEQ ID NO: 41); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody Exc23.5 (e.g., SEQ ID NO: 46); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody Exc23.5 (e.g., SEQ ID NO: 53), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody Exc23.5 (e.g., SEQ ID NO: 59); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody Exc23.5 (e.g., SEQ ID NO: 70); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody Exc23.5 (e.g., SEQ ID NO: 73).

In an embodiment, the antibody molecule comprises: (i) a VH comprising: an HCDR1 comprising the amino acid sequence of the HCDR1 of monoclonal antibody Exc23.5 (e.g., SEQ ID NO: 41); an HCDR2 comprising the amino acid sequence of the HCDR2 of monoclonal antibody Exc23.5 (e.g., SEQ ID NO: 46); and an HCDR3 comprising the amino acid sequence of the HCDR3 of monoclonal antibody Exc23.5 (e.g., SEQ ID NO: 53), and (ii) a VL comprising: an LCDR1 comprising the amino acid sequence of the LCDR1 of monoclonal antibody Exc23.5 (e.g., SEQ ID NO: 59); an LCDR2 comprising the amino acid sequence of the LCDR2 of monoclonal antibody Exc23.5 (e.g., SEQ ID NO: 70); and an LCDR3 comprising the amino acid sequence of the LCDR3 of monoclonal antibody Exc23.5 (e.g., SEQ ID NO: 73).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ExA11 (e.g., SEQ ID NO: 5).

In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ExA11 (e.g., SEQ ID NO: 19).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ExA11 (e.g., SEQ ID NO: 5); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ExA11 (e.g., SEQ ID NO: 19).

In an embodiment, the antibody molecule comprises: (i) a VH comprising the amino acid sequence of the VH of monoclonal antibody ExA11 (e.g., SEQ ID NO: 5); and (ii) a VL comprising the amino acid sequence of the VL of monoclonal antibody ExA11 (e.g., SEQ ID NO: 19).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ExA28 (e.g., SEQ ID NO: 7).

In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ExA28 (e.g., SEQ ID NO: 20).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ExA28 (e.g., SEQ ID NO: 7); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ExA28 (e.g., SEQ ID NO: 20).

In an embodiment, the antibody molecule comprises: (i) a VH comprising the amino acid sequence of the VH of monoclonal antibody ExA28 (e.g., SEQ ID NO: 7); and (ii) a VL comprising the amino acid sequence of the VL of monoclonal antibody ExA28 (e.g., SEQ ID NO: 20).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ExA35 (e.g., SEQ ID NO: 8).

In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ExA35 (e.g., SEQ ID NO: 19).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ExA35 (e.g., SEQ ID NO: 8); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ExA35 (e.g., SEQ ID NO: 19).

In an embodiment, the antibody molecule comprises: (i) a VH comprising the amino acid sequence of the VH of monoclonal antibody ExA35 (e.g., SEQ ID NO: 8); and (ii) a VL comprising the amino acid sequence of the VL of monoclonal antibody ExA35 (e.g., SEQ ID NO: 19).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ExA43 (e.g., SEQ ID NO: 9).

In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ExA43 (e.g., SEQ ID NO: 19).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ExA43 (e.g., SEQ ID NO: 9); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ExA43 (e.g., SEQ ID NO: 19).

In an embodiment, the antibody molecule comprises: (i) a VH comprising the amino acid sequence of the VH of monoclonal antibody ExA43 (e.g., SEQ ID NO: 9); and (ii) a VL comprising the amino acid sequence of the VL of monoclonal antibody ExA43 (e.g., SEQ ID NO: 19).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ExA60 (e.g., SEQ ID NO: 11).

In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ExA60 (e.g., SEQ ID NO: 20).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ExA60 (e.g., SEQ ID NO: 11); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ExA60 (e.g., SEQ ID NO: 20).

In an embodiment, the antibody molecule comprises: (i) a VH comprising the amino acid sequence of the VH of monoclonal antibody ExA60 (e.g., SEQ ID NO: 11); and (ii) a VL comprising the amino acid sequence of the VL of monoclonal antibody ExA60 (e.g., SEQ ID NO: 20).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ExC17 (e.g., SEQ ID NO: 7).

In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ExC17 (e.g., SEQ ID NO: 28).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ExC17 (e.g., SEQ ID NO: 7); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ExC17 (e.g., SEQ ID NO: 28).

In an embodiment, the antibody molecule comprises: (i) a VH comprising the amino acid sequence of the VH of monoclonal antibody ExC17 (e.g., SEQ ID NO: 7); and (ii) a VL comprising the amino acid sequence of the VL of monoclonal antibody ExC17 (e.g., SEQ ID NO: 28).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ExC50 (e.g., SEQ ID NO: 13).

In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ExC50 (e.g., SEQ ID NO: 25).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ExC50 (e.g., SEQ ID NO: 13); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ExC50 (e.g., SEQ ID NO: 25).

In an embodiment, the antibody molecule comprises: (i) a VH comprising the amino acid sequence of the VH of monoclonal antibody ExC50 (e.g., SEQ ID NO: 13); and (ii) a VL comprising the amino acid sequence of the VL of monoclonal antibody ExC50 (e.g., SEQ ID NO: 25).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody Exc23 (e.g., SEQ ID NO: 7).

In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody Exc23 (e.g., SEQ ID NO: 37).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody Exc23 (e.g., SEQ ID NO: 7); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody Exc23 (e.g., SEQ ID NO: 37).

In an embodiment, the antibody molecule comprises: (i) a VH comprising the amino acid sequence of the VH of monoclonal antibody Exc23 (e.g., SEQ ID NO: 7); and (ii) a VL comprising the amino acid sequence of the VL of monoclonal antibody Exc23 (e.g., SEQ ID NO: 37).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody Exc23.1 (e.g., SEQ ID NO: 7).

In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody Exc23.1 (e.g., SEQ ID NO: 34).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody Exc23.1 (e.g., SEQ ID NO: 7); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody Exc23.1 (e.g., SEQ ID NO: 34).

In an embodiment, the antibody molecule comprises: (i) a VH comprising the amino acid sequence of the VH of monoclonal antibody Exc23.1 (e.g., SEQ ID NO: 7); and (ii) a VL comprising the amino acid sequence of the VL of monoclonal antibody Exc23.1 (e.g., SEQ ID NO: 34).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody Exc23.2 (e.g., SEQ ID NO: 7).

In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody Exc23.2 (e.g., SEQ ID NO: 36).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody Exc23.2 (e.g., SEQ ID NO: 7); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody Exc23.2 (e.g., SEQ ID NO: 36).

In an embodiment, the antibody molecule comprises: (i) a VH comprising the amino acid sequence of the VH of monoclonal antibody Exc23.2 (e.g., SEQ ID NO: 7); and (ii) a VL comprising the amino acid sequence of the VL of monoclonal antibody Exc23.2 (e.g., SEQ ID NO: 36).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody Exc23.3 (e.g., SEQ ID NO: 7).

In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody Exc23.3 (e.g., SEQ ID NO: 94).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody Exc23.3 (e.g., SEQ ID NO: 7); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody Exc23.3 (e.g., SEQ ID NO: 94).

In an embodiment, the antibody molecule comprises: (i) a VH comprising the amino acid sequence of the VH of monoclonal antibody Exc23.3 (e.g., SEQ ID NO: 7); and (ii) a VL comprising the amino acid sequence of the VL of monoclonal antibody Exc23.3 (e.g., SEQ ID NO: 94).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody Exc23.4 (e.g., SEQ ID NO: 7).

In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody Exc23.4 (e.g., SEQ ID NO: 95).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody Exc23.4 (e.g., SEQ ID NO: 7); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody Exc23.4 (e.g., SEQ ID NO: 95).

In an embodiment, the antibody molecule comprises: (i) a VH comprising the amino acid sequence of the VH of monoclonal antibody Exc23.4 (e.g., SEQ ID NO: 7); and (ii) a VL comprising the amino acid sequence of the VL of monoclonal antibody Exc23.4 (e.g., SEQ ID NO: 95).

In an embodiment, the antibody molecule comprises a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody Exc23.5 (e.g., SEQ ID NO: 7).

In an embodiment, the antibody molecule comprises a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody Exc23.5 (e.g., SEQ ID NO: 96).

In an embodiment, the antibody molecule comprises: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody Exc23.5 (e.g., SEQ ID NO: 7); and (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody Exc23.5 (e.g., SEQ ID NO: 96).

In an embodiment, the antibody molecule comprises: (i) a VH comprising the amino acid sequence of the VH of monoclonal antibody Exc23.5 (e.g., SEQ ID NO: 7); and (ii) a VL comprising the amino acid sequence of the VL of monoclonal antibody Exc23.5 (e.g., SEQ ID NO: 96).

In an embodiment, the antibody molecule is monoclonal antibody ExA11, ExA28, ExA35, ExA43, ExA60, ExC17, ExC50, Exc23, Exc23.1, Exc23.2, Exc23.3, Exc23.4, or Exc23.5.

In an embodiment, the antibody molecule comprises a VH comprising the amino acid sequence of VH-1 as listed in Table 2 (SEQ ID NO: 1). In an embodiment, the antibody molecule comprises a VH comprising the amino acid sequence of VH-2 as listed in Table 2 (SEQ ID NO: 2). In an embodiment, the antibody molecule comprises a VH comprising the amino acid sequence of VH-3 as listed in Table 2 (SEQ ID NO: 3). In an embodiment, the antibody molecule comprises a VH comprising the amino acid sequence of VH-4 as listed in Table 2 (SEQ ID NO: 4). In an embodiment, the antibody molecule comprises a VH comprising the amino acid sequence of VH-5 as listed in Table 2 (SEQ ID NO: 5). In an embodiment, the antibody molecule comprises a VH comprising the amino acid sequence of VH-6 as listed in Table 2 (SEQ ID NO: 6). In an embodiment, the antibody molecule comprises a VH comprising the amino acid sequence of VH-7 as listed in Table 2 (SEQ ID NO: 7). In an embodiment, the antibody molecule comprises a VH comprising the amino acid sequence of VH-8 as listed in Table 2 (SEQ ID NO: 8). In an embodiment, the antibody molecule comprises a VH comprising the amino acid sequence of VH-9 as listed in Table 2 (SEQ ID NO: 9). In an embodiment, the antibody molecule comprises a VH comprising the amino acid sequence of VH-10 as listed in Table 2 (SEQ ID NO: 10). In an embodiment, the antibody molecule comprises a VH comprising the amino acid sequence of VH-11 as listed in Table 2 (SEQ ID NO: 11). In an embodiment, the antibody molecule comprises a VH comprising the amino acid sequence of VH-12 as listed in Table 2 (SEQ ID NO: 12). In an embodiment, the antibody molecule comprises a VH comprising the amino acid sequence of VH-13 as listed in Table 2 (SEQ ID NO: 13). In an embodiment, the antibody molecule comprises a VH comprising the amino acid sequence of VH-14 as listed in Table 2 (SEQ ID NO: 90). In an embodiment, the antibody molecule comprises a VH comprising the amino acid sequence of VH-15 as listed in Table 2 (SEQ ID NO: 91).

In an embodiment, the antibody molecule comprises a VH comprising the amino acid sequence of VL-1 as listed in Table 2 (SEQ ID NO: 14). In an embodiment, the antibody molecule comprises a VH comprising the amino acid sequence of VL-2 as listed in Table 2 (SEQ ID NO: 15). In an embodiment, the antibody molecule comprises a VH comprising the amino acid sequence of VL-3 as listed in Table 2 (SEQ ID NO: 16). In an embodiment, the antibody molecule comprises a VH comprising the amino acid sequence of VL-4 as listed in Table 2 (SEQ ID NO: 17). In an embodiment, the antibody molecule comprises a VH comprising the amino acid sequence of VL-5 as listed in Table 2 (SEQ ID NO: 18). In an embodiment, the antibody molecule comprises a VH comprising the amino acid sequence of VL-6 as listed in Table 2 (SEQ ID NO: 19). In an embodiment, the antibody molecule comprises a VH comprising the amino acid sequence of VL-7 as listed in Table 2 (SEQ ID NO: 20). In an embodiment, the antibody molecule comprises a VH comprising the amino acid sequence of VL-8 as listed in Table 2 (SEQ ID NO: 21). In an embodiment, the antibody molecule comprises a VH comprising the amino acid sequence of VL-9 as listed in Table 2 (SEQ ID NO: 22). In an embodiment, the antibody molecule comprises a VH comprising the amino acid sequence of VL-10 as listed in Table 2 (SEQ ID NO: 23). In an embodiment, the antibody molecule comprises a VH comprising the amino acid sequence of VL-11 as listed in Table 2 (SEQ ID NO: 24). In an embodiment, the antibody molecule comprises a VH comprising the amino acid sequence of VL-12 as listed in Table 2 (SEQ ID NO: 25). In an embodiment, the antibody molecule comprises a VH comprising the amino acid sequence of VL-13 as listed in Table 2 (SEQ ID NO: 26). In an embodiment, the antibody molecule comprises a VH comprising the amino acid sequence of VL-14 as listed in Table 2 (SEQ ID NO: 27). In an embodiment, the antibody molecule comprises a VH comprising the amino acid sequence of VL-15 as listed in Table 2 (SEQ ID NO: 28). In an embodiment, the antibody molecule comprises a VH comprising the amino acid sequence of VL-16 as listed in Table 2 (SEQ ID NO: 29). In an embodiment, the antibody molecule comprises a VH comprising the amino acid sequence of VL-17 as listed in Table 2 (SEQ ID NO: 30). In an embodiment, the antibody molecule comprises a VH comprising the amino acid sequence of VL-18 as listed in Table 2 (SEQ ID NO: 31). In an embodiment, the antibody molecule comprises a VH comprising the amino acid sequence of VL-19 as listed in Table 2 (SEQ ID NO: 32). In an embodiment, the antibody molecule comprises a VH comprising the amino acid sequence of VL-20 as listed in Table 2 (SEQ ID NO: 33). In an embodiment, the antibody molecule comprises a VH comprising the amino acid sequence of VL-21 as listed in Table 2 (SEQ ID NO: 34). In an embodiment, the antibody molecule comprises a VH comprising the amino acid sequence of VL-22 as listed in Table 2 (SEQ ID NO: 35). In an embodiment, the antibody molecule comprises a VH comprising the amino acid sequence of VL-23 as listed in Table 2 (SEQ ID NO: 36). In an embodiment, the antibody molecule comprises a VH comprising the amino acid sequence of VL-24 as listed in Table 2 (SEQ ID NO: 37). In an embodiment, the antibody molecule comprises a VH comprising the amino acid sequence of VL-25 as listed in Table 2 (SEQ ID NO: 38). In an embodiment, the antibody molecule comprises a VH comprising the amino acid sequence of VL-26 as listed in Table 2 (SEQ ID NO: 92). In an embodiment, the antibody molecule comprises a VH comprising the amino acid sequence of VL-28 as listed in Table 2 (SEQ ID NO: 93).

In an embodiment, the antibody molecule comprises: a VH comprising the amino acid sequence of SEQ ID NO: 1, and a VL comprising the amino acid sequence of SEQ ID NO: 14. In an embodiment, the antibody molecule comprises: a VH comprising the amino acid sequence of SEQ ID NO: 1, and a VL comprising the amino acid sequence of SEQ ID NO: 15. In an embodiment, the antibody molecule comprises: a VH comprising the amino acid sequence of SEQ ID NO: 1, and a VL comprising the amino acid sequence of SEQ ID NO: 16. In an embodiment, the antibody molecule comprises: a VH comprising the amino acid sequence of SEQ ID NO: 1, and a VL comprising the amino acid sequence of SEQ ID NO: 17. In an embodiment, the antibody molecule comprises: a VH comprising the amino acid sequence of SEQ ID NO: 1, and a VL comprising the amino acid sequence of SEQ ID NO: 18. In an embodiment, the antibody molecule comprises: a VH comprising the amino acid sequence of SEQ ID NO: 1, and a VL comprising the amino acid sequence of SEQ ID NO: 19. In an embodiment, the antibody molecule comprises: a VH comprising the amino acid sequence of SEQ ID NO: 1, and a VL comprising the amino acid sequence of SEQ ID NO: 20. In an embodiment, the antibody molecule comprises: a VH comprising the amino acid sequence of SEQ ID NO: 1, and a VL comprising the amino acid sequence of SEQ ID NO: 21. In an embodiment, the antibody molecule comprises: a VH comprising the amino acid sequence of SEQ ID NO: 1, and a VL comprising the amino acid sequence of SEQ ID NO: 22. In an embodiment, the antibody molecule comprises: a VH comprising the amino acid sequence of SEQ ID NO: 1, and a VL comprising the amino acid sequence of SEQ ID NO: 23. In an embodiment, the antibody molecule comprises: a VH comprising the amino acid sequence of SEQ ID NO: 1, and a VL comprising the amino acid sequence of SEQ ID NO: 24. In an embodiment, the antibody molecule comprises: a VH comprising the amino acid sequence of SEQ ID NO: 1, and a VL comprising the amino acid sequence of SEQ ID NO: 25. In an embodiment, the antibody molecule comprises: a VH comprising the amino acid sequence of SEQ ID NO: 1, and a VL comprising the amino acid sequence of SEQ ID NO: 26. In an embodiment, the antibody molecule comprises: a VH comprising the amino acid sequence of SEQ ID NO: 1, and a VL comprising the amino acid sequence of SEQ ID NO: 27. In an embodiment, the antibody molecule comprises: a VH comprising the amino acid sequence of SEQ ID NO: 1, and a VL comprising the amino acid sequence of SEQ ID NO: 28. In an embodiment, the antibody molecule comprises: a VH comprising the amino acid sequence of SEQ ID NO: 1, and a VL comprising the amino acid sequence of SEQ ID NO: 29. In an embodiment, the antibody molecule comprises: a VH comprising the amino acid sequence of SEQ ID NO: 1, and a VL comprising the amino acid sequence of SEQ ID NO: 30. In an embodiment, the antibody molecule comprises: a VH comprising the amino acid sequence of SEQ ID NO: 1, and a VL comprising the amino acid sequence of SEQ ID NO: 31. In an embodiment, the antibody molecule comprises: a VH comprising the amino acid sequence of SEQ ID NO: 1, and a VL comprising the amino acid sequence of SEQ ID NO: 32. In an embodiment, the antibody molecule comprises: a VH comprising the amino acid sequence of SEQ ID NO: 1, and a VL comprising the amino acid sequence of SEQ ID NO: 33. In an embodiment, the antibody molecule comprises: a VH comprising the amino acid sequence of SEQ ID NO: 1, and a VL comprising the amino acid sequence of SEQ ID NO: 34. In an embodiment, the antibody molecule comprises: a VH comprising the amino acid sequence of SEQ ID NO: 1, and a VL comprising the amino acid sequence of SEQ ID NO: 35. In an embodiment, the antibody molecule comprises: a VH comprising the amino acid sequence of SEQ ID NO: 1, and a VL comprising the amino acid sequence of SEQ ID NO: 36. In an embodiment, the antibody molecule comprises: a VH comprising the amino acid sequence of SEQ ID NO: 1, and a VL comprising the amino acid sequence of SEQ ID NO: 37. In an embodiment, the antibody molecule comprises: a VH comprising the amino acid sequence of SEQ ID NO: 1, and a VL comprising the amino acid sequence of SEQ ID NO: 38. In an embodiment, the antibody molecule comprises: a VH comprising the amino acid sequence of SEQ ID NO: 1, and a VL comprising the amino acid sequence of SEQ ID NO: 92. In an embodiment, the antibody molecule comprises: a VH comprising the amino acid sequence of SEQ ID NO: 1, and a VL comprising the amino acid sequence of SEQ ID NO: 93. In an embodiment, the antibody molecule comprises: a VH comprising the amino acid sequence of SEQ ID NO: 1, and a VL comprising the amino acid sequence of SEQ ID NO: 94. In an embodiment, the antibody molecule comprises: a VH comprising the amino acid sequence of SEQ ID NO: 1, and a VL comprising the amino acid sequence of SEQ ID NO: 95. In an embodiment, the antibody molecule comprises: a VH comprising the amino acid sequence of SEQ ID NO: 1, and a VL comprising the amino acid sequence of SEQ ID NO: 96.

In an embodiment, the antibody molecule comprises: a VH comprising the amino acid sequence of SEQ ID NO: 2, and a VL comprising the amino acid sequence of SEQ ID NO: 14. In an embodiment, the antibody molecule comprises: a VH comprising the amino acid sequence of SEQ ID NO: 2, and a VL comprising the amino acid sequence of SEQ ID NO: 15. In an embodiment, the antibody molecule comprises: a VH comprising the amino acid sequence of SEQ ID NO: 2, and a VL comprising the amino acid sequence of SEQ ID NO: 16. In an embodiment, the antibody molecule comprises: a VH comprising the amino acid sequence of SEQ ID NO: 2, and a VL comprising the amino acid sequence of SEQ ID NO: 17. In an embodiment, the antibody molecule comprises: a VH comprising the amino acid sequence of SEQ ID NO: 2, and a VL comprising the amino acid sequence of SEQ ID NO: 18. In an embodiment, the antibody molecule comprises: a VH comprising the amino acid sequence of SEQ ID NO: 2, and a VL comprising the amino acid sequence of SEQ ID NO: 19. In an embodiment, the antibody molecule comprises: a VH comprising the amino acid sequence of SEQ ID NO: 2, and a VL comprising the amino acid sequence of SEQ ID NO: 20. In an embodiment, the antibody molecule comprises: a VH comprising the amino acid sequence of SEQ ID NO: 2, and a VL comprising the amino acid sequence of SEQ ID NO: 21. In an embodiment, the antibody molecule comprises: a VH comprising the amino acid sequence of SEQ ID NO: 2, and a VL comprising the amino acid sequence of SEQ ID NO: 22. In an embodiment, the antibody molecule comprises: a VH comprising the amino acid sequence of SEQ ID NO: 2, and a VL comprising the amino acid sequence of SEQ ID NO: 23. In an embodiment, the antibody molecule comprises: a VH comprising the amino acid sequence of SEQ ID NO: 2, and a VL comprising the amino acid sequence of SEQ ID NO: 24. In an embodiment, the antibody molecule comprises: a VH comprising the amino acid sequence of SEQ ID NO: 2, and a VL comprising the amino acid sequence of SEQ ID NO: 25. In an embodiment, the antibody molecule comprises: a VH comprising the amino acid sequence of SEQ ID NO: 2, and a VL comprising the amino acid sequence of SEQ ID NO: 26. In an embodiment, the antibody molecule comprises: a VH comprising the amino acid sequence of SEQ ID NO: 2, and a VL comprising the amino acid sequence of SEQ ID NO: 27. In an embodiment, the antibody molecule comprises: a VH comprising the amino acid sequence of SEQ ID NO: 2, and a VL comprising the amino acid sequence of SEQ ID NO: 28. In an embodiment, the antibody molecule comprises: a VH comprising the amino acid sequence of SEQ ID NO: 2, and a VL comprising the amino acid sequence of SEQ ID NO: 29. In an embodiment, the antibody molecule comprises: a VH comprising the amino acid sequence of SEQ ID NO: 2, and a VL comprising the amino acid sequence of SEQ ID NO: 30. In an embodiment, the antibody molecule comprises: a VH comprising the amino acid sequence of SEQ ID NO: 2, and a VL comprising the amino acid sequence of SEQ ID NO: 31. In an embodiment, the antibody molecule comprises: a VH comprising the amino acid sequence of SEQ ID NO: 2, and a VL comprising the amino acid sequence of SEQ ID NO: 32. In an embodiment, the antibody molecule comprises: a VH comprising the amino acid sequence of SEQ ID NO: 2, and a VL comprising the amino acid sequence of SEQ ID NO: 33. In an embodiment, the antibody molecule comprises: a VH comprising the amino acid sequence of SEQ ID NO: 2, and a VL comprising the amino acid sequence of SEQ ID NO: 34. In an embodiment, the antibody molecule comprises: a VH comprising the amino acid sequence of SEQ ID NO: 2, and a VL comprising the amino acid sequence of SEQ ID NO: 35. In an embodiment, the antibody molecule comprises: a VH comprising the amino acid sequence of SEQ ID NO: 2, and a VL comprising the amino acid sequence of SEQ ID NO: 36. In an embodiment, the antibody molecule comprises: a VH comprising the amino acid sequence of SEQ ID NO: 2, and a VL comprising the amino acid sequence of SEQ ID NO: 37. In an embodiment, the antibody molecule comprises: a VH comprising the amino acid sequence of SEQ ID NO: 2, and a VL comprising the amino acid sequence of SEQ ID NO: 38. In an embodiment, the antibody molecule comprises: a VH comprising the amino acid sequence of SEQ ID NO: 2, and a VL comprising the amino acid sequence of SEQ ID NO: 92. In an embodiment, the antibody molecule comprises: a VH comprising the amino acid sequence of SEQ ID NO: 2, and a VL comprising the amino acid sequence of SEQ ID NO: 93. In an embodiment, the antibody molecule comprises: a VH comprising the amino acid sequence of SEQ ID NO: 2, and a VL comprising the amino acid sequence of SEQ ID NO: 94. In an embodiment, the antibody molecule comprises: a VH comprising the amino acid sequence of SEQ ID NO: 2, and a VL comprising the amino acid sequence of SEQ ID NO: 95. In an embodiment, the antibody molecule comprises: a VH comprising the amino acid sequence of SEQ ID NO: 2, and a VL comprising the amino acid sequence of SEQ ID NO: 96.

In an embodiment, the antibody molecule comprises: a VH comprising the amino acid sequence of SEQ ID NO: 3, and a VL comprising the amino acid sequence of SEQ ID NO: 14. In an embodiment, the antibody molecule comprises: a VH comprising the amino acid sequence of SEQ ID NO: 3, and a VL comprising the amino acid sequence of SEQ ID NO: 15. In an embodiment, the antibody molecule comprises: a VH comprising the amino acid sequence of SEQ ID NO: 3, and a VL comprising the amino acid sequence of SEQ ID NO: 16. In an embodiment, the antibody molecule comprises: a VH comprising the amino acid sequence of SEQ ID NO: 3, and a VL comprising the amino acid sequence of SEQ ID NO: 17. In an embodiment, the antibody molecule comprises: a VH comprising the amino acid sequence of SEQ ID NO: 3, and a VL comprising the amino acid sequence of SEQ ID NO: 18. In an embodiment, the antibody molecule comprises: a VH comprising the amino acid sequence of SEQ ID NO: 3, and a VL comprising the amino acid sequence of SEQ ID NO: 19. In an embodiment, the antibody molecule comprises: a VH comprising the amino acid sequence of SEQ ID NO: 3, and a VL comprising the amino acid sequence of SEQ ID NO: 20. In an embodiment, the antibody molecule comprises: a VH comprising the amino acid sequence of SEQ ID NO: 3, and a VL comprising the amino acid sequence of SEQ ID NO: 21. In an embodiment, the antibody molecule comprises: a VH comprising the amino acid sequence of SEQ ID NO: 3, and a VL comprising the amino acid sequence of SEQ ID NO: 22. In an embodiment, the antibody molecule comprises: a VH comprising the amino acid sequence of SEQ ID NO: 3, and a VL comprising the amino acid sequence of SEQ ID NO: 23. In an embodiment, the antibody molecule comprises: a VH comprising the amino acid sequence of SEQ ID NO: 3, and a VL comprising the amino acid sequence of SEQ ID NO: 24. In an embodiment, the antibody molecule comprises: a VH comprising the amino acid sequence of SEQ ID NO: 3, and a VL comprising the amino acid sequence of SEQ ID NO: 25. In an embodiment, the antibody molecule comprises: a VH comprising the amino acid sequence of SEQ ID NO: 3, and a VL comprising the amino acid sequence of SEQ ID NO: 26. In an embodiment, the antibody molecule comprises: a VH comprising the amino acid sequence of SEQ ID NO: 3, and a VL comprising the amino acid sequence of SEQ ID NO: 27. In an embodiment, the antibody molecule comprises: a VH comprising the amino acid sequence of SEQ ID NO: 3, and a VL comprising the amino acid sequence of SEQ ID NO: 28. In an embodiment, the antibody molecule comprises: a VH comprising the amino acid sequence of SEQ ID NO: 3, and a VL comprising the amino acid sequence of SEQ ID NO: 29. In an embodiment, the antibody molecule comprises: a VH comprising the amino acid sequence of SEQ ID NO: 3, and a VL comprising the amino acid sequence of SEQ ID NO: 30. In an embodiment, the antibody molecule comprises: a VH comprising the amino acid sequence of SEQ ID NO: 3, and a VL comprising the amino acid sequence of SEQ ID NO: 31. In an embodiment, the antibody molecule comprises: a VH comprising the amino acid sequence of SEQ ID NO: 3, and a VL comprising the amino acid sequence of SEQ ID NO: 32. In an embodiment, the antibody molecule comprises: a VH comprising the amino acid sequence of SEQ ID NO: 3, and a VL comprising the amino acid sequence of SEQ ID NO: 33. In an embodiment, the antibody molecule comprises: a VH comprising the amino acid sequence of SEQ ID NO: 3, and a VL comprising the amino acid sequence of SEQ ID NO: 34. In an embodiment, the antibody molecule comprises: a VH comprising the amino acid sequence of SEQ ID NO: 3, and a VL comprising the amino acid sequence of SEQ ID NO: 35. In an embodiment, the antibody molecule comprises: a VH comprising the amino acid sequence of SEQ ID NO: 3, and a VL comprising the amino acid sequence of SEQ ID NO: 36. In an embodiment, the antibody molecule comprises: a VH comprising the amino acid sequence of SEQ ID NO: 3, and a VL comprising the amino acid sequence of SEQ ID NO: 37. In an embodiment, the antibody molecule comprises: a VH comprising the amino acid sequence of SEQ ID NO: 3, and a VL comprising the amino acid sequence of SEQ ID NO: 38. In an embodiment, the antibody molecule comprises: a VH comprising the amino acid sequence of SEQ ID NO: 3, and a VL comprising the amino acid sequence of SEQ ID NO: 92. In an embodiment, the antibody molecule comprises: a VH comprising the amino acid sequence of SEQ ID NO: 3, and a VL comprising the amino acid sequence of SEQ ID NO: 93. In an embodiment, the antibody molecule comprises: a VH comprising the amino acid sequence of SEQ ID NO: 3, and a VL comprising the amino acid sequence of SEQ ID NO: 94. In an embodiment, the antibody molecule comprises: a VH comprising the amino acid sequence of SEQ ID NO: 3, and a VL comprising the amino acid sequence of SEQ ID NO: 95. In an embodiment, the antibody molecule comprises: a VH comprising the amino acid sequence of SEQ ID NO: 3, and a VL comprising the amino acid sequence of SEQ ID NO: 96.

In an embodiment, the antibody molecule comprises: a VH comprising the amino acid sequence of SEQ ID NO: 4, and a VL comprising the amino acid sequence of SEQ ID NO: 14. In an embodiment, the antibody molecule comprises: a VH comprising the amino acid sequence of SEQ ID NO: 4, and a VL comprising the amino acid sequence of SEQ ID NO: 15. In an embodiment, the antibody molecule comprises: a VH comprising the amino acid sequence of SEQ ID NO: 4, and a VL comprising the amino acid sequence of SEQ ID NO: 16. In an embodiment, the antibody molecule comprises: a VH comprising the amino acid sequence of SEQ ID NO: 4, and a VL comprising the amino acid sequence of SEQ ID NO: 17. In an embodiment, the antibody molecule comprises: a VH comprising the amino acid sequence of SEQ ID NO: 4, and a VL comprising the amino acid sequence of SEQ ID NO: 18. In an embodiment, the antibody molecule comprises: a VH comprising the amino acid sequence of SEQ ID NO: 4, and a VL comprising the amino acid sequence of SEQ ID NO: 19. In an embodiment, the antibody molecule comprises: a VH comprising the amino acid sequence of SEQ ID NO: 4, and a VL comprising the amino acid sequence of SEQ ID NO: 20. In an embodiment, the antibody molecule comprises: a VH comprising the amino acid sequence of SEQ ID NO: 4, and a VL comprising the amino acid sequence of SEQ ID NO: 21. In an embodiment, the antibody molecule comprises: a VH comprising the amino acid sequence of SEQ ID NO: 4, and a VL comprising the amino acid sequence of SEQ ID NO: 22. In an embodiment, the antibody molecule comprises: a VH comprising the amino acid sequence of SEQ ID NO: 4, and a VL comprising the amino acid sequence of SEQ ID NO: 23. In an embodiment, the antibody molecule comprises: a VH comprising the amino acid sequence of SEQ ID NO: 4, and a VL comprising the amino acid sequence of SEQ ID NO: 24. In an embodiment, the antibody molecule comprises: a VH comprising the amino acid sequence of SEQ ID NO: 4, and a VL comprising the amino acid sequence of SEQ ID NO: 25. In an embodiment, the antibody molecule comprises: a VH comprising the amino acid sequence of SEQ ID NO: 4, and a VL comprising the amino acid sequence of SEQ ID NO: 26. In an embodiment, the antibody molecule comprises: a VH comprising the amino acid sequence of SEQ ID NO: 4, and a VL comprising the amino acid sequence of SEQ ID NO: 27. In an embodiment, the antibody molecule comprises: a VH comprising the amino acid sequence of SEQ ID NO: 4, and a VL comprising the amino acid sequence of SEQ ID NO: 28. In an embodiment, the antibody molecule comprises: a VH comprising the amino acid sequence of SEQ ID NO: 4, and a VL comprising the amino acid sequence of SEQ ID NO: 29. In an embodiment, the antibody molecule comprises: a VH comprising the amino acid sequence of SEQ ID NO: 4, and a VL comprising the amino acid sequence of SEQ ID NO: 30. In an embodiment, the antibody molecule comprises: a VH comprising the amino acid sequence of SEQ ID NO: 4, and a VL comprising the amino acid sequence of SEQ ID NO: 31. In an embodiment, the antibody molecule comprises: a VH comprising the amino acid sequence of SEQ ID NO: 4, and a VL comprising the amino acid sequence of SEQ ID NO: 32. In an embodiment, the antibody molecule comprises: a VH comprising the amino acid sequence of SEQ ID NO: 4, and a VL comprising the amino acid sequence of SEQ ID NO: 33. In an embodiment, the antibody molecule comprises: a VH comprising the amino acid sequence of SEQ ID NO: 4, and a VL comprising the amino acid sequence of SEQ ID NO: 34. In an embodiment, the antibody molecule comprises: a VH comprising the amino acid sequence of SEQ ID NO: 4, and a VL comprising the amino acid sequence of SEQ ID NO: 35. In an embodiment, the antibody molecule comprises: a VH comprising the amino acid sequence of SEQ ID NO: 4, and a VL comprising the amino acid sequence of SEQ ID NO: 36. In an embodiment, the antibody molecule comprises: a VH comprising the amino acid sequence of SEQ ID NO: 4, and a VL comprising the amino acid sequence of SEQ ID NO: 37. In an embodiment, the antibody molecule comprises: a VH comprising the amino acid sequence of SEQ ID NO: 4, and a VL comprising the amino acid sequence of SEQ ID NO: 38. In an embodiment, the antibody molecule comprises: a VH comprising the amino acid sequence of SEQ ID NO: 4, and a VL comprising the amino acid sequence of SEQ ID NO: 92. In an embodiment, the antibody molecule comprises: a VH comprising the amino acid sequence of SEQ ID NO: 4, and a VL comprising the amino acid sequence of SEQ ID NO: 93. In an embodiment, the antibody molecule comprises: a VH comprising the amino acid sequence of SEQ ID NO: 4, and a VL comprising the amino acid sequence of SEQ ID NO: 94. In an embodiment, the antibody molecule comprises: a VH comprising the amino acid sequence of SEQ ID NO: 4, and a VL comprising the amino acid sequence of SEQ ID NO: 95. In an embodiment, the antibody molecule comprises: a VH comprising the amino acid sequence of SEQ ID NO: 4, and a VL comprising the amino acid sequence of SEQ ID NO: 96.

In an embodiment, the antibody molecule comprises: a VH comprising the amino acid sequence of SEQ ID NO: 5, and a VL comprising the amino acid sequence of SEQ ID NO: 14. In an embodiment, the antibody molecule comprises: a VH comprising the amino acid sequence of SEQ ID NO: 5, and a VL comprising the amino acid sequence of SEQ ID NO: 15. In an embodiment, the antibody molecule comprises: a VH comprising the amino acid sequence of SEQ ID NO: 5, and a VL comprising the amino acid sequence of SEQ ID NO: 16. In an embodiment, the antibody molecule comprises: a VH comprising the amino acid sequence of SEQ ID NO: 5, and a VL comprising the amino acid sequence of SEQ ID NO: 17. In an embodiment, the antibody molecule comprises: a VH comprising the amino acid sequence of SEQ ID NO: 5, and a VL comprising the amino acid sequence of SEQ ID NO: 18. In an embodiment, the antibody molecule comprises: a VH comprising the amino acid sequence of SEQ ID NO: 5, and a VL comprising the amino acid sequence of SEQ ID NO: 19. In an embodiment, the antibody molecule comprises: a VH comprising the amino acid sequence of SEQ ID NO: 5, and a VL comprising the amino acid sequence of SEQ ID NO: 20. In an embodiment, the antibody molecule comprises: a VH comprising the amino acid sequence of SEQ ID NO: 5, and a VL comprising the amino acid sequence of SEQ ID NO: 21. In an embodiment, the antibody molecule comprises: a VH comprising the amino acid sequence of SEQ ID NO: 5, and a VL comprising the amino acid sequence of SEQ ID NO: 22. In an embodiment, the antibody molecule comprises: a VH comprising the amino acid sequence of SEQ ID NO: 5, and a VL comprising the amino acid sequence of SEQ ID NO: 23. In an embodiment, the antibody molecule comprises: a VH comprising the amino acid sequence of SEQ ID NO: 5, and a VL comprising the amino acid sequence of SEQ ID NO: 24. In an embodiment, the antibody molecule comprises: a VH comprising the amino acid sequence of SEQ ID NO: 5, and a VL comprising the amino acid sequence of SEQ ID NO: 25. In an embodiment, the antibody molecule comprises: a VH comprising the amino acid sequence of SEQ ID NO: 5, and a VL comprising the amino acid sequence of SEQ ID NO: 26. In an embodiment, the antibody molecule comprises: a VH comprising the amino acid sequence of SEQ ID NO: 5, and a VL comprising the amino acid sequence of SEQ ID NO: 27. In an embodiment, the antibody molecule comprises: a VH comprising the amino acid sequence of SEQ ID NO: 5, and a VL comprising the amino acid sequence of SEQ ID NO: 28. In an embodiment, the antibody molecule comprises: a VH comprising the amino acid sequence of SEQ ID NO: 5, and a VL comprising the amino acid sequence of SEQ ID NO: 29. In an embodiment, the antibody molecule comprises: a VH comprising the amino acid sequence of SEQ ID NO: 5, and a VL comprising the amino acid sequence of SEQ ID NO: 30. In an embodiment, the antibody molecule comprises: a VH comprising the amino acid sequence of SEQ ID NO: 5, and a VL comprising the amino acid sequence of SEQ ID NO: 31. In an embodiment, the antibody molecule comprises: a VH comprising the amino acid sequence of SEQ ID NO: 5, and a VL comprising the amino acid sequence of SEQ ID NO: 32. In an embodiment, the antibody molecule comprises: a VH comprising the amino acid sequence of SEQ ID NO: 5, and a VL comprising the amino acid sequence of SEQ ID NO: 33. In an embodiment, the antibody molecule comprises: a VH comprising the amino acid sequence of SEQ ID NO: 5, and a VL comprising the amino acid sequence of SEQ ID NO: 34. In an embodiment, the antibody molecule comprises: a VH comprising the amino acid sequence of SEQ ID NO: 5, and a VL comprising the amino acid sequence of SEQ ID NO: 35. In an embodiment, the antibody molecule comprises: a VH comprising the amino acid sequence of SEQ ID NO: 5, and a VL comprising the amino acid sequence of SEQ ID NO: 36. In an embodiment, the antibody molecule comprises: a VH comprising the amino acid sequence of SEQ ID NO: 5, and a VL comprising the amino acid sequence of SEQ ID NO: 37. In an embodiment, the antibody molecule comprises: a VH comprising the amino acid sequence of SEQ ID NO: 5, and a VL comprising the amino acid sequence of SEQ ID NO: 38. In an embodiment, the antibody molecule comprises: a VH comprising the amino acid sequence of SEQ ID NO: 5, and a VL comprising the amino acid sequence of SEQ ID NO: 92. In an embodiment, the antibody molecule comprises: a VH comprising the amino acid sequence of SEQ ID NO: 5, and a VL comprising the amino acid sequence of SEQ ID NO: 93. In an embodiment, the antibody molecule comprises: a VH comprising the amino acid sequence of SEQ ID NO: 5, and a VL comprising the amino acid sequence of SEQ ID NO: 94. In an embodiment, the antibody molecule comprises: a VH comprising the amino acid sequence of SEQ ID NO: 5, and a VL comprising the amino acid sequence of SEQ ID NO: 95. In an embodiment, the antibody molecule comprises: a VH comprising the amino acid sequence of SEQ ID NO: 5, and a VL comprising the amino acid sequence of SEQ ID NO: 96.

In an embodiment, the antibody molecule comprises: a VH comprising the amino acid sequence of SEQ ID NO: 6, and a VL comprising the amino acid sequence of SEQ ID NO: 14. In an embodiment, the antibody molecule comprises: a VH comprising the amino acid sequence of SEQ ID NO: 6, and a VL comprising the amino acid sequence of SEQ ID NO: 15. In an embodiment, the antibody molecule comprises: a VH comprising the amino acid sequence of SEQ ID NO: 6, and a VL comprising the amino acid sequence of SEQ ID NO: 16. In an embodiment, the antibody molecule comprises: a VH comprising the amino acid sequence of SEQ ID NO: 6, and a VL comprising the amino acid sequence of SEQ ID NO: 17. In an embodiment, the antibody molecule comprises: a VH comprising the amino acid sequence of SEQ ID NO: 6, and a VL comprising the amino acid sequence of SEQ ID NO: 18. In an embodiment, the antibody molecule comprises: a VH comprising the amino acid sequence of SEQ ID NO: 6, and a VL comprising the amino acid sequence of SEQ ID NO: 19. In an embodiment, the antibody molecule comprises: a VH comprising the amino acid sequence of SEQ ID NO: 6, and a VL comprising the amino acid sequence of SEQ ID NO: 20. In an embodiment, the antibody molecule comprises: a VH comprising the amino acid sequence of SEQ ID NO: 6, and a VL comprising the amino acid sequence of SEQ ID NO: 21. In an embodiment, the antibody molecule comprises: a VH comprising the amino acid sequence of SEQ ID NO: 6, and a VL comprising the amino acid sequence of SEQ ID NO: 22. In an embodiment, the antibody molecule comprises: a VH comprising the amino acid sequence of SEQ ID NO: 6, and a VL comprising the amino acid sequence of SEQ ID NO: 23. In an embodiment, the antibody molecule comprises: a VH comprising the amino acid sequence of SEQ ID NO: 6, and a VL comprising the amino acid sequence of SEQ ID NO: 24. In an embodiment, the antibody molecule comprises: a VH comprising the amino acid sequence of SEQ ID NO: 6, and a VL comprising the amino acid sequence of SEQ ID NO: 25. In an embodiment, the antibody molecule comprises: a VH comprising the amino acid sequence of SEQ ID NO: 6, and a VL comprising the amino acid sequence of SEQ ID NO: 26. In an embodiment, the antibody molecule comprises: a VH comprising the amino acid sequence of SEQ ID NO: 6, and a VL comprising the amino acid sequence of SEQ ID NO: 27. In an embodiment, the antibody molecule comprises: a VH comprising the amino acid sequence of SEQ ID NO: 6, and a VL comprising the amino acid sequence of SEQ ID NO: 28. In an embodiment, the antibody molecule comprises: a VH comprising the amino acid sequence of SEQ ID NO: 6, and a VL comprising the amino acid sequence of SEQ ID NO: 29. In an embodiment, the antibody molecule comprises: a VH comprising the amino acid sequence of SEQ ID NO: 6, and a VL comprising the amino acid sequence of SEQ ID NO: 30. In an embodiment, the antibody molecule comprises: a VH comprising the amino acid sequence of SEQ ID NO: 6, and a VL comprising the amino acid sequence of SEQ ID NO: 31. In an embodiment, the antibody molecule comprises: a VH comprising the amino acid sequence of SEQ ID NO: 6, and a VL comprising the amino acid sequence of SEQ ID NO: 32. In an embodiment, the antibody molecule comprises: a VH comprising the amino acid sequence of SEQ ID NO: 6, and a VL comprising the amino acid sequence of SEQ ID NO: 33. In an embodiment, the antibody molecule comprises: a VH comprising the amino acid sequence of SEQ ID NO: 6, and a VL comprising the amino acid sequence of SEQ ID NO: 34. In an embodiment, the antibody molecule comprises: a VH comprising the amino acid sequence of SEQ ID NO: 6, and a VL comprising the amino acid sequence of SEQ ID NO: 35. In an embodiment, the antibody molecule comprises: a VH comprising the amino acid sequence of SEQ ID NO: 6, and a VL comprising the amino acid sequence of SEQ ID NO: 36. In an embodiment, the antibody molecule comprises: a VH comprising the amino acid sequence of SEQ ID NO: 6, and a VL comprising the amino acid sequence of SEQ ID NO: 37. In an embodiment, the antibody molecule comprises: a VH comprising the amino acid sequence of SEQ ID NO: 6, and a VL comprising the amino acid sequence of SEQ ID NO: 38. In an embodiment, the antibody molecule comprises: a VH comprising the amino acid sequence of SEQ ID NO: 6, and a VL comprising the amino acid sequence of SEQ ID NO: 92. In an embodiment, the antibody molecule comprises: a VH comprising the amino acid sequence of SEQ ID NO: 6, and a VL comprising the amino acid sequence of SEQ ID NO: 93. In an embodiment, the antibody molecule comprises: a VH comprising the amino acid sequence of SEQ ID NO: 6, and a VL comprising the amino acid sequence of SEQ ID NO: 94. In an embodiment, the antibody molecule comprises: a VH comprising the amino acid sequence of SEQ ID NO: 6, and a VL comprising the amino acid sequence of SEQ ID NO: 95. In an embodiment, the antibody molecule comprises: a VH comprising the amino acid sequence of SEQ ID NO: 6, and a VL comprising the amino acid sequence of SEQ ID NO: 96.

In an embodiment, the antibody molecule comprises: a VH comprising the amino acid sequence of SEQ ID NO: 7, and a VL comprising the amino acid sequence of SEQ ID NO: 14. In an embodiment, the antibody molecule comprises: a VH comprising the amino acid sequence of SEQ ID NO: 7, and a VL comprising the amino acid sequence of SEQ ID NO: 15. In an embodiment, the antibody molecule comprises: a VH comprising the amino acid sequence of SEQ ID NO: 7, and a VL comprising the amino acid sequence of SEQ ID NO: 16. In an embodiment, the antibody molecule comprises: a VH comprising the amino acid sequence of SEQ ID NO: 7, and a VL comprising the amino acid sequence of SEQ ID NO: 17. In an embodiment, the antibody molecule comprises: a VH comprising the amino acid sequence of SEQ ID NO: 7, and a VL comprising the amino acid sequence of SEQ ID NO: 18. In an embodiment, the antibody molecule comprises: a VH comprising the amino acid sequence of SEQ ID NO: 7, and a VL comprising the amino acid sequence of SEQ ID NO: 19. In an embodiment, the antibody molecule comprises: a VH comprising the amino acid sequence of SEQ ID NO: 7, and a VL comprising the amino acid sequence of SEQ ID NO: 20. In an embodiment, the antibody molecule comprises: a VH comprising the amino acid sequence of SEQ ID NO: 7, and a VL comprising the amino acid sequence of SEQ ID NO: 21. In an embodiment, the antibody molecule comprises: a VH comprising the amino acid sequence of SEQ ID NO: 7, and a VL comprising the amino acid sequence of SEQ ID NO: 22. In an embodiment, the antibody molecule comprises: a VH comprising the amino acid sequence of SEQ ID NO: 7, and a VL comprising the amino acid sequence of SEQ ID NO: 23. In an embodiment, the antibody molecule comprises: a VH comprising the amino acid sequence of SEQ ID NO: 7, and a VL comprising the amino acid sequence of SEQ ID NO: 24. In an embodiment, the antibody molecule comprises: a VH comprising the amino acid sequence of SEQ ID NO: 7, and a VL comprising the amino acid sequence of SEQ ID NO: 25. In an embodiment, the antibody molecule comprises: a VH comprising the amino acid sequence of SEQ ID NO: 7, and a VL comprising the amino acid sequence of SEQ ID NO: 26. In an embodiment, the antibody molecule comprises: a VH comprising the amino acid sequence of SEQ ID NO: 7, and a VL comprising the amino acid sequence of SEQ ID NO: 27. In an embodiment, the antibody molecule comprises: a VH comprising the amino acid sequence of SEQ ID NO: 7, and a VL comprising the amino acid sequence of SEQ ID NO: 28. In an embodiment, the antibody molecule comprises: a VH comprising the amino acid sequence of SEQ ID NO: 7, and a VL comprising the amino acid sequence of SEQ ID NO: 29. In an embodiment, the antibody molecule comprises: a VH comprising the amino acid sequence of SEQ ID NO: 7, and a VL comprising the amino acid sequence of SEQ ID NO: 30. In an embodiment, the antibody molecule comprises: a VH comprising the amino acid sequence of SEQ ID NO: 7, and a VL comprising the amino acid sequence of SEQ ID NO: 31. In an embodiment, the antibody molecule comprises: a VH comprising the amino acid sequence of SEQ ID NO: 7, and a VL comprising the amino acid sequence of SEQ ID NO: 32. In an embodiment, the antibody molecule comprises: a VH comprising the amino acid sequence of SEQ ID NO: 7, and a VL comprising the amino acid sequence of SEQ ID NO: 33. In an embodiment, the antibody molecule comprises: a VH comprising the amino acid sequence of SEQ ID NO: 7, and a VL comprising the amino acid sequence of SEQ ID NO: 34. In an embodiment, the antibody molecule comprises: a VH comprising the amino acid sequence of SEQ ID NO: 7, and a VL comprising the amino acid sequence of SEQ ID NO: 35. In an embodiment, the antibody molecule comprises: a VH comprising the amino acid sequence of SEQ ID NO: 7, and a VL comprising the amino acid sequence of SEQ ID NO: 36. In an embodiment, the antibody molecule comprises: a VH comprising the amino acid sequence of SEQ ID NO: 7, and a VL comprising the amino acid sequence of SEQ ID NO: 37. In an embodiment, the antibody molecule comprises: a VH comprising the amino acid sequence of SEQ ID NO: 7, and a VL comprising the amino acid sequence of SEQ ID NO: 38. In an embodiment, the antibody molecule comprises: a VH comprising the amino acid sequence of SEQ ID NO: 7, and a VL comprising the amino acid sequence of SEQ ID NO: 92. In an embodiment, the antibody molecule comprises: a VH comprising the amino acid sequence of SEQ ID NO: 7, and a VL comprising the amino acid sequence of SEQ ID NO: 93. In an embodiment, the antibody molecule comprises: a VH comprising the amino acid sequence of SEQ ID NO: 7, and a VL comprising the amino acid sequence of SEQ ID NO: 94. In an embodiment, the antibody molecule comprises: a VH comprising the amino acid sequence of SEQ ID NO: 7, and a VL comprising the amino acid sequence of SEQ ID NO: 95. In an embodiment, the antibody molecule comprises: a VH comprising the amino acid sequence of SEQ ID NO: 7, and a VL comprising the amino acid sequence of SEQ ID NO: 96.

In an embodiment, the antibody molecule comprises: a VH comprising the amino acid sequence of SEQ ID NO: 8, and a VL comprising the amino acid sequence of SEQ ID NO: 14. In an embodiment, the antibody molecule comprises: a VH comprising the amino acid sequence of SEQ ID NO: 8, and a VL comprising the amino acid sequence of SEQ ID NO: 15. In an embodiment, the antibody molecule comprises: a VH comprising the amino acid sequence of SEQ ID NO: 8, and a VL comprising the amino acid sequence of SEQ ID NO: 16. In an embodiment, the antibody molecule comprises: a VH comprising the amino acid sequence of SEQ ID NO: 8, and a VL comprising the amino acid sequence of SEQ ID NO: 17. In an embodiment, the antibody molecule comprises: a VH comprising the amino acid sequence of SEQ ID NO: 8, and a VL comprising the amino acid sequence of SEQ ID NO: 18. In an embodiment, the antibody molecule comprises: a VH comprising the amino acid sequence of SEQ ID NO: 8, and a VL comprising the amino acid sequence of SEQ ID NO: 19. In an embodiment, the antibody molecule comprises: a VH comprising the amino acid sequence of SEQ ID NO: 8, and a VL comprising the amino acid sequence of SEQ ID NO: 20. In an embodiment, the antibody molecule comprises: a VH comprising the amino acid sequence of SEQ ID NO: 8, and a VL comprising the amino acid sequence of SEQ ID NO: 21. In an embodiment, the antibody molecule comprises: a VH comprising the amino acid sequence of SEQ ID NO: 8, and a VL comprising the amino acid sequence of SEQ ID NO: 22. In an embodiment, the antibody molecule comprises: a VH comprising the amino acid sequence of SEQ ID NO: 8, and a VL comprising the amino acid sequence of SEQ ID NO: 23. In an embodiment, the antibody molecule comprises: a VH comprising the amino acid sequence of SEQ ID NO: 8, and a VL comprising the amino acid sequence of SEQ ID NO: 24. In an embodiment, the antibody molecule comprises: a VH comprising the amino acid sequence of SEQ ID NO: 8, and a VL comprising the amino acid sequence of SEQ ID NO: 25. In an embodiment, the antibody molecule comprises: a VH comprising the amino acid sequence of SEQ ID NO: 8, and a VL comprising the amino acid sequence of SEQ ID NO: 26. In an embodiment, the antibody molecule comprises: a VH comprising the amino acid sequence of SEQ ID NO: 8, and a VL comprising the amino acid sequence of SEQ ID NO: 27. In an embodiment, the antibody molecule comprises: a VH comprising the amino acid sequence of SEQ ID NO: 8, and a VL comprising the amino acid sequence of SEQ ID NO: 28. In an embodiment, the antibody molecule comprises: a VH comprising the amino acid sequence of SEQ ID NO: 8, and a VL comprising the amino acid sequence of SEQ ID NO: 29. In an embodiment, the antibody molecule comprises: a VH comprising the amino acid sequence of SEQ ID NO: 8, and a VL comprising the amino acid sequence of SEQ ID NO: 30. In an embodiment, the antibody molecule comprises: a VH comprising the amino acid sequence of SEQ ID NO: 8, and a VL comprising the amino acid sequence of SEQ ID NO: 31. In an embodiment, the antibody molecule comprises: a VH comprising the amino acid sequence of SEQ ID NO: 8, and a VL comprising the amino acid sequence of SEQ ID NO: 32. In an embodiment, the antibody molecule comprises: a VH comprising the amino acid sequence of SEQ ID NO: 8, and a VL comprising the amino acid sequence of SEQ ID NO: 33. In an embodiment, the antibody molecule comprises: a VH comprising the amino acid sequence of SEQ ID NO: 8, and a VL comprising the amino acid sequence of SEQ ID NO: 34. In an embodiment, the antibody molecule comprises: a VH comprising the amino acid sequence of SEQ ID NO: 8, and a VL comprising the amino acid sequence of SEQ ID NO: 35. In an embodiment, the antibody molecule comprises: a VH comprising the amino acid sequence of SEQ ID NO: 8, and a VL comprising the amino acid sequence of SEQ ID NO: 36. In an embodiment, the antibody molecule comprises: a VH comprising the amino acid sequence of SEQ ID NO: 8, and a VL comprising the amino acid sequence of SEQ ID NO: 37. In an embodiment, the antibody molecule comprises: a VH comprising the amino acid sequence of SEQ ID NO: 8, and a VL comprising the amino acid sequence of SEQ ID NO: 38. In an embodiment, the antibody molecule comprises: a VH comprising the amino acid sequence of SEQ ID NO: 8, and a VL comprising the amino acid sequence of SEQ ID NO: 92. In an embodiment, the antibody molecule comprises: a VH comprising the amino acid sequence of SEQ ID NO: 8, and a VL comprising the amino acid sequence of SEQ ID NO: 93. In an embodiment, the antibody molecule comprises: a VH comprising the amino acid sequence of SEQ ID NO: 8, and a VL comprising the amino acid sequence of SEQ ID NO: 94. In an embodiment, the antibody molecule comprises: a VH comprising the amino acid sequence of SEQ ID NO: 8, and a VL comprising the amino acid sequence of SEQ ID NO: 95. In an embodiment, the antibody molecule comprises: a VH comprising the amino acid sequence of SEQ ID NO: 8, and a VL comprising the amino acid sequence of SEQ ID NO: 96.

In an embodiment, the antibody molecule comprises: a VH comprising the amino acid sequence of SEQ ID NO: 9, and a VL comprising the amino acid sequence of SEQ ID NO: 14. In an embodiment, the antibody molecule comprises: a VH comprising the amino acid sequence of SEQ ID NO: 9, and a VL comprising the amino acid sequence of SEQ ID NO: 15. In an embodiment, the antibody molecule comprises: a VH comprising the amino acid sequence of SEQ ID NO: 9, and a VL comprising the amino acid sequence of SEQ ID NO: 16. In an embodiment, the antibody molecule comprises: a VH comprising the amino acid sequence of SEQ ID NO: 9, and a VL comprising the amino acid sequence of SEQ ID NO: 17. In an embodiment, the antibody molecule comprises: a VH comprising the amino acid sequence of SEQ ID NO: 9, and a VL comprising the amino acid sequence of SEQ ID NO: 18. In an embodiment, the antibody molecule comprises: a VH comprising the amino acid sequence of SEQ ID NO: 9, and a VL comprising the amino acid sequence of SEQ ID NO: 19. In an embodiment, the antibody molecule comprises: a VH comprising the amino acid sequence of SEQ ID NO: 9, and a VL comprising the amino acid sequence of SEQ ID NO: 20. In an embodiment, the antibody molecule comprises: a VH comprising the amino acid sequence of SEQ ID NO: 9, and a VL comprising the amino acid sequence of SEQ ID NO: 21. In an embodiment, the antibody molecule comprises: a VH comprising the amino acid sequence of SEQ ID NO: 9, and a VL comprising the amino acid sequence of SEQ ID NO: 22. In an embodiment, the antibody molecule comprises: a VH comprising the amino acid sequence of SEQ ID NO: 9, and a VL comprising the amino acid sequence of SEQ ID NO: 23. In an embodiment, the antibody molecule comprises: a VH comprising the amino acid sequence of SEQ ID NO: 9, and a VL comprising the amino acid sequence of SEQ ID NO: 24. In an embodiment, the antibody molecule comprises: a VH comprising the amino acid sequence of SEQ ID NO: 9, and a VL comprising the amino acid sequence of SEQ ID NO: 25. In an embodiment, the antibody molecule comprises: a VH comprising the amino acid sequence of SEQ ID NO: 9, and a VL comprising the amino acid sequence of SEQ ID NO: 26. In an embodiment, the antibody molecule comprises: a VH comprising the amino acid sequence of SEQ ID NO: 9, and a VL comprising the amino acid sequence of SEQ ID NO: 27. In an embodiment, the antibody molecule comprises: a VH comprising the amino acid sequence of SEQ ID NO: 9, and a VL comprising the amino acid sequence of SEQ ID NO: 28. In an embodiment, the antibody molecule comprises: a VH comprising the amino acid sequence of SEQ ID NO: 9, and a VL comprising the amino acid sequence of SEQ ID NO: 29. In an embodiment, the antibody molecule comprises: a VH comprising the amino acid sequence of SEQ ID NO: 9, and a VL comprising the amino acid sequence of SEQ ID NO: 30. In an embodiment, the antibody molecule comprises: a VH comprising the amino acid sequence of SEQ ID NO: 9, and a VL comprising the amino acid sequence of SEQ ID NO: 31. In an embodiment, the antibody molecule comprises: a VH comprising the amino acid sequence of SEQ ID NO: 9, and a VL comprising the amino acid sequence of SEQ ID NO: 32. In an embodiment, the antibody molecule comprises: a VH comprising the amino acid sequence of SEQ ID NO: 9, and a VL comprising the amino acid sequence of SEQ ID NO: 33. In an embodiment, the antibody molecule comprises: a VH comprising the amino acid sequence of SEQ ID NO: 9, and a VL comprising the amino acid sequence of SEQ ID NO: 34. In an embodiment, the antibody molecule comprises: a VH comprising the amino acid sequence of SEQ ID NO: 9, and a VL comprising the amino acid sequence of SEQ ID NO: 35. In an embodiment, the antibody molecule comprises: a VH comprising the amino acid sequence of SEQ ID NO: 9, and a VL comprising the amino acid sequence of SEQ ID NO: 36. In an embodiment, the antibody molecule comprises: a VH comprising the amino acid sequence of SEQ ID NO: 9, and a VL comprising the amino acid sequence of SEQ ID NO: 37. In an embodiment, the antibody molecule comprises: a VH comprising the amino acid sequence of SEQ ID NO: 9, and a VL comprising the amino acid sequence of SEQ ID NO: 38. In an embodiment, the antibody molecule comprises: a VH comprising the amino acid sequence of SEQ ID NO: 9, and a VL comprising the amino acid sequence of SEQ ID NO: 92. In an embodiment, the antibody molecule comprises: a VH comprising the amino acid sequence of SEQ ID NO: 9, and a VL comprising the amino acid sequence of SEQ ID NO: 93. In an embodiment, the antibody molecule comprises: a VH comprising the amino acid sequence of SEQ ID NO: 9, and a VL comprising the amino acid sequence of SEQ ID NO: 94. In an embodiment, the antibody molecule comprises: a VH comprising the amino acid sequence of SEQ ID NO: 9, and a VL comprising the amino acid sequence of SEQ ID NO: 95. In an embodiment, the antibody molecule comprises: a VH comprising the amino acid sequence of SEQ ID NO: 9, and a VL comprising the amino acid sequence of SEQ ID NO: 96.

In an embodiment, the antibody molecule comprises: a VH comprising the amino acid sequence of SEQ ID NO: 10, and a VL comprising the amino acid sequence of SEQ ID NO: 14. In an embodiment, the antibody molecule comprises: a VH comprising the amino acid sequence of SEQ ID NO: 10, and a VL comprising the amino acid sequence of SEQ ID NO: 15. In an embodiment, the antibody molecule comprises: a VH comprising the amino acid sequence of SEQ ID NO: 10, and a VL comprising the amino acid sequence of SEQ ID NO: 16. In an embodiment, the antibody molecule comprises: a VH comprising the amino acid sequence of SEQ ID NO: 10, and a VL comprising the amino acid sequence of SEQ ID NO: 17. In an embodiment, the antibody molecule comprises: a VH comprising the amino acid sequence of SEQ ID NO: 10, and a VL comprising the amino acid sequence of SEQ ID NO: 18. In an embodiment, the antibody molecule comprises: a VH comprising the amino acid sequence of SEQ ID NO: 10, and a VL comprising the amino acid sequence of SEQ ID NO: 19. In an embodiment, the antibody molecule comprises: a VH comprising the amino acid sequence of SEQ ID NO: 10, and a VL comprising the amino acid sequence of SEQ ID NO: 20. In an embodiment, the antibody molecule comprises: a VH comprising the amino acid sequence of SEQ ID NO: 10, and a VL comprising the amino acid sequence of SEQ ID NO: 21. In an embodiment, the antibody molecule comprises: a VH comprising the amino acid sequence of SEQ ID NO: 10, and a VL comprising the amino acid sequence of SEQ ID NO: 22. In an embodiment, the antibody molecule comprises: a VH comprising the amino acid sequence of SEQ ID NO: 10, and a VL comprising the amino acid sequence of SEQ ID NO: 23. In an embodiment, the antibody molecule comprises: a VH comprising the amino acid sequence of SEQ ID NO: 10, and a VL comprising the amino acid sequence of SEQ ID NO: 24. In an embodiment, the antibody molecule comprises: a VH comprising the amino acid sequence of SEQ ID NO: 10, and a VL comprising the amino acid sequence of SEQ ID NO: 25. In an embodiment, the antibody molecule comprises: a VH comprising the amino acid sequence of SEQ ID NO: 10, and a VL comprising the amino acid sequence of SEQ ID NO: 26. In an embodiment, the antibody molecule comprises: a VH comprising the amino acid sequence of SEQ ID NO: 10, and a VL comprising the amino acid sequence of SEQ ID NO: 27. In an embodiment, the antibody molecule comprises: a VH comprising the amino acid sequence of SEQ ID NO: 10, and a VL comprising the amino acid sequence of SEQ ID NO: 28. In an embodiment, the antibody molecule comprises: a VH comprising the amino acid sequence of SEQ ID NO: 10, and a VL comprising the amino acid sequence of SEQ ID NO: 29. In an embodiment, the antibody molecule comprises: a VH comprising the amino acid sequence of SEQ ID NO: 10, and a VL comprising the amino acid sequence of SEQ ID NO: 30. In an embodiment, the antibody molecule comprises: a VH comprising the amino acid sequence of SEQ ID NO: 10, and a VL comprising the amino acid sequence of SEQ ID NO: 31. In an embodiment, the antibody molecule comprises: a VH comprising the amino acid sequence of SEQ ID NO: 10, and a VL comprising the amino acid sequence of SEQ ID NO: 32. In an embodiment, the antibody molecule comprises: a VH comprising the amino acid sequence of SEQ ID NO: 10, and a VL comprising the amino acid sequence of SEQ ID NO: 33. In an embodiment, the antibody molecule comprises: a VH comprising the amino acid sequence of SEQ ID NO: 10, and a VL comprising the amino acid sequence of SEQ ID NO: 34. In an embodiment, the antibody molecule comprises: a VH comprising the amino acid sequence of SEQ ID NO: 10, and a VL comprising the amino acid sequence of SEQ ID NO: 35. In an embodiment, the antibody molecule comprises: a VH comprising the amino acid sequence of SEQ ID NO: 10, and a VL comprising the amino acid sequence of SEQ ID NO: 36. In an embodiment, the antibody molecule comprises: a VH comprising the amino acid sequence of SEQ ID NO: 10, and a VL comprising the amino acid sequence of SEQ ID NO: 37. In an embodiment, the antibody molecule comprises: a VH comprising the amino acid sequence of SEQ ID NO: 10, and a VL comprising the amino acid sequence of SEQ ID NO: 38. In an embodiment, the antibody molecule comprises: a VH comprising the amino acid sequence of SEQ ID NO: 10, and a VL comprising the amino acid sequence of SEQ ID NO: 92. In an embodiment, the antibody molecule comprises: a VH comprising the amino acid sequence of SEQ ID NO: 10, and a VL comprising the amino acid sequence of SEQ ID NO: 93. In an embodiment, the antibody molecule comprises: a VH comprising the amino acid sequence of SEQ ID NO: 10, and a VL comprising the amino acid sequence of SEQ ID NO: 94. In an embodiment, the antibody molecule comprises: a VH comprising the amino acid sequence of SEQ ID NO: 10, and a VL comprising the amino acid sequence of SEQ ID NO: 95. In an embodiment, the antibody molecule comprises: a VH comprising the amino acid sequence of SEQ ID NO: 10, and a VL comprising the amino acid sequence of SEQ ID NO: 96.

In an embodiment, the antibody molecule comprises: a VH comprising the amino acid sequence of SEQ ID NO: 11, and a VL comprising the amino acid sequence of SEQ ID NO: 14. In an embodiment, the antibody molecule comprises: a VH comprising the amino acid sequence of SEQ ID NO: 11, and a VL comprising the amino acid sequence of SEQ ID NO: 15. In an embodiment, the antibody molecule comprises: a VH comprising the amino acid sequence of SEQ ID NO: 11, and a VL comprising the amino acid sequence of SEQ ID NO: 16. In an embodiment, the antibody molecule comprises: a VH comprising the amino acid sequence of SEQ ID NO: 11, and a VL comprising the amino acid sequence of SEQ ID NO: 17. In an embodiment, the antibody molecule comprises: a VH comprising the amino acid sequence of SEQ ID NO: 11, and a VL comprising the amino acid sequence of SEQ ID NO: 18. In an embodiment, the antibody molecule comprises: a VH comprising the amino acid sequence of SEQ ID NO: 11, and a VL comprising the amino acid sequence of SEQ ID NO: 19. In an embodiment, the antibody molecule comprises: a VH comprising the amino acid sequence of SEQ ID NO: 11, and a VL comprising the amino acid sequence of SEQ ID NO: 20. In an embodiment, the antibody molecule comprises: a VH comprising the amino acid sequence of SEQ ID NO: 11, and a VL comprising the amino acid sequence of SEQ ID NO: 21. In an embodiment, the antibody molecule comprises: a VH comprising the amino acid sequence of SEQ ID NO: 11, and a VL comprising the amino acid sequence of SEQ ID NO: 22. In an embodiment, the antibody molecule comprises: a VH comprising the amino acid sequence of SEQ ID NO: 11, and a VL comprising the amino acid sequence of SEQ ID NO: 23. In an embodiment, the antibody molecule comprises: a VH comprising the amino acid sequence of SEQ ID NO: 11, and a VL comprising the amino acid sequence of SEQ ID NO: 24. In an embodiment, the antibody molecule comprises: a VH comprising the amino acid sequence of SEQ ID NO: 11, and a VL comprising the amino acid sequence of SEQ ID NO: 25. In an embodiment, the antibody molecule comprises: a VH comprising the amino acid sequence of SEQ ID NO: 11, and a VL comprising the amino acid sequence of SEQ ID NO: 26. In an embodiment, the antibody molecule comprises: a VH comprising the amino acid sequence of SEQ ID NO: 11, and a VL comprising the amino acid sequence of SEQ ID NO: 27. In an embodiment, the antibody molecule comprises: a VH comprising the amino acid sequence of SEQ ID NO: 11, and a VL comprising the amino acid sequence of SEQ ID NO: 28. In an embodiment, the antibody molecule comprises: a VH comprising the amino acid sequence of SEQ ID NO: 11, and a VL comprising the amino acid sequence of SEQ ID NO: 29. In an embodiment, the antibody molecule comprises: a VH comprising the amino acid sequence of SEQ ID NO: 11, and a VL comprising the amino acid sequence of SEQ ID NO: 30. In an embodiment, the antibody molecule comprises: a VH comprising the amino acid sequence of SEQ ID NO: 11, and a VL comprising the amino acid sequence of SEQ ID NO: 31. In an embodiment, the antibody molecule comprises: a VH comprising the amino acid sequence of SEQ ID NO: 11, and a VL comprising the amino acid sequence of SEQ ID NO: 32. In an embodiment, the antibody molecule comprises: a VH comprising the amino acid sequence of SEQ ID NO: 11, and a VL comprising the amino acid sequence of SEQ ID NO: 33. In an embodiment, the antibody molecule comprises: a VH comprising the amino acid sequence of SEQ ID NO: 11, and a VL comprising the amino acid sequence of SEQ ID NO: 34. In an embodiment, the antibody molecule comprises: a VH comprising the amino acid sequence of SEQ ID NO: 11, and a VL comprising the amino acid sequence of SEQ ID NO: 35. In an embodiment, the antibody molecule comprises: a VH comprising the amino acid sequence of SEQ ID NO: 11, and a VL comprising the amino acid sequence of SEQ ID NO: 36. In an embodiment, the antibody molecule comprises: a VH comprising the amino acid sequence of SEQ ID NO: 11, and a VL comprising the amino acid sequence of SEQ ID NO: 37. In an embodiment, the antibody molecule comprises: a VH comprising the amino acid sequence of SEQ ID NO: 11, and a VL comprising the amino acid sequence of SEQ ID NO: 38. In an embodiment, the antibody molecule comprises: a VH comprising the amino acid sequence of SEQ ID NO: 11, and a VL comprising the amino acid sequence of SEQ ID NO: 92. In an embodiment, the antibody molecule comprises: a VH comprising the amino acid sequence of SEQ ID NO: 11, and a VL comprising the amino acid sequence of SEQ ID NO: 93. In an embodiment, the antibody molecule comprises: a VH comprising the amino acid sequence of SEQ ID NO: 11, and a VL comprising the amino acid sequence of SEQ ID NO: 94. In an embodiment, the antibody molecule comprises: a VH comprising the amino acid sequence of SEQ ID NO: 11, and a VL comprising the amino acid sequence of SEQ ID NO: 95. In an embodiment, the antibody molecule comprises: a VH comprising the amino acid sequence of SEQ ID NO: 11, and a VL comprising the amino acid sequence of SEQ ID NO: 96.

In an embodiment, the antibody molecule comprises: a VH comprising the amino acid sequence of SEQ ID NO: 12, and a VL comprising the amino acid sequence of SEQ ID NO: 14. In an embodiment, the antibody molecule comprises: a VH comprising the amino acid sequence of SEQ ID NO: 12, and a VL comprising the amino acid sequence of SEQ ID NO: 15. In an embodiment, the antibody molecule comprises: a VH comprising the amino acid sequence of SEQ ID NO: 12, and a VL comprising the amino acid sequence of SEQ ID NO: 16. In an embodiment, the antibody molecule comprises: a VH comprising the amino acid sequence of SEQ ID NO: 12, and a VL comprising the amino acid sequence of SEQ ID NO: 17. In an embodiment, the antibody molecule comprises: a VH comprising the amino acid sequence of SEQ ID NO: 12, and a VL comprising the amino acid sequence of SEQ ID NO: 18. In an embodiment, the antibody molecule comprises: a VH comprising the amino acid sequence of SEQ ID NO: 12, and a VL comprising the amino acid sequence of SEQ ID NO: 19. In an embodiment, the antibody molecule comprises: a VH comprising the amino acid sequence of SEQ ID NO: 12, and a VL comprising the amino acid sequence of SEQ ID NO: 20. In an embodiment, the antibody molecule comprises: a VH comprising the amino acid sequence of SEQ ID NO: 12, and a VL comprising the amino acid sequence of SEQ ID NO: 21. In an embodiment, the antibody molecule comprises: a VH comprising the amino acid sequence of SEQ ID NO: 12, and a VL comprising the amino acid sequence of SEQ ID NO: 22. In an embodiment, the antibody molecule comprises: a VH comprising the amino acid sequence of SEQ ID NO: 12, and a VL comprising the amino acid sequence of SEQ ID NO: 23. In an embodiment, the antibody molecule comprises: a VH comprising the amino acid sequence of SEQ ID NO: 12, and a VL comprising the amino acid sequence of SEQ ID NO: 24. In an embodiment, the antibody molecule comprises: a VH comprising the amino acid sequence of SEQ ID NO: 12, and a VL comprising the amino acid sequence of SEQ ID NO: 25. In an embodiment, the antibody molecule comprises: a VH comprising the amino acid sequence of SEQ ID NO: 12, and a VL comprising the amino acid sequence of SEQ ID NO: 26. In an embodiment, the antibody molecule comprises: a VH comprising the amino acid sequence of SEQ ID NO: 12, and a VL comprising the amino acid sequence of SEQ ID NO: 27. In an embodiment, the antibody molecule comprises: a VH comprising the amino acid sequence of SEQ ID NO: 12, and a VL comprising the amino acid sequence of SEQ ID NO: 28. In an embodiment, the antibody molecule comprises: a VH comprising the amino acid sequence of SEQ ID NO: 12, and a VL comprising the amino acid sequence of SEQ ID NO: 29. In an embodiment, the antibody molecule comprises: a VH comprising the amino acid sequence of SEQ ID NO: 12, and a VL comprising the amino acid sequence of SEQ ID NO: 30. In an embodiment, the antibody molecule comprises: a VH comprising the amino acid sequence of SEQ ID NO: 12, and a VL comprising the amino acid sequence of SEQ ID NO: 31. In an embodiment, the antibody molecule comprises: a VH comprising the amino acid sequence of SEQ ID NO: 12, and a VL comprising the amino acid sequence of SEQ ID NO: 32. In an embodiment, the antibody molecule comprises: a VH comprising the amino acid sequence of SEQ ID NO: 12, and a VL comprising the amino acid sequence of SEQ ID NO: 33. In an embodiment, the antibody molecule comprises: a VH comprising the amino acid sequence of SEQ ID NO: 12, and a VL comprising the amino acid sequence of SEQ ID NO: 34. In an embodiment, the antibody molecule comprises: a VH comprising the amino acid sequence of SEQ ID NO: 12, and a VL comprising the amino acid sequence of SEQ ID NO: 35. In an embodiment, the antibody molecule comprises: a VH comprising the amino acid sequence of SEQ ID NO: 12, and a VL comprising the amino acid sequence of SEQ ID NO: 36. In an embodiment, the antibody molecule comprises: a VH comprising the amino acid sequence of SEQ ID NO: 12, and a VL comprising the amino acid sequence of SEQ ID NO: 37. In an embodiment, the antibody molecule comprises: a VH comprising the amino acid sequence of SEQ ID NO: 12, and a VL comprising the amino acid sequence of SEQ ID NO: 38. In an embodiment, the antibody molecule comprises: a VH comprising the amino acid sequence of SEQ ID NO: 12, and a VL comprising the amino acid sequence of SEQ ID NO: 92. In an embodiment, the antibody molecule comprises: a VH comprising the amino acid sequence of SEQ ID NO: 12, and a VL comprising the amino acid sequence of SEQ ID NO: 93. In an embodiment, the antibody molecule comprises: a VH comprising the amino acid sequence of SEQ ID NO: 12, and a VL comprising the amino acid sequence of SEQ ID NO: 94. In an embodiment, the antibody molecule comprises: a VH comprising the amino acid sequence of SEQ ID NO: 12, and a VL comprising the amino acid sequence of SEQ ID NO: 95. In an embodiment, the antibody molecule comprises: a VH comprising the amino acid sequence of SEQ ID NO: 12, and a VL comprising the amino acid sequence of SEQ ID NO: 96.

In an embodiment, the antibody molecule comprises: a VH comprising the amino acid sequence of SEQ ID NO: 13, and a VL comprising the amino acid sequence of SEQ ID NO: 14. In an embodiment, the antibody molecule comprises: a VH comprising the amino acid sequence of SEQ ID NO: 13, and a VL comprising the amino acid sequence of SEQ ID NO: 15. In an embodiment, the antibody molecule comprises: a VH comprising the amino acid sequence of SEQ ID NO: 13, and a VL comprising the amino acid sequence of SEQ ID NO: 16. In an embodiment, the antibody molecule comprises: a VH comprising the amino acid sequence of SEQ ID NO: 13, and a VL comprising the amino acid sequence of SEQ ID NO: 17. In an embodiment, the antibody molecule comprises: a VH comprising the amino acid sequence of SEQ ID NO: 13, and a VL comprising the amino acid sequence of SEQ ID NO: 18. In an embodiment, the antibody molecule comprises: a VH comprising the amino acid sequence of SEQ ID NO: 13, and a VL comprising the amino acid sequence of SEQ ID NO: 19. In an embodiment, the antibody molecule comprises: a VH comprising the amino acid sequence of SEQ ID NO: 13, and a VL comprising the amino acid sequence of SEQ ID NO: 20. In an embodiment, the antibody molecule comprises: a VH comprising the amino acid sequence of SEQ ID NO: 13, and a VL comprising the amino acid sequence of SEQ ID NO: 21. In an embodiment, the antibody molecule comprises: a VH comprising the amino acid sequence of SEQ ID NO: 13, and a VL comprising the amino acid sequence of SEQ ID NO: 22. In an embodiment, the antibody molecule comprises: a VH comprising the amino acid sequence of SEQ ID NO: 13, and a VL comprising the amino acid sequence of SEQ ID NO: 23. In an embodiment, the antibody molecule comprises: a VH comprising the amino acid sequence of SEQ ID NO: 13, and a VL comprising the amino acid sequence of SEQ ID NO: 24. In an embodiment, the antibody molecule comprises: a VH comprising the amino acid sequence of SEQ ID NO: 13, and a VL comprising the amino acid sequence of SEQ ID NO: 25. In an embodiment, the antibody molecule comprises: a VH comprising the amino acid sequence of SEQ ID NO: 13, and a VL comprising the amino acid sequence of SEQ ID NO: 26. In an embodiment, the antibody molecule comprises: a VH comprising the amino acid sequence of SEQ ID NO: 13, and a VL comprising the amino acid sequence of SEQ ID NO: 27. In an embodiment, the antibody molecule comprises: a VH comprising the amino acid sequence of SEQ ID NO: 13, and a VL comprising the amino acid sequence of SEQ ID NO: 28. In an embodiment, the antibody molecule comprises: a VH comprising the amino acid sequence of SEQ ID NO: 13, and a VL comprising the amino acid sequence of SEQ ID NO: 29. In an embodiment, the antibody molecule comprises: a VH comprising the amino acid sequence of SEQ ID NO: 13, and a VL comprising the amino acid sequence of SEQ ID NO: 30. In an embodiment, the antibody molecule comprises: a VH comprising the amino acid sequence of SEQ ID NO: 13, and a VL comprising the amino acid sequence of SEQ ID NO: 31. In an embodiment, the antibody molecule comprises: a VH comprising the amino acid sequence of SEQ ID NO: 13, and a VL comprising the amino acid sequence of SEQ ID NO: 32. In an embodiment, the antibody molecule comprises: a VH comprising the amino acid sequence of SEQ ID NO: 13, and a VL comprising the amino acid sequence of SEQ ID NO: 33. In an embodiment, the antibody molecule comprises: a VH comprising the amino acid sequence of SEQ ID NO: 13, and a VL comprising the amino acid sequence of SEQ ID NO: 34. In an embodiment, the antibody molecule comprises: a VH comprising the amino acid sequence of SEQ ID NO: 13, and a VL comprising the amino acid sequence of SEQ ID NO: 35. In an embodiment, the antibody molecule comprises: a VH comprising the amino acid sequence of SEQ ID NO: 13, and a VL comprising the amino acid sequence of SEQ ID NO: 36. In an embodiment, the antibody molecule comprises: a VH comprising the amino acid sequence of SEQ ID NO: 13, and a VL comprising the amino acid sequence of SEQ ID NO: 37. In an embodiment, the antibody molecule comprises: a VH comprising the amino acid sequence of SEQ ID NO: 13, and a VL comprising the amino acid sequence of SEQ ID NO: 38. In an embodiment, the antibody molecule comprises: a VH comprising the amino acid sequence of SEQ ID NO: 13, and a VL comprising the amino acid sequence of SEQ ID NO: 92. In an embodiment, the antibody molecule comprises: a VH comprising the amino acid sequence of SEQ ID NO: 13, and a VL comprising the amino acid sequence of SEQ ID NO: 93. In an embodiment, the antibody molecule comprises: a VH comprising the amino acid sequence of SEQ ID NO: 13, and a VL comprising the amino acid sequence of SEQ ID NO: 94. In an embodiment, the antibody molecule comprises: a VH comprising the amino acid sequence of SEQ ID NO: 13, and a VL comprising the amino acid sequence of SEQ ID NO: 95. In an embodiment, the antibody molecule comprises: a VH comprising the amino acid sequence of SEQ ID NO: 13, and a VL comprising the amino acid sequence of SEQ ID NO: 96.

In an embodiment, the antibody molecule comprises: a VH comprising the amino acid sequence of SEQ ID NO: 90, and a VL comprising the amino acid sequence of SEQ ID NO: 14. In an embodiment, the antibody molecule comprises: a VH comprising the amino acid sequence of SEQ ID NO: 90, and a VL comprising the amino acid sequence of SEQ ID NO: 15. In an embodiment, the antibody molecule comprises: a VH comprising the amino acid sequence of SEQ ID NO: 90, and a VL comprising the amino acid sequence of SEQ ID NO: 16. In an embodiment, the antibody molecule comprises: a VH comprising the amino acid sequence of SEQ ID NO: 90, and a VL comprising the amino acid sequence of SEQ ID NO: 17. In an embodiment, the antibody molecule comprises: a VH comprising the amino acid sequence of SEQ ID NO: 90, and a VL comprising the amino acid sequence of SEQ ID NO: 18. In an embodiment, the antibody molecule comprises: a VH comprising the amino acid sequence of SEQ ID NO: 90, and a VL comprising the amino acid sequence of SEQ ID NO: 19. In an embodiment, the antibody molecule comprises: a VH comprising the amino acid sequence of SEQ ID NO: 90, and a VL comprising the amino acid sequence of SEQ ID NO: 20. In an embodiment, the antibody molecule comprises: a VH comprising the amino acid sequence of SEQ ID NO: 90, and a VL comprising the amino acid sequence of SEQ ID NO: 21. In an embodiment, the antibody molecule comprises: a VH comprising the amino acid sequence of SEQ ID NO: 90, and a VL comprising the amino acid sequence of SEQ ID NO: 22. In an embodiment, the antibody molecule comprises: a VH comprising the amino acid sequence of SEQ ID NO: 90, and a VL comprising the amino acid sequence of SEQ ID NO: 23. In an embodiment, the antibody molecule comprises: a VH comprising the amino acid sequence of SEQ ID NO: 90, and a VL comprising the amino acid sequence of SEQ ID NO: 24. In an embodiment, the antibody molecule comprises: a VH comprising the amino acid sequence of SEQ ID NO: 90, and a VL comprising the amino acid sequence of SEQ ID NO: 25. In an embodiment, the antibody molecule comprises: a VH comprising the amino acid sequence of SEQ ID NO: 90, and a VL comprising the amino acid sequence of SEQ ID NO: 26. In an embodiment, the antibody molecule comprises: a VH comprising the amino acid sequence of SEQ ID NO: 90, and a VL comprising the amino acid sequence of SEQ ID NO: 27. In an embodiment, the antibody molecule comprises: a VH comprising the amino acid sequence of SEQ ID NO: 90, and a VL comprising the amino acid sequence of SEQ ID NO: 28. In an embodiment, the antibody molecule comprises: a VH comprising the amino acid sequence of SEQ ID NO: 90, and a VL comprising the amino acid sequence of SEQ ID NO: 29. In an embodiment, the antibody molecule comprises: a VH comprising the amino acid sequence of SEQ ID NO: 90, and a VL comprising the amino acid sequence of SEQ ID NO: 30. In an embodiment, the antibody molecule comprises: a VH comprising the amino acid sequence of SEQ ID NO: 90, and a VL comprising the amino acid sequence of SEQ ID NO: 31. In an embodiment, the antibody molecule comprises: a VH comprising the amino acid sequence of SEQ ID NO: 90, and a VL comprising the amino acid sequence of SEQ ID NO: 32. In an embodiment, the antibody molecule comprises: a VH comprising the amino acid sequence of SEQ ID NO: 90, and a VL comprising the amino acid sequence of SEQ ID NO: 33. In an embodiment, the antibody molecule comprises: a VH comprising the amino acid sequence of SEQ ID NO: 90, and a VL comprising the amino acid sequence of SEQ ID NO: 34. In an embodiment, the antibody molecule comprises: a VH comprising the amino acid sequence of SEQ ID NO: 90, and a VL comprising the amino acid sequence of SEQ ID NO: 35. In an embodiment, the antibody molecule comprises: a VH comprising the amino acid sequence of SEQ ID NO: 90, and a VL comprising the amino acid sequence of SEQ ID NO: 36. In an embodiment, the antibody molecule comprises: a VH comprising the amino acid sequence of SEQ ID NO: 90, and a VL comprising the amino acid sequence of SEQ ID NO: 37. In an embodiment, the antibody molecule comprises: a VH comprising the amino acid sequence of SEQ ID NO: 90, and a VL comprising the amino acid sequence of SEQ ID NO: 38. In an embodiment, the antibody molecule comprises: a VH comprising the amino acid sequence of SEQ ID NO: 90, and a VL comprising the amino acid sequence of SEQ ID NO: 92. In an embodiment, the antibody molecule comprises: a VH comprising the amino acid sequence of SEQ ID NO: 90, and a VL comprising the amino acid sequence of SEQ ID NO: 93. In an embodiment, the antibody molecule comprises: a VH comprising the amino acid sequence of SEQ ID NO: 90, and a VL comprising the amino acid sequence of SEQ ID NO: 94. In an embodiment, the antibody molecule comprises: a VH comprising the amino acid sequence of SEQ ID NO: 90, and a VL comprising the amino acid sequence of SEQ ID NO: 95. In an embodiment, the antibody molecule comprises: a VH comprising the amino acid sequence of SEQ ID NO: 90, and a VL comprising the amino acid sequence of SEQ ID NO: 96.

In an embodiment, the antibody molecule comprises: a VH comprising the amino acid sequence of SEQ ID NO: 91, and a VL comprising the amino acid sequence of SEQ ID NO: 14. In an embodiment, the antibody molecule comprises: a VH comprising the amino acid sequence of SEQ ID NO: 91, and a VL comprising the amino acid sequence of SEQ ID NO: 15. In an embodiment, the antibody molecule comprises: a VH comprising the amino acid sequence of SEQ ID NO: 91, and a VL comprising the amino acid sequence of SEQ ID NO: 16. In an embodiment, the antibody molecule comprises: a VH comprising the amino acid sequence of SEQ ID NO: 91, and a VL comprising the amino acid sequence of SEQ ID NO: 17. In an embodiment, the antibody molecule comprises: a VH comprising the amino acid sequence of SEQ ID NO: 91, and a VL comprising the amino acid sequence of SEQ ID NO: 18. In an embodiment, the antibody molecule comprises: a VH comprising the amino acid sequence of SEQ ID NO: 91, and a VL comprising the amino acid sequence of SEQ ID NO: 19. In an embodiment, the antibody molecule comprises: a VH comprising the amino acid sequence of SEQ ID NO: 91, and a VL comprising the amino acid sequence of SEQ ID NO: 20. In an embodiment, the antibody molecule comprises: a VH comprising the amino acid sequence of SEQ ID NO: 91, and a VL comprising the amino acid sequence of SEQ ID NO: 21. In an embodiment, the antibody molecule comprises: a VH comprising the amino acid sequence of SEQ ID NO: 91, and a VL comprising the amino acid sequence of SEQ ID NO: 22. In an embodiment, the antibody molecule comprises: a VH comprising the amino acid sequence of SEQ ID NO: 91, and a VL comprising the amino acid sequence of SEQ ID NO: 23. In an embodiment, the antibody molecule comprises: a VH comprising the amino acid sequence of SEQ ID NO: 91, and a VL comprising the amino acid sequence of SEQ ID NO: 24. In an embodiment, the antibody molecule comprises: a VH comprising the amino acid sequence of SEQ ID NO: 91, and a VL comprising the amino acid sequence of SEQ ID NO: 25. In an embodiment, the antibody molecule comprises: a VH comprising the amino acid sequence of SEQ ID NO: 91, and a VL comprising the amino acid sequence of SEQ ID NO: 26. In an embodiment, the antibody molecule comprises: a VH comprising the amino acid sequence of SEQ ID NO: 91, and a VL comprising the amino acid sequence of SEQ ID NO: 27. In an embodiment, the antibody molecule comprises: a VH comprising the amino acid sequence of SEQ ID NO: 91, and a VL comprising the amino acid sequence of SEQ ID NO: 28. In an embodiment, the antibody molecule comprises: a VH comprising the amino acid sequence of SEQ ID NO: 91, and a VL comprising the amino acid sequence of SEQ ID NO: 29. In an embodiment, the antibody molecule comprises: a VH comprising the amino acid sequence of SEQ ID NO: 91, and a VL comprising the amino acid sequence of SEQ ID NO: 30. In an embodiment, the antibody molecule comprises: a VH comprising the amino acid sequence of SEQ ID NO: 91, and a VL comprising the amino acid sequence of SEQ ID NO: 31. In an embodiment, the antibody molecule comprises: a VH comprising the amino acid sequence of SEQ ID NO: 91, and a VL comprising the amino acid sequence of SEQ ID NO: 32. In an embodiment, the antibody molecule comprises: a VH comprising the amino acid sequence of SEQ ID NO: 91, and a VL comprising the amino acid sequence of SEQ ID NO: 33. In an embodiment, the antibody molecule comprises: a VH comprising the amino acid sequence of SEQ ID NO: 91, and a VL comprising the amino acid sequence of SEQ ID NO: 34. In an embodiment, the antibody molecule comprises: a VH comprising the amino acid sequence of SEQ ID NO: 91, and a VL comprising the amino acid sequence of SEQ ID NO: 35. In an embodiment, the antibody molecule comprises: a VH comprising the amino acid sequence of SEQ ID NO: 91, and a VL comprising the amino acid sequence of SEQ ID NO: 36. In an embodiment, the antibody molecule comprises: a VH comprising the amino acid sequence of SEQ ID NO: 91, and a VL comprising the amino acid sequence of SEQ ID NO: 37. In an embodiment, the antibody molecule comprises: a VH comprising the amino acid sequence of SEQ ID NO: 91, and a VL comprising the amino acid sequence of SEQ ID NO: 38. In an embodiment, the antibody molecule comprises: a VH comprising the amino acid sequence of SEQ ID NO: 91, and a VL comprising the amino acid sequence of SEQ ID NO: 92. In an embodiment, the antibody molecule comprises: a VH comprising the amino acid sequence of SEQ ID NO: 91, and a VL comprising the amino acid sequence of SEQ ID NO: 93. In an embodiment, the antibody molecule comprises: a VH comprising the amino acid sequence of SEQ ID NO: 91, and a VL comprising the amino acid sequence of SEQ ID NO: 94. In an embodiment, the antibody molecule comprises: a VH comprising the amino acid sequence of SEQ ID NO: 91, and a VL comprising the amino acid sequence of SEQ ID NO: 95. In an embodiment, the antibody molecule comprises: a VH comprising the amino acid sequence of SEQ ID NO: 91, and a VL comprising the amino acid sequence of SEQ ID NO: 96.

In an embodiment, the antibody molecule comprises one or more framework regions derived from a human framework germline sequence.

In an embodiment, the antibody molecule comprises a VH described in Table 1. In an embodiment, the antibody molecule comprises a VL described in Table 1. In an embodiment, the antibody molecule comprises a VH and a VL described in Table 1. In an embodiment, the antibody molecule comprises one, two, or three CDRs of a VH described in Table 1. In an embodiment, the antibody molecule comprises one, two, or three CDRs of a VL described in Table 1. In an embodiment, the antibody molecule comprises one, two, or three CDRs of a VH described in Table 1, and one, two, or three CDRs of a VL described in Table 1.

In an embodiment, the antibody molecule comprises two VHs and two VLs. In an embodiment, the antibody molecule comprises an antigen-binding fragment. In an embodiment, the antibody molecule comprises a Fab, F(ab′)2, Fv, scFv, sc(Fv)2, or Fd.

In an embodiment, the antibody molecule is an IgG antibody molecule, e.g., comprising a heavy chain constant region of IgG, e.g., chosen from IgG1, IgG2, IgG3, or IgG4, e.g., IgG2 or IgG4. In an embodiment, the antibody molecule is an IgG1 antibody molecule, e.g., having an IgG1 constant region described herein. In another embodiment, the antibody molecule is an IgG2 antibody molecule e.g., having an IgG2 constant region described herein. In an embodiment, the antibody molecule is an IgG3 antibody molecule, e.g., having an IgG3 constant region described herein. In another embodiment, the antibody molecule is an IgG4 antibody molecule e.g., having an IgG4 constant region described herein. In another embodiment, the antibody molecule has a chimeric constant region comprising of IgG2, IgG3 and/or IgG4 isotypes. In an embodiment, the heavy chain constant region comprises one or more amino acid modifications in the hinge, CH2 or CH3 region. In an embodiment, the antibody molecule comprises a light chain constant region of kappa or lambda light chain. In an embodiment, the antibody molecule comprises an Fc region.

In an aspect, the disclosure features an anti-FGF23 antibody molecule described herein, e.g., a synthetic or isolated anti-FGF23 antibody molecule described herein.

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ExA11 (e.g., SEQ ID NO: 41); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ExA11 (e.g., SEQ ID NO: 48); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ExA11 (e.g., SEQ ID NO: 53), and

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ExA11 (e.g., SEQ ID NO: 61); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ExA11 (e.g., SEQ ID NO: 67); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ExA11 (e.g., SEQ ID NO: 74).

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ExA28 (e.g., SEQ ID NO: 41); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ExA28 (e.g., SEQ ID NO: 46); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ExA28 (e.g., SEQ ID NO: 53), and

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ExA28 (e.g., SEQ ID NO: 61); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ExA28 (e.g., SEQ ID NO: 67); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ExA28 (e.g., SEQ ID NO: 74).

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ExA35 (e.g., SEQ ID NO: 41); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ExA35 (e.g., SEQ ID NO: 49); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ExA35 (e.g., SEQ ID NO: 53), and

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ExA35 (e.g., SEQ ID NO: 61); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ExA35 (e.g., SEQ ID NO: 67); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ExA35 (e.g., SEQ ID NO: 74).

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ExA43 (e.g., SEQ ID NO: 41); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ExA43 (e.g., SEQ ID NO: 46); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ExA43 (e.g., SEQ ID NO: 57), and

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ExA43 (e.g., SEQ ID NO: 61); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ExA43 (e.g., SEQ ID NO: 67); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ExA43 (e.g., SEQ ID NO: 74).

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ExA60 (e.g., SEQ ID NO: 41); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ExA60 (e.g., SEQ ID NO: 50); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ExA60 (e.g., SEQ ID NO: 54), and

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ExA60 (e.g., SEQ ID NO: 61); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ExA60 (e.g., SEQ ID NO: 67); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ExA60 (e.g., SEQ ID NO: 74).

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ExC17 (e.g., SEQ ID NO: 41); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ExC17 (e.g., SEQ ID NO: 46); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ExC17 (e.g., SEQ ID NO: 53), and

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ExC17 (e.g., SEQ ID NO: 61); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ExC17 (e.g., SEQ ID NO: 69); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ExC17 (e.g., SEQ ID NO: 89).

In an embodiment, the antibody molecule comprises:

(i) a heavy chain variable region (VH), wherein the heavy chain variable region comprises three heavy chain complementarity determining regions (HCDR1, HCDR2, and HCDR3), wherein the heavy chain variable region comprises one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ExC50 (e.g., SEQ ID NO: 41); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ExC50 (e.g., SEQ ID NO: 49); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ExC50 (e.g., SEQ ID NO: 55), and

(ii) a light chain variable region (VL), wherein the light chain variable region comprises three light chain complementarity determining regions (LCDR1, LCDR2, and LCDR3), wherein the light chain variable region comprises one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ExC50 (e.g., SEQ ID NO: 61); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ExC50 (e.g., SEQ ID NO: 69); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ExC50 (e.g., SEQ ID NO: 74).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ExA11 (e.g., SEQ ID NO: 41); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ExA11 (e.g., SEQ ID NO: 48); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ExA11 (e.g., SEQ ID NO: 53), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ExA11 (e.g., SEQ ID NO: 61); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ExA11 (e.g., SEQ ID NO: 67); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ExA11 (e.g., SEQ ID NO: 74).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ExA28 (e.g., SEQ ID NO: 41); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ExA28 (e.g., SEQ ID NO: 46); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ExA28 (e.g., SEQ ID NO: 53), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ExA28 (e.g., SEQ ID NO: 61); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ExA28 (e.g., SEQ ID NO: 67); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ExA28 (e.g., SEQ ID NO: 74).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ExA35 (e.g., SEQ ID NO: 41); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ExA35 (e.g., SEQ ID NO: 49); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ExA35 (e.g., SEQ ID NO: 53), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ExA35 (e.g., SEQ ID NO: 61); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ExA35 (e.g., SEQ ID NO: 67); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ExA35 (e.g., SEQ ID NO: 74).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ExA43 (e.g., SEQ ID NO: 41); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ExA43 (e.g., SEQ ID NO: 46); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ExA43 (e.g., SEQ ID NO: 57), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ExA43 (e.g., SEQ ID NO: 61); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ExA43 (e.g., SEQ ID NO: 67); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ExA43 (e.g., SEQ ID NO: 74).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ExA60 (e.g., SEQ ID NO: 41); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ExA60 (e.g., SEQ ID NO: 50); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ExA60 (e.g., SEQ ID NO: 54), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ExA60 (e.g., SEQ ID NO: 61); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ExA60 (e.g., SEQ ID NO: 67); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ExA60 (e.g., SEQ ID NO: 74).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ExC17 (e.g., SEQ ID NO: 41); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ExC17 (e.g., SEQ ID NO: 46); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ExC17 (e.g., SEQ ID NO: 53), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ExC17 (e.g., SEQ ID NO: 61); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ExC17 (e.g., SEQ ID NO: 69); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ExC17 (e.g., SEQ ID NO: 89).

In an embodiment, the antibody molecule comprises:

(i) a VH comprising one, two, or all of the following: an HCDR1 comprising an amino acid sequence that differs by no more than 1, 2, or 3 amino acid residues from, or has at least 85, 90, 95, 99 or 100% homology with, the amino acid sequence of the HCDR1 of monoclonal antibody ExC50 (e.g., SEQ ID NO: 41); an HCDR2 comprising an amino acid sequence that differs by no more than 1, 2, or 3 amino acid residues from, or has at least 85, 90, 95, 99 or 100% homology with, the amino acid sequence of the HCDR2 of monoclonal antibody ExC50 (e.g., SEQ ID NO: 46); or an HCDR3 comprising an amino acid sequence that differs by no more than 1, 2, or 3 amino acid residues from, or has at least 85, 90, 95, 99 or 100% homology with, the amino acid sequence of the HCDR3 of monoclonal antibody ExC50 (e.g., SEQ ID NO: 53), and

(ii) a VL comprising one, two, or all of the following: an LCDR1 comprising an amino acid sequence that differs by no more than 1, 2, or 3 amino acid residues from, or has at least 85, 90, 95, 99 or 100% homology with, the amino acid sequence of the LCDR1 of monoclonal antibody ExC50 (e.g., SEQ ID NO: 61); an LCDR2 comprising an amino acid sequence that differs by no more than 1, 2, or 3 amino acid residues from, or has at least 85, 90, 95, 99 or 100% homology with, the amino acid sequence of the LCDR2 of monoclonal antibody ExC50 (e.g., SEQ ID NO: 69); or an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody ExC50 (e.g., SEQ ID NO: 89).

In an embodiment, the antibody molecule comprises: (i) a VH comprising: an HCDR1 comprising the amino acid sequence of the HCDR1 of monoclonal antibody ExA11 (e.g., SEQ ID NO: 41); an HCDR2 comprising the amino acid sequence of the HCDR2 of monoclonal antibody ExA11 (e.g., SEQ ID NO: 48); and an HCDR3 comprising the amino acid sequence of the HCDR3 of monoclonal antibody ExA11 (e.g., SEQ ID NO: 53), and (ii) a VL comprising: an LCDR1 comprising the amino acid sequence of the LCDR1 of monoclonal antibody ExA11 (e.g., SEQ ID NO: 61); an LCDR2 comprising the amino acid sequence of the LCDR2 of monoclonal antibody ExA11 (e.g., SEQ ID NO: 67); and an LCDR3 comprising the amino acid sequence of the LCDR3 of monoclonal antibody ExA11 (e.g., SEQ ID NO: 74).

In an embodiment, the antibody molecule comprises: (i) a VH comprising: an HCDR1 comprising the amino acid sequence of the HCDR1 of monoclonal antibody ExA28 (e.g., SEQ ID NO: 41); an HCDR2 comprising the amino acid sequence of the HCDR2 of monoclonal antibody ExA28 (e.g., SEQ ID NO: 46); and an HCDR3 comprising the amino acid sequence of the HCDR3 of monoclonal antibody ExA28 (e.g., SEQ ID NO: 53), and (ii) a VL comprising: an LCDR1 comprising the amino acid sequence of the LCDR1 of monoclonal antibody ExA28 (e.g., SEQ ID NO: 61); an LCDR2 comprising the amino acid sequence of the LCDR2 of monoclonal antibody ExA28 (e.g., SEQ ID NO: 67); and an LCDR3 comprising the amino acid sequence of the LCDR3 of monoclonal antibody ExA28 (e.g., SEQ ID NO: 74).

In an embodiment, the antibody molecule comprises: (i) a VH comprising: an HCDR1 comprising the amino acid sequence of the HCDR1 of monoclonal antibody ExA35 (e.g., SEQ ID NO: 41); an HCDR2 comprising the amino acid sequence of the HCDR2 of monoclonal antibody ExA35 (e.g., SEQ ID NO: 49); and an HCDR3 comprising the amino acid sequence of the HCDR3 of monoclonal antibody ExA35 (e.g., SEQ ID NO: 53), and (ii) a VL comprising: an LCDR1 comprising the amino acid sequence of the LCDR1 of monoclonal antibody ExA35 (e.g., SEQ ID NO: 61); an LCDR2 comprising the amino acid sequence of the LCDR2 of monoclonal antibody ExA35 (e.g., SEQ ID NO: 67); and an LCDR3 comprising the amino acid sequence of the LCDR3 of monoclonal antibody ExA35 (e.g., SEQ ID NO: 74).

In an embodiment, the antibody molecule comprises: (i) a VH comprising: an HCDR1 comprising the amino acid sequence of the HCDR1 of monoclonal antibody ExA43 (e.g., SEQ ID NO: 41); an HCDR2 comprising the amino acid sequence of the HCDR2 of monoclonal antibody ExA43 (e.g., SEQ ID NO: 46); and an HCDR3 comprising the amino acid sequence of the HCDR3 of monoclonal antibody ExA43 (e.g., SEQ ID NO: 57), and (ii) a VL comprising: an LCDR1 comprising the amino acid sequence of the LCDR1 of monoclonal antibody ExA43 (e.g., SEQ ID NO: 61); an LCDR2 comprising the amino acid sequence of the LCDR2 of monoclonal antibody ExA43 (e.g., SEQ ID NO: 67); and an LCDR3 comprising the amino acid sequence of the LCDR3 of monoclonal antibody ExA43 (e.g., SEQ ID NO: 74).

In an embodiment, the antibody molecule comprises: (i) a VH comprising: an HCDR1 comprising the amino acid sequence of the HCDR1 of monoclonal antibody ExA60 (e.g., SEQ ID NO: 41); an HCDR2 comprising the amino acid sequence of the HCDR2 of monoclonal antibody ExA60 (e.g., SEQ ID NO: 50); and an HCDR3 comprising the amino acid sequence of the HCDR3 of monoclonal antibody ExA60 (e.g., SEQ ID NO: 54), and (ii) a VL comprising: an LCDR1 comprising the amino acid sequence of the LCDR1 of monoclonal antibody ExA60 (e.g., SEQ ID NO: 61); an LCDR2 comprising the amino acid sequence of the LCDR2 of monoclonal antibody ExA60 (e.g., SEQ ID NO: 67); and an LCDR3 comprising the amino acid sequence of the LCDR3 of monoclonal antibody ExA60 (e.g., SEQ ID NO: 74).

In an embodiment, the antibody molecule comprises: (i) a VH comprising: an HCDR1 comprising the amino acid sequence of the HCDR1 of monoclonal antibody ExC17 (e.g., SEQ ID NO: 41); an HCDR2 comprising the amino acid sequence of the HCDR2 of monoclonal antibody ExC17 (e.g., SEQ ID NO: 46); and an HCDR3 comprising the amino acid sequence of the HCDR3 of monoclonal antibody ExC17 (e.g., SEQ ID NO: 53), and (ii) a VL comprising: an LCDR1 comprising the amino acid sequence of the LCDR1 of monoclonal antibody ExC17 (e.g., SEQ ID NO: 61); an LCDR2 comprising the amino acid sequence of the LCDR2 of monoclonal antibody ExC17 (e.g., SEQ ID NO: 69); and an LCDR3 comprising the amino acid sequence of the LCDR3 of monoclonal antibody ExC17 (e.g., SEQ ID NO: 89).

In an embodiment, the antibody molecule comprises: (i) a VH comprising: an HCDR1 comprising the amino acid sequence of the HCDR1 of monoclonal antibody ExC50 (e.g., SEQ ID NO: 41); an HCDR2 comprising the amino acid sequence of the HCDR2 of monoclonal antibody ExC50 (e.g., SEQ ID NO: 49); and an HCDR3 comprising the amino acid sequence of the HCDR3 of monoclonal antibody ExC50 (e.g., SEQ ID NO: 55), and (ii) a VL comprising: an LCDR1 comprising the amino acid sequence of the LCDR1 of monoclonal antibody ExC50 (e.g., SEQ ID NO: 61); an LCDR2 comprising the amino acid sequence of the LCDR2 of monoclonal antibody ExC50 (e.g., SEQ ID NO: 67); and an LCDR3 comprising the amino acid sequence of the LCDR3 of monoclonal antibody ExC50 (e.g., SEQ ID NO: 74).

In an embodiment, the antibody molecule comprises: (i) a VH comprising: an HCDR1 comprising the amino acid sequence of the HCDR1 of monoclonal antibody Exc23 (e.g., SEQ ID NO: 41); an HCDR2 comprising the amino acid sequence of the HCDR2 of monoclonal antibody Exc23 (e.g., SEQ ID NO: 46); and an HCDR3 comprising the amino acid sequence of the HCDR3 of monoclonal antibody Exc23 (e.g., SEQ ID NO: 53), and (ii) a VL comprising: an LCDR1 comprising the amino acid sequence of the LCDR1 of monoclonal antibody Exc23 (e.g., SEQ ID NO: 59); an LCDR2 comprising the amino acid sequence of the LCDR2 of monoclonal antibody Exc23 (e.g., SEQ ID NO: 70); and an LCDR3 comprising the amino acid sequence of the LCDR3 of monoclonal antibody Exc23 (e.g., SEQ ID NO: 73).

In an embodiment, the antibody molecule comprises: (i) a VH comprising: an HCDR1 comprising the amino acid sequence of the HCDR1 of monoclonal antibody Exc23.1 (e.g., SEQ ID NO: 41); an HCDR2 comprising the amino acid sequence of the HCDR2 of monoclonal antibody Exc23.1 (e.g., SEQ ID NO: 46); and an HCDR3 comprising the amino acid sequence of the HCDR3 of monoclonal antibody Exc23.1 (e.g., SEQ ID NO: 53), and (ii) a VL comprising: an LCDR1 comprising the amino acid sequence of the LCDR1 of monoclonal antibody Exc23.1 (e.g., SEQ ID NO: 59); an LCDR2 comprising the amino acid sequence of the LCDR2 of monoclonal antibody Exc23.1 (e.g., SEQ ID NO: 68); and an LCDR3 comprising the amino acid sequence of the LCDR3 of monoclonal antibody Exc23.1 (e.g., SEQ ID NO: 73).

In an embodiment, the antibody molecule comprises: (i) a VH comprising: an HCDR1 comprising the amino acid sequence of the HCDR1 of monoclonal antibody Exc23.2 (e.g., SEQ ID NO: 41); an HCDR2 comprising the amino acid sequence of the HCDR2 of monoclonal antibody Exc23.2 (e.g., SEQ ID NO: 46); and an HCDR3 comprising the amino acid sequence of the HCDR3 of monoclonal antibody Exc23.2 (e.g., SEQ ID NO: 53), and (ii) a VL comprising: an LCDR1 comprising the amino acid sequence of the LCDR1 of monoclonal antibody Exc23.2 (e.g., SEQ ID NO: 59); an LCDR2 comprising the amino acid sequence of the LCDR2 of monoclonal antibody Exc23.2 (e.g., SEQ ID NO: 69); and an LCDR3 comprising the amino acid sequence of the LCDR3 of monoclonal antibody Exc23.2 (e.g., SEQ ID NO: 73).

In an embodiment, the antibody molecule comprises: (i) a VH comprising: an HCDR1 comprising the amino acid sequence of the HCDR1 of monoclonal antibody Exc23.3 (e.g., SEQ ID NO: 41); an HCDR2 comprising the amino acid sequence of the HCDR2 of monoclonal antibody Exc23.3 (e.g., SEQ ID NO: 46); and an HCDR3 comprising the amino acid sequence of the HCDR3 of monoclonal antibody Exc23.3 (e.g., SEQ ID NO: 53), and (ii) a VL comprising: an LCDR1 comprising the amino acid sequence of the LCDR1 of monoclonal antibody Exc23.3 (e.g., SEQ ID NO: 59); an LCDR2 comprising the amino acid sequence of the LCDR2 of monoclonal antibody Exc23.3 (e.g., SEQ ID NO: 70); and an LCDR3 comprising the amino acid sequence of the LCDR3 of monoclonal antibody Exc23.3 (e.g., SEQ ID NO: 74).

In an embodiment, the antibody molecule comprises: (i) a VH comprising: an HCDR1 comprising the amino acid sequence of the HCDR1 of monoclonal antibody Exc23.4 (e.g., SEQ ID NO: 41); an HCDR2 comprising the amino acid sequence of the HCDR2 of monoclonal antibody Exc23.4 (e.g., SEQ ID NO: 46); and an HCDR3 comprising the amino acid sequence of the HCDR3 of monoclonal antibody Exc23.4 (e.g., SEQ ID NO: 53), and (ii) a VL comprising: an LCDR1 comprising the amino acid sequence of the LCDR1 of monoclonal antibody Exc23.4 (e.g., SEQ ID NO: 61); an LCDR2 comprising the amino acid sequence of the LCDR2 of monoclonal antibody Exc23.4 (e.g., SEQ ID NO: 70); and an LCDR3 comprising the amino acid sequence of the LCDR3 of monoclonal antibody Exc23.4 (e.g., SEQ ID NO: 73).

In an embodiment, the antibody molecule comprises: (i) a VH comprising: an HCDR1 comprising the amino acid sequence of the HCDR1 of monoclonal antibody Exc23.5 (e.g., SEQ ID NO: 41); an HCDR2 comprising the amino acid sequence of the HCDR2 of monoclonal antibody Exc23.5 (e.g., SEQ ID NO: 46); and an HCDR3 comprising the amino acid sequence of the HCDR3 of monoclonal antibody Exc23.5 (e.g., SEQ ID NO: 53), and (ii) a VL comprising: an LCDR1 comprising the amino acid sequence of the LCDR1 of monoclonal antibody Exc23.5 (e.g., SEQ ID NO: 59); an LCDR2 comprising the amino acid sequence of the LCDR2 of monoclonal antibody Exc23.5 (e.g., SEQ ID NO: 70); and an LCDR3 comprising the amino acid sequence of the LCDR3 of monoclonal antibody Exc23.5 (e.g., SEQ ID NO: 73).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ExA11 (e.g., SEQ ID NO: 5); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ExA11 (e.g., SEQ ID NO: 19).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ExA28 (e.g., SEQ ID NO: 7); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ExA28 (e.g., SEQ ID NO: 20).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ExA35 (e.g., SEQ ID NO: 8); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ExA35 (e.g., SEQ ID NO: 19).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ExA43 (e.g., SEQ ID NO: 9); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ExA43 (e.g., SEQ ID NO: 19).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ExA60 (e.g., SEQ ID NO: 11); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ExA60 (e.g., SEQ ID NO: 20).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ExC17 (e.g., SEQ ID NO: 7); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ExC17 (e.g., SEQ ID NO: 28).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody ExC50 (e.g., SEQ ID NO: 13); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody ExC50 (e.g., SEQ ID NO: 25).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody Exc23 (e.g., SEQ ID NO: 7); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody Exc23 (e.g., SEQ ID NO: 37).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody Exc23.1 (e.g., SEQ ID NO: 7); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody Exc23.1 (e.g., SEQ ID NO: 34).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody Exc23.2 (e.g., SEQ ID NO: 7); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody Exc23.2 (e.g., SEQ ID NO: 36).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody Exc23.3 (e.g., SEQ ID NO: 7); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody Exc23.3 (e.g., SEQ ID NO: 94).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody Exc23.4 (e.g., SEQ ID NO: 7); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody Exc23.4 (e.g., SEQ ID NO: 95).

In an embodiment, the antibody molecule comprises one or both of: (i) a VH comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VH of monoclonal antibody Exc23.5 (e.g., SEQ ID NO: 7); or (ii) a VL comprising an amino acid sequence that differs by no more than 1, 2, 3, 4, 5, 6, 7, 8, 9, 10, 11, 12, 13, 14, or 15 amino acid residues from, or has at least 85, 90, 95, 96, 97, 98, 99, or 100% homology with, the amino acid sequence of the VL of monoclonal antibody Exc23.5 (e.g., SEQ ID NO: 96).

In an embodiment, the antibody molecule is monoclonal antibody ExA11, ExA28, ExA35, ExA43, ExA60, ExC17, ExC50, Exc23, Exc23.1, Exc23.2, Exc23.3, Exc23.4, or Exc23.5. In an embodiment, the antibody molecule comprises a VH comprising the amino acid sequence of any of SEQ ID NOs: 1-9, a VL comprising the amino acid sequence of any of SEQ ID NOs: 10-15, or both.

In an embodiment, the antibody molecule is any of antibodies ExA11, ExA28, ExA35, ExA43, ExA60, ExC17, ExC50, Exc23, Exc23.1, Exc23.2, Exc23.3, Exc23.4, or Exc23.5.

In an embodiment, the antibody molecule is a synthetic antibody molecule. In an embodiment, the antibody molecule is an isolated antibody molecule. In an embodiment, the antibody molecule is a recombinant antibody molecule. In an embodiment, the antibody molecule is a humanized antibody. In an embodiment, the antibody molecule is a mono-specific antibody molecule. In an embodiment, the antibody molecule is a multi-specific antibody molecule.

In an embodiment, the antibody molecule comprises two heavy chain variable regions and two light chain variable regions. In an embodiment, the antibody molecule comprises an antigen-binding fragment. In an embodiment, the antibody molecule comprises a Fab, F(ab′)2, Fv, scFv, sc(Fv)2, or Fd.

In an embodiment, the antibody molecule is an IgG antibody molecule, e.g., comprising a heavy chain constant region of IgG, e.g., chosen from IgG1, IgG2, IgG3, or IgG4, e.g., IgG2 or IgG4. In an embodiment, the antibody molecule is an IgG1 antibody molecule, e.g., having an IgG1 constant region described herein. In another embodiment, the antibody molecule is an IgG2 antibody molecule e.g., having an IgG2 constant region described herein. In an embodiment, the antibody molecule is an IgG3 antibody molecule, e.g., having an IgG3 constant region described herein. In another embodiment, the antibody molecule is an IgG4 antibody molecule e.g., having an IgG4 constant region described herein. In another embodiment, the antibody molecule has a chimeric constant region comprising of IgG2, IgG3 and/or IgG4 isotypes. In an embodiment, the heavy chain constant region comprises one or more amino acid modifications in the hinge, CH2 or CH3 region. In an embodiment, the antibody molecule comprises a light chain constant region of kappa or lambda light chain. In an embodiment, the antibody molecule comprises an Fc region.

In an aspect, the disclosure features an antibody molecule, which:

a) competes for binding to FGF23 with an anti-FGF23 antibody molecule comprising the heavy chain complementary determining regions (HCDR1, HCDR2 and HCDR3) and the light chain complementary determining regions (LCDR1, LCDR2 and LCDR3) of any of monoclonal antibodies ExA11, ExA28, ExA35, ExA43, ExA60, ExC17, ExC50, Exc23, Exc23.1, Exc23.2, Exc23.3, Exc23.4, or Exc23.5, e.g., as described in Table 1; or

b) binds, or substantially binds, to an epitope that completely or partially overlaps with the epitope of an anti-FGF23 antibody molecule comprising the heavy chain complementary determining regions (HCDR1, HCDR2 and HCDR3) and the light chain complementary determining regions (LCDR1, LCDR2 and LCDR3) of any of monoclonal antibodies ExA11 (e.g., SEQ ID NOS: 41 48, 53, 61, 67, and/or 74, respectively), ExA28 (e.g., SEQ ID NOS: 41, 46, 53, 61, 67, and/or 74, respectively), ExA35 (e.g., SEQ ID NOS: 41, 49, 53, 61, 67, and/or 74, respectively), ExA43 (e.g., SEQ ID NOS: 41, 46, 57, 61, 67, and/or 74, respectively), ExA60 (e.g., SEQ ID NOS: 41, 50, 54, 61, 67, and/or 74, respectively), ExC17 (e.g., SEQ ID NOS: 41, 46, 53, 61, 69, and/or 89, respectively), ExC50 (e.g., SEQ ID NOS: 41, 49, 55, 61, 69 and/or 74, respectively), Exc23 (e.g., SEQ ID NOS: 41, 46, 53, 59, 70, and/or 73, respectively), Exc23.1 (e.g., SEQ ID NOS: 41, 46, 53, 59, 68, and/or 73, respectively), Exc23.2 (e.g., SEQ ID NOS: 41, 46, 53, 59, 69, and/or 73, respectively), Exc23.3 (e.g., SEQ ID NOS: 41, 46, 53, 59, 70, and/or 74, respectively), Exc23.4 (e.g., SEQ ID NOS: 41, 46, 53, 61, 70, and/or 73, respectively), Exc23.5 (e.g., SEQ ID NOS: 41, 46, 53, 59, 70, and/or 73, respectively), e.g., as described in Table 1.

In an embodiment, the antibody molecule competes for binding with an anti-FGF23 antibody molecule that comprises a VH and a VL of any of monoclonal antibodies ExA11, ExA28, ExA35, ExA43, ExA60, ExC17, ExC50, Exc23, Exc23.1, Exc23.2, Exc23.3, Exc23.4, or Exc23.5.

In an embodiment, the antibody molecule binds, or substantially binds, to an epitope that completely or partially overlaps with the epitope of an anti-FGF23 antibody molecule that comprises a VH and a VL of any of monoclonal antibodies ExA11, ExA28, ExA35, ExA43, ExA60, ExC17, ExC50, Exc23, Exc23.1, Exc23.2, Exc23.3, Exc23.4, or Exc23.5.

In an embodiment, the antibody molecule is a synthetic antibody molecule. In an embodiment, the antibody molecule is an isolated antibody molecule. In an embodiment, the antibody molecule is a recombinant antibody molecule. In an embodiment, the antibody molecule is a humanized antibody. In an embodiment, the antibody molecule is a mono-specific antibody molecule. In an embodiment, the antibody molecule is a multi-specific antibody molecule.

In an embodiment, the antibody molecule competes for binding with two, three, four, five, six, seven, or all of the anti-FGF23 antibody molecules that comprise the HCDR1, HCDR2, HCDR3, LCDR1, LCDR2, and LCDR3 of any of monoclonal antibodies ExA11, ExA28, ExA35, ExA43, ExA60, ExC17, ExC50, Exc23, Exc23.1, Exc23.2, Exc23.3, Exc23.4, or Exc23.5.

In an embodiment, the antibody molecule competes for binding with two, three, four, five, six, seven, or all of the anti-FGF23 antibody molecules that comprises a VH and a VL of any of monoclonal antibodies ExA11, ExA28, ExA35, ExA43, ExA60, ExC17, ExC50, Exc23, Exc23.1, Exc23.2, Exc23.3, Exc23.4, or Exc23.5.

In an embodiment, the antibody molecule binds, or substantially binds, to an epitope that completely or partially overlaps with the epitopes of two, three, four, five, six, seven, or all of the anti-FGF23 antibody molecules that comprise the HCDR1, HCDR2, HCDR3, LCDR1, LCDR2, and LCDR3 of any of monoclonal antibodies ExA11, ExA28, ExA35, ExA43, ExA60, ExC17, ExC50, Exc23, Exc23.1, Exc23.2, Exc23.3, Exc23.4, or Exc23.5.

In an embodiment, the antibody molecule binds, or substantially binds, to an epitope that completely or partially overlaps with the epitopes of two, three, four, five, six, seven, or all of the anti-FGF23 antibody molecules that comprises a VH and a VL of any of monoclonal antibodies ExA11, ExA28, ExA35, ExA43, ExA60, ExC17, ExC50, Exc23, Exc23.1, Exc23.2, Exc23.3, Exc23.4, or Exc23.5.

In an embodiment, the antibody molecule binds, or substantially binds, to an epitope that completely or partially overlaps with the epitopes of two, three, four, five, six, seven, or all of the antibody molecules that comprise the HCDR1, HCDR2, HCDR3, LCDR1, LCDR2, and LCDR3 of any of monoclonal antibodies ExA11, ExA28, ExA35, ExA43, ExA60, ExC17, ExC50, Exc23, Exc23.1, Exc23.2, Exc23.3, Exc23.4, or Exc23.5.

In an embodiment, the antibody molecule that comprises the HCDR1, HCDR2, HCDR3, LCDR1, LCDR2, and LCDR3 of any of monoclonal antibodies ExA11, ExA28, ExA35, ExA43, ExA60, ExC17, ExC50, Exc23, Exc23.1, Exc23.2, Exc23.3, Exc23.4, or Exc23.5.

In an embodiment, the antibody molecule binds, or substantially binds, to an epitope that completely or partially overlaps with the epitopes of two, three, four, five, six, seven, or all of the antibody molecules that comprises a VH and a VL of any of monoclonal antibodies ExA11, ExA28, ExA35, ExA43, ExA60, ExC17, ExC50, Exc23, Exc23.1, Exc23.2, Exc23.3, Exc23.4, or Exc23.5.

In an embodiment, the antibody molecule is a synthetic antibody molecule. In an embodiment, the antibody molecule is an isolated antibody molecule. In an embodiment, the antibody molecule is a recombinant antibody molecule. In an embodiment, the antibody molecule is a humanized antibody. In an embodiment, the antibody molecule is a mono-specific antibody molecule. In an embodiment, the antibody molecule is a multi-specific antibody molecule.

In an embodiment, the antibody molecule comprises two heavy chain variable regions and two light chain variable regions. In an embodiment, the antibody molecule comprises an antigen-binding fragment. In an embodiment, the antibody molecule comprises a Fab, F(ab′)2, Fv, scFv, sc(Fv)2, or Fd.

In an embodiment, the antibody molecule is an IgG antibody molecule, e.g., comprising a heavy chain constant region of IgG, e.g., chosen from IgG1, IgG2, IgG3, or IgG4, e.g., IgG2 or IgG4. In an embodiment, the antibody molecule is an IgG1 antibody molecule, e.g., having an IgG1 constant region described herein. In another embodiment, the antibody molecule is an IgG2 antibody molecule e.g., having an IgG2 constant region described herein. In an embodiment, the antibody molecule is an IgG3 antibody molecule, e.g., having an IgG3 constant region described herein. In another embodiment, the antibody molecule is an IgG4 antibody molecule e.g., having an IgG4 constant region described herein. In another embodiment, the antibody molecule has a chimeric constant region comprising of IgG2, IgG3 and/or IgG4 isotypes. In an embodiment, the heavy chain constant region comprises one or more amino acid modifications in the hinge, CH2 or CH3 region. In an embodiment, the antibody molecule comprises a light chain constant region of kappa or lambda light chain. In an embodiment, the antibody molecule comprises an Fc region.

In an aspect, the disclosure features a composition, e.g., pharmaceutical composition, comprising an antibody molecule described herein. In an embodiment, the composition further comprises a pharmaceutical acceptable carrier.

In an aspect, the disclosure features a nucleic acid molecule encoding a heavy chain variable region (VH), a light chain variable region (VL), or both, of an antibody molecule described herein.

In an aspect, the disclosure features a vector comprising a nucleic acid molecule described herein.

In an aspect, the disclosure features a cell, e.g., an isolated cell, comprising a nucleic acid molecule described herein or a vector described herein.

In an aspect, the disclosure features a kit comprising an antibody molecule described herein and instructions to use of the antibody molecule.

In an aspect, the disclosure features a container comprising an antibody molecule described herein.

In an aspect, the disclosure features a method of producing an anti-FGF23 antibody molecule, the method comprising culturing a cell described herein under conditions that allow production of an antibody molecule, thereby producing the antibody molecule.

In an embodiment, the method further comprises isolating the antibody molecule.

In an aspect, the disclosure features a method of treating an FGF23-associated disorder, e.g., an FGF23-associated disorder described herein, the method comprising administering to a subject in need thereof an effective amount of an antibody molecule described herein or a composition described herein, thereby treating the FGF23-associated disorder.

In an embodiment, the FGF23-associated disorder comprises a hypophosphatemic disorder (e.g., a hypophosphatemic disorder with a skeletal abnormality). In an embodiment, the FGF23-associated disorder is chosen from X-linked hypophosphatemic rickets (XLH), autosomal recessive hypophosphatemic rickets (ARHR) (e.g., ARHR 1 or ARHR2), autosomal dominant hypophosphatemic rickets (ADHR), osteoglophonic dysplasia, Jansen-type metaphyseal chondrodysplasia, hypophosphatemia with dental abnormality and ectopic calcification, McCune-Albright syndrome, epidermal nevus syndrome (ENS), or tumor-induced osteomalacia (TIO).

In an embodiment, the subject is a human. In an embodiment, the antibody molecule is administered to the subject intravenously.

In an embodiment, the antibody molecule is administered to the subject at a dose between 0.1 mg/kg and 50 mg/kg, e.g., between 0.2 mg/kg and 25 mg/kg, between 0.5 mg/kg and 10 mg/kg, between 0.5 mg/kg and 5 mg/kg, between 0.5 mg/kg and 3 mg/kg, between 0.5 mg/kg and 2.5 mg/kg, between 0.5 mg/kg and 2 mg/kg, between 0.5 mg/kg and 1.5 mg/kg, between 0.5 mg/kg and 1 mg/kg, between 1 mg/kg and 1.5 mg/kg, between 1 mg/kg and 2 mg/kg, between 1 mg/kg and 2.5 mg/kg, between 1 mg/kg and 3 mg/kg, between 1 mg/kg and 2.5 mg/kg, or between 1 mg/kg and 5 mg/kg.

In an embodiment, the antibody molecule is administered to the subject at a fixed dose between 10 mg and 1000 mg, e.g., between 10 mg and 500 mg, between 10 mg and 250 mg, between 10 mg and 150 mg, between 10 mg and 100 mg, between 10 mg and 50 mg, between 250 mg and 500 mg, between 150 mg and 500 mg, between 100 mg and 500 mg, between 50 mg and 500 mg, between 25 mg and 250 mg, between 50 mg and 150 mg, between 50 mg and 100 mg, between 100 mg and 150 mg. between 100 mg and 200 mg, or between 150 mg and 250 mg.

In an embodiment, the antibody molecule is administered once a week, twice a week, once every two weeks, once every three weeks, once every four weeks, once every eight weeks, once a month, once every two months, or once every three months.

In an embodiment, administration of the antibody molecule reduces an activity of FGF23 in a tissue, e.g., in parathyroid chief cells, renal tubular epithelial cells, renal fibroblasts, cardiac myocytes, cardiac fibroblasts, hepatocytes, macrophages, and neutrophils.

In an embodiment, the method further comprises administering to the subject a second therapy for the FGF23-associated disorder.

In an aspect, the disclosure features a method of reducing an activity of FGF23 in a cell or subject, the method comprising contacting the cell or subject, or administering to a subject in need thereof an effective amount of, an antibody molecule described herein or a composition described herein, thereby reducing the activity of FGF23.

In an embodiment, the cell is a human cell. In an embodiment, the subject is a human.

In an embodiment, the contacting step occurs in vitro, ex vivo, or in vivo. In an embodiment, the antibody molecule is administered to the subject intravenously.

In an embodiment, the antibody molecule is administered to the subject at a dose between 0.1 mg/kg and 50 mg/kg, e.g., between 0.2 mg/kg and 25 mg/kg, between 0.5 mg/kg and 10 mg/kg, between 0.5 mg/kg and 5 mg/kg, between 0.5 mg/kg and 3 mg/kg, between 0.5 mg/kg and 2.5 mg/kg, between 0.5 mg/kg and 2 mg/kg, between 0.5 mg/kg and 1.5 mg/kg, between 0.5 mg/kg and 1 mg/kg, between 1 mg/kg and 1.5 mg/kg, between 1 mg/kg and 2 mg/kg, between 1 mg/kg and 2.5 mg/kg, between 1 mg/kg and 3 mg/kg, between 1 mg/kg and 2.5 mg/kg, or between 1 mg/kg and 5 mg/kg.

In an embodiment, the antibody molecule is administered to the subject at a fixed dose between 10 mg and 1000 mg, e.g., between 10 mg and 500 mg, between 10 mg and 250 mg, between 10 mg and 150 mg, between 10 mg and 100 mg, between 10 mg and 50 mg, between 250 mg and 500 mg, between 150 mg and 500 mg, between 100 mg and 500 mg, between 50 mg and 500 mg, between 25 mg and 250 mg, between 50 mg and 150 mg, between 50 mg and 100 mg, between 100 mg and 150 mg. between 100 mg and 200 mg, or between 150 mg and 250 mg.

In an embodiment, the antibody molecule is administered once a week, twice a week, once every two weeks, once every three weeks, once every four weeks, once every eight weeks, once a month, once every two months, once every three months.

In an embodiment, administration of the antibody molecule reduces the activity of FGF23 in a tissue, e.g., in parathyroid chief cells, renal tubular epithelial cells, renal fibroblasts, cardiac myocytes, cardiac fibroblasts, hepatocytes, macrophages, and neutrophils.

In an aspect, the disclosure features use of an antibody molecule described herein or a composition described herein in the treatment, or in the manufacture of a medicament for the treatment, of a disorder described herein.

In another aspect, the disclosure features an antibody molecule described herein or a composition described herein for use in the treatment of a disorder described herein.

In an aspect, the disclosure features a method of detecting a FGF23 molecule, the method comprising contacting a cell or a sample from a subject with an antibody molecule described herein, thereby detecting the FGF23 molecule.

In an embodiment, the antibody molecule is coupled with a detectable label. In an embodiment, the FGF23 molecule is detected in vitro or ex vivo. In another embodiment, the FGF23 molecule is detected in vivo.

The disclosure contemplates all combinations of any one or more of the foregoing aspects and/or embodiments, as well as combinations with any one or more of the embodiments set forth in the detailed description and examples.

Other features, objects, and advantages of the compositions and methods herein will be apparent from the description and drawings, and from the claims.

### BRIEF DESCRIPTION OF THE DRAWINGS

FIG. 1 is a series of graphs showing analytical size exclusion chromatography (SEC) traces for exemplary anti-FGF23 monoclonal antibodies ExA11, ExA35, ExA28, ExA60, ExC17, ExC50, Exc23.1, Exc23.2, Exc23.3, Exc23.4 and Exc23.5.

FIGS. 2A-2B are a series of graphs showing ELISA binding of human FGF23 by exemplary anti-FGF23 antibodies. (A) ELISA binding of human FGF23 by exemplary anti-FGF23 antibodies Exa11, Exa28, Exa35, Exa43, Exa60, Exc17, Exc50, and a reference anti-FGF23 antibody (+ control). (B) ELISA binding of human FGF23 by exemplary anti-FGF23 antibodies Exc23, Exc23.1, Exc23.2, Exc23.3, Exc23.4, Exc23.5, and a reference anti-FGF23 antibody (+ control).

FIGS. 3A-3B are a series of graphs showing in vitro neutralization of FGF23 signaling by exemplary anti-FGF23 antibodies. (A) In vitro neutralization of FGF23 signaling by exemplary anti-FGF23 antibodies ExA11, ExA28, ExA35, ExA43, ExA60, ExC17, ExC50, and a reference anti-FGF23 antibody control. (B) In vitro neutralization of FGF23 signaling by exemplary anti-FGF23 antibodies Exc23.1, Exc23.3, a reference anti-FGF23 antibody (positive control), and an isotype control (negative control).

### DETAILED DESCRIPTION

Disclosed herein are antibody molecules that bind to FGF23, e.g., human FGF23, with high affinity and specificity. Advantageously, several of the antibody molecules describe herein have improved ability to reduce (e.g., inhibit, block, or neutralize) one or more biological activities of FGF23. Nucleic acid molecules encoding the antibody molecules, expression vectors, host cells, compositions (e.g., pharmaceutical compositions), kits, and methods for making the antibody molecules, are also provided. The antibody molecules and pharmaceutical compositions disclosed herein can be used (alone or in combination with other agents or therapeutic modalities) to treat, prevent and/or diagnose disorders and conditions, e.g., disorders and conditions associated with activation of FGF23.

### Definitions

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% (e.g., within 4%, 3%, 2%, or 1%) of a given value or range of values.

The compositions and methods disclosed herein encompass polypeptides and nucleic acids having the sequences specified, or sequences substantially identical or similar thereto, e.g., sequences at least 80%, 85%, 90%, 91%, 92%, 93%, 94%, 95%, 96%, 97%, 98% or 99% identical or higher to the sequence specified.

In the context of an amino acid sequence, the term “substantially identical” is used herein to refer to a first amino acid that contains a sufficient or minimum number of amino acid residues that are a) identical to, or b) conservative substitutions of aligned amino acid residues in a second amino acid sequence such that the first and second amino acid sequences can have a common structural domain and/or common functional activity. For example, amino acid sequences that contain a common structural domain having at least about 80%, 85%, 90%, 91%, 92%, 93%, 94%, 95%, 96%, 97%, 98% or 99% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 80%, 85%, 90%, 91%, 92%, 93%, 94%, 95%, 96%, 97%, 98% or 99% identity to a reference sequence, e.g., a sequence provided herein.

The term “functional variant” refers polypeptides that have a substantially identical amino acid sequence to the naturally-occurring sequence, or are encoded by a substantially identical nucleotide sequence, and are capable of having one or more activities of the naturally-occurring sequence.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a typical embodiment, the length of a reference sequence aligned for comparison purposes is at least 30%, e.g., at least 40%, 50%, 60%, 70%, 80%, 90%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In an embodiment, the percent identify between two amino acid or nucleotide sequences is determined using Clustal Omega (Sievers et al. Mol Syst Biol. 2011; 7:539). In an embodiment, the percent identify between two amino acid or nucleotide sequences is determined using Kalign2 (Lassmann et al. Nucleic Acids Res. 2009; 37(3):858-65; Lassmann and Sonnhammer BMC Bioinformatics. 2005; 6:298). In an embodiment, the percent identify between two amino acid or nucleotide sequences is determined using MAFFT (Katoh and Standley Mol Biol Evol. 2013; 30(4):772-80). In an embodiment, the percent identify between two amino acid or nucleotide sequences is determined using MUSCLE (Edgar Nucleic Acids Res. 2004; 32(5):1792-7; Edgar BMC Bioinformatics. 2004; 5:113). In an embodiment, the percent identify between two amino acid or nucleotide sequences is determined using MView (Brown et al. Bioinformatics. 1998; 14(4): 380-1). Other methods for determining the percent identify between two sequences are also described, e.g., in Li et al. Nucleic Acids Res. 2015; 43(W1):W580-4; McWilliam et al. Nucleic Acids Res. 2013; 41(Web Server issue):W597-600.

In an embodiment, the percent identity between two amino acid sequences is determined using the Needleman and Wunsch (J Mol Biol. 1970; 48(3):443-53) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6. In an embodiment, the percent identity between two nucleotide sequences is determined using the GAP program in the GCG software package (available at www.gcg.com), using an NWSgapdna. CMP matrix and a gap weight of 40, 50, 60, 70, or 80 and a length weight of 1, 2, 3, 4, 5, or 6. One suitable set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of Meyers and Miller (Comput Appl Biosci. 1988; 4(1):11-7) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases, for example, to identify other family members or related sequences. Such searches can be performed using the NBLAST and XBLAST programs (version 2.0) of Altschul, et al. 1990; J. Mol. Biol. 215:403-10. BLAST nucleotide searches can be performed with the NBLAST program, score=100, wordlength=12 to obtain nucleotide sequences homologous to a nucleic acid as described herein. BLAST protein searches can be performed with the XBLAST program, score=50, wordlength=3 to obtain amino acid sequences homologous to protein molecules described herein. To obtain gapped alignments for comparison purposes, Gapped BLAST can be utilized as described in Altschul et al., Nucleic Acids Res. 1997; 25:3389-3402. When utilizing BLAST and gapped BLAST programs, the default parameters of the respective programs (e.g., XBLAST and NBLAST) can be used. See www.ncbi.nlm.nih.gov.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6×sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions 4) are suitable conditions and the ones that should be used unless otherwise specified.

It is understood that the molecules described herein may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

The terms “polypeptide,” “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified, for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid,” “nucleic acid sequence,” “nucleotide sequence,” or “polynucleotide sequence,” and “polynucleotide” are used interchangeably. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a non-natural arrangement.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

As used herein, the term “treat,” e.g., a FGF23-associated disorder, means that a subject (e.g., a human) who has a disorder, e.g., a FGF23-associated disorder, and/or experiences a symptom of a disorder, e.g., a FGF23-associated disorder, will, in an embodiment, suffer less a severe symptom and/or recover faster when an antibody molecule is administered than if the antibody molecule were never administered. In an embodiment, when a FGF23-associated disorder, is treated, the level of FGF23 may be lower in a treated subject compared to a comparable untreated subject. For example, a diagnostic assay using immunofluorescence or electron microscopy will detect FGF23 in a biological sample of a subject after administration of an antibody molecule described herein for the effective treatment of the inflammatory disorder. Other assays, e.g., urine tests, blood tests, ultrasound, X-rays, or cystoscopy, can also be used to monitor treatment in a patient, or to detect the presence, e.g., decreased presence (or absence), of a symptom of the disorder, e.g., the FGF23-associated disorder, after treatment of the disorder in the subject. Treatment can, e.g., partially or completely, alleviate, ameliorate, relieve, inhibit, or reduce the severity of, and/or reduce incidence, and optionally, delay onset of, one or more manifestations of the effects or symptoms, features, and/or causes of a disorder, e.g., a FGF23-associated disorder. In an embodiment, treatment is of a subject who does not exhibit certain signs of a disorder, e.g., a FGF23-associated disorder, and/or of a subject who exhibits only early signs of a disorder, e.g., a FGF23-associated disorder. In an embodiment, treatment is of a subject who exhibits one or more established signs of a disorder, e.g., a FGF23-associated disorder. In an embodiment, treatment is of a subject diagnosed as suffering from a disorder, e.g., a FGF23-associated disorder. In an embodiment, the disorder is a FGF23-associated disorder described herein.

As used herein, the term “prevent,” a disorder, e.g., a FGF23-associated disorder, means that a subject (e.g., a human) is less likely to have the disorder, e.g., a FGF23-associated disorder, if the subject receives the antibody molecule. In an embodiment, the subject is at risk of developing the disorder, e.g., a FGF23-associated disorder. In an embodiment, the disorder is a FGF23-associated disorder described herein.

Various aspects of the compositions and methods herein are described in further detail below. Additional definitions are set out throughout the specification.

### FGF23

Fibroblast Growth Factor 23 (FGF23) is a protein that in humans is encoded by the FGF23 gene. FGF23 is a member of the fibroblast growth factor family and is involved in phosphate and vitamin D metabolism. FGF23 is secreted by osteocytes in response to calcitriol, and then acts upon the kidneys to reduce proximal tubule expression of NPT2, thereby decreasing reabsorption and increasing secretion of phosphate. Increased FGF23 activity is associated with renal phosphate loss in diseases such as hypophosphatemic rickets.

Exemplary amino acid and nucleotide sequences of human FGF23 are known, e.g., as described, in SEQ ID NO: 82, or Yamashita et al. Biochem Biophys Res Commun. 2000; 277(2): 494-498; Shimada et al. Proc Natl Acad Sci USA. 2001; 98(11): 6500-6505; or Shimada et al. Endocrinology. 2002;143(8):3179-82.

The exemplary amino acid sequence of human FGF23 is provided as follows.

Human FGF23
(SEQ ID NO: 82)
MLGARLRLWVCALCSVCSMSVLRAYPNASPLLGSSWGGLIHLYTATARNSY
HLQIHKNGHVDGAPHQTIYSALMIRSEDAGFVVITGVMSRRYLCMDFRGNI
FGSHYFDPENCRFQHQTLENGYDVYHSPQYHFLVSLGRAKRAFLPGMNPPP
YSQFLSRRNEIPLIHFNTPIPRRHTRSAEDDSERDPLNVLKPRARMTPAPA
SCSQELPSAEDNSPMASDPLGVVRGGRVNTHAGGTGPEGCRPFAKFI

Other variant and alternative sequences of human FGF23 are provided, e.g., as Genbank Accession No. NP_065689.1, as listed as of Feb. 1, 2019. The respective nucleotide sequences encoding the above-listed human FGF23 sequences are provided, e.g., as Genbank Accession No. NM_020638.2, as listed as of Feb. 1, 2019.

In an embodiment, when an anti-FGF23 antibody molecule binds, or substantially binds, to FGF23, it binds, or substantially binds, to one or more isoforms of FGF23, e.g., one or more isoforms of human FGF23 described herein. In an embodiment, the antibody molecule binds or substantially binds to FGF23 having the amino acid sequence of SEQ ID NO: 82.

### Antibody Molecules

Disclosed herein are antibody molecules that bind to FGF23, e.g., an anti-FGF23 antibody molecule described herein.

As used herein, the term “antibody molecule” refers to a protein, e.g., an immunoglobulin chain or a fragment thereof, comprising at least one immunoglobulin variable domain sequence. The term “antibody molecule” includes, for example, a full-length antibody and an antigen-binding fragment of an antibody.

For example, an antibody molecule can include a heavy (H) chain variable domain sequence (abbreviated herein as VH), and a light (L) chain variable domain sequence (abbreviated herein as VL). In another example, an antibody molecule includes two heavy (H) chain variable domain sequences and two light (L) chain variable domain sequence, thereby forming two antigen binding sites, such as Fab, Fab′, F(ab′)2, Fc, Fd, Fd′, Fv, single chain antibodies (scFv or sc(Fv)2, for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments retain the ability to selectively bind with their respective antigen or receptor. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The antibody molecules can be monoclonal or polyclonal. In an embodiment, the antibody molecule is a whole IgG antibody. The antibody molecule can also be a human, humanized, CDR-grafted, or in vitro generated antibody. The antibody molecule can have a heavy chain constant region chosen from, e.g., IgG1, IgG2, IgG3, IgG4, or a chimera of two or more isotypes. The antibody molecule can also have a light chain chosen from, e.g., kappa or lambda. The term “immunoglobulin” (Ig) is used interchangeably with the term “antibody” herein. In an embodiment, the antibody molecule is a multispecific antibody molecule (e.g., a bispecific antibody molecule).

Examples of antigen-binding fragments include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); (viii) a single domain antibody. These antibody fragments may be obtained using any suitable method, including several conventional techniques known to those with skill in the art, and the fragments can be screened for utility in the same manner as are intact antibodies.

The term “antibody” includes intact molecules as well as functional fragments thereof. Constant regions of the antibodies can be altered, e.g., mutated, to modify the properties of the antibody (e.g., to increase or decrease one or more of: Fc receptor binding, antibody glycosylation, the number of cysteine residues, effector cell function, or complement function).

In an embodiment, the antibody molecule is a single chain antibody. A single-chain antibody (scFv) may be engineered (see, for example, Colcher, D. et al. (1999) Ann N Y Acad Sci 880:263-80; and Reiter, Y. (1996) Clin Cancer Res 2:245-52). The single chain antibody can be dimerized or multimerized to generate multivalent antibodies having specificities for different epitopes of the same target protein.

In an embodiment, the antibody molecule is a single domain antibody. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, fish, shark, goat, rabbit, and bovine. In an embodiment, a single domain antibody is a naturally occurring single domain antibody known as heavy chain antibody devoid of light chains. Such single domain antibodies are disclosed in WO 94/04678, for example. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are also within the scope of the invention.

The VH and VL regions can be subdivided into regions of hypervariability, termed “complementarity determining regions” (CDR), interspersed with regions that are more conserved, termed “framework regions” (FR or FW). The terms “complementarity determining region,” and “CDR,” as used herein refer to the sequences of amino acids within antibody variable regions which confer antigen specificity and binding affinity. In general, there are three CDRs in each heavy chain variable region (HCDR1, HCDR2, HCDR3) and three CDRs in each light chain variable region (LCDR1, LCDR2, LCDR3). As used herein, the terms “framework,” “FW” and “FR” are used interchangeably.

The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242 (“Kabat” numbering scheme); Chothia, C. et al. (1987) J. Mol. Biol. 196:901-917 (“Chothia” numbering scheme); and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg). As used herein, the CDRs defined according the “Chothia” number scheme are also sometimes referred to as “hypervariable loops.” Under all definitions, each VH and VL typically includes three CDRs and four FRs, arranged from amino-terminus to carboxy-terminus in the following order: FR1, CDR1, FR2, CDR2, FR3, CDR3, FR4.

For example, under Kabat, the CDR amino acid residues in the heavy chain variable domain (VH) can be numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia the CDR amino acids in the VH can be numbered 26-32 (HCDR1), 52-56 (HCDR2), and 95-102 (HCDR3); and the amino acid residues in VL can be numbered 26-32 (LCDR1), 50-52 (LCDR2), and 91-96 (LCDR3). By combining the CDR definitions of both Kabat and Chothia, the CDRs can consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL.

Generally, unless specifically indicated, the anti-FGF23 antibody molecules described herein can include any combination of one or more Kabat CDRs and/or Chothia hypervariable loops, e.g., described in Table 1.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

The term “antigen-binding region” refers to the part of an antibody molecule that comprises determinants that form an interface that binds to an antigen, e.g., FGF23, e.g., human FGF23, or an epitope thereof. With respect to proteins (or protein mimetics), the antigen-binding region typically includes one or more loops (of at least, e.g., four amino acids or amino acid mimics) that form an interface that binds to the antigen, e.g., FGF23, e.g., human FGF23. Typically, the antigen-binding region of an antibody molecule includes at least one or two CDRs and/or hypervariable loops, or more typically at least three, four, five or six CDRs and/or hypervariable loops.

The terms “compete” or “cross-compete” are used interchangeably herein to refer to the ability of an antibody molecule to interfere with binding of an anti-FGF23 antibody molecule, e.g., an anti-FGF23 antibody molecule provided herein, to a target, e.g., FGF23, e.g., human FGF23. The interference with binding can be direct or indirect (e.g., through an allosteric modulation of the antibody molecule or the target). The extent to which an antibody molecule is able to interfere with the binding of another antibody molecule to the target, and therefore whether it can be said to compete, can be determined using a competition binding assay, for example, a FACS assay, an ELISA, or a BIACORE assay. In an embodiment, a competition binding assay is a quantitative competition assay. In an embodiment, a first anti-FGF23 antibody molecule is said to compete for binding to the target with a second anti-FGF23 antibody molecule when the binding of the first antibody molecule to the target is reduced by 10% or more, e.g., 20% or more, 30% or more, 40% or more, 50% or more, 55% or more, 60% or more, 65% or more, 70% or more, 75% or more, 80% or more, 85% or more, 90% or more, 95% or more, 98% or more, 99% or more in a competition binding assay (e.g., a competition assay described herein).

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

An “effectively human” protein is a protein that does not evoke a neutralizing antibody response, e.g., the human anti-murine antibody (HAMA) response. HAMA can be problematic in a number of circumstances, e.g., if the antibody molecule is administered repeatedly, e.g., in treatment of a chronic or recurrent disease condition. A HAMA response can make repeated antibody administration potentially ineffective because of an increased antibody clearance from the serum and potential allergic reactions (see, e.g., Saleh et al., Cancer Immunol. Immunother., 32:180-190 (1990); LoBuglio et al., Hybridoma, 5:5117-5123 (1986)).

The antibody molecule can be a polyclonal or a monoclonal antibody. In an embodiment, the antibody can be recombinantly produced, e.g., produced by any suitable phage display or combinatorial methods.

Various phage display and combinatorial methods for generating antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

In an embodiment, the antibody molecule is a fully human antibody (e.g., an antibody made in a mouse which has been genetically engineered to produce an antibody from a human immunoglobulin sequence), or a non-human antibody, e.g., a rodent (e.g., mouse or rat), goat, primate (e.g., monkey), camel antibody. In an embodiment, the non-human antibody is a rodent (e.g., mouse or rat antibody). Methods of producing rodent antibodies are known in the art.

Human monoclonal antibodies can be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see e.g., Wood et al. International Application WO 91/00906, Kucherlapati et al. PCT publication WO 91/10741; Lonberg et al. International Application WO 92/03918; Kay et al. International Application 92/03917; Lonberg, N. et al. 1994 Nature 368:856-859; Green, L.L. et al. 1994 Nature Genet. 7:13-21; Morrison, S. L. et al. 1994 Proc. Natl. Acad. Sci. USA 81:6851-6855; Bruggeman et al. 1993 Year Immunol 7:33-40; Tuaillon et al. 1993 PNAS 90:3720-3724; Bruggeman et al. 1991 Eur J Immunol 21:1323-1326).

An antibody can be one in which the variable region, or a portion thereof, e.g., the CDRs, are generated in a non-human organism, e.g., a rat or mouse. Chimeric, CDR-grafted, and humanized antibodies are within the invention. Antibodies generated in a non-human organism, e.g., a rat or mouse, and then modified, e.g., in the variable framework or constant region, to decrease antigenicity in a human are within the invention.

Chimeric antibodies can be produced by any suitable recombinant DNA technique. Several are known in the art (see Robinson et al., International Patent Application Publication No. WO1987/002671; Akira, et al., European Patent Application Publication No. 184,187; Taniguchi, M., European Patent Application Publication No. 171,496; Morrison et al., European Patent Application Publication No. 173,494; Neuberger et al., International Patent Application Publication No. WO 86/01533; Cabilly et al. U.S. Pat. No. 4,816,567; Cabilly et al., European Patent Application Publication No. 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

A humanized or CDR-grafted antibody will have at least one or two but generally all three recipient CDRs (of heavy and or light immunoglobulin chains) replaced with a donor CDR. The antibody may be replaced with at least a portion of a non-human CDR or only some of the CDRs may be replaced with non-human CDRs. It is only necessary to replace the number of CDRs required for binding of the humanized antibody to lipopolysaccharide. In an embodiment, the donor will be a rodent antibody, e.g., a rat or mouse antibody, and the recipient will be a human framework or a human consensus framework. Typically, the immunoglobulin providing the CDRs is called the “donor” and the immunoglobulin providing the framework is called the “acceptor.” In an embodiment, the donor immunoglobulin is a non-human (e.g., rodent). The acceptor framework is typically a naturally-occurring (e.g., a human) framework or a consensus framework, or a sequence about 85% or higher, e.g., 90%, 95%, 99% or higher identical thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody can be humanized by any suitable method, and several such methods known in the art (see e.g., Morrison, S. L., 1985, Science 229:1202-1207, by Oi et al., 1986, BioTechniques 4:214, and by Queen et al. U.S. Pat. Nos. 5,585,089, 5,693,761 and 5,693,762, the contents of all of which are hereby incorporated by reference).

Humanized or CDR-grafted antibodies can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare humanized antibodies (UK Patent Application GB 2188638A, filed on Mar. 26, 1987; Winter U.S. Pat. No. 5,225,539), the contents of which is expressly incorporated by reference.

Also provided are humanized antibodies in which specific amino acids have been substituted, deleted or added. Criteria for selecting amino acids from the donor are described in, e.g., U.S. Pat. No. 5,585,089, e.g., columns 12-16 of U.S. Pat. No. 5,585,089, the contents of which are hereby incorporated by reference. Other techniques for humanizing antibodies are described in Padlan et al. EP 519596 A1, published on Dec. 23, 1992.

In an embodiment, the antibody molecule has a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody molecule (e.g., to increase or decrease one or more of: Fc receptor binding, antibody glycosylation, the number of cysteine residues, effector cell function, and/or complement function). In an embodiment, the antibody molecule has effector function and can fix complement. In another embodiment, the antibody molecule does not recruit effector cells or fix complement. In an embodiment, the antibody molecule has reduced or no ability to bind an Fc receptor. For example, it may be an isotype or subtype, fragment or other mutant, which does not support binding to an Fc receptor, e.g., it has a mutated or deleted Fc receptor binding region.

In an embodiment, a constant region of the antibody molecule is altered. Methods for altering an antibody constant region are known in the art. Antibody molecules s with altered function, e.g. altered affinity for an effector ligand, such as FcR on a cell, or the C1 component of complement can be produced by replacing at least one amino acid residue in the constant portion of the antibody with a different residue (see, e.g., EP 388,151 A1, U.S. Pat. Nos. 5,624,821 and 5,648,260, the contents of all of which are hereby incorporated by reference) Amino acid mutations which stabilize antibody structure, such as S228P (EU nomenclature, S241P in Kabat nomenclature) in human IgG4 are also contemplated. Similar type of alterations could be described which if applied to the murine, or other species immunoglobulin would reduce or eliminate these functions.

In an embodiment, the only amino acids in the antibody molecule are canonical amino acids. In an embodiment, the antibody molecule comprises naturally-occurring amino acids; analogs, derivatives and congeners thereof; amino acid analogs having variant side chains; and/or all stereoisomers of any of any of the foregoing. The antibody molecule may comprise the D- or L-optical isomers of amino acids and peptidomimetics.

In an embodiment, the antibody molecule comprises a monoclonal antibody (e.g., a full length antibody which has an immunoglobulin Fc region). In an embodiment, the antibody molecule comprises a full length antibody or full length immunoglobulin chain. In an embodiment, the antibody molecule comprises an antigen binding or functional fragment of a full length antibody or full length immunoglobulin chain.

In an embodiment, the antibody molecule is a monospecific antibody molecule, e.g., it binds a single epitope. For example, a monospecific antibody molecule can have a plurality of immunoglobulin variable region sequences, each of which binds the same epitope.

In an embodiment, the antibody molecule is a multispecific antibody molecule, e.g., it comprises a plurality of immunoglobulin variable region sequences, wherein a first immunoglobulin variable region sequence of the plurality has binding specificity for a first epitope and a second immunoglobulin variable region sequence of the plurality has binding specificity for a second epitope. In an embodiment, the first and second epitopes are on the same antigen, e.g., the same protein (or subunit of a multimeric protein). In an embodiment, the first and second epitopes overlap. In an embodiment, the first and second epitopes do not overlap. In an embodiment, the first and second epitopes are on different antigens, e.g., the different proteins (or different subunits of a multimeric protein). In an embodiment, a multispecific antibody molecule comprises a third, fourth or fifth immunoglobulin variable domain. In an embodiment, a multispecific antibody molecule is a bispecific antibody molecule, a trispecific antibody molecule, or tetraspecific antibody molecule.

In an embodiment, a multispecific antibody molecule is a bispecific antibody molecule. A bispecific antibody has specificity for no more than two antigens. A bispecific antibody molecule is typically characterized by a first immunoglobulin variable domain sequence which has binding specificity for a first epitope and a second immunoglobulin variable domain sequence that has binding specificity for a second epitope. In an embodiment the first and second epitopes are on the same antigen, e.g., the same protein (or subunit of a multimeric protein). In an embodiment the first and second epitopes overlap. In an embodiment, the first and second epitopes do not overlap. In an embodiment, the first and second epitopes are on different antigens, e.g., the different proteins (or different subunits of a multimeric protein). In an embodiment, a bispecific antibody molecule comprises a heavy chain variable region sequence and a light chain variable region sequence which have binding specificity for a first epitope, and a heavy chain variable region sequence and a light chain variable region sequence which have binding specificity for a second epitope. In an embodiment, a bispecific antibody molecule comprises a half antibody having binding specificity for a first epitope and a half antibody having binding specificity for a second epitope. In an embodiment, a bispecific antibody molecule comprises a half antibody, or a fragment thereof, having binding specificity for a first epitope, and a half antibody, or fragment thereof, having binding specificity for a second epitope. In an embodiment a bispecific antibody molecule comprises an scFv, or a fragment thereof, have binding specificity for a first epitope, and an scFv, or a fragment thereof, have binding specificity for a second epitope.

Protocols for generating bispecific or heterodimeric antibody molecules are known in the art; including but not limited to, for example, the “knob in a hole” approach described in, e.g., U.S. Pat. No. 5,731,168; the electrostatic steering Fc pairing as described in, e.g., WO 09/089004, WO 06/106905 and WO 2010/129304; Strand Exchange Engineered Domains (SEED) heterodimer formation as described in, e.g., WO 07/110205; Fab arm exchange as described in, e.g., WO 08/119353, WO 2011/131746, and WO 2013/060867; double antibody conjugate, e.g., by antibody cross-linking to generate a bi-specific structure using a heterobifunctional reagent having an amine-reactive group and a sulfhydryl reactive group as described in, e.g., U.S. Pat. No. 4,433,059; bispecific antibody determinants generated by recombining half antibodies (heavy-light chain pairs or Fabs) from different antibodies through cycle of reduction and oxidation of disulfide bonds between the two heavy chains, as described in, e.g., U.S. Pat. No. 4,444,878; trifunctional antibodies, e.g., three Fab′ fragments cross-linked through sulfhydryl reactive groups, as described in, e.g., U.S. Pat. No. 5,273,743; biosynthetic binding proteins, e.g., pair of scFvs cross-linked through C-terminal tails preferably through disulfide or amine-reactive chemical cross-linking, as described in, e.g., U.S. Pat. No. 5,534,254; bifunctional antibodies, e.g., Fab fragments with different binding specificities dimerized through leucine zippers (e.g., c-fos and c-jun) that have replaced the constant domain, as described in, e.g., U.S. Pat. No. 5,582,996; bispecific and oligospecific mono- and oligovalent receptors, e.g., VH-CH1 regions of two antibodies (two Fab fragments) linked through a polypeptide spacer between the CH1 region of one antibody and the VH region of the other antibody typically with associated light chains, as described in, e.g., U.S. Pat. No. 5,591,828; bispecific DNA-antibody conjugates, e.g., crosslinking of antibodies or Fab fragments through a double stranded piece of DNA, as described in, e.g., U.S. Pat. No. 5,635,602; bispecific fusion proteins, e.g., an expression construct containing two scFvs with a hydrophilic helical peptide linker between them and a full constant region, as described in, e.g., U.S. Pat. No. 5,637,481; multivalent and multispecific binding proteins, e.g., dimer of polypeptides having first domain with binding region of Ig heavy chain variable region, and second domain with binding region of Ig light chain variable region, generally termed diabodies (higher order structures are also disclosed creating bispecific, trispecific, or tetraspecific molecules, as described in, e.g., U.S. Pat. No. 5,837,242; minibody constructs with linked VL and VH chains further connected with peptide spacers to an antibody hinge region and CH3 region, which can be dimerized to form bispecific/multivalent molecules, as described in, e.g., U.S. Pat. No. 5,837,821; VH and VL domains linked with a short peptide linker (e.g., 5 or 10 amino acids) or no linker at all in either orientation, which can form dimers to form bispecific diabodies; trimers and tetramers, as described in, e.g., U.S. Pat. No. 5,844,094; String of VH domains (or VL domains in family members) connected by peptide linkages with crosslinkable groups at the C-terminus further associated with VL domains to form a series of FVs (or scFvs), as described in, e.g., U.S. Pat. No. 5,864,019; and single chain binding polypeptides with both a VH and a VL domain linked through a peptide linker are combined into multivalent structures through non-covalent or chemical crosslinking to form, e.g., homobivalent, heterobivalent, trivalent, and tetravalent structures using both scFV or diabody type format, as described in, e.g., U.S. Pat. No. 5,869,620. The contents of the above-referenced applications are incorporated herein by reference in their entirety.

Additional methods of making multispecific or bispecific antibody molecules can be found, for example, in U.S. Pat. Nos. 5,910,573, 5,932,448, 5,959,083, 5,989,830, 6,005,079, 6,239,259, 6,294,353, 6,333,396, 6,476,198, 6,511,663, 6,670,453, 6,743,896, 6,809,185, 6,833,441, 7,129,330, 7,183,076, 7,521,056, 7,527,787, 7,534,866, 7,612,181, US2002/004587, US2002/076406, US2002/103345, US2003/207346, US2003/211078, US2004/219643, US2004/220388, US2004/242847, US2005/003403, US2005/004352, US2005/069552, US2005/079170, US2005/100543, US2005/136049, US2005/136051, US2005/163782, US2005/266425, US2006/083747, US2006/120960, US2006/204493, US2006/263367, US2007/004909, US2007/087381, US2007/128150, US2007/141049, US2007/154901, US2007/274985, US2008/050370, US2008/069820, US2008/152645, US2008/171855, US2008/241884, US2008/254512, US2008/260738, US2009/130106, US2009/148905, US2009/155275, US2009/162359, US2009/162360, US2009/175851, US2009/175867, US2009/232811, US2009/234105, US2009/263392, US2009/274649, EP346087, WO00/06605, WO02/072635, WO04/081051, WO06/020258, WO2007/044887, WO2007/095338A2, WO2007/137760A2, WO2008/119353, WO2009/021754, WO2009/068630, WO91/03493, WO93/23537, WO94/09131, WO94/12625, WO95/09917, WO96/37621, WO99/64460. The contents of the above-referenced applications are incorporated herein by reference in their entirety.

A polypeptide of an antibody molecule described herein may be linear or branched, it may comprise modified amino acids, and it may be interrupted by non-amino acids. The antibody molecule may also be modified; for example, by disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The antibody molecule described herein can be used alone in unconjugated form, or can be bound to a substance, e.g., a toxin or moiety (e.g., a therapeutic drug; a compound emitting radiation; molecules of plant, fungal, or bacterial origin; or a biological protein (e.g., a protein toxin) or particle (e.g., a recombinant viral particle, e.g., via a viral coat protein). For example, the anti-FGF23 antibody can be coupled to a radioactive isotope such as an α-, β-, or γ-emitter, or a β- and γ-emitter.

An antibody molecule can be derivatized or linked to another functional molecule (e.g., another peptide or protein). As used herein, a “derivatized” antibody molecule is one that has been modified. Methods of derivatization include but are not limited to the addition of a fluorescent moiety, a radionucleotide, a toxin, an enzyme or an affinity ligand such as biotin. Accordingly, the antibody molecules are intended to include derivatized and otherwise modified forms of the antibodies described herein, including immunoadhesion molecules. For example, an antibody molecule can be functionally linked (by chemical coupling, genetic fusion, noncovalent association or otherwise) to one or more other molecular entities, such as another antibody (e.g., a bispecific antibody or a diabody), a detectable agent, a toxin, a pharmaceutical agent, and/or a protein or peptide that can mediate association of the antibody or antibody portion with another molecule (such as a streptavidin core region or a polyhistidine tag).

Some types of derivatized antibody molecule are produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Useful detectable agents with which an anti-dengue antibody molecule may be derivatized (or labeled) to include fluorescent compounds, various enzymes, prosthetic groups, luminescent materials, bioluminescent materials, fluorescent emitting metal atoms, e.g., europium (Eu), and other anthanides, and radioactive materials (described below). Exemplary fluorescent detectable agents include fluorescein, fluorescein isothiocyanate, rhodamine, 5dimethylamine-1-napthalenesulfonyl chloride, phycoerythrin and the like. An antibody may also be derivatized with detectable enzymes, such as alkaline phosphatase, horseradish peroxidase, β-galactosidase, acetylcholinesterase, glucose oxidase and the like. When an antibody is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. An antibody molecule may also be derivatized with a prosthetic group (e.g., streptavidin/biotin and avidin/biotin). For example, an antibody may be derivatized with biotin, and detected through indirect measurement of avidin or streptavidin binding. Examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of bioluminescent materials include luciferase, luciferin, and aequorin.

Labeled antibody molecules can be used, for example, diagnostically and/or experimentally in a number of contexts, including (i) to isolate a predetermined antigen by standard techniques, such as affinity chromatography or immunoprecipitation; (ii) to detect a predetermined antigen (e.g., in a cellular lysate or cell supernatant) in order to evaluate the abundance and pattern of expression of the protein; (iii) to monitor protein levels in tissue as part of a clinical testing procedure, e.g., to determine the efficacy of a given treatment regimen.

An antibody molecule described herein can be conjugated to another molecular entity, typically a label or a therapeutic (e.g., antimicrobial (e.g., antibacterial or bactericidal), immunomodulatory, immunostimulatory, cytotoxic, or cytostatic) agent or moiety. Radioactive isotopes can be used in diagnostic or therapeutic applications. Radioactive isotopes that can be coupled to the antibody molecules include, but are not limited to α-, β-, or γ-emitters, or β- and γ-emitters. Such radioactive isotopes include, but are not limited to iodine (¹³¹I or ¹²⁵I), yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), indium (¹¹¹In), technetium (⁹⁹mTc), phosphorus (³²P), rhodium (¹⁸⁸Rh), sulfur (³⁵S), carbon (¹⁴C), tritium (³H), chromium (⁵¹Cr), chlorine (³⁶Cl), cobalt (⁵⁷Co or ⁵⁸Co), iron (⁵⁹Fe), selenium (⁷⁵Se), or gallium (⁶⁷Ga). Radioisotopes useful as therapeutic agents include yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), and rhodium (¹⁸⁸Rh). Radioisotopes useful as labels, e.g., for use in diagnostics, include iodine (¹³¹I or ¹²⁵I), indium (¹¹¹In), technetium (⁹⁹mTc), phosphorus (³²P), carbon (¹⁴C), and tritium (³H), or one or more of the therapeutic isotopes listed above.

The present disclosure provides radiolabeled antibody molecules and methods of labeling the same. In an embodiment, a method of labeling an antibody molecule is disclosed. The method includes contacting an antibody molecule, with a chelating agent, to thereby produce a conjugated antibody. The conjugated antibody is radiolabeled with a radioisotope, e.g., ¹¹¹Indium, ⁹⁰Yttrium and ¹⁷⁷Lutetium, to thereby produce a labeled antibody molecule.

In an embodiment, the antibody molecule is conjugated to a therapeutic agent. Therapeutically active radioisotopes are disclosed herein. Examples of other therapeutic agents include, but are not limited to, taxol, cytochalasin B, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, maytansinoids, e.g., maytansinol (see e.g., U.S. Pat. No. 5,208,020), CC-1065 (see e.g., U.S. Pat. Nos. 5,475,092, 5,585,499, 5,846,545) and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065, melphalan, carmustine (BSNU) and lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol and maytansinoids).

In an embodiment, the anti-FGF23 antibody molecule (e.g., a monospecific, bispecific, or multispecific antibody molecule) is covalently linked, e.g., fused, to another partner e.g., a protein, e.g., as a fusion molecule (e.g., a fusion protein).

As used herein, a “fusion protein” and “fusion polypeptide” refer to a polypeptide having at least two portions covalently linked together, where each of the portions is a polypeptide. In an embodiment, each of the portions is a polypeptide that has a different property. The property can be a biological property, such as activity in vitro or in vivo. The property can also be simple chemical or physical property, such as binding to a target molecule, catalysis of a reaction, etc. The two portions can be linked directly by a single peptide bond or through a linker (e.g., peptide linker), but are in reading frame with each other.

In one aspect, the invention features a method of providing a target binding agent that specifically binds to FGF23 (e.g., human FGF23). For example, the target binding molecule is an antibody molecule. The method includes: providing a target protein that comprises at least a portion of non-human protein, the portion being homologous to (e.g., at least 70, 75, 80, 85, 87, 90, 92, 94, 95, 96, 97, 98% identical to) a corresponding portion of a human target protein, but differing by at least one amino acid (e.g., at least one, two, three, four, five, six, seven, eight, or nine amino acids); obtaining a binding agent (e.g., an antibody molecule) that specifically binds to the target protein; and evaluating efficacy of the binding agent in modulating an activity of the target protein. The method can further include administering the binding agent (e.g., antibody molecule) or a derivative (e.g., a humanized antibody molecule) to a subject (e.g., a human subject).

In another aspect, this disclosure provides a method of making an antibody molecule disclosed herein. The method includes: providing an antigen, e.g., FGF23 (e.g., human FGF23) or a fragment thereof; obtaining an antibody molecule that specifically binds to the antigen; evaluating efficacy of the antibody molecule in modulating activity of the antigen and/or organism expressing the antigen, e.g., FGF23, e.g., human FGF23. The method can further include administering the antibody molecule, including a derivative thereof (e.g., a humanized antibody molecule) to a subject, e.g., a human.

This disclosure provides an isolated nucleic acid molecule encoding the above antibody molecule, vectors and host cells thereof. The nucleic acid molecule includes, but is not limited to, RNA, genomic DNA and cDNA.

Amino acid and nucleotide sequences of exemplary antibody molecules are described in Tables 1-5.

TABLE 1
Amino acid sequences of heavy chain variable regions (VHs) and light
chain variable regions (VLs) of exemplary antibody molecules

| | | | SEQ | | | SEQ |
|---|---|---|---|---|---|---|
| Antibody | Chain | Amino Acid Sequence | ID NO | CDR Sequence | | ID NO |
| ExA11 | VH | QVQLVQSGAELKKPGASVKVSCKASGYTFTNHYMHWVRQAP | 5 | HCDR1 | NHYMH | 41 |
| | | GQGLEWMGIINPISGSTTYAQKFQGRVTMTRDTSSSTAYMD | | HCDR2 | IINPISGSTTYAQKFQG | 48 |
| | | LSRLTSDDTAVYYCARDIVDAFDFWGQGTTVTVSS | | HCDR3 | DIVDAFDF | 53 |
| | VL | AIQLTQSPSSLSASVGDRVTITCRASQGISSALVWYQQKPG | 19 | LCDR1 | RASQGISSALV | 61 |
| | | QAPRLLIYDASSLESGIPARFSGSGSGTDFTLTISSLEPED | | LCDR2 | DASSLES | 67 |
| | | FAVYYCQQFNDYFTFGQGTKLEIK | | LCDR3 | QQFNDYFT | 74 |
| ExA28 | VH | EVQLVQSGAEVRKPGASVKVSCKASGYTFTNHYMHWVRQAP | 7 | HCDR1 | NHYMH | 41 |
| | | GQGLEWMGIINPISGSTSNAQKLQGRVTFTRDTSANTAYME | | HCDR2 | IINPISGSTSNAQKLQG | 46 |
| | | LSGLIPEDTAIYYCARDIVDAFDFWGQGTLVTVSS | | HCDR3 | DIVDAFDF | 53 |
| | VL | AIQMTQSPSSLSASVGDRVTITCRASQGISSALVWYQQKPG | 20 | LCDR1 | RASQGISSALV | 61 |
| | | KAPKLLIYDASSLESGVPSRFSGSGSGTDFTFTISSLQPED | | LCDR2 | DASSLES | 67 |
| | | IATYYCQQFNDYFTFGPGTKLEIK | | LCDR3 | QQFNDYFT | 74 |
| ExA35 | VH | QVQLLESGAVLARPGTSVKISCKASGYTFTNHYMHWVKQRP | 8 | HCDR1 | NHYMH | 41 |
| | | GQGLEWIGIINPITGSSSNAQKFQGRVTITRDTSTSTVFME | | HCDR2 | IINPITGSSSNAQKFQG | 49 |
| | | LSSLRSEDTAVYYCARDIVDAFDFWGQGTLVTVSS | | HCDR3 | DIVDAFDF | 53 |
| | VL | AIQLTQSPSSLSASVGDRVTITCRASQGISSALVWYQQKPG | 19 | LCDR1 | RASQGISSALV | 61 |
| | | QAPRLLIYDASSLESGIPARFSGSGSGTDFTLTISSLEPED | | LCDR2 | DASSLES | 67 |
| | | FAVYYCQQFNDYFTFGQGTKLEIK | | LCDR3 | QQFNDYFT | 74 |
| ExA43 | VH | EVQLVQSGAEVRKPGASVKVSCKASGYTFTNHYMHWVRQAP | 9 | HCDR1 | NHYMH | 41 |
| | | GQGLEWMGIINPISGSTSNAQKLQGRVTFTRDTSANTAYME | | HCDR2 | IINPISGSTSNAQKLQG | 46 |
| | | LSGLIPEDTAIYYCAREIVDAFDFWGQGTLVTVSS | | HCDR3 | EIVDAFDF | 57 |
| | VL | AIQLTQSPSSLSASVGDRVTITCRASQGISSALVWYQQKPG | 19 | LCDR1 | RASQGISSALV | 61 |
| | | QAPRLLIYDASSLESGIPARFSGSGSGTDFTLTISSLEPED | | LCDR2 | DASSLES | 67 |
| | | FAVYYCQQFNDYFTFGQGTKLEIK | | LCDR3 | QQFNDYFT | 74 |
| ExA60 | VH | EVQLVQSGAEVRKPGASVKVSCKASGYSFTNHYMHWVRQAP | 11 | HCDR1 | NHYMH | 41 |
| | | GQGLEWMGIINPNSGSTSNAQKLQGRVTFTRDTSANTAYME | | HCDR2 | IINPNSGSTSNAQKLQG | 50 |
| | | LSGLIPEDTAIYYCARDIVDAFDYWGQGTLVTVSS | | HCDR3 | DIVDAFDY | 54 |
| | VL | AIQMTQSPSSLSASVGDRVTITCRASQGISSALVWYQQKPG | 20 | LCDR1 | RASQGISSALV | 61 |
| | | KAPKLLIYDASSLESGVPSRFSGSGSGTDFTFTISSLQPED | | LCDR2 | DASSLES | 67 |
| | | IATYYCQQFNDYFTFGPGTKLEIK | | LCDR3 | QQFNDYFT | 74 |
| ExC17 | VH | EVQLVQSGAEVRKPGASVKVSCKASGYTFTNHYMHWVRQAP | 7 | HCDR1 | NHYMH | 41 |
| | | GQGLEWMGIINPISGSTSNAQKLQGRVTFTRDTSANTAYME | | HCDR2 | IINPISGSTSNAQKLQG | 46 |
| | | LSGLIPEDTAIYYCARDIVDAFDFWGQGTLVTVSS | | HCDR3 | DIVDAFDF | 53 |
| | VL | DIQLTQSPSSLSASVGDRVTITCRASQGISSALVWYQQKPG | 28 | LCDR1 | RASQGISSALV | 61 |
| | | QAPRLLIYDASSLQSGIPARFSGSGSGTDFTLTISSLEPED | | LCDR2 | DASSLQS | 69 |
| | | FAVYYCQQFNDLFTFGQGTKLEIK | | LCDR3 | QQFNDLFT | 89 |
| ExC50 | VH | QVQLLESGAVLARPGTSVKISCKASGYSFTNHYMHWVKQRP | 13 | HCDR1 | NHYMH | 41 |
| | | GQGLEWIGIINPITGSSSNAQKFQGRVTITRDTSTSTVFME | | HCDR2 | IINPITGSSSNAQKFQG | 49 |
| | | LSSLRSEDTAVYYCARDLVDAFDFWGQGTLVTVSS | | HCDR3 | DLVDAFDF | 55 |
| | VL | DIQLTQSPSSLSASVGDRVTITCRASQGISSALVWYQQKPG | 25 | LCDR1 | RASQGISSALV | 61 |
| | | QAPRLLIYDASSLQSGIPARFSGSGSGTDFTLTISSLEPED | | LCDR2 | DASSLQS | 69 |
| | | FAVYYCQQFNDYFTFGQGTKLEIK | | LCDR3 | QQFNDYFT | 74 |
| Exc23 | VH | EVQLVQSGAEVRKPGASVKVSCKASGYTFTNHYMHWVRQAP | 7 | HCDR1 | NHYMH | 41 |
| | | GQGLEWMGIINPISGSTSNAQKLQGRVTFTRDTSANTAYME | | | | |
| | | LSGLIPEDTAIYYCARDIVDAFDFWGQGTLVTVSS | | | | |
| | | | | HCDR2 | IINPISGSTSNAQKLQG | 46 |
| | | | | HCDR3 | DIVDAFDF | 53 |
| | VL | DIQMTQSPSSLSASVGDRVTITCKASQGISSALVWYQQKPG | 37 | LCDR1 | KASQGISSALV | 59 |
| | | KAPKLLIYDASSLQTGVPSRFSGSGSGTDFTFTISSLQPED | | LCDR2 | DASSLQT | 70 |
| | | IATYYCQQFNDYFSFGPGTKLEIK | | LCDR3 | QQFNDYFS | 73 |
| Exc23.1 | VH | EVQLVQSGAEVRKPGASVKVSCKASGYTFTNHYMHWVRQAP | 7 | HCDR1 | NHYMH | 41 |
| | | GQGLEWMGIINPISGSTSNAQKLQGRVTFTRDTSANTAYME | | HCDR2 | IINPISGSTSNAQKLQG | 46 |
| | | LSGLIPEDTAIYYCARDIVDAFDFWGQGTLVTVSS | | HCDR3 | DIVDAFDF | 53 |
| | VL | DIQMTQSPSSLSASVGDRVTITCKASQGISSALVWYQQKPG | 34 | LCDR1 | KASQGISSALV | 59 |
| | | KAPKLLIYDASSLETGVPSRFSGSGSGTDFTFTISSLQPED | | LCDR2 | DASSLET | 68 |
| | | IATYYCQQFNDYFSFGPGTKLEIK | | LCDR3 | QQFNDYFS | 73 |
| Exc23.2 | VH | EVQLVQSGAEVRKPGASVKVSCKASGYTFTNHYMHWVRQAP | 7 | HCDR1 | NHYMH | 41 |
| | | GQGLEWMGIINPISGSTSNAQKLQGRVTFTRDTSANTAYME | | HCDR2 | IINPISGSTSNAQKLQG | 46 |
| | | LSGLIPEDTAIYYCARDIVDAFDFWGQGTLVTVSS | | HCDR3 | DIVDAFDF | 53 |
| | VL | DIQMTQSPSSLSASVGDRVTITCKASQGISSALVWYQQKPG | 36 | LCDR1 | KASQGISSALV | 59 |
| | | KAPKLLIYDASSLQSGVPSRFSGSGSGTDFTFTISSLQPED | | LCDR2 | DASSLQS | 69 |
| | | IATYYCQQFNDYFSFGPGTKLEIK | | LCDR3 | QQFNDYFS | 73 |
| Exc23.3 | VH | EVQLVQSGAEVRKPGASVKVSCKASGYTFTNHYMHWVRQAP | 7 | HCDR1 | NHYMH | 41 |
| | | GQGLEWMGIINPISGSTSNAQKLQGRVTFTRDTSANTAYME | | HCDR2 | IINPISGSTSNAQKLQG | 46 |
| | | LSGLIPEDTAIYYCARDIVDAFDFWGQGTLVTVSS | | HCDR3 | DIVDAFDF | 53 |
| | VL | DIQMTQSPSSLSASVGDRVTITCKASQGISSALVWYQQKPG | 94 | LCDR1 | KASQGISSALV | 59 |
| | | KAPKLLIYDASSLQTGVPSRFSGSGSGTDFTFTISSLQPED | | LCDR2 | DASSLQT | 70 |
| | | IATYYCQQFNDYFTFGPGTKLEIK | | LCDR3 | QQFNDYFT | 74 |
| Exc23.4 | VH | EVQLVQSGAEVRKPGASVKVSCKASGYTFTNHYMHWVRQAP | 7 | HCDR1 | NHYMH | 41 |
| | | GQGLEWMGIINPISGSTSNAQKLQGRVTFTRDTSANTAYME | | HCDR2 | IINPISGSTSNAQKLQG | 46 |
| | | LSGLIPEDTAIYYCARDIVDAFDFWGQGTLVTVSS | | HCDR3 | DIVDAFDF | 53 |
| | VL | DIQMTQSPSSLSASVGDRVTITCRASQGISSALVWYQQKPG | 95 | LCDR1 | RASQGISSALV | 61 |
| | | KAPKLLIYDASSLQTGVPSRFSGSGSGTDFTFTISSLQPED | | LCDR2 | DASSLQT | 70 |
| | | IATYYCQQFNDYFSFGPGTKLEIK | | LCDR3 | QQFNDYFS | 73 |
| Exc23.5 | VH | EVQLVQSGAEVRKPGASVKVSCKASGYTFTNHYMHWVRQAP | 7 | HCDR1 | NHYMH | 41 |
| | | GQGLEWMGIINPISGSTSNAQKLQGRVTFTRDTSANTAYME | | HCDR2 | IINPISGSTSNAQKLQG | 46 |
| | | LSGLIPEDTAIYYCARDIVDAFDFWGQGTLVTVSS | | HCDR3 | DIVDAFDF | 53 |
| | VL | AIQMTQSPSSLSASVGDRVTITCKASQGISSALVWYQQKPG | 96 | LCDR1 | KASQGISSALV | 59 |
| | | KAPKLLIYDASSLQTGVPSRFSGSGSGTDFTFTISSLQPED | | LCDR2 | DASSLQT | 70 |
| | | IATYYCQQFNDYFSFGPGTKLEIK | | LCDR3 | QQFNDYFS | 73 |

TABLE 2
List of Exemplary VH and VL sequences. Underlined Bolding indicates CDR sequences.

| Description | SEQ ID NO: | Amino Acid Sequence |
|---|---|---|
| VH-1 | 1 | EVQLLEQSGAELKKPGASVKVSCKASGYTFTNHYMHWVRQAPGQGLEWMGIINPISGSTSYAQKFQGRVT |
| | | MTRDTSSSTAYMDLSRLTSDDTAVYYCARDIVDAFDFWGQGTTVTVSS |
| VH-2 | 2 | EVQLLEQSGAELKKPGASVKVSCKASGYTFTSHYMHWVRQAPGQGLEWMGIINPISGSTTYAQKFQGRVT |
| | | MTRDTSSSTAYMDLSRLTSDDTAVYYCARDIVDAFDYWGQGTTVTVSS |
| VH-3 | 3 | EVQLLEQSGAELKKPGASVKVSCKASGYTFTNHFMHWVRQAPGQGLEWMGTINPVSGSTSYAQKFQGRVT |
| | | MTRDTSSSTAYMDLSRLTSDDTAVYYCAREILDAFDYWGQGTTVTVSS |
| VH-4 | 4 | EVQLLEQSGAELKKPGASVKVSCKASGYTFTNHFIHWVRQAPGQGLEWMGTINPVSGSTNYAQKFQGRVT |
| | | MTRDTSSSTAYMDLSRLTSDDTAVYYCAREILDAFDYWGQGTTVTVSS |
| VH-5 | 5 | QVQLVQSGAELKKPGASVKVSCKASGYTFTNHYMHWVRQAPGQGLEWMGIINPISGSTTYAQKFQGRVTM |
| | | TRDTSSSTAYMDLSRLTSDDTAVYYCARDIVDAFDFWGQGTTVTVSS |
| VH-6 | 6 | QVQLLESGAVLARPGTSVKISCKASGYTFTNHYMHWVKQRPGQGLEWIGIINPISGSSSNAQKFQGRVTI |
| | | TRDTSTSTVFMELSSLRSEDTAVYYCARDIVDAFDFWGQGTLVTVSS |
| VH-7 | 7 | EVQLVQSGAEVRKPGASVKVSCKASGYTFTNHYMHWVRQAPGQGLEWMGIINPISGSTSNAQKLQGRVTF |
| | | TRDTSANTAYMELSGLIPEDTAIYYCARDIVDAFDFWGQGTLVTVSS |
| VH-8 | 8 | QVQLLESGAVLARPGTSVKISCKASGYTFTNHYMHWVKQRPGQGLEWIGIINPITGSSSNAQKFQGRVTI |
| | | TRDTSTSTVFMELSSLRSEDTAVYYCARDIVDAFDFWGQGTLVTVSS |
| VH-9 | 9 | EVQLVQSGAEVRKPGASVKVSCKASGYTFTNHYMHWVRQAPGQGLEWMGIINPISGSTSNAQKLQGRVTF |
| | | TRDTSANTAYMELSGLIPEDTAIYYCAREIVDAFDFWGQGTLVTVSS |
| VH-10 | 10 | QVQLLESGAVLARPGTSVKISCKASGYTFTNYYMHWVKQRPGQGLEWIGIINPITGSSSNAQKFQGRVTI |
| | | TRDTSTSTVFMELSSLRSEDTAVYYCAREIVDAFDFWGQGTLVTVSS |
| VH-11 | 11 | EVQLVQSGAEVRKPGASVKVSCKASGYSFTNHYMHWVRQAPGQGLEWMGIINPNSGSTSNAQKLQGRVTF |
| | | TRDTSANTAYMELSGLIPEDTAIYYCARDIVDAFDYWGQGTLVTVSS |
| VH-12 | 12 | QVQLLESGAVLARPGTSVKISCKASGYSFTNHYMHWVKQRPGQGLEWIGIINPITGSSSNAQKFQGRVTI |
| | | TRDTSTSTVFMELSSLRSEDTAVYYCARDIVDAFDFWGQGTLVTVSS |
| VH-13 | 13 | QVQLLESGAVLARPGTSVKISCKASGYSFTNHYMHWVKQRPGQGLEWIGIINPITGSSSNAQKFQGRVTI |
| | | TRDTSTSTVFMELSSLRSEDTAVYYCARDLVDAFDFWGQGTLVTVSS |
| VH-14 | 90 | QVQLLESGAVLARPGTSVKISCKASGYSFTAHYMHWVKQRPGQGLEWIGIINPITGSSSNAQKFQGRVTI |
| | | TRDTSTSTVFMELSSLRSEDTAVYYCARDLVDAFDFWGQGTLVTVSS |
| VH-15 | 91 | EVQLVQSGAEVRKPGASVKVSCKASGYTFTAHYMHWVRQAPGQGLEWMGIINPISGSTSNAQKLQGRVTF |
| | | TRDTSANTAYMELSGLIPEDTAIYYCARDIVDAFDFWGQGTLVTVSS |
| VL-1 | 14 | EIQLTQSPATLSLSPGERATLSCRASQGISSALVWYQQKPGQAPRLLIYDASSLESGIPARFSGSGSGTD |
| | | FTLTISSLEPEDFAVYYCQQFNDYFTFGQGTKLEIK |
| VL-2 | 15 | EIQLTQSPATLSLSPGERATLSCRASQGVSSALVWYQQKPGQAPRLLIYAASSLESGIPARFSGSGSGTD |
| | | FTLTISSLEPEDFAVYYCQQFNSYFTFGQGTKLEIK |
| VL-3 | 16 | EIQLTQSPATLSLSPGERATLSCRASQGISSALAWYQQKPGQAPRLLIYAASNLESGIPARFSGSGSGTD |
| | | FTLTISSLEPEDFAVYYCQQYNSYYTFGQGTKLEIK |
| VL-4 | 17 | EIQLTQSPATLSLSPGERATLSCKASQGISSALVWYQQKPGQAPRLLIYDASSRATGIPARFSGSGSGTD |
| | | FTLTISSLEPEDFAVYYCQQFNNYFTFGQGTKLEIK |
| VL-5 | 18 | AIQLTQSPATLSLSPGERATLSCRASQGISSALVWYQQKPGQAPRLLIYDASSLESGIPARFSGSGSGTD |
| | | FTLTISSLEPEDFAVYYCQQFNDYFTFGQGTKLEIK |
| VL-6 | 19 | AIQLTQSPSSLSASVGDRVTITCRASQGISSALVWYQQKPGQAPRLLIYDASSLESGIPARFSGSGSGTD |
| | | FTLTISSLEPEDFAVYYCQQFNDYFTFGQGTKLEIK |
| VL-7 | 20 | AIQMTQSPSSLSASVGDRVTITCRASQGISSALVWYQQKPGKAPKLLIYDASSLESGVPSRFSGSGSGTD |
| | | FTFTISSLQPEDIATYYCQQFNDYFTFGPGTKLEIK |
| VL-8 | 21 | AIQLTQSPATLSVSPGERATLSCRASQGISSALVWYQQKPGQAPRLLIYDASSLESGIPARFSGSGSGTE |
| | | FTLTISSLQSEDFAVYYCQQFNDYFTFGPGTKVEIK |
| VL-9 | 22 | AIQLTQSPATLSVSPGERATLSCRASQGISSALVWYQQKPGQAPRLLIYKASSLESGIPARFSGSGSGTE |
| | | FTLTISSLQSEDFAVYYCQQFNDYFTFGPGTKVEIK |
| VL-10 | 23 | AIQMTQSPSSLSASVGDRVTITCKASQGISSALAWYQQKPGKAPKLLIYDASNLESGVPSRFSGSGSGTD |
| | | FTFTISSLQPEDIATYYCQQYNDYFTFGPGTKLEIK |
| VL-11 | 24 | AIQLTQSPATLSVSPGERATLSCRASQGISSYLVWYQQKPGQAPRLLIYDASNLESGIPARFSGSGSGTE |
| | | FTLTISSLQSEDFAVYYCQQFSDYFTFGPGTKVEIK |
| VL-12 | 25 | DIQLTQSPSSLSASVGDRVTITCRASQGISSALVWYQQKPGQAPRLLIYDASSLQSGIPARFSGSGSGTD |
| | | FTLTISSLEPEDFAVYYCQQFNDYFTFGQGTKLEIK |
| VL-13 | 26 | DIQLTQSPSSLSASVGDRVTITCRASQGISSALVWYQQKPGQAPRLLIYDASSLQSGIPARFSGSGSGTD |
| | | FTLTISSLEPEDFAVYYCQQFNDYFSFGQGTKLEIK |
| VL-14 | 27 | DIQLTQSPSSLSASVGDRVTITCKASQGISSALVWYQQKPGQAPRLLIYDASSLQSGIPARFSGSGSGTD |
| | | FTLTISSLEPEDFAVYYCQQFNDYFSFGQGTKLEIK |
| VL-15 | 28 | DIQLTQSPSSLSASVGDRVTITCRASQGISSALVWYQQKPGQAPRLLIYDASSLQSGIPARFSGSGSGTD |
| | | FTLTISSLEPEDFAVYYCQQFNDLFTFGQGTKLEIK |
| VL-16 | 29 | DIQLTQSPSSLSASVGDRVTITCKASQGISSALVWYQQKPGQAPRLLIYDASSLETGIPARFSGSGSGTD |
| | | FTLTISSLEPEDFAVYYCQQFNDYFSFGQGTKLEIK |
| VL-17 | 30 | DIQLTQSPSSLSASVGDRVTITCKASQGISSALVWYQQKPGQAPRLLIYDASSLQTGIPARFSGSGSGTD |
| | | FTLTISSLEPEDFAVYYCQQFNDYFSFGQGTKLEIK |
| VL-18 | 31 | DIQLTQSPSSLSASVGDRVTITCKASQGISSALVWYQQKPGQAPRLLIYDASSLQTGIPARFSGSGSGTD |
| | | FTLTISSLEPEDFAVYYCQQFNDLFTFGQGTKLEIK |
| VL-19 | 32 | DIQMTQSPSSLSASVGDRVTITCRASQGISSALVWYQQKPGKAPKLLIYDASSLETGVPSRFSGSGSGTD |
| | | FTFTISSLQPEDIATYYCQQFNDYFTFGPGTKLEIK |
| VL-20 | 33 | DIQMTQSPSSLSASVGDRVTITCRASQGISSALVWYQQKPGKAPKLLIYDASSLETGVPSRFSGSGSGTD |
| | | FTFTISSLQPEDIATYYCQQFNDYFSFGPGTKLEIK |
| VL-21 | 34 | DIQMTQSPSSLSASVGDRVTITCKASQGISSALVWYQQKPGKAPKLLIYDASSLETGVPSRFSGSGSGTD |
| | | FTFTISSLQPEDIATYYCQQFNDYFSFGPGTKLEIK |
| VL-22 | 35 | DIQMTQSPSSLSASVGDRVTITCRASQGISSALVWYQQKPGKAPKLLIYDASSLETGVPSRFSGSGSGTD |
| | | FTFTISSLQPEDIATYYCQQFNDLFTFGPGTKLEIK |
| VL-23 | 36 | DIQMTQSPSSLSASVGDRVTITCKASQGISSALVWYQQKPGKAPKLLIYDASSLQSGVPSRFSGSGSGTD |
| | | FTFTISSLQPEDIATYYCQQFNDYFSFGPGTKLEIK |
| VL-24 | 37 | DIQMTQSPSSLSASVGDRVTITCKASQGISSALVWYQQKPGKAPKLLIYDASSLQTGVPSRFSGSGSGTD |
| | | FTFTISSLQPEDIATYYCQQFNDYFSFGPGTKLEIK |
| VL-25 | 38 | DIQMTQSPSSLSASVGDRVTITCKASQGISSALVWYQQKPGKAPKLLIYDASSLQTGVPSRFSGSGSGTD |
| | | FTFTISSLQPEDIATYYCQQFNDLFTFGPGTKLEIK |
| VL-26 | 92 | DIQLTQSPSSLSASVGDRVTITCRASAGISSALVWYQQKPGQAPRLLIYDASSLQSGIPARFSGSGSGTD |
| | | FTLTISSLEPEDFAVYYCQQFNDLFTFGQGTKLEIK |
| VL-27 | 93 | AIQLTQSPSSLSASVGDRVTITCRASAGISSALVWYQQKPGQAPRLLIYDASSLQSGIPARFSGSGSGTD |
| | | FTLTISSLEPEDFAVYYCQQFNDLFTFGQGTKLEIK |
| VL-28 | 94 | DIQMTQSPSSLSASVGDRVTITCKASQGISSALVWYQQKPGKAPKLLIYDASSLQTGVPSRFSGSGSGTD |
| | | FTFTISSLQPEDIATYYCQQFNDYFTFGPGTKLEIK |
| VL-29 | 95 | DIQMTQSPSSLSASVGDRVTITCRASQGISSALVWYQQKPGKAPKLLIYDASSLQTGVPSRFSGSGSGTD |
| | | FTFTISSLQPEDIATYYCQQFNDYFSFGPGTKLEIK |
| VL-30 | 96 | AIQMTQSPSSLSASVGDRVTITCKASQGISSALVWYQQKPGKAPKLLIYDASSLQTGVPSRFSGSGSGTD |
| | | FTFTISSLQPEDIATYYCQQFNDYFSFGPGTKLEIK |

TABLE 3
List of Exemplary CDR sequences

| Description | SEQ ID NO: | Amino Acid Sequence |
|---|---|---|
| HCDR1 | 39 | NHFIH |
| Sequences | 40 | NHFMH |
| | 41 | NHYMH |
| | 42 | NYYMH |
| | 43 | SHYMH |
| | 110 | AHYMH |
| HCDR2 | 44 | IINPISGSSSNAQKFQG |
| Sequences | 45 | IINPISGSTSNAQKFQG |
| | 46 | IINPISGSTSNAQKLQG |
| | 47 | IINPISGSTSYAQKFQG |
| | 48 | IINPISGSTTYAQKFQG |
| | 49 | IINPITGSSSNAQKFQG |
| | 50 | IINPNSGSTSNAQKLQG |
| | 51 | TINPVSGSTNYAQKFQG |
| | 52 | TINPVSGSTSYAQKFQG |
| HCDR3 | 53 | DIVDAFDF |
| Sequences | 54 | DIVDAFDY |
| | 55 | DLVDAFDF |
| | 56 | EILDAFDY |
| | 57 | EIVDAFDF |
| LCDR1 | 58 | KASQGISSALA |
| Sequences | 59 | KASQGISSALV |
| | 60 | RASQGISSALA |
| | 61 | RASQGISSALV |
| | 62 | RASQGISSYLV |
| | 63 | RASQGVSSALV |
| | 109 | RASAGISSALV |
| LCDR2 | 64 | AASNLES |
| Sequences | 65 | AASSLES |
| | 66 | DASNLES |
| | 67 | DASSLES |
| | 68 | DASSLET |
| | 69 | DASSLQS |
| | 70 | DASSLQT |
| | 71 | DASSRAT |
| | 72 | KASSLES |
| LCDR3 | 73 | QQFNDYFS |
| Sequences | 74 | QQFNDYFT |
| | 75 | QQFNNYFT |
| | 76 | QQFNSYFT |
| | 77 | QQFSDYFT |
| | 78 | QQYNDYFT |
| | 79 | QQYNSYYT |
| | 89 | QQFNDLFT |

TABLE 5
Nucleotide sequences of heavy chain variable regions (VHs) and light
chain variable regions (VLs) of exemplary antibody molecules

| Antibody | Chain | Nucleotide Sequence | SEQ ID NO |
|---|---|---|---|
| ExA11 | VH | TCTAGAGGATCGAACCCTTCACCATGGAAACCGACACTTTGCTCCTCTGGGTCCTCCTGCTTTGGGTGCCTGGCTCGA | 111 |
| | | CTGGACAAGTGCAGCTGGTGCAGTCCGGGGCCGAACTGAAGAAGCCAGGCGCCTCGGTCAAAGTGTCCTGCAAAGCCA | |
| | | GCGGATACACCTTCACCAACCATTACATGCACTGGGTCAGACAGGCCCCGGGACAGGGTCTGGAGTGGATGGGTATCA | |
| | | TCAACCCCATTTCCGGCTCCACCACCTACGCGCAAAAGTTTCAGGGCCGCGTGACAATGACTCGGGATACCTCCAGCT | |
| | | CCACCGCTTATATGGACCTGTCGAGGCTGACGAGCGACGATACTGCCGTGTACTACTGTGCCCGGGACATCGTGGACG | |
| | | CGTTCGATTTCTGGGGACAGGGGACTACCGTGACCGTGTCATCAGCATCCACCAAGGGGCCC | |
| | VL | TCTAGAGGATCGAACCCTTCACCATGGAAACTGACACATTGCTGCTTTGGGTGTTGCTCCTTTGGGTCCCCGGTTCAA | 112 |
| | | CTGGCGCTATCCAGCTGACCCAGAGCCCGTCATCCCTGTCCGCCTCCGTGGGAGACAGAGTCACCATTACTTGCCGGG | |
| | | CCTCCCAAGGGATTTCCAGCGCGCTCGTGTGGTATCAGCAGAAACCGGGACAGGCACCCAGGCTGCTGATCTACGACG | |
| | | CCAGCTCGCTGGAGAGCGGAATCCCTGCCCGCTTTTCGGGGTCGGGTTCTGGCACCGATTTCACCCTGACCATCTCCT | |
| | | CCCTGGAACCAGAGGATTTCGCCGTGTACTACTGCCAACAGTTCAACGACTACTTCACGTTCGGCCAGGGAACCAAGC | |
| | | TCGAAATCAAGGGATCC | |
| ExA28 | VH | TCTAGAGGATCGAACCCTTCACCATGGAAACTGACACTCTCCTGCTTTGGGTCTTGCTGCTCTGGGTGCCTGGTTCAA | 97 |
| | | CCGGCGAAGTGCAACTGGTGCAGTCCGGTGCCGAGGTCCGCAAGCCCGGAGCCAGCGTGAAAGTGTCCTGCAAGGCCT | |
| | | CCGGGTACACCTTCACCAATCACTACATGCATTGGGTCAGACAGGCCCCGGGCCAGGGACTGGAGTGGATGGGCATCA | |
| | | TCAACCCAATTTCGGGGTCAACCTCGAACGCTCAAAAGCTGCAGGGCCGCGTGACCTTTACTCGGGACACCTCCGCAA | |
| | | ACACAGCGTACATGGAACTGTCCGGACTGATTCCCGAGGATACCGCCATCTACTATTGTGCCCGGGATATCGTGGACG | |
| | | CCTTCGACTTCTGGGGACAGGGAACTCTCGTGACGGTGTCCAGCGCGAGCACCAAGGGGCCC | |
| | VL | TCTAGAGGATCGAACCCTTCACCATGGAAACTGATACTCTGCTTCTCTGGGTCTTGCTGCTCTGGGTCCCTGGTTCAA | 98 |
| | | CTGGCGCTATCCAGATGACCCAGTCCCCGTCGTCACTGTCCGCCTCCGTGGGTGATCGCGTGACCATCACGTGTCGGG | |
| | | CCAGCCAGGGAATCTCCTCCGCACTCGTGTGGTATCAGCAGAAGCCTGGAAAAGCCCCGAAGCTGCTGATCTACGACG | |
| | | CCTCCTCCCTGGAATCGGGAGTGCCATCGAGATTCTCCGGCTCTGGGAGCGGGACCGACTTCACCTTCACAATTAGCA | |
| | | GCCTGCAGCCCGAGGACATCGCGACCTACTACTGCCAACAATTCAACGACTACTTTACCTTCGGACCCGGCACCAAGC | |
| | | TCGAGATTAAGGGATCC | |
| ExA35 | VH | TCTAGAGGATCGAACCCTTCACCATGGAAACTGACACTCTCTTGCTCTGGGTACTTCTCCTCTGGGTCCCTGGTTCCA | 99 |
| | | CCGGCCAAGTGCAGCTGCTGGAATCGGGAGCGGTGCTGGCCCGGCCGGGAACTTCCGTGAAGATCAGCTGCAAAGCAT | |
| | | CAGGGTACACCTTCACCAACCACTACATGCATTGGGTCAAGCAGAGGCCCGGCCAGGGACTGGAGTGGATCGGCATCA | |
| | | TCAACCCAATTACCGGCAGCTCCTCCAACGCTCAAAAGTTCCAGGGTCGCGTGACCATTACCAGAGATACCTCCACCT | |
| | | CGACCGTGTTCATGGAACTGTCATCCCTGCGGTCCGAGGACACTGCCGTGTATTACTGCGCCCGCGATATCGTGGACG | |
| | | CCTTTGACTTCTGGGGACAGGGGACGCTGGTCACAGTGTCGAGCGCCAGCACCAAGGGGCCC | |
| | VL | TCTAGAGGATCGAACCCTTCACCATGGAAACTGACACATTGCTGCTTTGGGTGTTGCTCCTTTGGGTCCCCGGTTCAA | 100 |
| | | CTGGCGCTATCCAGCTGACCCAGAGCCCGTCATCCCTGTCCGCCTCCGTGGGAGACAGAGTCACCATTACTTGCCGGG | |
| | | CCTCCCAAGGGATTTCCAGCGCGCTCGTGTGGTATCAGCAGAAACCGGGACAGGCACCCAGGCTGCTGATCTACGACG | |
| | | CCAGCTCGCTGGAGAGCGGAATCCCTGCCCGCTTTTCGGGGTCGGGTTCTGGCACCGATTTCACCCTGACCATCTCCT | |
| | | CCCTGGAACCAGAGGATTTCGCCGTGTACTACTGCCAACAGTTCAACGACTACTTCACGTTCGGCCAGGGAACCAAGC | |
| | | TCGAAATCAAGGGATCC | |
| ExA43 | VH | TCTAGAGGATCGAACCCTTCACCATGGAAACTGATACTTTGCTCCTTTGGGTCCTCCTGCTTTGGGTGCCTGGCTCGA | 101 |
| | | CCGGAGAAGTGCAACTGGTGCAGTCCGGAGCCGAAGTCCGGAAGCCCGGAGCCTCCGTGAAAGTGTCCTGCAAGGCCT | |
| | | CGGGCTATACCTTCACCAACCACTACATGCATTGGGTCCGCCAAGCACCAGGACAGGGCCTGGAGTGGATGGGTATCA | |
| | | TTAACCCGATCAGCGGGAGCACTTCAAACGCTCAGAAGCTGCAGGGCAGAGTGACCTTTACCCGCGACACCAGCGCCA | |
| | | ATACGGCGTACATGGAACTGTCCGGGCTGATCCCCGAGGACACAGCGATCTACTACTGTGCCCGGGAGATTGTGGATG | |
| | | CCTTCGACTTCTGGGGACAGGGTACCCTCGTGACCGTGTCATCCGCCTCCACTAAGGGGCCC | |
| | VL | TCTAGAGGATCGAACCCTTCACCATGGAAACTGACACATTGCTGCTTTGGGTGTTGCTCCTTTGGGTCCCCGGTTCAA | 102 |
| | | CTGGCGCTATCCAGCTGACCCAGAGCCCGTCATCCCTGTCCGCCTCCGTGGGAGACAGAGTCACCATTACTTGCCGGG | |
| | | CCTCCCAAGGGATTTCCAGCGCGCTCGTGTGGTATCAGCAGAAACCGGGACAGGCACCCAGGCTGCTGATCTACGACG | |
| | | CCAGCTCGCTGGAGAGCGGAATCCCTGCCCGCTTTTCGGGGTCGGGTTCTGGCACCGATTTCACCCTGACCATCTCCT | |
| | | CCCTGGAACCAGAGGATTTCGCCGTGTACTACTGCCAACAGTTCAACGACTACTTCACGTTCGGCCAGGGAACCAAGC | |
| | | TCGAAATCAAGGGATCC | |
| ExA60 | VH | TCTAGAGGATCGAACCCTTCACCATGGAAACCGACACGCTGCTCCTCTGGGTCCTTCTTCTCTGGGTGCCTGGTTCAA | 103 |
| | | CTGGAGAGGTGCAGCTGGTGCAGTCCGGCGCAGAAGTCCGGAAGCCCGGCGCCAGCGTGAAAGTGTCCTGCAAGGCCT | |
| | | CCGGGTACTCATTCACCAACCATTACATGCACTGGGTCCGCCAAGCGCCGGGACAAGGGCTGGAGTGGATGGGAATCA | |
| | | TTAACCCAAACAGCGGTTCCACCTCGAACGCCCAGAAGCTGCAGGGCAGAGTGACCTTCACTCGCGACACCTCCGCTA | |
| | | ATACCGCCTACATGGAATTGTCGGGCCTGATTCCCGAGGATACCGCGATCTACTACTGTGCCCGGGACATCGTGGATG | |
| | | CCTTTGACTATTGGGGACAGGGAACTCTGGTCACCGTGTCGAGCGCCTCCACTAAGGGGCCC | |
| | VL | TCTAGAGGATCGAACCCTTCACCATGGAAACTGATACTCTGCTTCTCTGGGTCTTGCTGCTCTGGGTCCCTGGTTCAA | 104 |
| | | CTGGCGCTATCCAGATGACCCAGTCCCCGTCGTCACTGTCCGCCTCCGTGGGTGATCGCGTGACCATCACGTGTCGGG | |
| | | CCAGCCAGGGAATCTCCTCCGCACTCGTGTGGTATCAGCAGAAGCCTGGAAAAGCCCCGAAGCTGCTGATCTACGACG | |
| | | CCTCCTCCCTGGAATCGGGAGTGCCATCGAGATTCTCCGGCTCTGGGAGCGGGACCGACTTCACCTTCACAATTAGCA | |
| | | GCCTGCAGCCCGAGGACATCGCGACCTACTACTGCCAACAATTCAACGACTACTTTACCTTCGGACCCGGCACCAAGC | |
| | | TCGAGATTAAGGGATCC | |
| ExC17 | VH | TCTAGAGGATCGAACCCTTCACCATGGAAACTGACACTCTCCTGCTTTGGGTCTTGCTGCTCTGGGTGCCTGGTTCAA | 105 |
| | | CCGGCGAAGTGCAACTGGTGCAGTCCGGTGCCGAGGTCCGCAAGCCCGGAGCCAGCGTGAAAGTGTCCTGCAAGGCCT | |
| | | CCGGGTACACCTTCACCAATCACTACATGCATTGGGTCAGACAGGCCCCGGGCCAGGGACTGGAGTGGATGGGCATCA | |
| | | TCAACCCAATTTCGGGGTCAACCTCGAACGCTCAAAAGCTGCAGGGCCGCGTGACCTTTACTCGGGACACCTCCGCAA | |
| | | ACACAGCGTACATGGAACTGTCCGGACTGATTCCCGAGGATACCGCCATCTACTATTGTGCCCGGGATATCGTGGACG | |
| | | CCTTCGACTTCTGGGGACAGGGAACTCTCGTGACGGTGTCCAGCGCGAGCACCAAGGGGCCC | |
| | VL | TCTAGAGGATCGAACCCTTCACCATGGAAACTGATACTTTGCTGCTCTGGGTGCTTCTGCTTTGGGTCCCTGGCTCGA | 106 |
| | | CCGGCGACATCCAGCTGACCCAGTCGCCGTCATCTCTGTCCGCCTCCGTGGGCGATAGAGTCACTATCACGTGTCGCG | |
| | | CAAGCCAGGGAATTAGCTCCGCCCTCGTGTGGTATCAGCAGAAGCCCGGGCAGGCCCCTAGGCTGCTGATCTACGACG | |
| | | CGTCCAGCCTGCAATCCGGAATCCCAGCCCGGTTCTCCGGTTCCGGATCGGGAACCGACTTTACCCTGACCATCTCAA | |
| | | GCCTGGAACCCGAGGACTTCGCTGTGTACTACTGCCAGCAATTCAACGACCTCTTCACCTTCGGGCAGGGTACTAAGC | |
| | | TCGAGATTAAGGGATCC | |
| ExC50 | VH | TCTAGAGGATCGAACCCTTCACCATGGAAACCGATACTCTGCTCCTCTGGGTCTTGCTGCTCTGGGTCCCCGGTTCGA | 107 |
| | | CTGGCCAAGTGCAGCTGCTGGAGTCAGGGGCCGTGCTGGCCCGCCCTGGGACCAGCGTGAAGATCAGCTGCAAGGCCT | |
| | | CTGGATACTCCTTTACCAACCACTACATGCATTGGGTCAAGCAGAGGCCGGGCCAGGGTCTTGAGTGGATCGGAATTA | |
| | | TCAACCCAATCACCGGCTCGTCCAGCAACGCACAAAAGTTCCAGGGCAGAGTGACGATCACCCGGGACACCTCGACCT | |
| | | CCACCGTGTTCATGGAACTGAGCTCCCTGCGGTCCGAGGATACTGCCGTGTATTACTGTGCCCGCGACCTGGTGGACG | |
| | | CTTTCGACTTCTGGGGACAGGGAACACTCGTGACTGTGTCCTCCGCGTCAACCAAAGGGCCC | |
| | VL | TCTAGAGGATCGAACCCTTCACCATGGAAACTGATACTTTGCTGCTCTGGGTGCTTCTGCTTTGGGTCCCTGGCTCGA | 108 |
| | | CCGGCGACATCCAGCTGACCCAGTCGCCGTCATCTCTGTCCGCCTCCGTGGGCGATAGAGTCACTATCACGTGTCGCG | |
| | | CAAGCCAGGGAATTAGCTCCGCCCTCGTGTGGTATCAGCAGAAGCCCGGGCAGGCCCCTAGGCTGCTGATCTACGACG | |
| | | CGTCCAGCCTGCAATCCGGAATCCCAGCCCGGTTCTCCGGTTCCGGATCGGGAACCGACTTTACCCTGACCATCTCAA | |
| | | GCCTGGAACCCGAGGACTTCGCTGTGTACTACTGCCAGCAATTCAACGACTACTTCACCTTCGGGCAGGGTACTAAGC | |
| | | TCGAGATTAAGGGATCC | |
| Exc23 | VH | GAAGTGCAGCTGGTGCAGAGCGGCGCGGAAGTGCGCAAACCGGGCGCGAGCGTGAAAGTG | 113 |
| | | AGCTGCAAAGCGAGCGGCTATACCTTTACCAACCATTATATGCATTGGGTGCGCCAGGCG | |
| | | CCGGGCCAGGGCCTGGAATGGATGGGCATTATTAACCCGATTAGCGGCAGCACCAGCAAC | |
| | | GCGCAGAAACTGCAGGGCCGCGTGACCTTTACCCGCGATACCAGCGCGAACACCGCGTAT | |
| | | ATGGAACTGAGCGGCCTGATTCCGGAAGATACCGCGATTTATTATTGCGCGCGCGATATT | |
| | | GTGGATGCGTTTGATTTTTGGGGCCAGGGCACCCTGGTGACCGTGAGCAGC | |
| | VL | GATATTCAGATGACCCAGAGCCCGAGCAGCCTGAGCGCGAGCGTGGGCGATCGCGTGACC | 114 |
| | | ATTACCTGCAAAGCGAGCCAGGGCATTAGCAGCGCGCTGGTGTGGTATCAGCAGAAACCG | |
| | | GGCAAAGCGCCGAAACTGCTGATTTATGATGCGAGCAGCCTGCAGACCGGCGTGCCGAGC | |
| | | CGCTTTAGCGGCAGCGGCAGCGGCACCGATTTTACCTTTACCATTAGCAGCCTGCAGCCG | |
| | | GAAGATATTGCGACCTATTATTGCCAGCAGTTTAACGATTATTTTAGCTTTGGCCCGGGC | |
| | | ACCAAACTGGAAATTAAA | |
| Exc23.1 | VH | GAAGTGCAGCTGGTGCAGAGCGGCGCGGAAGTGCGCAAACCGGGCGCGAGCGTGAAAGTG | 115 |
| | | AGCTGCAAAGCGAGCGGCTATACCTTTACCAACCATTATATGCATTGGGTGCGCCAGGCG | |
| | | CCGGGCCAGGGCCTGGAATGGATGGGCATTATTAACCCGATTAGCGGCAGCACCAGCAAC | |
| | | GCGCAGAAACTGCAGGGCCGCGTGACCTTTACCCGCGATACCAGCGCGAACACCGCGTAT | |
| | | ATGGAACTGAGCGGCCTGATTCCGGAAGATACCGCGATTTATTATTGCGCGCGCGATATT | |
| | | GTGGATGCGTTTGATTTTTGGGGCCAGGGCACCCTGGTGACCGTGAGCAGC | |
| | VL | GATATTCAGATGACCCAGAGCCCGAGCAGCCTGAGCGCGAGCGTGGGCGATCGCGTGACC | 116 |
| | | ATTACCTGCAAAGCGAGCCAGGGCATTAGCAGCGCGCTGGTGTGGTATCAGCAGAAACCG | |
| | | GGCAAAGCGCCGAAACTGCTGATTTATGATGCGAGCAGCCTGGAAACCGGCGTGCCGAGC | |
| | | CGCTTTAGCGGCAGCGGCAGCGGCACCGATTTTACCTTTACCATTAGCAGCCTGCAGCCG | |
| | | GAAGATATTGCGACCTATTATTGCCAGCAGTTTAACGATTATTTTAGCTTTGGCCCGGGC | |
| | | ACCAAACTGGAAATTAAA | |
| Exc23.2 | VH | GAAGTGCAGCTGGTGCAGAGCGGCGCGGAAGTGCGCAAACCGGGCGCGAGCGTGAAAGTG | 117 |
| | | AGCTGCAAAGCGAGCGGCTATACCTTTACCAACCATTATATGCATTGGGTGCGCCAGGCG | |
| | | CCGGGCCAGGGCCTGGAATGGATGGGCATTATTAACCCGATTAGCGGCAGCACCAGCAAC | |
| | | GCGCAGAAACTGCAGGGCCGCGTGACCTTTACCCGCGATACCAGCGCGAACACCGCGTAT | |
| | | ATGGAACTGAGCGGCCTGATTCCGGAAGATACCGCGATTTATTATTGCGCGCGCGATATT | |
| | | GTGGATGCGTTTGATTTTTGGGGCCAGGGCACCCTGGTGACCGTGAGCAGC | |
| | VL | GATATTCAGATGACCCAGAGCCCGAGCAGCCTGAGCGCGAGCGTGGGCGATCGCGTGACC | 118 |
| | | ATTACCTGCAAAGCGAGCCAGGGCATTAGCAGCGCGCTGGTGTGGTATCAGCAGAAACCG | |
| | | GGCAAAGCGCCGAAACTGCTGATTTATGATGCGAGCAGCCTGCAGAGCGGCGTGCCGAGC | |
| | | CGCTTTAGCGGCAGCGGCAGCGGCACCGATTTTACCTTTACCATTAGCAGCCTGCAGCCG | |
| | | GAAGATATTGCGACCTATTATTGCCAGCAGTTTAACGATTATTTTAGCTTTGGCCCGGGC | |
| | | ACCAAACTGGAAATTAAA | |
| Exc23.3 | VH | GAAGTGCAGCTGGTGCAGAGCGGCGCGGAAGTGCGCAAACCGGGCGCGAGCGTGAAAGTG | 119 |
| | | AGCTGCAAAGCGAGCGGCTATACCTTTACCAACCATTATATGCATTGGGTGCGCCAGGCG | |
| | | CCGGGCCAGGGCCTGGAATGGATGGGCATTATTAACCCGATTAGCGGCAGCACCAGCAAC | |
| | | GCGCAGAAACTGCAGGGCCGCGTGACCTTTACCCGCGATACCAGCGCGAACACCGCGTAT | |
| | | ATGGAACTGAGCGGCCTGATTCCGGAAGATACCGCGATTTATTATTGCGCGCGCGATATT | |
| | | GTGGATGCGTTTGATTTTTGGGGCCAGGGCACCCTGGTGACCGTGAGCAGC | |
| | VL | GATATTCAGATGACCCAGAGCCCGAGCAGCCTGAGCGCGAGCGTGGGCGATCGCGTGACC | 120 |
| | | ATTACCTGCAAAGCGAGCCAGGGCATTAGCAGCGCGCTGGTGTGGTATCAGCAGAAACCG | |
| | | GGCAAAGCGCCGAAACTGCTGATTTATGATGCGAGCAGCCTGCAGACCGGCGTGCCGAGC | |
| | | CGCTTTAGCGGCAGCGGCAGCGGCACCGATTTTACCTTTACCATTAGCAGCCTGCAGCCG | |
| | | GAAGATATTGCGACCTATTATTGCCAGCAGTTTAACGATTATTTTACCTTTGGCCCGGGC | |
| | | ACCAAACTGGAAATTAAA | |
| Exc23.4 | VH | GAAGTGCAGCTGGTGCAGAGCGGCGCGGAAGTGCGCAAACCGGGCGCGAGCGTGAAAGTG | 121 |
| | | AGCTGCAAAGCGAGCGGCTATACCTTTACCAACCATTATATGCATTGGGTGCGCCAGGCG | |
| | | CCGGGCCAGGGCCTGGAATGGATGGGCATTATTAACCCGATTAGCGGCAGCACCAGCAAC | |
| | | GCGCAGAAACTGCAGGGCCGCGTGACCTTTACCCGCGATACCAGCGCGAACACCGCGTAT | |
| | | ATGGAACTGAGCGGCCTGATTCCGGAAGATACCGCGATTTATTATTGCGCGCGCGATATT | |
| | | GTGGATGCGTTTGATTTTTGGGGCCAGGGCACCCTGGTGACCGTGAGCAGC | |
| | VL | GATATTCAGATGACCCAGAGCCCGAGCAGCCTGAGCGCGAGCGTGGGCGATCGCGTGACC | 122 |
| | | ATTACCTGCCGCGCGAGCCAGGGCATTAGCAGCGCGCTGGTGTGGTATCAGCAGAAACCG | |
| | | GGCAAAGCGCCGAAACTGCTGATTTATGATGCGAGCAGCCTGCAGACCGGCGTGCCGAGC | |
| | | CGCTTTAGCGGCAGCGGCAGCGGCACCGATTTTACCTTTACCATTAGCAGCCTGCAGCCG | |
| | | GAAGATATTGCGACCTATTATTGCCAGCAGTTTAACGATTATTTTAGCTTTGGCCCGGGC | |
| | | ACCAAACTGGAAATTAAA | |
| Exc23.5 | VH | GAAGTGCAGCTGGTGCAGAGCGGCGCGGAAGTGCGCAAACCGGGCGCGAGCGTGAAAGTG | 123 |
| | | AGCTGCAAAGCGAGCGGCTATACCTTTACCAACCATTATATGCATTGGGTGCGCCAGGCG | |
| | | CCGGGCCAGGGCCTGGAATGGATGGGCATTATTAACCCGATTAGCGGCAGCACCAGCAAC | |
| | | GCGCAGAAACTGCAGGGCCGCGTGACCTTTACCCGCGATACCAGCGCGAACACCGCGTAT | |
| | | ATGGAACTGAGCGGCCTGATTCCGGAAGATACCGCGATTTATTATTGCGCGCGCGATATT | |
| | | GTGGATGCGTTTGATTTTTGGGGCCAGGGCACCCTGGTGACCGTGAGCAGC | |
| | VL | GCGATTCAGATGACCCAGAGCCCGAGCAGCCTGAGCGCGAGCGTGGGCGATCGCGTGACC | 124 |
| | | ATTACCTGCAAAGCGAGCCAGGGCATTAGCAGCGCGCTGGTGTGGTATCAGCAGAAACCG | |
| | | GGCAAAGCGCCGAAACTGCTGATTTATGATGCGAGCAGCCTGCAGACCGGCGTGCCGAGC | |
| | | CGCTTTAGCGGCAGCGGCAGCGGCACCGATTTTACCTTTACCATTAGCAGCCTGCAGCCG | |
| | | GAAGATATTGCGACCTATTATTGCCAGCAGTTTAACGATTATTTTAGCTTTGGCCCGGGC | |
| | | ACCAAACTGGAAATTAAA | |

In an embodiment, the antibody molecule comprises one, two, or three CDRs of the VH region of an antibody molecule described herein, e.g., in Table 1 (e.g., any of monoclonal antibodies ExA11, ExA28, ExA35, ExA43, ExA60, ExC17, ExC50, Exc23, Exc23.1, Exc23.2, Exc23.3, Exc23.4, or Exc23.5), using the Kabat or Chothia definitions of CDRs. In an embodiment, the antibody molecule comprises one, two, or three CDRs of the VL region of an antibody molecule described herein, e.g., in Table 1 (e.g., any of monoclonal antibodies ExA11, ExA28, ExA35, ExA43, ExA60, ExC17, ExC50, Exc23, Exc23.1, Exc23.2, Exc23.3, Exc23.4, or Exc23.5), using the Kabat or Chothia definitions of CDRs. In an embodiment, the antibody molecule comprises one or more (e.g., two or three) CDRs of the VH region and/or one or more (e.g., two or three) CDRs of the VL region of an antibody molecule described herein, e.g., in Table 1 (e.g., any of monoclonal antibodies ExA11, ExA28, ExA35, ExA43, ExA60, ExC17, ExC50, Exc23, Exc23.1, Exc23.2, Exc23.3, Exc23.4, or Exc23.5), using the Kabat or Chothia definitions of CDRs.

In an embodiment, the antibody molecule comprises one, two, or three VH CDRs described in Table 1. In an embodiment, the antibody molecule comprises one, two, or three VL CDRs described in Table 1. In an embodiment, the antibody molecule comprises one or more (e.g., two or three) VH CDRs and/or one or more (e.g., two or three) VL CDRs described in Table 1.

In an embodiment, the antibody molecule comprises one, two, three, or four frameworks of the VH region of an antibody molecule described in Table 1 (e.g., any of monoclonal antibodies ExA11, ExA28, ExA35, ExA43, ExA60, ExC17, ExC50, Exc23, Exc23.1, Exc23.2, Exc23.3, Exc23.4, or Exc23.5). In an embodiment, the antibody molecule comprises one, two, three, or four frameworks of the VL region of an antibody molecule described in Table 1 (e.g., any of monoclonal antibodies ExA11, ExA28, ExA35, ExA43, ExA60, ExC17, ExC50, Exc23, Exc23.1, Exc23.2, Exc23.3, Exc23.4, or Exc23.5). In an embodiment, the antibody molecule comprises one or more (e.g., two, three, or four) frameworks of the VH region and/or one or more (e.g., two, three, or four) frameworks of the VL region of an antibody molecule described in Table 1 (e.g., any of monoclonal antibodies ExA11, ExA28, ExA35, ExA43, ExA60, ExC17, ExC50, Exc23, Exc23.1, Exc23.2, Exc23.3, Exc23.4, or Exc23.5).

In an embodiment, the antibody molecule comprises a heavy chain variable region of an antibody molecule described herein, e.g., in Table 1 (e.g., any of monoclonal antibodies ExA11, ExA28, ExA35, ExA43, ExA60, ExC17, ExC50, Exc23, Exc23.1, Exc23.2, Exc23.3, Exc23.4, or Exc23.5). In an embodiment, the antibody molecule comprises a light chain variable region of an antibody molecule described herein, e.g., in Table 1 (e.g., any of monoclonal antibodies ExA11, ExA28, ExA35, ExA43, ExA60, ExC17, ExC50, Exc23, Exc23.1, Exc23.2, Exc23.3, Exc23.4, or Exc23.5). In an embodiment, the antibody molecule comprises a heavy chain variable region and a light chain variable region of an antibody molecule described herein, e.g., in Table 1 (e.g., any of monoclonal antibodies ExA11, ExA28, ExA35, ExA43, ExA60, ExC17, ExC50, Exc23, Exc23.1, Exc23.2, Exc23.3, Exc23.4, or Exc23.5).

In an embodiment, the antibody molecule comprises a heavy chain variable region having an amino acid sequence described in Table 1, or an amino acid sequence substantially identical thereof. In an embodiment, the antibody molecule comprises a light chain variable region having an amino acid sequence described in Table 1, or an amino acid sequence substantially identical thereof. In an embodiment, the antibody molecule comprises a heavy chain variable region having an amino acid sequence described in Table 1 (or an amino acid sequence substantially identical thereof) and a light chain variable region having an amino acid sequences described in Table 1 (or an amino acid sequence substantially identical thereof).

Exemplary VH and VL amino acid sequences are also described in Table 2. Exemplary CDR amino acid sequences are also described in Tables 3-4, respectively.

In an embodiment, the antibody molecule comprises a heavy chain variable region encoded by a nucleotide sequence described in Table 5, or a nucleotide sequence substantially identical thereof. In an embodiment, the antibody molecule comprises a light chain variable region encoded by a nucleotide sequence described in Table 5, or a nucleotide sequence substantially identical thereof. In an embodiment, the antibody molecule comprises a heavy chain variable region encoded by a nucleotide sequence described in Table 5 (or a nucleotide sequence substantially identical thereof) and a light chain variable region encoded by a nucleotide sequence described in Table 5 (or a nucleotide sequence substantially identical thereof).

In an embodiment, the antibody molecule further comprises a heavy chain constant region. In an embodiment, the heavy chain constant region is an IgG1 constant region or a functional portion thereof. In another embodiment, the heavy chain constant region is an IgG2 constant region or a functional portion thereof. In an embodiment, the antibody molecule further comprises a light chain constant region. In an embodiment, the antibody molecule further comprises a heavy chain constant region. In an embodiment, the heavy chain constant region is an IgG3 constant region or a functional portion thereof. In an embodiment, the antibody molecule further comprises a heavy chain constant region. In an embodiment, the heavy chain constant region is an IgG4 constant region or a functional portion thereof. In an embodiment, the antibody molecule has a chimeric constant region comprising of IgG2, IgG3 and/or IgG4 isotypes. In an embodiment, the antibody molecule further comprises a heavy chain constant region and a light chain constant region. In an embodiment, the antibody molecule comprises a heavy chain constant region, a light chain constant region, and heavy and light chain variable regions of an antibody molecule described in Table 1. In an embodiment, the antibody molecule comprises a heavy chain constant region, a light chain constant region, and variable regions that comprise one, two, three, four, five, or six CDRs of an antibody molecule described in Table 1.

Exemplary heavy and light chain constant regions (e.g., human heavy and light chain constant regions) are described below.

Exemplary IgG1 heavy chain constant region (“IgG1”)
(SEQ ID NO: 80)
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELLGGPSVFLFPPKPKDTLMISRTP
EVTCVVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
Exemplary IgG1 heavy chain constant region with Met-252-Tyr,
Ser-254-Thr and Thr-256-Glu substitutions (“IgG1-YTE”)
(SEQ ID NO: 125)
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELLGGPSVFLFPPKPKDTLYITREP
EVTCVVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
Exemplary IgG4 heavy chain constant region (“IgG4”)
(SEQ ID NO: 126)
ASTKGPSVFPLAPCSRSTSESTAALGCLVKDYFPEPVTVSWNSGALTSGVHTFPAVLQSSGLYSLSSVVT
VPSSSLGTKTYTCNVDHKPSNTKVDKRVESKYGPPCPPCPAPEFLGGPSVFLFPPKPKDTLMISRTPEVT
CVVVDVSQEDPEVQFNWYVDGVEVHNAKTKPREEQFNSTYRVVSVLTVLHQDWLNGKEYKCKVSNKGLPS
SIEKTISKAKGQPREPQVYTLPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLG
Exemplary IgG4 heavy chain constant region with Met-252-Tyr,
Ser-254-Thr and Thr-256-Glu substitutions (“IgG4-YTE”)
(SEQ ID NO: 127)
ASTKGPSVFPLAPCSRSTSESTAALGCLVKDYFPEPVTVSWNSGALTSGVHTFPAVLQSSGLYSLSSVVT
VPSSSLGTKTYTCNVDHKPSNTKVDKRVESKYGPPCPPCPAPEFLGGPSVFLFPPKPKDTLYITREPEVT
CVVVDVSQEDPEVQFNWYVDGVEVHNAKTKPREEQFNSTYRVVSVLTVLHQDWLNGKEYKCKVSNKGLPS
SIEKTISKAKGQPREPQVYTLPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLG
Exemplary IgG2/4 heavy chain constant region (“IgG2/4”)
(SEQ ID NO: 128)
ASTKGPSVFPLAPCSRSTSESTAALGCLVKDYFPEPVTVSWNSGALTSGVHTFPAVLQSSGLYSLSSVVT
VPSSNFGTQTYTCNVDHKPSNTKVDKTVERKCCVECPPCPAPPVAGPSVFLFPPKPKDTLMISRTPEVTC
VVVDVSQEDPEVQFNWYVDGVEVHNAKTKPREEQFNSTYRVVSVLTVLHQDWLNGKEYKCKVSNKGLPSS
IEKTISKAKGQPREPQVYTLPPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGK
Exemplary IgG2/4 heavy chain constant region with Met-252-Tyr,
Ser-254-Thr and Thr-256-Glu substitutions (“IgG2/4-YTE”)
(SEQ ID NO: 129)
ASTKGPSVFPLAPCSRSTSESTAALGCLVKDYFPEPVTVSWNSGALTSGVHTFPAVLQSSGLYSLSSVVT
VPSSNFGTQTYTCNVDHKPSNTKVDKTVERKCCVECPPCPAPPVAGPSVFLFPPKPKDTLYITREPEVTC
VVVDVSQEDPEVQFNWYVDGVEVHNAKTKPREEQFNSTYRVVSVLTVLHQDWLNGKEYKCKVSNKGLPSS
IEKTISKAKGQPREPQVYTLPPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGK
Exemplary light chain constant region
(SEQ ID NO: 81)
RTVAAPSVFIFPPSDEQLKSGTASVVCLLNNFYPREAKVQWKVDNALQSGNSQESVTEQDSKDSTYSLSS
TLTLSKADYEKHKVYACEVTHQGLSSPVTKSFNRGEC

In an embodiment, the antibody molecule comprises one or more (e.g., 2, 3, 4, 5, or all) of the CDRs of ExA11, ExA28, ExA35, ExA43, ExA60, ExC17, ExC50, Exc23, Exc23.1, Exc23.2, Exc23.3, Exc23.4, or Exc23.5, and a human IgG1 constant region as described herein (e.g., wild-type or comprising YTE substitution substitutions). In an embodiment, the antibody molecule comprises one or more (e.g., all) of the CDRs of ExA11, ExA28, ExA35, ExA43, ExA60, ExC17, ExC50, Exc23, Exc23.1, Exc23.2, Exc23.3, Exc23.4, or Exc23.5, and a human IgG4 constant region as described herein (e.g., wild-type or comprising YTE substitution substitutions). In an embodiment, the antibody molecule comprises one or more (e.g., all) of the CDRs of ExA11, ExA28, ExA35, ExA43, ExA60, ExC17, ExC50, Exc23, Exc23.1, Exc23.2, Exc23.3, Exc23.4, or Exc23.5, and a human IgG2/4 constant region as described herein (e.g., wild-type or comprising YTE substitution substitutions). In an embodiment, the antibody molecule comprises one or more (e.g., all) of the CDRs of ExA11, ExA28, ExA35, ExA43, ExA60, ExC17, ExC50, Exc23, Exc23.1, Exc23.2, Exc23.3, Exc23.4, or Exc23.5, and a human light constant region as described herein.

In an embodiment, the antibody molecule comprises a heavy chain variable region (VH) of ExA11, ExA28, ExA35, ExA43, ExA60, ExC17, ExC50, Exc23, Exc23.1, Exc23.2, Exc23.3, Exc23.4, or Exc23.5, and a human IgG1 constant region as described herein (e.g., wild-type or comprising YTE substitution substitutions). In an embodiment, the antibody molecule comprises a heavy chain variable region (VH) of ExA11, ExA28, ExA35, ExA43, ExA60, ExC17, ExC50, Exc23, Exc23.1, Exc23.2, Exc23.3, Exc23.4, or Exc23.5, and a human IgG4 constant region as described herein (e.g., wild-type or comprising YTE substitution substitutions). In an embodiment, the antibody molecule comprises a heavy chain variable region (VH) of ExA11, ExA28, ExA35, ExA43, ExA60, ExC17, ExC50, Exc23, Exc23.1, Exc23.2, Exc23.3, Exc23.4, or Exc23.5, and a human IgG2/4 constant region as described herein (e.g., wild-type or comprising YTE substitution substitutions). In an embodiment, the antibody molecule comprises a heavy chain variable region (VH) of ExA11, ExA28, ExA35, ExA43, ExA60, ExC17, ExC50, Exc23, Exc23.1, Exc23.2, Exc23.3, Exc23.4, or Exc23.5, and a human light constant region as described herein. In an embodiment, the antibody molecule comprises a light chain variable region (VL) of ExA11, ExA28, ExA35, ExA43, ExA60, ExC17, ExC50, Exc23, Exc23.1, Exc23.2, Exc23.3, Exc23.4, or Exc23.5, and a human light chain constant region as described herein.

In an embodiment, the antibody molecule comprises a heavy chain variable region (VH) and a light chain variable region (VL) of ExA11, ExA28, ExA35, ExA43, ExA60, ExC17, ExC50, Exc23, Exc23.1, Exc23.2, Exc23.3, Exc23.4, or Exc23.5, and a human IgG1 constant region described herein (e.g., wild-type or comprising YTE substitution substitutions) and a human light chain constant region as described herein. In an embodiment, the antibody molecule comprises a heavy chain variable region (VH) and a light chain variable region (VL) of ExA11, ExA28, ExA35, ExA43, ExA60, ExC17, ExC50, Exc23, Exc23.1, Exc23.2, Exc23.3, Exc23.4, or Exc23.5, and a human IgG4 constant region described herein (e.g., wild-type or comprising YTE substitution substitutions) and a human light chain constant region as described herein. In an embodiment, the antibody molecule comprises a heavy chain variable region (VH) and a light chain variable region (VL) of ExA11, ExA28, ExA35, ExA43, ExA60, ExC17, ExC50, Exc23, Exc23.1, Exc23.2, Exc23.3, Exc23.4, or Exc23.5, and a human IgG2/4 constant region described herein (e.g., wild-type or comprising YTE substitution substitutions) and a human light chain constant region as described herein.

In an embodiment, the IgG1 constant region comprises the amino acid sequence of SEQ ID NO: 80 or 125. In an embodiment, the IgG4 constant region comprises the amino acid sequence of SEQ ID NO: 126 or 127. In an embodiment, the IgG2/4 constant region comprises the amino acid sequence of SEQ ID NO: 128 or 129. In an embodiment, the light chain constant region comprises the amino acid sequence of SEQ ID NO: 81.

In some embodiments, the antibody molecule comprises a heavy chain variable region (VH) and a light chain variable region (VL), wherein the VH comprises three heavy chain complementarity determining regions (HCDR1, HCDR2, and HCDR3), wherein the VL comprises three light chain complementarity determining regions (LCDR1, LCDR2, and LCDR3),

wherein the VH comprises one, two, or all of the following:

(i) an HCDR1 comprising the amino acid sequence:
X₁X₂X₃X₄H,
wherein: X₁ is N, S, or A;
   X₂ is H or Y;
   X₃ is F or Y; and
   X₄ is I or M;
(SEQ ID NO: 83);

(ii) an HCDR2 comprising the amino acid sequence:
X₁INPX₂X₃GSX₄X₅X₆AQKX₇QG,
wherein: X₁ is I or T;
   X₂ is I, N, or V;
   X₃ is S or T;
   X₄ is S or T;
   X₅ is S, T, or N;
   X₆ is N or Y; and
   X₇ is F or L;
(SEQ ID NO: 84);

(iii) an HCDR3 comprising the amino acid sequence:
X₁X₂X₃DAFDX_{4,}
wherein: X₁ is D or E;
   X₂ is L or I;
   X₃ is V or L; and
   X₄ is F or Y;
(SEQ ID NO: 85); and/or

wherein the VL comprises one, two, or all of the following:

(iv) an LCDR1 comprising the amino acid sequence:
X₁ASX₂GX₃SSX₄LX_{5,}
wherein: X₁ is K or R;
   X₂ is Q or A;
   X₃ is I or V;
   X₄ is A or Y; and
   X₅ is A or V;
(SEQ ID NO: 86);

(v) an LCDR2 comprising the amino acid sequence:
X₁ASX₂X₃X₄X_{5,}
wherein: X₁ is A, D, or K;
   X₂ is N or S;
   X₃ is L or R;
   X₄ is E, Q, or A; and
   X₅ is S or T;
(SEQ ID NO: 87); and

(vi) an LCDR3 comprising the amino acid sequence:
QQX₁X₂X₃X₄X₅X_{6,}
wherein: X₁ is F or Y;
   X₂ is N or S;
   X₃ is D, N, or S;
   X₄ is Y or L;
   X₅ is F or Y; and
   X₆ is S or T;
(SEQ ID NO: 88).

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the CDR sequences listed in Table 2 for VH-1. In an embodiment, the VH comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 41; an HCDR2 comprising an amino acid sequence that differs by no more than 1, 2, or 3 amino acid residues from, or has at least 85, 90, 95, 99 or 100% homology with, the amino acid sequence of SEQ ID NO: 47; or an HCDR3 comprising an amino acid sequence that differs by no more than 1, 2, or 3 amino acid residues from, or has at least 85, 90, 95, 99 or 100% homology with, the amino acid sequence of SEQ ID NO: 53.

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the CDR sequences listed in Table 2 for VH-2. In an embodiment, the VH comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 43; an HCDR2 comprising an amino acid sequence that differs by no more than 1, 2, or 3 amino acid residues from, or has at least 85, 90, 95, 99 or 100% homology with, the amino acid sequence of SEQ ID NO: 48; or an HCDR3 comprising an amino acid sequence that differs by no more than 1, 2, or 3 amino acid residues from, or has at least 85, 90, 95, 99 or 100% homology with, the amino acid sequence of SEQ ID NO: 54.

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the CDR sequences listed in Table 2 for VH-3. In an embodiment, the VH comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 40; an HCDR2 comprising an amino acid sequence that differs by no more than 1, 2, or 3 amino acid residues from, or has at least 85, 90, 95, 99 or 100% homology with, the amino acid sequence of SEQ ID NO: 52; or an HCDR3 comprising an amino acid sequence that differs by no more than 1, 2, or 3 amino acid residues from, or has at least 85, 90, 95, 99 or 100% homology with, the amino acid sequence of SEQ ID NO: 56.

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the CDR sequences listed in Table 2 for VH-4. In an embodiment, the VH comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 39; an HCDR2 comprising an amino acid sequence that differs by no more than 1, 2, or 3 amino acid residues from, or has at least 85, 90, 95, 99 or 100% homology with, the amino acid sequence of SEQ ID NO: 51; or an HCDR3 comprising an amino acid sequence that differs by no more than 1, 2, or 3 amino acid residues from, or has at least 85, 90, 95, 99 or 100% homology with, the amino acid sequence of SEQ ID NO: 56.

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the CDR sequences listed in Table 2 for VH-5. In an embodiment, the VH comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 41; an HCDR2 comprising an amino acid sequence that differs by no more than 1, 2, or 3 amino acid residues from, or has at least 85, 90, 95, 99 or 100% homology with, the amino acid sequence of SEQ ID NO: 48; or an HCDR3 comprising an amino acid sequence that differs by no more than 1, 2, or 3 amino acid residues from, or has at least 85, 90, 95, 99 or 100% homology with, the amino acid sequence of SEQ ID NO: 53.

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the CDR sequences listed in Table 2 for VH-6. In an embodiment, the VH comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 41; an HCDR2 comprising an amino acid sequence that differs by no more than 1, 2, or 3 amino acid residues from, or has at least 85, 90, 95, 99 or 100% homology with, the amino acid sequence of SEQ ID NO: 44; or an HCDR3 comprising an amino acid sequence that differs by no more than 1, 2, or 3 amino acid residues from, or has at least 85, 90, 95, 99 or 100% homology with, the amino acid sequence of SEQ ID NO: 53.

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the CDR sequences listed in Table 2 for VH-7. In an embodiment, the VH comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 41; an HCDR2 comprising an amino acid sequence that differs by no more than 1, 2, or 3 amino acid residues from, or has at least 85, 90, 95, 99 or 100% homology with, the amino acid sequence of SEQ ID NO: 46; or an HCDR3 comprising an amino acid sequence that differs by no more than 1, 2, or 3 amino acid residues from, or has at least 85, 90, 95, 99 or 100% homology with, the amino acid sequence of SEQ ID NO: 53.

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the CDR sequences listed in Table 2 for VH-8. In an embodiment, the VH comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 41; an HCDR2 comprising an amino acid sequence that differs by no more than 1, 2, or 3 amino acid residues from, or has at least 85, 90, 95, 99 or 100% homology with, the amino acid sequence of SEQ ID NO: 49; or an HCDR3 comprising an amino acid sequence that differs by no more than 1, 2, or 3 amino acid residues from, or has at least 85, 90, 95, 99 or 100% homology with, the amino acid sequence of SEQ ID NO: 53.

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the CDR sequences listed in Table 2 for VH-9. In an embodiment, the VH comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 41; an HCDR2 comprising an amino acid sequence that differs by no more than 1, 2, or 3 amino acid residues from, or has at least 85, 90, 95, 99 or 100% homology with, the amino acid sequence of SEQ ID NO: 46; or an HCDR3 comprising an amino acid sequence that differs by no more than 1, 2, or 3 amino acid residues from, or has at least 85, 90, 95, 99 or 100% homology with, the amino acid sequence of SEQ ID NO: 57.

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the CDR sequences listed in Table 2 for VH-10. In an embodiment, the VH comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 42; an HCDR2 comprising an amino acid sequence that differs by no more than 1, 2, or 3 amino acid residues from, or has at least 85, 90, 95, 99 or 100% homology with, the amino acid sequence of SEQ ID NO: 49; or an HCDR3 comprising an amino acid sequence that differs by no more than 1, 2, or 3 amino acid residues from, or has at least 85, 90, 95, 99 or 100% homology with, the amino acid sequence of SEQ ID NO: 57.

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the CDR sequences listed in Table 2 for VH-11. In an embodiment, the VH comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 41; an HCDR2 comprising an amino acid sequence that differs by no more than 1, 2, or 3 amino acid residues from, or has at least 85, 90, 95, 99 or 100% homology with, the amino acid sequence of SEQ ID NO: 50; or an HCDR3 comprising an amino acid sequence that differs by no more than 1, 2, or 3 amino acid residues from, or has at least 85, 90, 95, 99 or 100% homology with, the amino acid sequence of SEQ ID NO: 54.

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the CDR sequences listed in Table 2 for VH-12. In an embodiment, the VH comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 41; an HCDR2 comprising an amino acid sequence that differs by no more than 1, 2, or 3 amino acid residues from, or has at least 85, 90, 95, 99 or 100% homology with, the amino acid sequence of SEQ ID NO: 49; or an HCDR3 comprising an amino acid sequence that differs by no more than 1, 2, or 3 amino acid residues from, or has at least 85, 90, 95, 99 or 100% homology with, the amino acid sequence of SEQ ID NO: 53.

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the CDR sequences listed in Table 2 for VH-13. In an embodiment, the VH comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 41; an HCDR2 comprising an amino acid sequence that differs by no more than 1, 2, or 3 amino acid residues from, or has at least 85, 90, 95, 99 or 100% homology with, the amino acid sequence of SEQ ID NO: 49; or an HCDR3 comprising an amino acid sequence that differs by no more than 1, 2, or 3 amino acid residues from, or has at least 85, 90, 95, 99 or 100% homology with, the amino acid sequence of SEQ ID NO: 55.

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the CDR sequences listed in Table 2 for VH-14. In an embodiment, the VH comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 110; an HCDR2 comprising an amino acid sequence that differs by no more than 1, 2, or 3 amino acid residues from, or has at least 85, 90, 95, 99 or 100% homology with, the amino acid sequence of SEQ ID NO: 49; or an HCDR3 comprising an amino acid sequence that differs by no more than 1, 2, or 3 amino acid residues from, or has at least 85, 90, 95, 99 or 100% homology with, the amino acid sequence of SEQ ID NO: 55.

In an embodiment, the antibody molecule comprises a heavy chain variable region (VH), wherein the heavy chain variable region comprises three heavy chain complementarity determining regions (HCDR1, HCDR2, and HCDR3), wherein the heavy chain variable region comprises one, two, or all of the CDR sequences listed in Table 2 for VH-15. In an embodiment, the VH comprises one, two, or all of the following: an HCDR1 comprising an amino acid sequence that differs by no more than 1, 2, or 3 amino acid residues from, or has at least 85, 90, 95, 99 or 100% homology with, the amino acid sequence of SEQ ID NO: 110; an HCDR2 comprising an amino acid sequence that differs by no more than 1, 2, or 3 amino acid residues from, or has at least 85, 90, 95, 99 or 100% homology with, the amino acid sequence of SEQ ID NO: 46; or an HCDR3 comprising an amino acid sequence that differs by no more than 1, 2, or 3 amino acid residues from, or has at least 85, 90, 95, 99 or 100% homology with, the amino acid sequence of SEQ ID NO: 53.

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the CDR sequences listed in Table 2 for VL-1. In an embodiment, the VL comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 61; an LCDR2 comprising an amino acid sequence that differs by no more than 1, 2, or 3 amino acid residues from, or has at least 85, 90, 95, 99 or 100% homology with, the amino acid sequence of SEQ ID NO: 67; or an LCDR3 comprising an amino acid sequence that differs by no more than 1, 2, or 3 amino acid residues from, or has at least 85, 90, 95, 99 or 100% homology with, the amino acid sequence of SEQ ID NO: 74.

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the CDR sequences listed in Table 2 for VL-2. In an embodiment, the VL comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 63; an LCDR2 comprising an amino acid sequence that differs by no more than 1, 2, or 3 amino acid residues from, or has at least 85, 90, 95, 99 or 100% homology with, the amino acid sequence of SEQ ID NO: 65; or an LCDR3 comprising an amino acid sequence that differs by no more than 1, 2, or 3 amino acid residues from, or has at least 85, 90, 95, 99 or 100% homology with, the amino acid sequence of SEQ ID NO: 76.

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the CDR sequences listed in Table 2 for VL-3. In an embodiment, the VL comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 60; an LCDR2 comprising an amino acid sequence that differs by no more than 1, 2, or 3 amino acid residues from, or has at least 85, 90, 95, 99 or 100% homology with, the amino acid sequence of SEQ ID NO: 64; or an LCDR3 comprising an amino acid sequence that differs by no more than 1, 2, or 3 amino acid residues from, or has at least 85, 90, 95, 99 or 100% homology with, the amino acid sequence of SEQ ID NO: 79.

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the CDR sequences listed in Table 2 for VL-4. In an embodiment, the VL comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 59; an LCDR2 comprising an amino acid sequence that differs by no more than 1, 2, or 3 amino acid residues from, or has at least 85, 90, 95, 99 or 100% homology with, the amino acid sequence of SEQ ID NO: 71; or an LCDR3 comprising an amino acid sequence that differs by no more than 1, 2, or 3 amino acid residues from, or has at least 85, 90, 95, 99 or 100% homology with, the amino acid sequence of SEQ ID NO: 75.

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the CDR sequences listed in Table 2 for VL-5. In an embodiment, the VL comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 61; an LCDR2 comprising an amino acid sequence that differs by no more than 1, 2, or 3 amino acid residues from, or has at least 85, 90, 95, 99 or 100% homology with, the amino acid sequence of SEQ ID NO: 67; or an LCDR3 comprising an amino acid sequence that differs by no more than 1, 2, or 3 amino acid residues from, or has at least 85, 90, 95, 99 or 100% homology with, the amino acid sequence of SEQ ID NO: 74.

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the CDR sequences listed in Table 2 for VL-6. In an embodiment, the VL comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 61; an LCDR2 comprising an amino acid sequence that differs by no more than 1, 2, or 3 amino acid residues from, or has at least 85, 90, 95, 99 or 100% homology with, the amino acid sequence of SEQ ID NO: 67; or an LCDR3 comprising an amino acid sequence that differs by no more than 1, 2, or 3 amino acid residues from, or has at least 85, 90, 95, 99 or 100% homology with, the amino acid sequence of SEQ ID NO: 74.

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the CDR sequences listed in Table 2 for VL-7. In an embodiment, the VL comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 61; an LCDR2 comprising an amino acid sequence that differs by no more than 1, 2, or 3 amino acid residues from, or has at least 85, 90, 95, 99 or 100% homology with, the amino acid sequence of SEQ ID NO: 67; or an LCDR3 comprising an amino acid sequence that differs by no more than 1, 2, or 3 amino acid residues from, or has at least 85, 90, 95, 99 or 100% homology with, the amino acid sequence of SEQ ID NO: 74.

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the CDR sequences listed in Table 2 for VL-8. In an embodiment, the VL comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 61; an LCDR2 comprising an amino acid sequence that differs by no more than 1, 2, or 3 amino acid residues from, or has at least 85, 90, 95, 99 or 100% homology with, the amino acid sequence of SEQ ID NO: 67; or an LCDR3 comprising an amino acid sequence that differs by no more than 1, 2, or 3 amino acid residues from, or has at least 85, 90, 95, 99 or 100% homology with, the amino acid sequence of SEQ ID NO: 74.

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the CDR sequences listed in Table 2 for VL-9. In an embodiment, the VL comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 61; an LCDR2 comprising an amino acid sequence that differs by no more than 1, 2, or 3 amino acid residues from, or has at least 85, 90, 95, 99 or 100% homology with, the amino acid sequence of SEQ ID NO: 72; or an LCDR3 comprising an amino acid sequence that differs by no more than 1, 2, or 3 amino acid residues from, or has at least 85, 90, 95, 99 or 100% homology with, the amino acid sequence of SEQ ID NO: 74.

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the CDR sequences listed in Table 2 for VL-10. In an embodiment, the VL comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 58; an LCDR2 comprising an amino acid sequence that differs by no more than 1, 2, or 3 amino acid residues from, or has at least 85, 90, 95, 99 or 100% homology with, the amino acid sequence of SEQ ID NO: 66; or an LCDR3 comprising an amino acid sequence that differs by no more than 1, 2, or 3 amino acid residues from, or has at least 85, 90, 95, 99 or 100% homology with, the amino acid sequence of SEQ ID NO: 78.

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the CDR sequences listed in Table 2 for VL-11. In an embodiment, the VL comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 62; an LCDR2 comprising an amino acid sequence that differs by no more than 1, 2, or 3 amino acid residues from, or has at least 85, 90, 95, 99 or 100% homology with, the amino acid sequence of SEQ ID NO: 66; or an LCDR3 comprising an amino acid sequence that differs by no more than 1, 2, or 3 amino acid residues from, or has at least 85, 90, 95, 99 or 100% homology with, the amino acid sequence of SEQ ID NO: 77.

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the CDR sequences listed in Table 2 for VL-12. In an embodiment, the VL comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 61; an LCDR2 comprising an amino acid sequence that differs by no more than 1, 2, or 3 amino acid residues from, or has at least 85, 90, 95, 99 or 100% homology with, the amino acid sequence of SEQ ID NO: 69; or an LCDR3 comprising an amino acid sequence that differs by no more than 1, 2, or 3 amino acid residues from, or has at least 85, 90, 95, 99 or 100% homology with, the amino acid sequence of SEQ ID NO: 74.

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the CDR sequences listed in Table 2 for VL-13. In an embodiment, the VL comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 61; an LCDR2 comprising an amino acid sequence that differs by no more than 1, 2, or 3 amino acid residues from, or has at least 85, 90, 95, 99 or 100% homology with, the amino acid sequence of SEQ ID NO: 69; or an LCDR3 comprising an amino acid sequence that differs by no more than 1, 2, or 3 amino acid residues from, or has at least 85, 90, 95, 99 or 100% homology with, the amino acid sequence of SEQ ID NO: 73.

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the CDR sequences listed in Table 2 for VL-14. In an embodiment, the VL comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 59; an LCDR2 comprising an amino acid sequence that differs by no more than 1, 2, or 3 amino acid residues from, or has at least 85, 90, 95, 99 or 100% homology with, the amino acid sequence of SEQ ID NO: 69; or an LCDR3 comprising an amino acid sequence that differs by no more than 1, 2, or 3 amino acid residues from, or has at least 85, 90, 95, 99 or 100% homology with, the amino acid sequence of SEQ ID NO: 73.

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the CDR sequences listed in Table 2 for VL-15. In an embodiment, the VL comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 61; an LCDR2 comprising an amino acid sequence that differs by no more than 1, 2, or 3 amino acid residues from, or has at least 85, 90, 95, 99 or 100% homology with, the amino acid sequence of SEQ ID NO: 69; or an LCDR3 comprising an amino acid sequence that differs by no more than 1, 2, or 3 amino acid residues from, or has at least 85, 90, 95, 99 or 100% homology with, the amino acid sequence of SEQ ID NO: 89.

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the CDR sequences listed in Table 2 for VL-16. In an embodiment, the VL comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 59; an LCDR2 comprising an amino acid sequence that differs by no more than 1, 2, or 3 amino acid residues from, or has at least 85, 90, 95, 99 or 100% homology with, the amino acid sequence of SEQ ID NO: 68; or an LCDR3 comprising an amino acid sequence that differs by no more than 1, 2, or 3 amino acid residues from, or has at least 85, 90, 95, 99 or 100% homology with, the amino acid sequence of SEQ ID NO: 73.

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the CDR sequences listed in Table 2 for VL-17. In an embodiment, the VL comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 59; an LCDR2 comprising an amino acid sequence that differs by no more than 1, 2, or 3 amino acid residues from, or has at least 85, 90, 95, 99 or 100% homology with, the amino acid sequence of SEQ ID NO: 70; or an LCDR3 comprising an amino acid sequence that differs by no more than 1, 2, or 3 amino acid residues from, or has at least 85, 90, 95, 99 or 100% homology with, the amino acid sequence of SEQ ID NO: 73.

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the CDR sequences listed in Table 2 for VL-18. In an embodiment, the VL comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 59; an LCDR2 comprising an amino acid sequence that differs by no more than 1, 2, or 3 amino acid residues from, or has at least 85, 90, 95, 99 or 100% homology with, the amino acid sequence of SEQ ID NO: 70; or an LCDR3 comprising an amino acid sequence that differs by no more than 1, 2, or 3 amino acid residues from, or has at least 85, 90, 95, 99 or 100% homology with, the amino acid sequence of SEQ ID NO: 89.

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the CDR sequences listed in Table 2 for VL-19. In an embodiment, the VL comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 61; an LCDR2 comprising an amino acid sequence that differs by no more than 1, 2, or 3 amino acid residues from, or has at least 85, 90, 95, 99 or 100% homology with, the amino acid sequence of SEQ ID NO: 68; or an LCDR3 comprising an amino acid sequence that differs by no more than 1, 2, or 3 amino acid residues from, or has at least 85, 90, 95, 99 or 100% homology with, the amino acid sequence of SEQ ID NO: 74.

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the CDR sequences listed in Table 2 for VL-20. In an embodiment, the VL comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 61; an LCDR2 comprising an amino acid sequence that differs by no more than 1, 2, or 3 amino acid residues from, or has at least 85, 90, 95, 99 or 100% homology with, the amino acid sequence of SEQ ID NO: 68; or an LCDR3 comprising an amino acid sequence that differs by no more than 1, 2, or 3 amino acid residues from, or has at least 85, 90, 95, 99 or 100% homology with, the amino acid sequence of SEQ ID NO: 73.

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the CDR sequences listed in Table 2 for VL-21. In an embodiment, the VL comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 59; an LCDR2 comprising an amino acid sequence that differs by no more than 1, 2, or 3 amino acid residues from, or has at least 85, 90, 95, 99 or 100% homology with, the amino acid sequence of SEQ ID NO: 68; or an LCDR3 comprising an amino acid sequence that differs by no more than 1, 2, or 3 amino acid residues from, or has at least 85, 90, 95, 99 or 100% homology with, the amino acid sequence of SEQ ID NO: 73.

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the CDR sequences listed in Table 2 for VL-22. In an embodiment, the VL comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 61; an LCDR2 comprising an amino acid sequence that differs by no more than 1, 2, or 3 amino acid residues from, or has at least 85, 90, 95, 99 or 100% homology with, the amino acid sequence of SEQ ID NO: 68; or an LCDR3 comprising an amino acid sequence that differs by no more than 1, 2, or 3 amino acid residues from, or has at least 85, 90, 95, 99 or 100% homology with, the amino acid sequence of SEQ ID NO: 89.

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the CDR sequences listed in Table 2 for VL-23. In an embodiment, the VL comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 59; an LCDR2 comprising an amino acid sequence that differs by no more than 1, 2, or 3 amino acid residues from, or has at least 85, 90, 95, 99 or 100% homology with, the amino acid sequence of SEQ ID NO: 69; or an LCDR3 comprising an amino acid sequence that differs by no more than 1, 2, or 3 amino acid residues from, or has at least 85, 90, 95, 99 or 100% homology with, the amino acid sequence of SEQ ID NO: 73.

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the CDR sequences listed in Table 2 for VL-24. In an embodiment, the VL comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 59; an LCDR2 comprising an amino acid sequence that differs by no more than 1, 2, or 3 amino acid residues from, or has at least 85, 90, 95, 99 or 100% homology with, the amino acid sequence of SEQ ID NO: 70; or an LCDR3 comprising an amino acid sequence that differs by no more than 1, 2, or 3 amino acid residues from, or has at least 85, 90, 95, 99 or 100% homology with, the amino acid sequence of SEQ ID NO: 73.

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the CDR sequences listed in Table 2 for VL-25. In an embodiment, the VL comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 59; an LCDR2 comprising an amino acid sequence that differs by no more than 1, 2, or 3 amino acid residues from, or has at least 85, 90, 95, 99 or 100% homology with, the amino acid sequence of SEQ ID NO: 70; or an LCDR3 comprising an amino acid sequence that differs by no more than 1, 2, or 3 amino acid residues from, or has at least 85, 90, 95, 99 or 100% homology with, the amino acid sequence of SEQ ID NO: 89.

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the CDR sequences listed in Table 2 for VL-26. In an embodiment, the VL comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 109; an LCDR2 comprising an amino acid sequence that differs by no more than 1, 2, or 3 amino acid residues from, or has at least 85, 90, 95, 99 or 100% homology with, the amino acid sequence of SEQ ID NO: 69; or an LCDR3 comprising an amino acid sequence that differs by no more than 1, 2, or 3 amino acid residues from, or has at least 85, 90, 95, 99 or 100% homology with, the amino acid sequence of SEQ ID NO: 89.

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the CDR sequences listed in Table 2 for VL-27. In an embodiment, the VL comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 109; an LCDR2 comprising an amino acid sequence that differs by no more than 1, 2, or 3 amino acid residues from, or has at least 85, 90, 95, 99 or 100% homology with, the amino acid sequence of SEQ ID NO: 69; or an LCDR3 comprising an amino acid sequence that differs by no more than 1, 2, or 3 amino acid residues from, or has at least 85, 90, 95, 99 or 100% homology with, the amino acid sequence of SEQ ID NO: 89.

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the CDR sequences listed in Table 2 for VL-28. In an embodiment, the VL comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 59; an LCDR2 comprising an amino acid sequence that differs by no more than 1, 2, or 3 amino acid residues from, or has at least 85, 90, 95, 99 or 100% homology with, the amino acid sequence of SEQ ID NO: 70; or an LCDR3 comprising an amino acid sequence that differs by no more than 1, 2, or 3 amino acid residues from, or has at least 85, 90, 95, 99 or 100% homology with, the amino acid sequence of SEQ ID NO: 74.

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the CDR sequences listed in Table 2 for VL-29. In an embodiment, the VL comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 61; an LCDR2 comprising an amino acid sequence that differs by no more than 1, 2, or 3 amino acid residues from, or has at least 85, 90, 95, 99 or 100% homology with, the amino acid sequence of SEQ ID NO: 70; or an LCDR3 comprising an amino acid sequence that differs by no more than 1, 2, or 3 amino acid residues from, or has at least 85, 90, 95, 99 or 100% homology with, the amino acid sequence of SEQ ID NO: 73.

In an embodiment, the antibody molecule comprises a light chain variable region (VL), wherein the light chain variable region comprises three light chain complementarity determining regions (LCDR1, LCDR2, and LCDR3), wherein the light chain variable region comprises one, two, or all of the CDR sequences listed in Table 2 for VL-30. In an embodiment, the VL comprises one, two, or all of the following: an LCDR1 comprising an amino acid sequence that differs by no more than 1, 2, or 3 amino acid residues from, or has at least 85, 90, 95, 99 or 100% homology with, the amino acid sequence of SEQ ID NO: 59; an LCDR2 comprising an amino acid sequence that differs by no more than 1, 2, or 3 amino acid residues from, or has at least 85, 90, 95, 99 or 100% homology with, the amino acid sequence of SEQ ID NO: 70; or an LCDR3 comprising an amino acid sequence that differs by no more than 1, 2, or 3 amino acid residues from, or has at least 85, 90, 95, 99 or 100% homology with, the amino acid sequence of SEQ ID NO: 73.

In an embodiment, the antibody molecule comprises a VH comprising an amino acid sequence at least 85%, 90%, 95%, 96%, 97%, 98%, or 99% identical to, or differing by no more than 1, 2, 3, 4, 5, 6, 7, 8, 9, 10, 11, 12, 13, 14, or 15 amino acid residues from, any of SEQ ID NOs: 1-13, 90, or 91. In an embodiment, the antibody molecule comprises a VH comprising an amino acid sequence of any of SEQ ID NOs: 1-13, 90, or 91.

In an embodiment, the antibody molecule comprises a VL comprising an amino acid sequence at least 85%, 90%, 95%, 96%, 97%, 98%, or 99% identical to, or differing by no more than 1, 2, 3, 4, 5, 6, 7, 8, 9, 10, 11, 12, 13, 14, or 15 amino acid residues from, any of SEQ ID NOs: 14-38 or 92-96. In an embodiment, the antibody molecule comprises a VL comprising an amino acid sequence of any of SEQ ID NOs: 14-38 or 92-96.

In an embodiment, the antibody molecule comprises a VH comprising an amino acid sequence at least 85%, 90%, 95%, 96%, 97%, 98%, or 99% identical to, or differing by no more than 1, 2, 3, 4, 5, 6, 7, 8, 9, 10, 11, 12, 13, 14, or 15 amino acid residues from, any of SEQ ID NOs: 1-13, 90, or 91 and a VL comprising an amino acid sequence at least 85%, 90%, 95%, 96%, 97%, 98%, or 99% identical to, or differing by no more than 1, 2, 3, 4, 5, 6, 7, 8, 9, 10, 11, 12, 13, 14, or 15 amino acid residues from, any of SEQ ID NOs: 14-38 or 92-96. In an embodiment, the antibody molecule comprises a VH comprising an amino acid sequence of any of SEQ ID NOs: 1-13, 90, or 91 and a VL comprising an amino acid sequence of any of SEQ ID NOs: 14-38 or 92-96.

In an embodiment, the antibody molecule comprises a VH, wherein the VH comprises three heavy chain complementarity determining regions (HCDR1, HCDR2, and HCDR3), wherein the VH comprises one, two, or all of the following: (i) an HCDR1 comprising an amino acid sequence that differs by no more than 1, 2, or 3 amino acid residues from, or has at least 85, 90, 95, 99 or 100% homology with, the amino acid sequence of SEQ ID NO: 41; (ii) an HCDR2 comprising an amino acid sequence that differs by no more than 1, 2, or 3 amino acid residues from, or has at least 85, 90, 95, 99 or 100% homology with, the amino acid sequence of SEQ ID NO: 46; or (ii) an HCDR3 comprising an amino acid sequence that differs by no more than 1, 2, or 3 amino acid residues from, or has at least 85, 90, 95, 99 or 100% homology with, the amino acid sequence of SEQ ID NO: 53.

In an embodiment, the antibody molecule comprises a VL, wherein the VL comprises three light chain complementarity determining regions (LCDR1, LCDR2, and LCDR3), wherein the light chain variable region comprises one, two, or all of the following: (i) an LCDR1 comprising an amino acid sequence that differs by no more than 1, 2, or 3 amino acid residues from, or has at least 85, 90, 95, 99 or 100% homology with, the amino acid sequence of SEQ ID NO: 59 or 61; (ii) an LCDR2 comprising an amino acid sequence that differs by no more than 1, 2, or 3 amino acid residues from, or has at least 85, 90, 95, 99 or 100% homology with, the amino acid sequence of any of SEQ ID NOS: 68-70; or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody SEQ ID NO: 73 or 74.

In an embodiment, the antibody molecule comprises:

(i) a VH comprising one, two, or all of the following: (i) an HCDR1 comprising an amino acid sequence that differs by no more than 1, 2, or 3 amino acid residues from, or has at least 85, 90, 95, 99 or 100% homology with, the amino acid sequence of SEQ ID NO: 41; (ii) an HCDR2 comprising an amino acid sequence that differs by no more than 1, 2, or 3 amino acid residues from, or has at least 85, 90, 95, 99 or 100% homology with, the amino acid sequence of SEQ ID NO: 46; or (ii) an HCDR3 comprising an amino acid sequence that differs by no more than 1, 2, or 3 amino acid residues from, or has at least 85, 90, 95, 99 or 100% homology with, the amino acid sequence of SEQ ID NO: 53, and

(ii) a VL comprising one, two, or all of the following: (i) an LCDR1 comprising an amino acid sequence that differs by no more than 1, 2, or 3 amino acid residues from, or has at least 85, 90, 95, 99 or 100% homology with, the amino acid sequence of SEQ ID NO: 59 or 61; (ii) an LCDR2 comprising an amino acid sequence that differs by no more than 1, 2, or 3 amino acid residues from, or has at least 85, 90, 95, 99 or 100% homology with, the amino acid sequence of any of SEQ ID NOS: 68-70; or (iii) an LCDR3 comprising an amino acid sequence that differs by no more than 1, 2, or 3 amino acid residues from, or has at least 85, 90, 95, 99 or 100% homology with, the amino acid sequence of the LCDR3 of monoclonal antibody SEQ ID NO: 73 or 74.

In an embodiment, the antibody molecule comprises: (i) a VH comprising: an HCDR1 comprising the amino acid sequence of SEQ ID NO: 41; an HCDR2 comprising the amino acid sequence of SEQ ID NO: 46; and an HCDR3 comprising the amino acid sequence of SEQ ID NO: 53, and (ii) a VL comprising: an LCDR1 comprising the amino acid sequence of the LCDR1 of SEQ ID NO: 59 or 61; an LCDR2 comprising the amino acid sequence of any of SEQ ID NOS: 68-70; and an LCDR3 comprising the amino acid sequence of SEQ ID NO: 73 or 74.

In an embodiment, the antibody molecule comprises: (i) a VH comprising: an HCDR1 comprising the consensus amino acid sequence of the HCDR1 of Exc23, Exc23.1, Exc23.2, Exc23.3, Exc23.4, and Exc23.5; an HCDR2 comprising the consensus amino acid sequence of the HCDR2 of Exc23, Exc23.1, Exc23.2, Exc23.3, Exc23.4, and Exc23.5; and an HCDR3 comprising the consensus amino acid sequence of the HCDR3 of Exc23, Exc23.1, Exc23.2, Exc23.3, Exc23.4, and Exc23.5, and/or (ii) a VL comprising: an LCDR1 comprising the consensus amino acid sequence of the LCDR1 of Exc23, Exc23.1, Exc23.2, Exc23.3, Exc23.4, and Exc23.5; an LCDR2 comprising the consensus amino acid sequence of the LCDR2 of Exc23, Exc23.1, Exc23.2, Exc23.3, Exc23.4, and Exc23.5; and an LCDR3 comprising the consensus amino acid sequence of the HCDR3 of Exc23, Exc23.1, Exc23.2, Exc23.3, Exc23.4, and Exc23.5.

In an embodiment, the antibody molecule comprises a VH comprising an amino acid sequence at least 85%, 90%, 95%, 96%, 97%, 98%, or 99% identical to, or differing by no more than 1, 2, 3, 4, 5, 6, 7, 8, 9, 10, 11, 12, 13, 14, or 15 amino acid residues from, of SEQ ID NO: 7. In an embodiment, the antibody molecule comprises a VH comprising an amino acid sequence of SEQ ID NO: 7.

In an embodiment, the antibody molecule comprises a VL comprising an amino acid sequence at least 85%, 90%, 95%, 96%, 97%, 98%, or 99% identical to, or differing by no more than 1, 2, 3, 4, 5, 6, 7, 8, 9, 10, 11, 12, 13, 14, or 15 amino acid residues from, any of SEQ ID NOs: 34, 36, 37, or 94-96. In an embodiment, the antibody molecule comprises a VL comprising an amino acid sequence of any of SEQ ID NOs: 34, 36, 37, or 94-96.

In an embodiment, the antibody molecule comprises a VH comprising an amino acid sequence at least 85%, 90%, 95%, 96%, 97%, 98%, or 99% identical to, or differing by no more than 1, 2, 3, 4, 5, 6, 7, 8, 9, 10, 11, 12, 13, 14, or 15 amino acid residues from, of SEQ ID NO: 7 and a VL comprising an amino acid sequence at least 85%, 90%, 95%, 96%, 97%, 98%, or 99% identical to, or differing by no more than 1, 2, 3, 4, 5, 6, 7, 8, 9, 10, 11, 12, 13, 14, or 15 amino acid residues from, any of SEQ ID NOs: 34, 36, 37, or 94-96. In an embodiment, the antibody molecule comprises a VH comprising an amino acid sequence of any of SEQ ID NO: 7 and a VL comprising an amino acid sequence of any of SEQ ID NOs: 34, 36, 37, or 94-96.

In an embodiment, the antibody molecule comprises a VH comprising an amino acid sequence of SEQ ID NO: 5 and/or a VL comprising an amino acid sequence of SEQ ID NO: 19. In an embodiment, the antibody molecule comprises a VH comprising an amino acid sequence of SEQ ID NO: 7 and/or a VL comprising an amino acid sequence of SEQ ID NO: 20. In an embodiment, the antibody molecule comprises a VH comprising an amino acid sequence of SEQ ID NO: 8 and/or a VL comprising an amino acid sequence of SEQ ID NO: 19. In an embodiment, the antibody molecule comprises a VH comprising an amino acid sequence of SEQ ID NO: 9 and/or a VL comprising an amino acid sequence of SEQ ID NO: 19. In an embodiment, the antibody molecule comprises a VH comprising an amino acid sequence of SEQ ID NO: 11 and/or a VL comprising an amino acid sequence of SEQ ID NO: 20. In an embodiment, the antibody molecule comprises a VH comprising an amino acid sequence of SEQ ID NO: 7 and/or a VL comprising an amino acid sequence of SEQ ID NO: 28. In an embodiment, the antibody molecule comprise a VH comprising an amino acid sequence of SEQ ID NO: 13 and/or a VL comprising an amino acid sequence of SEQ ID NO: 25. In an embodiment, the antibody molecule comprise a VH comprising an amino acid sequence of SEQ ID NO: 7 and/or a VL comprising an amino acid sequence of SEQ ID NO: 37. In an embodiment, the antibody molecule comprise a VH comprising an amino acid sequence of SEQ ID NO: 7 and/or a VL comprising an amino acid sequence of SEQ ID NO: 34. In an embodiment, the antibody molecule comprise a VH comprising an amino acid sequence of SEQ ID NO: 7 and/or a VL comprising an amino acid sequence of SEQ ID NO: 36. In an embodiment, the antibody molecule comprise a VH comprising an amino acid sequence of SEQ ID NO: 7 and/or a VL comprising an amino acid sequence of SEQ ID NO: 94. In an embodiment, the antibody molecule comprise a VH comprising an amino acid sequence of SEQ ID NO: 7 and/or a VL comprising an amino acid sequence of SEQ ID NO: 95. In an embodiment, the antibody molecule comprise a VH comprising an amino acid sequence of SEQ ID NO: 7 and/or a VL comprising an amino acid sequence of SEQ ID NO: 96.

In an embodiment, the antibody molecule further comprises a heavy chain constant region, e.g., a heavy chain constant region described herein. In an embodiment, the antibody molecule further comprises a light chain constant region, e.g., a light chain constant region described herein. In an embodiment, the antibody molecule further comprises a heavy chain constant region, e.g., a heavy chain constant region described herein, and a light chain constant region, e.g., a light chain constant region described herein. The constant region can be a wild-type or contain one or more mutations (e.g., YTE substitutions).

In an embodiment, the antibody molecule described herein has one or more (e.g., 2, 3, 4, 5, or all) of the following properties: specifically binds to FGF23 (e.g., human FGF23); prevents cleavage of FGF23, e.g., into FGF23a and FGF23b; prevents FGF23-based destruction of red blood cells; prevents chronic red blood cell destruction or hemolysis; reduces inflammation; or any combination thereof. In an embodiment, the antibody molecule comprises one or more (e.g., 2, 3, 4, 5, or all) CDRs, one or both of heavy chain variable region or light chain variable regions, or one or both of heavy chain or light chain, of any of antibody molecules ExA11, ExA28, ExA35, ExA43, ExA60, ExC17, ExC50, Exc23, Exc23.1, Exc23.2, Exc23.3, Exc23.4, or Exc23.5. In an embodiment, the antibody molecule is suitable for use in treating a disease or disorder, e.g., as described herein. In an embodiment, the disease or disorder is selected from X-linked hypophosphatemic rickets (XLH), autosomal recessive hypophosphatemic rickets (ARHR) (e.g., ARHR 1 or ARHR2), autosomal dominant hypophosphatemic rickets (ADHR), osteoglophonic dysplasia, Jansen-type metaphyseal chondrodysplasia, hypophosphatemia with dental abnormality and ectopic calcification, McCune-Albright syndrome, epidermal nevus syndrome (ENS), or tumor-induced osteomalacia (TIO). In another embodiment, the antibody molecule is suitable for use in treating a disease or disorder, e.g., a FGF23-associated disorder, e.g., a FGF23-associated disorder described herein.

The antibody molecules described herein can have several advantageous properties. For example, the antibody molecules can be used to effectively treat, prevent or diagnose a disorder associated with FGF23, e.g., a disorder described herein, e.g., a FGF23-associated disorder, e.g., a FGF23-associated disorder described herein.

In an embodiment, the antibody molecule binds to FGF23, e.g., human FGF23, with high affinity, e.g., with a K_{D}′ of about 50 nM or less, e.g., about 20 nM or less, 10 nM or less, 9 nM or less, 8 nM or less, 7 nM or less, 6 nM or less, 5 nM or less, 4 nM or less, 3 nM or less, 2 nM or less, 1 nM or less, 0.5 nM or less, 0.2 nM or less, 0.1 nM or less, 0.05 nM or less, 0.02 nM or less, 0.01 nM or less, 0.005 nM or less, 0.002 nM or less, or 0.001 nM or less, e.g., between 0.001 nM and 10 nM, between 0.001 nM and 5 nM, between 0.001 nM and 2 nM, between 0.001 nM and 1 nM, between 0.001 nM and 0.5 nM, between 0.001 nM and 0.2 nM, between 0.001 nM and 0.1 nM, between 0.001 and 0.05 nM, between 0.001 and 0.02 nM, between 0.001 and 0.005 nM, between 5 nM and 10 nM, between 2 nM and 10 nM, between 1 nM and 10 nM, between 0.5 nM and 10 nM, between 0.2 nM and 10 nM, between 0.1 nM and 10 nM, between 0.05 nM and 10 nM, between 0.02 nM and 10 nM, between 0.01 nM and 10 nM, between 0.005 nM and 10 nM, between 0.002 and 10 nM, between 0.002 nM and 5 nM, between 0.005 nM and 2 nM, between 0.01 nM and 1 nM, between 0.02 nM and 0.5 nM, between 0.05 nM and 0.2 nM, between 0.001 nM and 0.002 nM, between 0.002 nM and 0.005 nM, between 0.005 nM and 0.01 nM, between 0.01 nM and 0.02 nM, between 0.02 nM and 0.05 nM, between 0.05 nM and 0.1 nM, between 0.1 nM and 0.2 nM, between 0.2 nM and 0.5 nM, between 0.5 nM and 1 nM, between 1 nM and 2 nM, between 2 nM and 5 nM, or between 5 nM and 10 nM.

In an embodiment, the antibody molecule binds to FGF23 with a K_{off} slower than 1×10⁻⁴, 5×10⁻⁵, or 1×10⁻⁵ s⁻¹. In an embodiment, the antibody molecule binds to FGF23 with a Kₒₙ faster than 1×10⁴, 5×10⁴, 1×10⁵, or 5×10⁵ M⁻¹s⁻¹.

In an embodiment, the antibody molecule binds to FGF23, e.g., human FGF23, with high affinity, e.g., with an EFGF23₀ of about 2 μg/ml or less, e.g., about 1 μg/ml or less, 0.9 μg/ml or less, 0.8 μg/ml or less, 0.7 μg/ml or less, 0.6 μg/ml or less, 0.5 μg/ml or less, 0.4 μg/ml or less, 0.3 μg/ml or less, 0.2 μg/ml or less, 0.1 μg/ml or less, 0.09 μg/ml or less, 0.08 μg/ml or less, 0.07 μg/ml or less, 0.06 μg/ml or less, 0.05 μg/ml or less, 0.04 μg/ml or less, 0.03 μg/ml or less, 0.02 μg/ml or less, 0.01 μg/ml or less, 0.005 μg/ml or less, 0.002 μg/ml or less, 0.001 μg/ml or less, e.g., between 0.001 μg/ml and 2 μg/ml, e.g., between 0.001 μg/ml and 1 μg/ml, between 0.001 μg/ml and 0.5 μg/ml, between 0.001 μg/ml and 0.2 μg/ml, between 0.001 μg/ml and 0.1 μg/ml, between 0.001 μg/ml and 0.05 μg/ml, between 0.001 μg/ml and 0.02 μg/ml, between 0.001 μg/ml and 0.01 μg/ml, between 0.001 μg/ml and 0.005 μg/ml, between 0.002 μg/ml and 1 μg/ml, between 0.005 μg/ml and 1 μg/ml, between 0.01 μg/ml and 1 μg/ml, between 0.02 μg/ml and 1 μg/ml, between 0.05 μg/ml and 1 μg/ml, between 0.1 μg/ml and 1 μg/ml, between 0.2 μg/ml and 1 μg/ml, between 0.5 μg/ml and 1 μg/ml, between 0.001 μg/ml and 1 μg/ml, between 0.002 μg/ml and 0.5 μg/ml, between 0.005 μg/ml and 0.2 μg/ml, between 0.01 μg/ml and 0.1 μg/ml, or between 0.02 μg/ml and 0.05 μg/ml, e.g., as determined by a method described herein.

In an embodiment, the antibody molecule reduces (e.g., inhibits, blocks, or neutralizes) one or more biological activities of FGF23 (e.g., human FGF23), e.g., at an IC₅₀ of about 50 μg/ml or less, e.g., about 20 μg/ml or less, 10 μg/ml or less, 9 μg/ml or less, 8 μg/ml or less, 7 μg/ml or less, 6 μg/ml or less, 5 μg/ml or less, 4 μg/ml or less, 3 μg/ml or less, 2 μg/ml or less, 1 μg/ml or less, 0.5 μg/ml or less, 0.2 μg/ml or less, 0.1 μg/ml or less, 0.05 μg/ml or less, 0.02 μg/ml or less, 0.01 μg/ml or less, 0.005 μg/ml or less, 0.002 μg/ml or less, or 0.001 μg/ml or less, e.g., between 0.001 μg/ml and 10 μg/ml, between 0.001 μg/ml and 5 μg/ml, between 0.001 μg/ml and 2 μg/ml, between 0.001 μg/ml and 1 μg/ml, between 0.001 μg/ml and 0.5 μg/ml, between 0.001 μg/ml and 0.2 μg/ml, between 0.001 μg/ml and 0.1 μg/ml, between 0.001 and 0.05 μg/ml, between 0.001 and 0.02 μg/ml, between 0.001 and 0.005 μg/ml, between 5 μg/ml and 10 μg/ml, between 2 μg/ml and 10 μg/ml, between 1 μg/ml and 10 μg/ml, between 0.5 μg/ml and 10 μg/ml, between 0.2 μg/ml and 10 μg/ml, between 0.1 μg/ml and 10 μg/ml, between 0.05 μg/ml and 10 μg/ml, between 0.02 μg/ml and 10 μg/ml, between 0.01 μg/ml and 10 μg/ml, between 0.005 μg/ml and 10 μg/ml, between 0.002 and 10 μg/ml, between 0.002 μg/ml and 5 μg/ml, between 0.005 μg/ml and 2 μg/ml, between 0.01 μg/ml and 1 μg/ml, between 0.02 μg/ml and 0.5 μg/ml, between 0.05 μg/ml and 0.2 μg/ml, between 0.001 μg/ml and 0.002 μg/ml, between 0.002 μg/ml and 0.005 μg/ml, between 0.005 μg/ml and 0.01 μg/ml, between 0.01 μg/ml and 0.02 μg/ml, between 0.02 μg/ml and 0.05 μg/ml, between 0.05 μg/ml and 0.1 μg/ml, between 0.1 μg/ml and 0.2 μg/ml, between 0.2 μg/ml and 0.5 μg/ml, between 0.5 μg/ml and 1 μg/ml, between 1 μg/ml and 2 μg/ml, between 2 μg/ml and 5 μg/ml, or between 5 μg/ml and 10 μg/ml, e.g., as determined by a method described herein.

In an embodiment, the antibody molecule binds to a linear or conformational epitope on FGF23. In an embodiment, the antibody molecule binds to an epitope conserved between human FGF23 and mouse FGF23. In an embodiment, the antibody molecule binds, or substantially binds, to the same, similar, or overlapping epitope on FGF23, as a second antibody molecule (e.g., a monoclonal antibody described in Table 1). In an embodiment, the antibody molecule competes with a second antibody molecule (e.g., a monoclonal antibody described in Table 1) for binding to FGF23. In an embodiment, the epitope is a conformational epitope.

In an embodiment, LCDR1, LCDR2, LCDR3, HCDR1 and HCDR2 belong to Chothia CDR canonical classes 2, 1, 3, 1 and 3, respectively.

### Animal Models

The antibody molecules described herein can be evaluated in vivo, e.g., using various animal models. For example, an animal model can be used to test the pharmacokinetic and/or pharmacodynamics properties of an antibody molecule described herein in inhibiting FGF23 cleavage and/or in treating or preventing a disorder described herein, e.g., a FGF23-associated disorder, e.g., a FGF23-associated disorder described herein Animal models can also be used, e.g., to investigate for side effects, measure concentrations of antibody molecules in situ, demonstrate correlations between a FGF23 function and a FGF23-associated disorder, e.g., a FGF23-associated disorder described herein.

Exemplary animal models for a FGF23-associated disorder, e.g., a FGF23-associated disorder described herein that can be used for evaluating an antibody molecule described herein include, but are not limited to, FGF23 deficient mice, e.g., reconstituted with human FGF23.

Exemplary animal models for other disorders described herein are also known in the art. Exemplary types of animals that can be used to evaluate the antibody molecules described herein include, but are not limited to, mice, rats, rabbits, guinea pigs, and monkeys.

### Pharmaceutical Compositions and Kits

In an aspect, this disclosure provides compositions, e.g., pharmaceutically acceptable compositions, which include an antibody molecule described herein (e.g., a humanized antibody molecule described herein), formulated together with a pharmaceutically acceptable carrier.

As used herein, “pharmaceutically acceptable carrier” includes any and all solvents, dispersion media, isotonic and absorption delaying agents, and the like that are physiologically compatible. The carrier can be suitable for intravenous, intramuscular, subcutaneous, parenteral, rectal, spinal or epidermal administration (e.g., by injection or infusion). In an embodiment, less than about 5%, e.g., less than about 4%, 3%, 2%, or 1% of the antibody molecules in the pharmaceutical composition are present as aggregates. In other embodiments, at least about 95%, e.g., at least about 96%, 97%, 98%, 98.5%, 99%, 99.5%, 99.8%, or more of the antibody molecules in the pharmaceutical composition are present as monomers. In an embodiment, the level of aggregates or monomers is determined by chromatography, e.g., high performance size exclusion chromatography (HP-SEC).

The compositions set out herein may be in a variety of forms. These include, for example, liquid, semi-solid and solid dosage forms, such as liquid solutions (e.g., injectable and infusible solutions), dispersions or suspensions, liposomes, and suppositories. A suitable form depends on the intended mode of administration and therapeutic application. Typical suitable compositions are in the form of injectable or infusible solutions. One suitable mode of administration is parenteral (e.g., intravenous, subcutaneous, intraperitoneal, intramuscular). In an embodiment, the antibody molecule is administered by intravenous infusion or injection. In an embodiment, the antibody is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Therapeutic compositions typically should be sterile and stable under the conditions of manufacture and storage. The composition can be formulated as a solution, microemulsion, dispersion, liposome, or other ordered structure suitable to high antibody concentration. Sterile injectable solutions can be prepared by incorporating the active compound (i.e., antibody or antibody portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The antibody molecules described herein can be administered by a variety of methods. Several are known in the art, and for many therapeutic, prophylactic, or diagnostic applications, an appropriate route/mode of administration is intravenous injection or infusion. For example, the antibody molecules can be administered by intravenous infusion at a rate of less than 10 mg/min; preferably less than or equal to 5 mg/min to reach a dose of about 1 to 100 mg/m², preferably about 5 to 50 mg/m², about 7 to 25 mg/m² and more preferably, about 10 mg/m². As will be appreciated by the skilled artisan, the route and/or mode of administration will vary depending upon the desired results. In an embodiment, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In an embodiment, an antibody molecule can be orally administered, for example, with an inert diluent or an assimilable edible carrier. The antibody molecule (and other ingredients, if desired) may also be enclosed in a hard or soft shell gelatin capsule, compressed into tablets, or incorporated directly into the subject's diet. For oral therapeutic administration, the antibody molecule may be incorporated with excipients and used in the form of ingestible tablets, buccal tablets, troches, capsules, elixirs, suspensions, syrups, wafers, and the like. To administer an antibody molecule by other than parenteral administration, it may be necessary to coat the compound with, or co-administer the compound with, a material to prevent its inactivation. Therapeutic, prophylactic, or diagnostic compositions can also be administered with medical devices, and several are known in the art.

Dosage regimens are adjusted to provide the desired response (e.g., a therapeutic, prophylactic, or diagnostic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms are dictated by and directly dependent on (a) the unique characteristics of the antibody molecule and the particular therapeutic, prophylactic, or diagnostic effect to be achieved, and (b) the limitations inherent in the art of compounding such an antibody molecule for the treatment of sensitivity in individuals.

An exemplary, non-limiting range for a therapeutically, prophylactically, or diagnostically effective amount of an antibody molecule is about 0.1-50 mg/kg body weight of a subject, e.g., about 0.1-30 mg/kg, e.g., about 1-30, 1-15, 1-10, 1-5, 5-10, or 1-3 mg/kg, e.g., about 1, 2, 3, 4, 5, 6, 7, 8, 9, 10, 15, 20, 30, 40, or 50 mg/kg. The antibody molecule can be administered by intravenous infusion at a rate of less than 10 mg/min, e.g., less than or equal to 5 mg/min to reach a dose of about 1 to 100 mg/m², e.g., about 5 to 50 mg/m², about 7 to 25 mg/m², e.g., about 10 mg/m². It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed compositions.

The pharmaceutical compositions herein may include a “therapeutically effective amount,” “prophylactically effective amount,” or “diagnostically effectively amount” of an antibody molecule described herein.

A “therapeutically effective amount” refers to an amount effective, at dosages and for periods of time necessary, to achieve the desired therapeutic result. A therapeutically effective amount of the antibody molecule may vary according to factors such as the disease state, age, sex, and weight of the individual, and the ability of the antibody or antibody portion to elicit a desired response in the individual. A therapeutically effective amount is also one in which any toxic or detrimental effect of the antibody molecule is outweighed by the therapeutically beneficial effects. A “therapeutically effective dosage” typically inhibits a measurable parameter by at least about 20%, e.g., by at least about 40%, by at least about 60%, or by at least about 80% relative to untreated subjects. The measurable parameter may be, e.g., hematuria, colored urine, foamy urine, pain, swelling (edema) in the hands and feet, or high blood pressure. The ability of an antibody molecule to inhibit a measurable parameter can be evaluated in an animal model system predictive of efficacy in treating or preventing IgA nephropathy. Alternatively, this property of a composition can be evaluated by examining the ability of the antibody molecule to inhibit FGF23 cleavage, e.g., by an in vitro assay, e.g., by measuring FGF23b levels.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

A “diagnostically effective amount” refers to an amount effective, at dosages and for periods of time necessary, to achieve the desired diagnostic result. Typically, a diagnostically effective amount is one in which a disorder, e.g., a disorder described herein, e.g., IgA nephropathy, can be diagnosed in vitro, ex vivo, or in vivo.

Also within this disclosure is a kit that comprises an antibody molecule, described herein. The kit can include one or more other elements including: instructions for use; other reagents, e.g., a label, a therapeutic agent, or an agent useful for chelating, or otherwise coupling, an antibody molecule to a label or therapeutic agent, or a radioprotective composition; devices or other materials for preparing the antibody molecule for administration; pharmaceutically acceptable carriers; and devices or other materials for administration to a subject.

### Nucleic Acids

The present disclosure also features nucleic acids comprising nucleotide sequences that encode the antibody molecules (e.g., heavy and light chain variable regions and CDRs of the antibody molecules), as described herein.

For example, the present disclosure features a first and second nucleic acid encoding heavy and light chain variable regions, respectively, of an antibody molecule chosen from one or more of the antibody molecules disclosed herein, e.g., an antibody molecule of Table 1, or a portion of an antibody molecule, e.g., the variable regions of Table 1. The nucleic acid can comprise a nucleotide sequence encoding any one of the amino acid sequences in the tables herein, or a sequence substantially identical thereto (e.g., a sequence at least about 85%, 90%, 95%, 99% or more identical thereto, or which differs by no more than 3, 6, 15, 30, or 45 nucleotides from the sequences shown in the tables herein).

In an embodiment, the nucleic acid can comprise a nucleotide sequence encoding at least one, two, or three CDRs from a heavy chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In an embodiment, the nucleic acid can comprise a nucleotide sequence encoding at least one, two, or three CDRs from a light chain variable region having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions). In an embodiment, the nucleic acid can comprise a nucleotide sequence encoding at least one, two, three, four, five, or six CDRs from heavy and light chain variable regions having an amino acid sequence as set forth in the tables herein, or a sequence substantially homologous thereto (e.g., a sequence at least about 85%, 90%, 95%, 99% or more identical thereto, and/or having one or more substitutions, e.g., conserved substitutions).

In an embodiment, the nucleic acid can comprise a nucleotide sequence encoding at least one, two, or three CDRs from a heavy chain variable region having the nucleotide sequence as set forth in Table 5, a sequence substantially homologous thereto (e.g., a sequence at least about 85%, 90%, 95%, 99% or more identical thereto, and/or capable of hybridizing under the stringency conditions described herein). In an embodiment, the nucleic acid can comprise a nucleotide sequence encoding at least one, two, or three CDRs from a light chain variable region having the nucleotide sequence as set forth in Table 5, or a sequence substantially homologous thereto (e.g., a sequence at least about 85%, 90%, 95%, 99% or more identical thereto, and/or capable of hybridizing under the stringency conditions described herein). In an embodiment, the nucleic acid can comprise a nucleotide sequence encoding at least one, two, three, four, five, or six CDRs from heavy and light chain variable regions having the nucleotide sequence as set forth in Table 5, or a sequence substantially homologous thereto (e.g., a sequence at least about 85%, 90%, 95%, 99% or more identical thereto, and/or capable of hybridizing under the stringency conditions described herein).

In an embodiment, the nucleic acid comprises a nucleotide sequence as set forth in Table 5 or a sequence substantially homologous thereto (e.g., a sequence at least about 85%, 90%, 95%, 99% or more identical thereto, and/or capable of hybridizing under the stringency conditions described herein). In an embodiment, the nucleic acid comprises a portion of a nucleotide sequence as set forth in Table 5 or a sequence substantially homologous thereto (e.g., a sequence at least about 85%, 90%, 95%, 99% or more identical thereto, and/or capable of hybridizing under the stringency conditions described herein). The portion may encode, for example, a variable region (e.g., VH or VL); one, two, or three or more CDRs; or one, two, three, or four or more framework regions.

The nucleic acids disclosed herein include deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a non-natural arrangement.

In an aspect, the application features host cells and vectors containing the nucleic acids described herein. The nucleic acids may be present in a single vector or separate vectors present in the same host cell or separate host cell, as described in more detail below.

### Vectors

Further provided herein are vectors that comprise nucleotide sequences encoding an antibody molecule described herein.

In an embodiment, the vector comprises a nucleotide encoding an antibody molecule described herein, e.g., as described in Table 1. In another embodiment, the vector comprises a nucleotide sequence described herein, e.g., in Table 5. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes may be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed, or introduced into the same cell by cotransformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors may be transfected or introduced into an appropriate host cell. Various techniques may be employed to achieve this, such as, for example, protoplast fusion, calcium phosphate precipitation, electroporation, retroviral transduction, viral transfection, gene gun, lipid based transfection or other conventional techniques. In the case of protoplast fusion, the cells are grown in media and screened for the appropriate activity.

Methods and conditions for culturing the resulting transfected cells and for recovering the antibody molecule produced are known to those skilled in the art, and may be varied or optimized depending upon the specific expression vector and mammalian host cell employed, based upon the present description.

### Cells

The present disclosure also provides cells (e.g., host cells) comprising a nucleic acid encoding an antibody molecule as described herein. For example, the host cells may comprise a nucleic acid molecule having a nucleotide sequence described in Table 5, a sequence substantially homologous thereto (e.g., a sequence at least about 85%, 90%, 95%, 99% or more identical thereto, and/or capable of hybridizing under the stringency conditions described herein), or a portion of one of said nucleic acids. Additionally, the host cells may comprise a nucleic acid molecule encoding an amino acid sequence of Table 1, a sequence substantially homologous thereto (e.g., a sequence at least about 80%, 85%, 90%, 95%, 99% or more identical thereto), or a portion of one of said sequences.

In an embodiment, the host cells are genetically engineered to comprise nucleic acids encoding the antibody molecule described herein.

In an embodiment, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The disclosure also provides host cells comprising the vectors described herein.

The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells. In an embodiment, the cell (e.g., host cell) is an isolated cell.

### Uses of Antibody Molecules

The antibody molecules disclosed herein, as well as the pharmaceutical compositions disclosed herein, have in vitro, ex vivo, and in vivo therapeutic, prophylactic, and/or diagnostic utilities.

In an embodiment, the antibody molecule reduces (e.g., inhibits, blocks, or neutralizes) one or more biological activities of FGF23 (e.g., cleavage of FGF23). For example, these antibodies molecules can be administered to cells in culture, in vitro or ex vivo, or to a subject, e.g., a human subject, e.g., in vivo, to reduce (e.g., inhibits, blocks, or neutralizes) one or more biological activities of FGF23. In an embodiment, the antibody molecule inhibits, or substantially inhibit, cleavage of FGF23, e.g., human FGF23, e.g., to form FGF23a and FGF23b. Accordingly, in an aspect, the disclosure provides a method of treating, preventing, or diagnosing a disorder, e.g., a disorder described herein (e.g., IgA nephropathy), in a subject, comprising administering to the subject an antibody molecule described herein, such that the disorder is treated, prevented, or diagnosed. For example, the disclosure provides a method comprising contacting the antibody molecule described herein with cells in culture, e.g. in vitro or ex vivo, or administering the antibody molecule described herein to a subject, e.g., in vivo, to treat, prevent, or diagnose a disorder, e.g., a disorder associated with a FGF23-associated disorder, e.g., a FGF23-associated disorder described herein.

As used herein, the term “subject” is intended to include human and non-human animals. In an embodiment, the subject is a human subject, e.g., a human patient having a FGF23-associated disorder, e.g., a FGF23-associated disorder described herein, or at risk of having a FGF23-associated disorder, e.g., a FGF23-associated disorder described herein. The term “non-human animals” includes mammals and non-mammals, such as non-human primates. In an embodiment, the subject is a human The methods and compositions described herein are suitable for treating human patients a FGF23-associated disorder, e.g., a FGF23-associated disorder described herein. Patients having a FGF23-associated disorder, e.g., a FGF23-associated disorder described herein, include those who have developed a FGF23-associated disorder, e.g., a FGF23-associated disorder described herein, but are (at least temporarily) asymptomatic, patients who have exhibited a symptom of a FGF23-associated disorder, e.g., a FGF23-associated disorder described herein, or patients having a disorder related to or associated with a FGF23-associated disorder, e.g., a FGF23-associated disorder described herein.

In an embodiment, the subject has, or is at risk of having, X-linked hypophosphatemic rickets (XLH). In an embodiment, the subject is treated for XLH. In an embodiment, the subject has, or is at risk of having, autosomal recessive hypophosphatemic rickets (ARHR). In an embodiment, the subject is treated for ARHR (e.g., ARHR1 or ARHR2). In an embodiment, the subject has, or is at risk of having, autosomal dominant hypophosphatemic rickets (ADHR). In an embodiment, the subject is treated for ADHR. In an embodiment, the subject has, or is at risk of having, osteoglophonic dysplasia. In an embodiment, the subject is treated for osteoglophonic dysplasia. In an embodiment, the subject has, or is at risk of having, Jansen-type metaphyseal chondrodysplasia. In an embodiment, the subject is treated for Jansen-type metaphyseal chondrodysplasia. In an embodiment, the subject has, or is at risk of having, hypophosphatemia with dental abnormality and ectopic calcification. In an embodiment, the subject is treated for hypophosphatemia with dental abnormality and ectopic calcification. In an embodiment, the subject has, or is at risk of having, McCune-Albright syndrome. In an embodiment, the subject is treated for McCune-Albright syndrome. In an embodiment, the subject has, or is at risk of having, epidermal nevus syndrome (ENS). In an embodiment, the subject is treated for ENS. In an embodiment, the subject has, or is at risk of having, tumor-induced osteomalacia (TIO). In an embodiment, the subject is treated for TIO.

### Methods of Treating or Preventing Disorders

The antibody molecules described herein can be used to treat or prevent FGF23-associated disorders or symptoms thereof.

In an embodiment, the disorder is associated with aberrant levels of FGF23. In an embodiment, the antibody molecule is used to treat a subject having a disorder described herein, or is at risk of developing a disorder described herein.

Exemplary FGF23-associated disorders include, but are not limited to, hypophosphatemic disorders (e.g., hereditary hypophosphatemic disorders), e.g., with skeletal abnormalities, e.g., X-linked hypophosphatemic rickets (XLH), autosomal recessive hypophosphatemic rickets (ARHR) (e.g., ARHR 1 or ARHR2), autosomal dominant hypophosphatemic rickets (ADHR), osteoglophonic dysplasia, Jansen-type metaphyseal chondrodysplasia, hypophosphatemia with dental abnormality and ectopic calcification, McCune-Albright syndrome, epidermal nevus syndrome (ENS), or tumor-induced osteomalacia (TIO).

In an embodiment, the disorder is X-linked hypophosphatemic rickets (XLH). In an embodiment, the disorder is autosomal recessive hypophosphatemic rickets (ARHR) (e.g., ARHR 1 or ARHR2). In an embodiment, the disorder is autosomal dominant hypophosphatemic rickets (ADHR). In an embodiment, the disorder is osteoglophonic dysplasia. In an embodiment, the disorder is Jansen-type metaphyseal chondrodysplasia. In an embodiment, the disorder is hypophosphatemia with dental abnormality and ectopic calcification. In an embodiment, the disorder is McCune-Albright syndrome. In an embodiment, the disorder is epidermal nevus syndrome (ENS). In an embodiment, the disorder is tumor-induced osteomalacia (TIO).

In some embodiments, the FGF23-associated disorder is X-linked hypophosphatemic rickets (XLH), also known as X-linked hypophosphatemia or X-linked Vitamin D-resistant rickets. XLH is an X-linked dominant form of rickets associated with reduction in activity of the PHEX protein, which generally cannot be addressed by vitamin D supplementation. XLH can lead to, e.g., bone deformities, pain, short stature, genu varum, hearing loss, and/or osteoarthritis. The PHEX protein is encoded by the PHEX gene, which is located on the human X chromosome at location Xp22.2-p22.1. The PHEX protein is known to downregulate FGF23 activity; as such, reduction in PHEX activity (e.g., by a mutation that alters the PHEX amino acid sequence or prevents PHEX expression) can result in hyperactivation of FGF23. In certain embodiments, treatment of a subject having XLH with an antibody molecule described herein may reduce FGF23 activity, e.g., counteracting the hyperactivation of FGF23 induced by loss of PHEX function.

The antibody molecules described herein are typically administered at a frequency that keeps a therapeutically effective level of antibody molecules in the patient's system until the patient recovers. For example, the antibody molecules may be administered at a frequency that achieves a serum concentration sufficient for at least about 1, 2, 5, 10, 20, 30, or 40 antibody molecules to bind each FGF23 molecule. In an embodiment, the antibody molecules are administered every 1, 2, 3, 4, 5, 6, or 7 days, every 1, 2, 3, 4, 5, or 6 weeks, or every 1, 2, 3, 4, 5, or 6 months.

Methods of administering various antibody molecules are known in the art and are described below. Suitable dosages of the antibody molecules used will depend on the age and weight of the subject and the particular drug used.

In an embodiment, the antibody molecule is administered to the subject (e.g., a human subject) intravenously. In an embodiment, the antibody molecule is administered to the subject at a dose between 0.1 mg/kg and 50 mg/kg, e.g., between 0.2 mg/kg and 25 mg/kg, between 0.5 mg/kg and 10 mg/kg, between 0.5 mg/kg and 5 mg/kg, between 0.5 mg/kg and 3 mg/kg, between 0.5 mg/kg and 2.5 mg/kg, between 0.5 mg/kg and 2 mg/kg, between 0.5 mg/kg and 1.5 mg/kg, between 0.5 mg/kg and 1 mg/kg, between 1 mg/kg and 1.5 mg/kg, between 1 mg/kg and 2 mg/kg, between 1 mg/kg and 2.5 mg/kg, between 1 mg/kg and 3 mg/kg, between 1 mg/kg and 2.5 mg/kg, or between 1 mg/kg and 5 mg/kg. In an embodiment, the antibody molecule is administered to the subject at a fixed dose between 10 mg and 1000 mg, e.g., between 10 mg and 500 mg, between 10 mg and 250 mg, between 10 mg and 150 mg, between 10 mg and 100 mg, between 10 mg and 50 mg, between 250 mg and 500 mg, between 150 mg and 500 mg, between 100 mg and 500 mg, between 50 mg and 500 mg, between 25 mg and 250 mg, between 50 mg and 150 mg, between 50 mg and 100 mg, between 100 mg and 150 mg. between 100 mg and 200 mg, or between 150 mg and 250 mg. In an embodiment, the antibody molecule is administered once a week, twice a week, once every two weeks, once every three weeks, once every four weeks, once every eight weeks, once a month, once every two months, or once every three months. In an embodiment, the antibody molecule is administered between 0.5 mg/kg and 3 mg/kg or between 50 mg and 150 mg, once a week, twice a week, once every two weeks, or once every four weeks.

The antibody molecules can be used by themselves or conjugated to a second agent, e.g., a bacterial agent, toxin, or protein, e.g., a second anti-FGF23 antibody molecule. This method includes: administering the antibody molecule, alone or conjugated to a second agent, to a subject requiring such treatment. The antibody molecules can be used to deliver a variety of therapeutic agents, e.g., a toxin, or mixtures thereof.

### Combination Therapies

The antibody molecules can be used in combination with other therapies. For example, the combination therapy can include an antibody molecule co-formulated with, and/or co-administered with, one or more additional therapeutic agents, e.g., one or more additional therapeutic agents described herein. In other embodiments, the antibody molecules are administered in combination with other therapeutic treatment modalities, e.g., other therapeutic treatment modalities described herein. Such combination therapies may advantageously utilize lower dosages of the administered therapeutic agents, thus avoiding possible toxicities or complications associated with the various monotherapies.

Administered “in combination”, as used herein, means that two (or more) different treatments are delivered to the subject before, or during the course of the subject's affliction with a disorder. In an embodiment, two or more treatments are delivered prophylactically, e.g., before the subject has the disorder or is diagnosed with the disorder. In another embodiment, the two or more treatments are delivered after the subject has developed or diagnosed with the disorder. In an embodiment, the delivery of one treatment is still occurring when the delivery of the second begins, so that there is overlap. This is sometimes referred to herein as “simultaneous” or “concurrent delivery.” In other embodiments, the delivery of one treatment ends before the delivery of the other treatment begins. In an embodiment of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In an embodiment, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In an embodiment, the additional agent is a second antibody molecule, e.g., an antibody molecule different from a first antibody molecule. Exemplary antibody molecules that can be used in combination include, but are not limited to, any combination of the antibody molecules listed in Table 1.

In an embodiment, the antibody molecule is administered in combination with a second therapy to treat or prevent a FGF23-associated disorder, e.g., a FGF23-associated disorder described herein.

Exemplary therapies that can be used in combination with an antibody molecule or composition described herein to treat or prevent other disorders are also described in the section of “Methods of Treating or Preventing Disorders” herein.

### Methods of Diagnosis

In some aspects, the present disclosure provides a diagnostic method for detecting the presence of FGF23 (e.g., human FGF23) in vitro (e.g., in a biological sample, such as a biopsy or blood sample) or in vivo (e.g., in vivo imaging in a subject). The method includes: (i) contacting the sample with an antibody molecule described herein, or administering to the subject, the antibody molecule; (optionally) (ii) contacting a reference sample, e.g., a control sample (e.g., a control biological sample, such as a biopsy or blood sample) or a control subject with an antibody molecule described herein; and (iii) detecting formation of a complex between the antibody molecule and FGF23 in the sample or subject, or the control sample or subject, wherein a change, e.g., a statistically significant change, in the formation of the complex in the sample or subject relative to the control sample or subject is indicative of the presence of FGF23 in the sample. The antibody molecule can be directly or indirectly labeled with a detectable substance to facilitate detection of the bound or unbound antibody. Suitable detectable substances include various enzymes, prosthetic groups, fluorescent materials, luminescent materials and radioactive materials, as described above and described in more detail below.

The term “sample,” as it refers to samples used for detecting a polypeptide (e.g., FGF23) or a nucleic acid encoding the polypeptide includes, but is not limited to, cells, cell lysates, proteins or membrane extracts of cells, body fluids such as blood, or tissue samples such as biopsies.

Complex formation between the antibody molecule, and FGF23, can be detected by measuring or visualizing either the antibody molecule bound to FGF23 or unbound antibody molecule. Any suitable detection assays can be used, and conventional detection assays include an enzyme-linked immunosorbent assays (ELISA), a radioimmunoassay (RIA) or tissue immunohistochemistry. Alternative to labeling the antibody molecule, the presence of FGF23 can be assayed in a sample by a competition immunoassay utilizing standards labeled with a detectable substance and an unlabeled antibody molecule. In this assay, the biological sample, the labeled standards and the antibody molecule are combined and the amount of labeled standard bound to the unlabeled binding molecule is determined. The amount of FGF23 in the sample is inversely proportional to the amount of labeled standard bound to the antibody molecule.

The antibody molecules described herein can be used to diagnose disorders that can be treated or prevented by the antibody molecules described herein. The detection or diagnostic methods described herein can be used in combination with other methods described herein to treat or prevent a disorder described herein.

The present disclosure also includes any of the following numbered paragraphs:

1. An isolated antibody molecule capable of binding to FGF23, comprising:

(a) a heavy chain variable region (VH) comprising an HCDR1 amino acid sequence of SEQ ID NO: 41, an HCDR2 amino acid sequence of SEQ ID NO: 48, and an HCDR3 amino acid sequence of SEQ ID NO: 53; and a light chain variable region (VL) comprising an LCDR1 amino acid sequence of SEQ ID NO: 61, an LCDR2 amino acid sequence of SEQ ID NO: 67, and an LCDR3 amino acid sequence of SEQ ID NO: 74;

(b) a VH comprising an HCDR1 amino acid sequence of SEQ ID NO: 41, an HCDR2 amino acid sequence of SEQ ID NO: 46, and an HCDR3 amino acid sequence of SEQ ID NO: 53; and a light chain variable region (VL) comprising an LCDR1 amino acid sequence of SEQ ID NO: 61, an LCDR2 amino acid sequence of SEQ ID NO: 67, and an LCDR3 amino acid sequence of SEQ ID NO: 74;

(c) a VH comprising an HCDR1 amino acid sequence of SEQ ID NO: 41, an HCDR2 amino acid sequence of SEQ ID NO: 49, and an HCDR3 amino acid sequence of SEQ ID NO: 53; and a light chain variable region (VL) comprising an LCDR1 amino acid sequence of SEQ ID NO: 61, an LCDR2 amino acid sequence of SEQ ID NO: 67, and an LCDR3 amino acid sequence of SEQ ID NO: 74;

(d) a VH comprising an HCDR1 amino acid sequence of SEQ ID NO: 41, an HCDR2 amino acid sequence of SEQ ID NO: 46, and an HCDR3 amino acid sequence of SEQ ID NO: 57; and a light chain variable region (VL) comprising an LCDR1 amino acid sequence of SEQ ID NO: 61, an LCDR2 amino acid sequence of SEQ ID NO: 67, and an LCDR3 amino acid sequence of SEQ ID NO: 74;

(e) a VH comprising an HCDR1 amino acid sequence of SEQ ID NO: 41, an HCDR2 amino acid sequence of SEQ ID NO: 50, and an HCDR3 amino acid sequence of SEQ ID NO: 54; and a light chain variable region (VL) comprising an LCDR1 amino acid sequence of SEQ ID NO: 61, an LCDR2 amino acid sequence of SEQ ID NO: 67, and an LCDR3 amino acid sequence of SEQ ID NO: 74;

(f) a VH comprising an HCDR1 amino acid sequence of SEQ ID NO: 41, an HCDR2 amino acid sequence of SEQ ID NO: 46, and an HCDR3 amino acid sequence of SEQ ID NO: 53; and a light chain variable region (VL) comprising an LCDR1 amino acid sequence of SEQ ID NO: 61, an LCDR2 amino acid sequence of SEQ ID NO: 69, and an LCDR3 amino acid sequence of SEQ ID NO: 89;

(g) a VH comprising an HCDR1 amino acid sequence of SEQ ID NO: 41, an HCDR2 amino acid sequence of SEQ ID NO: 49, and an HCDR3 amino acid sequence of SEQ ID NO: 55; and a light chain variable region (VL) comprising an LCDR1 amino acid sequence of SEQ ID NO: 61, an LCDR2 amino acid sequence of SEQ ID NO: 69, and an LCDR3 amino acid sequence of SEQ ID NO: 74;

(h) a VH comprising an HCDR1 amino acid sequence of SEQ ID NO: 41, an HCDR2 amino acid sequence of SEQ ID NO: 46, and an HCDR3 amino acid sequence of SEQ ID NO: 53; and a light chain variable region (VL) comprising an LCDR1 amino acid sequence of SEQ ID NO: 59, an LCDR2 amino acid sequence of SEQ ID NO: 70, and an LCDR3 amino acid sequence of SEQ ID NO: 73;

(i) a VH comprising an HCDR1 amino acid sequence of SEQ ID NO: 41, an HCDR2 amino acid sequence of SEQ ID NO: 46, and an HCDR3 amino acid sequence of SEQ ID NO: 53; and a light chain variable region (VL) comprising an LCDR1 amino acid sequence of SEQ ID NO: 59, an LCDR2 amino acid sequence of SEQ ID NO: 68, and an LCDR3 amino acid sequence of SEQ ID NO: 73;

(j) a VH comprising an HCDR1 amino acid sequence of SEQ ID NO: 41, an HCDR2 amino acid sequence of SEQ ID NO: 46, and an HCDR3 amino acid sequence of SEQ ID NO: 53; and a light chain variable region (VL) comprising an LCDR1 amino acid sequence of SEQ ID NO: 59, an LCDR2 amino acid sequence of SEQ ID NO: 69, and an LCDR3 amino acid sequence of SEQ ID NO: 73;

(k) a VH comprising an HCDR1 amino acid sequence of SEQ ID NO: 41, an HCDR2 amino acid sequence of SEQ ID NO: 46, and an HCDR3 amino acid sequence of SEQ ID NO: 53; and a light chain variable region (VL) comprising an LCDR1 amino acid sequence of SEQ ID NO: 59, an LCDR2 amino acid sequence of SEQ ID NO: 70, and an LCDR3 amino acid sequence of SEQ ID NO: 74;

(l) a VH comprising an HCDR1 amino acid sequence of SEQ ID NO: 41, an HCDR2 amino acid sequence of SEQ ID NO: 46, and an HCDR3 amino acid sequence of SEQ ID NO: 53; and a light chain variable region (VL) comprising an LCDR1 amino acid sequence of SEQ ID NO: 61, an LCDR2 amino acid sequence of SEQ ID NO: 70, and an LCDR3 amino acid sequence of SEQ ID NO: 73; or

(m) a VH comprising an HCDR1 amino acid sequence of SEQ ID NO: 41, an HCDR2 amino acid sequence of SEQ ID NO: 46, and an HCDR3 amino acid sequence of SEQ ID NO: 53; and a light chain variable region (VL) comprising an LCDR1 amino acid sequence of SEQ ID NO: 59, an LCDR2 amino acid sequence of SEQ ID NO: 70, and an LCDR3 amino acid sequence of SEQ ID NO: 73.

2. An isolated antibody molecule capable of binding to FGF23, comprising a heavy chain variable region (VH) and a light chain variable region (VL), wherein the VH comprises an HCDR1 amino acid sequence of any of SEQ ID NOs: 39-43 or 110; an HCDR2 amino acid sequence of any of SEQ ID NOs: 44-52; and an HCDR3 amino acid sequence of any of SEQ ID NOs: 53-57; and the VL comprises an LCDR1 amino acid sequence of any of SEQ ID NOs: 58-63 and 109, an LCDR2 amino acid sequence of any of SEQ ID NOs: 64-72, and an LCDR3 amino acid sequence of any of SEQ ID NOs: 73-89.

3. The antibody molecule of paragraph 1 or 2, which comprises a VH comprising an amino acid sequence at least 85%, 90%, or 95% identical to any of SEQ ID NOs: 1-13, 90, or 91.

4. The antibody molecule of paragraph 3, which comprises a VH comprising an amino acid sequence of any of SEQ ID NOs: 1-13, 90, or 91.

5. The antibody molecule of any of paragraphs 1-4, which comprises a VL comprising an amino acid sequence at least 85%, 90%, or 95% identical to any of SEQ ID NOs: 14-38 or 92-96.

6. The antibody molecule of paragraph 5, which comprises a VL comprising an amino acid sequence of any of SEQ ID NOs: 14-38 or 92-96.

7. The antibody molecule of paragraph 1, which comprises a VH comprising an amino acid sequence at least 85%, 90%, or 95% identical to any of SEQ ID NOs: 1-13, 90, or 91 and a VL comprising an amino acid sequence at least 85%, 90%, or 95% identical to any of SEQ ID NOs: 14-38 or 92-96.

8. The antibody molecule of paragraph 7, which comprises a VH comprising an amino acid sequence of any of SEQ ID NOs: 1-13, 90, or 91 and a VL comprising an amino acid sequence of any of SEQ ID NOs: 14-38 or 92-96.

9. The antibody molecule of any of paragraphs 1-8, which comprise a VH comprising an amino acid sequence of SEQ ID NO: 5 and a VL comprising an amino acid sequence of SEQ ID NO: 19.

10. The antibody molecule of any of paragraphs 1-8, which comprise a VH comprising an amino acid sequence of SEQ ID NO: 7 and a VL comprising an amino acid sequence of SEQ ID NO: 20.

11. The antibody molecule of any of paragraphs 1-8, which comprise a VH comprising an amino acid sequence of SEQ ID NO: 8 and a VL comprising an amino acid sequence of SEQ ID NO: 19.

12. The antibody molecule of any of paragraphs 1-8, which comprise a VH comprising an amino acid sequence of SEQ ID NO: 9 and a VL comprising an amino acid sequence of SEQ ID NO: 19.

13. The antibody molecule of any of paragraphs 1-8, which comprise a VH comprising an amino acid sequence of SEQ ID NO: 11 and a VL comprising an amino acid sequence of SEQ ID NO: 20.

14. The antibody molecule of any of paragraphs 1-8, which comprise a VH comprising an amino acid sequence of SEQ ID NO: 7 and a VL comprising an amino acid sequence of SEQ ID NO: 28.

15. The antibody molecule of any of paragraphs 1-8, which comprise a VH comprising an amino acid sequence of SEQ ID NO: 13 and a VL comprising an amino acid sequence of SEQ ID NO: 25.

16. The antibody molecule of any of paragraphs 1-8, which comprise a VH comprising an amino acid sequence of SEQ ID NO: 7 and a VL comprising an amino acid sequence of SEQ ID NO: 37.

17. The antibody molecule of any of paragraphs 1-8, which comprise a VH comprising an amino acid sequence of SEQ ID NO: 7 and a VL comprising an amino acid sequence of SEQ ID NO: 34.

18. The antibody molecule of any of paragraphs 1-8, which comprise a VH comprising an amino acid sequence of SEQ ID NO: 7 and a VL comprising an amino acid sequence of SEQ ID NO: 36.

19. The antibody molecule of any of paragraphs 1-8, which comprise a VH comprising an amino acid sequence of SEQ ID NO: 7 and a VL comprising an amino acid sequence of SEQ ID NO: 94.

20. The antibody molecule of any of paragraphs 1-8, which comprise a VH comprising an amino acid sequence of SEQ ID NO: 7 and a VL comprising an amino acid sequence of SEQ ID NO: 95.

21. The antibody molecule of any of paragraphs 1-8, which comprise a VH comprising an amino acid sequence of SEQ ID NO: 7 and a VL comprising an amino acid sequence of SEQ ID NO: 96.

22. The antibody molecule of any of the preceding paragraphs, which comprises an antigen-binding fragment.

23. The antibody molecule of paragraph 22, wherein the antigen-binding fragment comprises a Fab, F(ab′)2, Fv, scFv, or sc(Fv)2.

24. The antibody molecule of any of the preceding paragraphs, which comprises a light chain constant region chosen from the light chain constant regions of kappa or lambda.

25. The antibody molecule of any of the preceding paragraphs, which comprises a heavy chain constant region chosen from the heavy chain constant regions of IgG1, IgG2, IgG3, IgG4, or a chimera of two or more isotypes (e.g. IgG2 and IgG4), and a light chain constant region chosen from the light chain constant regions of kappa or lambda.

26. The antibody molecule of any of the preceding paragraphs, which comprises a heavy chain constant region chosen from the heavy chain constant regions of IgG1, IgG4, or a chimera of IgG2 and IgG4 (e.g. “IgG2/4”), and optionally, wherein the heavy chain constant region comprises one or more amino acid modifications in the hinge, CH2, and/or CH3 region (e.g., IgG1-YTE, IgG4-YTE or IgG2/4-YTE).

27. The antibody molecule of any of the preceding paragraphs, which comprises an Fc region.

28. The antibody molecule of any of the preceding paragraphs, wherein said antibody molecule is a humanized antibody molecule.

29. The antibody molecule of any of the preceding paragraphs, wherein said antibody molecule is a monoclonal antibody molecule.

30. The antibody molecule of any of the preceding paragraphs, wherein said antibody molecule is a synthetic antibody molecule.

31. The antibody molecule of any of the preceding paragraphs, wherein said antibody molecule is a monospecific antibody molecule.

32. The antibody molecule of any of the preceding paragraphs, wherein the FGF23 is a human FGF23.

33. The antibody molecule of any of the preceding paragraphs, which binds to human FGF23 at an EC₅₀ of less than 0.04 μg/ml (e.g., less than about 0.04, 0.03, 0.029, 0.028, 0.027, 0.026, 0.025, 0.024, 0.023, 0.022, or 0.021 μg/ml), e.g., as determined by ELISA.

34. The antibody molecule of any of the preceding paragraphs, which binds to human FGF23 at an EC₅₀ of between 0.01 μg/ml and 0.04 μg/ml (e.g., between 0.02 μg/ml and 0.04 μg/ml, between 0.02 μg/ml and 0.03 μg/ml, between 0.02 μg/ml and 0.025 μg/ml, or between 0.025 μg/ml and 0.03 μg/ml), e.g., as determined by ELISA.

35. The antibody molecule of any of the preceding paragraphs, which inhibits cell proliferation at an IC₅₀ of less than 10 μg/ml (e.g., less than about 10, 9, 8, 7, 6, 5, 4, 3, 2.8, 2.5, 2, 1.7, 1.6, 1.5, 1.4, 1.3, 1, 0.9, 0.8, 0.7, 0.6, 0.5, 0.4, 0.3, 0.2, or 0.1 μg/ml), e.g., as determined by a cell-based assay, e.g., as described in Example 2.

36. The antibody molecule of any of the preceding paragraphs, which inhibits cell proliferation at an IC₅₀ of between 0.1 μg/ml and 3 μg/ml (e.g., between 0.1 μg/ml and 0.3 μg/ml, between 0.3 μg/ml and 0.6 μg/ml, between 0.6 μg/ml and 1 μg/ml, between 1 μg/ml and 2 μg/ml, or between 2 μg/ml and 3 μg/ml) as determined by a cell-based assay, e.g., as described in Example 2.

37. The antibody molecule of any of the preceding paragraphs, which binds to human FGF23 comprising the amino acid sequence of SEQ ID NO: 82.

38. The antibody molecule of any of the preceding paragraphs, wherein LCDR1, LCDR2, LCDR3, HCDR1 and HCDR2 belong to Chothia CDR canonical classes 2, 1, 3, 1 and 3, respectively.

39. An antibody molecule capable of binding to FGF23, comprising a VH comprising an HCDR1, an HCDR2, and an HCDR3, and a VL comprising an LCDR1, an LCDR2, and an LCDR3, wherein LCDR1, LCDR2, LCDR3, HCDR1 and HCDR2 belong to Chothia CDR canonical classes 2, 1, 3, 1 and 3, respectively.

40. An antibody molecule that competes for binding to FGF23 with an antibody molecule of any of the preceding paragraphs.

41. An antibody molecule that binds to the same or overlapping epitope as the epitope recognized by an antibody molecule of any of the preceding paragraphs.

42. A pharmaceutical composition comprising the isolated antibody molecule of any of the preceding paragraphs and a pharmaceutically acceptable carrier, excipient or stabilizer.

43. An isolated nucleic acid encoding the VH, VL, or both, of the antibody molecule of any of paragraphs 1-41.

44. The isolated nucleic acid of paragraph 36, wherein the nucleic acid comprises:

(a) the nucleic acid sequence of SEQ ID NO: 111 and/or the nucleic acid sequence of SEQ ID NO: 112;

(b) the nucleic acid sequence of SEQ ID NO: 97 and/or the nucleic acid sequence of SEQ ID NO: 98;

(c) the nucleic acid sequence of SEQ ID NO: 99 and/or the nucleic acid sequence of SEQ ID NO: 100;

(d) the nucleic acid sequence of SEQ ID NO: 101 and/or the nucleic acid sequence of SEQ ID NO: 102;

(e) the nucleic acid sequence of SEQ ID NO: 103 and/or the nucleic acid sequence of SEQ ID NO: 104;

(f) the nucleic acid sequence of SEQ ID NO: 105 and/or the nucleic acid sequence of SEQ ID NO: 106;

(g) the nucleic acid sequence of SEQ ID NO: 107 and/or the nucleic acid sequence of SEQ ID NO: 108;

(h) the nucleic acid sequence of SEQ ID NO: 113 and/or the nucleic acid sequence of SEQ ID NO: 114;

(i) the nucleic acid sequence of SEQ ID NO: 115 and/or the nucleic acid sequence of SEQ ID NO: 116;

(j) the nucleic acid sequence of SEQ ID NO: 117 and/or the nucleic acid sequence of SEQ ID NO: 118;

(k) the nucleic acid sequence of SEQ ID NO: 119 and/or the nucleic acid sequence of SEQ ID NO: 120;

(l) the nucleic acid sequence of SEQ ID NO: 121 and/or the nucleic acid sequence of SEQ ID NO: 122; or

(m) the nucleic acid sequence of SEQ ID NO: 123 and/or the nucleic acid sequence of SEQ ID NO: 124.

45. An expression vector comprising the nucleic acid of paragraph 43 or 44.

46. A host cell comprising the nucleic acid of paragraph 43 or 44 or the vector of paragraph 45.

47. A method of producing an antibody molecule, comprising culturing the host cell of paragraph 46 under conditions suitable for gene expression.

48. A method of inhibiting FGF23, comprising contacting FGF23 with an antibody molecule of any of paragraphs 1-41, or a pharmaceutical composition of paragraph 42.

49. The method of paragraph 48, wherein the contacting step occurs in vitro, ex vivo, or in vivo.

50. A method of treating a disorder, comprising administering to a subject in need thereof an antibody molecule of any of paragraphs 1-41, or a pharmaceutical composition of paragraph 42, in an amount effective to treat the disorder.

51. The method of paragraph 50, wherein the disorder is a FGF23-associated disorder, optionally, wherein the FGF23-associated disorder is chosen from X-linked hypophosphatemic rickets (XLH), autosomal recessive hypophosphatemic rickets (ARHR) (e.g., ARHR 1 or ARHR2), autosomal dominant hypophosphatemic rickets (ADHR), osteoglophonic dysplasia, Jansen-type metaphyseal chondrodysplasia, hypophosphatemia with dental abnormality and ectopic calcification, McCune-Albright syndrome, epidermal nevus syndrome (ENS), or tumor-induced osteomalacia (TIO).

52. The method of paragraph 50 or 51, wherein the antibody molecule is administered to the subject at a dose between 0.1 mg/kg and 50 mg/kg.

53. The method of any of paragraphs 50-52, further comprising administering a second therapeutic agent or modality.

54. The method of paragraph 53, wherein the second therapeutic agent or modality is administered before, during, or after the antibody molecule is administered.

55. A method of preventing a disorder, comprising administering to a subject in need thereof an antibody molecule of any of paragraphs 1-41, or a pharmaceutical composition of paragraph 42, in an amount effective to treat the disorder.

56. The method of paragraph 55, wherein the disorder is a FGF23-associated disorder, optionally, wherein the FGF23-associated disorder is chosen from X-linked hypophosphatemic rickets (XLH), autosomal recessive hypophosphatemic rickets (ARHR) (e.g., ARHR 1 or ARHR2), autosomal dominant hypophosphatemic rickets (ADHR), osteoglophonic dysplasia, Jansen-type metaphyseal chondrodysplasia, hypophosphatemia with dental abnormality and ectopic calcification, McCune-Albright syndrome, epidermal nevus syndrome (ENS), or tumor-induced osteomalacia (TIO).

57. A method of detecting FGF23, comprising (i) contacting a sample or a subject with an antibody molecule of any of paragraphs 1-41 under conditions that allow interaction of the antibody molecule and FGF23 to occur, and (ii) detecting formation of a complex between the antibody molecule and the sample or subject.

58. The method of paragraph 58, further comprising contacting a reference sample or subject with an antibody molecule of any of paragraphs 1-41 under conditions that allow interaction of the antibody molecule and FGF23 to occur, and (ii) detecting formation of a complex between the antibody molecule and the sample or subject.

59. An antibody molecule of any of paragraphs 1-41, or a pharmaceutical composition of paragraph 42, for use in treating a disorder in a subject.

60. The antibody molecule, or pharmaceutical composition, for use of paragraph 59, wherein the disorder is a FGF23-associated disorder, optionally, wherein the FGF23-associated disorder is chosen from X-linked hypophosphatemic rickets (XLH), autosomal recessive hypophosphatemic rickets (ARHR) (e.g., ARHR 1 or ARHR2), autosomal dominant hypophosphatemic rickets (ADHR), osteoglophonic dysplasia, Jansen-type metaphyseal chondrodysplasia, hypophosphatemia with dental abnormality and ectopic calcification, McCune-Albright syndrome, epidermal nevus syndrome (ENS), or tumor-induced osteomalacia (TIO).

61. Use of an antibody molecule of any of paragraphs 1-41, or a pharmaceutical composition of paragraph 42, in the manufacture of a medicament for treating a disorder in a subject.

62. The use of paragraph 61, wherein the disorder is a FGF23-associated disorder, optionally, wherein the FGF23-associated disorder is chosen from X-linked hypophosphatemic rickets (XLH), autosomal recessive hypophosphatemic rickets (ARHR) (e.g., ARHR 1 or ARHR2), autosomal dominant hypophosphatemic rickets (ADHR), osteoglophonic dysplasia, Jansen-type metaphyseal chondrodysplasia, hypophosphatemia with dental abnormality and ectopic calcification, McCune-Albright syndrome, epidermal nevus syndrome (ENS), or tumor-induced osteomalacia (TIO).

### EXAMPLES

### Example 1

### Analytical Characterization of Anti-FGF23 Antibodies

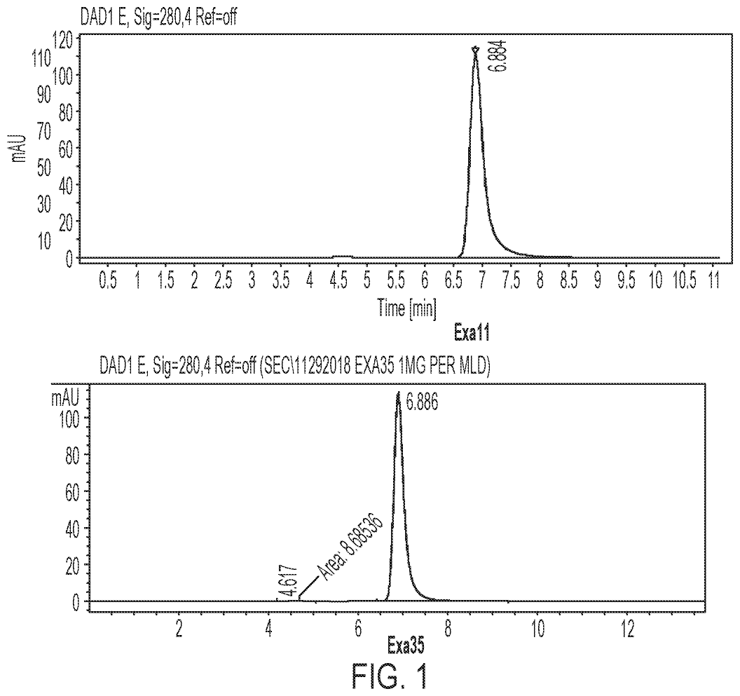

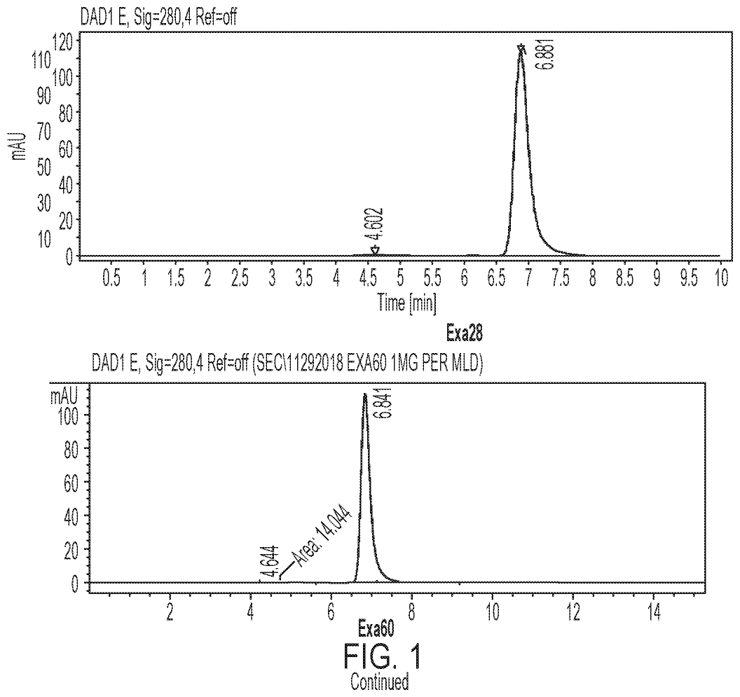

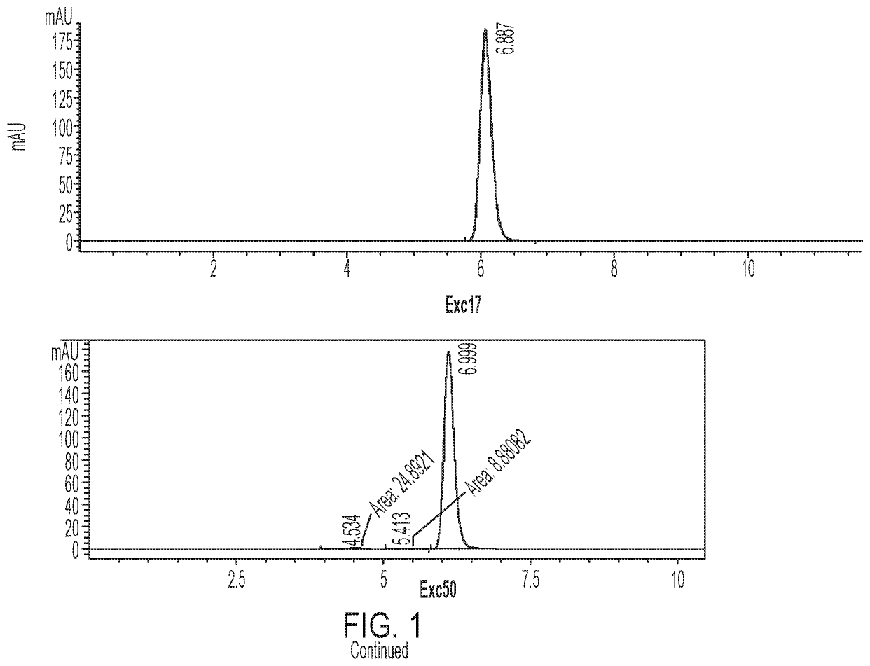

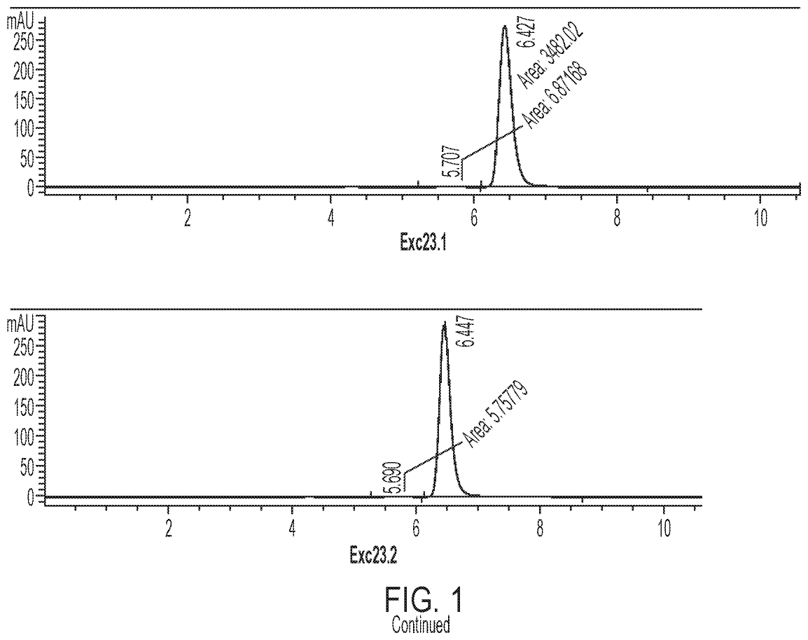

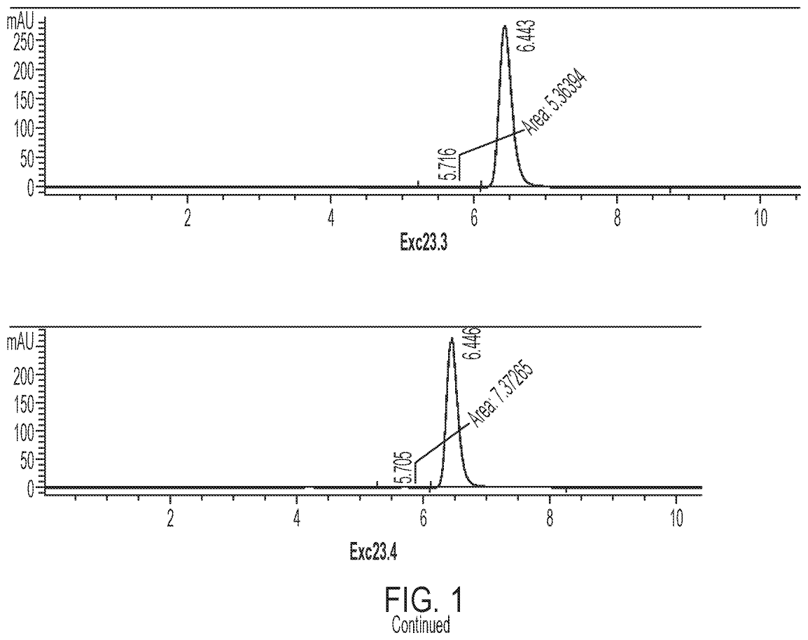

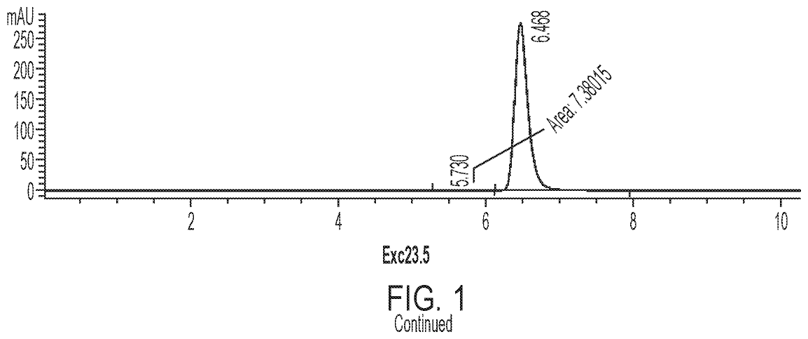

Analytical size exclusion chromatography (SEC) was used to characterize monoclonal anti-FGF23 antibodies ExA11, ExA35, ExA28, ExA60, Exc17, Exc50, Exc23.1, Exc23.2, Exc23.3, Exc23.4, and Exc23.5 for their monomeric and higher-order states. The column used for analytical SEC was the Agilent BioMab SEC column (4.6×50 mm) with a 3 μm particle size. The mobile phase used for the SEC analysis was 1× PBS pH 7.4. As shown in FIG. 1, all monoclonal anti-FGF23 antibodies were found to be in a monomeric state.

### Example 2

### Neutralization of FGF23 Signaling by Anti-FGF23 Antibodies

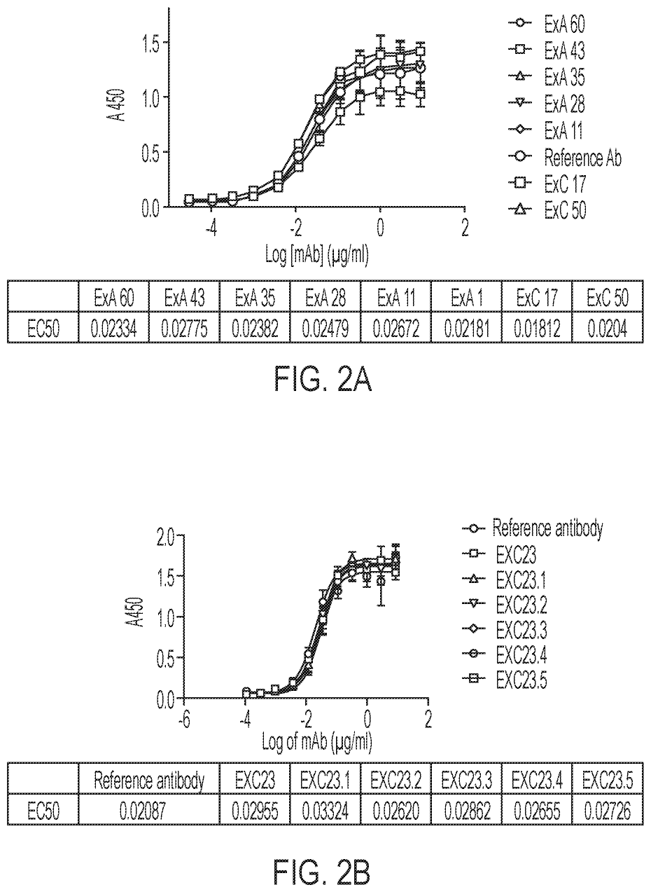

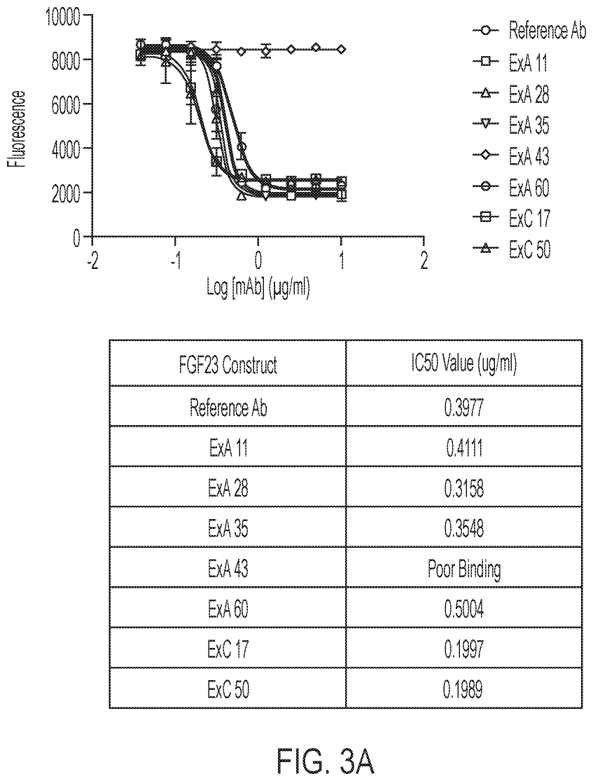

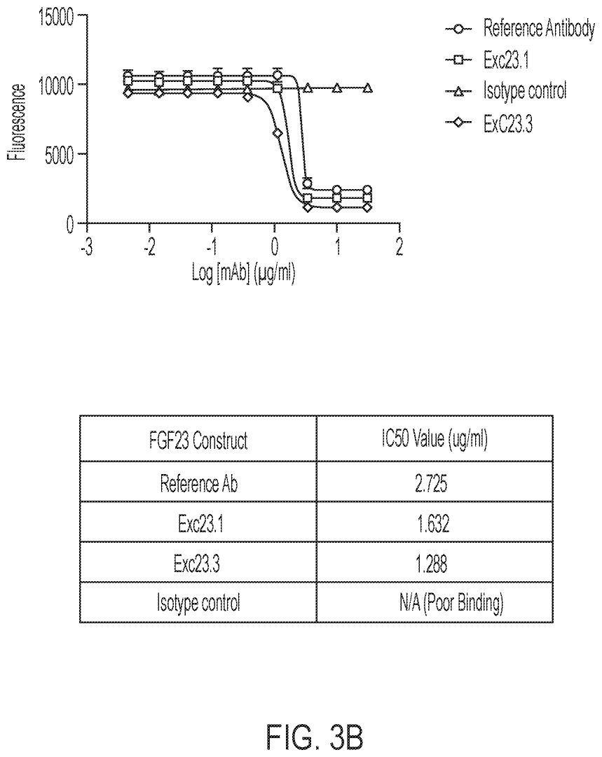

In this example, a series of anti-FGF23-antibodies was shown to inhibited FGF23-mediated signaling in cell-based assays. In the presence of recombinant mouse klotho and heparin, recombinant human FGF¬23 stimulated proliferation in the NIH¬3T3 mouse embryonic fibroblast cell line in a dose dependent manner Under these conditions, proliferation elicited by recombinant human FGF-23 is neutralized by increasing concentrations of anti-FGF23 antibodies. The half maximal inhibitory concentration (IC50) is a measure of the potency of the antibody to inhibit FGF23 signaling. Along with a reference FGF23 antibody, the potency of a series of engineered anti-FGF23 antibodies (ExA11, ExA28, ExA35, ExA43, ExA60, ExC17, ExC50, Exc23, Exc23.1, Exc23.2, Exc23.3, Exc23.4, or Exc23.5) was tested. These antibodies bound to human FGF23 with EC50 values between 0.01-0.03 ug/ml (FIGS. 2A-2B). As shown in FIGS. 3A-3B, antibodies ExA11, ExA28, ExA35, ExA60, ExC17, ExC50, Exc23.1, and Exc23.3 showed efficacy comparable to the reference FGF23 antibody, whereas ExA43 did not show neutralization of FGF23 signaling. The neutralization was highly sensitive as the inhibition potential of the antibodies strongly correlated with the affinity of the antibodies to the FGF23 protein. The IC50 values of the anti-FGF23 antibodies varied between 0.3-0.6 ug/ml.

### INCORPORATION BY REFERENCE

All publications, patents, and Accession numbers mentioned herein are hereby incorporated by reference in their entirety as if each individual publication or patent was specifically and individually indicated to be incorporated by reference.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

What is claimed is:

1. An isolated antibody molecule capable of binding to FGF23, comprising:
(a) a heavy chain variable region (VH) comprising an HCDR1 amino acid sequence of SEQ ID NO: 41, an HCDR2 amino acid sequence of SEQ ID NO: 48, and an HCDR3 amino acid sequence of SEQ ID NO: 53; and a light chain variable region (VL) comprising an LCDR1 amino acid sequence of SEQ ID NO: 61, an LCDR2 amino acid sequence of SEQ ID NO: 67, and an LCDR3 amino acid sequence of SEQ ID NO: 74;
(b) a VH comprising an HCDR1 amino acid sequence of SEQ ID NO: 41, an HCDR2 amino acid sequence of SEQ ID NO: 46, and an HCDR3 amino acid sequence of SEQ ID NO: 53; and a light chain variable region (VL) comprising an LCDR1 amino acid sequence of SEQ ID NO: 61, an LCDR2 amino acid sequence of SEQ ID NO: 67, and an LCDR3 amino acid sequence of SEQ ID NO: 74;
(c) a VH comprising an HCDR1 amino acid sequence of SEQ ID NO: 41, an HCDR2 amino acid sequence of SEQ ID NO: 49, and an HCDR3 amino acid sequence of SEQ ID NO: 53; and a light chain variable region (VL) comprising an LCDR1 amino acid sequence of SEQ ID NO: 61, an LCDR2 amino acid sequence of SEQ ID NO: 67, and an LCDR3 amino acid sequence of SEQ ID NO: 74;
(d) a VH comprising an HCDR1 amino acid sequence of SEQ ID NO: 41, an HCDR2 amino acid sequence of SEQ ID NO: 46, and an HCDR3 amino acid sequence of SEQ ID NO: 57; and a light chain variable region (VL) comprising an LCDR1 amino acid sequence of SEQ ID NO: 61, an LCDR2 amino acid sequence of SEQ ID NO: 67, and an LCDR3 amino acid sequence of SEQ ID NO: 74;
(e) a VH comprising an HCDR1 amino acid sequence of SEQ ID NO: 41, an HCDR2 amino acid sequence of SEQ ID NO: 50, and an HCDR3 amino acid sequence of SEQ ID NO: 54; and a light chain variable region (VL) comprising an LCDR1 amino acid sequence of SEQ ID NO: 61, an LCDR2 amino acid sequence of SEQ ID NO: 67, and an LCDR3 amino acid sequence of SEQ ID NO: 74;
(f) a VH comprising an HCDR1 amino acid sequence of SEQ ID NO: 41, an HCDR2 amino acid sequence of SEQ ID NO: 46, and an HCDR3 amino acid sequence of SEQ ID NO: 53; and a light chain variable region (VL) comprising an LCDR1 amino acid sequence of SEQ ID NO: 61, an LCDR2 amino acid sequence of SEQ ID NO: 69, and an LCDR3 amino acid sequence of SEQ ID NO: 89;
(g) a VH comprising an HCDR1 amino acid sequence of SEQ ID NO: 41, an HCDR2 amino acid sequence of SEQ ID NO: 49, and an HCDR3 amino acid sequence of SEQ ID NO: 55; and a light chain variable region (VL) comprising an LCDR1 amino acid sequence of SEQ ID NO: 61, an LCDR2 amino acid sequence of SEQ ID NO: 69, and an LCDR3 amino acid sequence of SEQ ID NO: 74;
(h) a VH comprising an HCDR1 amino acid sequence of SEQ ID NO: 41, an HCDR2 amino acid sequence of SEQ ID NO: 46, and an HCDR3 amino acid sequence of SEQ ID NO: 53; and a light chain variable region (VL) comprising an LCDR1 amino acid sequence of SEQ ID NO: 59, an LCDR2 amino acid sequence of SEQ ID NO: 70, and an LCDR3 amino acid sequence of SEQ ID NO: 73;
(i) a VH comprising an HCDR1 amino acid sequence of SEQ ID NO: 41, an HCDR2 amino acid sequence of SEQ ID NO: 46, and an HCDR3 amino acid sequence of SEQ ID NO: 53; and a light chain variable region (VL) comprising an LCDR1 amino acid sequence of SEQ ID NO: 59, an LCDR2 amino acid sequence of SEQ ID NO: 68, and an LCDR3 amino acid sequence of SEQ ID NO: 73;
(j) a VH comprising an HCDR1 amino acid sequence of SEQ ID NO: 41, an HCDR2 amino acid sequence of SEQ ID NO: 46, and an HCDR3 amino acid sequence of SEQ ID NO: 53; and a light chain variable region (VL) comprising an LCDR1 amino acid sequence of SEQ ID NO: 59, an LCDR2 amino acid sequence of SEQ ID NO: 69, and an LCDR3 amino acid sequence of SEQ ID NO: 73;
(k) a VH comprising an HCDR1 amino acid sequence of SEQ ID NO: 41, an HCDR2 amino acid sequence of SEQ ID NO: 46, and an HCDR3 amino acid sequence of SEQ ID NO: 53; and a light chain variable region (VL) comprising an LCDR1 amino acid sequence of SEQ ID NO: 59, an LCDR2 amino acid sequence of SEQ ID NO: 70, and an LCDR3 amino acid sequence of SEQ ID NO: 74;
(l) a VH comprising an HCDR1 amino acid sequence of SEQ ID NO: 41, an HCDR2 amino acid sequence of SEQ ID NO: 46, and an HCDR3 amino acid sequence of SEQ ID NO: 53; and a light chain variable region (VL) comprising an LCDR1 amino acid sequence of SEQ ID NO: 61, an LCDR2 amino acid sequence of SEQ ID NO: 70, and an LCDR3 amino acid sequence of SEQ ID NO: 73; or
(m) a VH comprising an HCDR1 amino acid sequence of SEQ ID NO: 41, an HCDR2 amino acid sequence of SEQ ID NO: 46, and an HCDR3 amino acid sequence of SEQ ID NO: 53; and a light chain variable region (VL) comprising an LCDR1 amino acid sequence of SEQ ID NO: 59, an LCDR2 amino acid sequence of SEQ ID NO: 70, and an LCDR3 amino acid sequence of SEQ ID NO: 73.

2. An isolated antibody molecule capable of binding to FGF23, comprising a heavy chain variable region (VH) and a light chain variable region (VL), wherein the VH comprises an HCDR1 amino acid sequence of any of SEQ ID NOs: 39-43 or 110; an HCDR2 amino acid sequence of any of SEQ ID NOs: 44-52; and an HCDR3 amino acid sequence of any of SEQ ID NOs: 53-57; and the VL comprises an LCDR1 amino acid sequence of any of SEQ ID NOs: 58-63 and 109, an LCDR2 amino acid sequence of any of SEQ ID NOs: 64-72, and an LCDR3 amino acid sequence of any of SEQ ID NOs: 73-89.

3. The antibody molecule of claim 1 or 2, which comprises a VH comprising an amino acid sequence at least 85%, 90%, or 95% identical to any of SEQ ID NOs: 1-13, 90, or 91.

4. The antibody molecule of claim 3, which comprises a VH comprising an amino acid sequence of any of SEQ ID NOs: 1-13, 90, or 91.

5. The antibody molecule of any of claims 1-4, which comprises a VL comprising an amino acid sequence at least 85%, 90%, or 95% identical to any of SEQ ID NOs: 14-38 or 92-96.

6. The antibody molecule of claim 5, which comprises a VL comprising an amino acid sequence of any of SEQ ID NOs: 14-38 or 92-96.

7. The antibody molecule of claim 1, which comprises a VH comprising an amino acid sequence at least 85%, 90%, or 95% identical to any of SEQ ID NOs: 1-13, 90, or 91 and a VL comprising an amino acid sequence at least 85%, 90%, or 95% identical to any of SEQ ID NOs: 14-38 or 92-96.

8. The antibody molecule of claim 7, which comprises a VH comprising an amino acid sequence of any of SEQ ID NOs: 1-13, 90, or 91 and a VL comprising an amino acid sequence of any of SEQ ID NOs: 14-38 or 92-96.

9. The antibody molecule of any of claims 1-8, which comprise a VH comprising an amino acid sequence of SEQ ID NO: 5 and a VL comprising an amino acid sequence of SEQ ID NO: 19.

10. The antibody molecule of any of claims 1-8, which comprise a VH comprising an amino acid sequence of SEQ ID NO: 7 and a VL comprising an amino acid sequence of SEQ ID NO: 20.

11. The antibody molecule of any of claims 1-8, which comprise a VH comprising an amino acid sequence of SEQ ID NO: 8 and a VL comprising an amino acid sequence of SEQ ID NO: 19.

12. The antibody molecule of any of claims 1-8, which comprise a VH comprising an amino acid sequence of SEQ ID NO: 9 and a VL comprising an amino acid sequence of SEQ ID NO: 19.

13. The antibody molecule of any of claims 1-8, which comprise a VH comprising an amino acid sequence of SEQ ID NO: 11 and a VL comprising an amino acid sequence of SEQ ID NO: 20.

14. The antibody molecule of any of claims 1-8, which comprise a VH comprising an amino acid sequence of SEQ ID NO: 7 and a VL comprising an amino acid sequence of SEQ ID NO: 28.

15. The antibody molecule of any of claims 1-8, which comprise a VH comprising an amino acid sequence of SEQ ID NO: 13 and a VL comprising an amino acid sequence of SEQ ID NO: 25.

16. The antibody molecule of any of claims 1-8, which comprise a VH comprising an amino acid sequence of SEQ ID NO: 7 and a VL comprising an amino acid sequence of SEQ ID NO: 37.

17. The antibody molecule of any of claims 1-8, which comprise a VH comprising an amino acid sequence of SEQ ID NO: 7 and a VL comprising an amino acid sequence of SEQ ID NO: 34.

18. The antibody molecule of any of claims 1-8, which comprise a VH comprising an amino acid sequence of SEQ ID NO: 7 and a VL comprising an amino acid sequence of SEQ ID NO: 36.

19. The antibody molecule of any of claims 1-8, which comprise a VH comprising an amino acid sequence of SEQ ID NO: 7 and a VL comprising an amino acid sequence of SEQ ID NO: 94.

20. The antibody molecule of any of claims 1-8, which comprise a VH comprising an amino acid sequence of SEQ ID NO: 7 and a VL comprising an amino acid sequence of SEQ ID NO: 95.

21. The antibody molecule of any of claims 1-8, which comprise a VH comprising an amino acid sequence of SEQ ID NO: 7 and a VL comprising an amino acid sequence of SEQ ID NO: 96.

22. The antibody molecule of any of the preceding claims, which comprises an antigen-binding fragment.

23. The antibody molecule of claim 22, wherein the antigen-binding fragment comprises a Fab, F(ab′)2, Fv, scFv, or sc(Fv)2.

24. The antibody molecule of any of the preceding claims, which comprises a light chain constant region chosen from the light chain constant regions of kappa or lambda.

25. The antibody molecule of any of the preceding claims, which comprises a heavy chain constant region chosen from the heavy chain constant regions of IgG1, IgG2, IgG3, IgG4, or a chimera of two or more isotypes (e.g. IgG2 and IgG4), and a light chain constant region chosen from the light chain constant regions of kappa or lambda.

26. The antibody molecule of any of the preceding claims, which comprises a heavy chain constant region chosen from the heavy chain constant regions of IgG1, IgG4, or a chimera of IgG2 and IgG4 (e.g. “IgG2/4”), and optionally, wherein the heavy chain constant region comprises one or more amino acid modifications in the hinge, CH2, and/or CH3 region (e.g., IgG1-YTE, IgG4-YTE or IgG2/4-YTE).

27. The antibody molecule of any of the preceding claims, which comprises an Fc region.

28. The antibody molecule of any of the preceding claims, wherein said antibody molecule is a humanized antibody molecule.

29. The antibody molecule of any of the preceding claims, wherein said antibody molecule is a monoclonal antibody molecule.

30. The antibody molecule of any of the preceding claims, wherein said antibody molecule is a synthetic antibody molecule.

31. The antibody molecule of any of the preceding claims, wherein said antibody molecule is a monospecific antibody molecule.

32. The antibody molecule of any of the preceding claims, wherein the FGF23 is a human FGF23.

33. The antibody molecule of any of the preceding claims, which binds to human FGF23 at an EC₅₀ of less than 0.04 μg/ml (e.g., less than about 0.04, 0.03, 0.029, 0.028, 0.027, 0.026, 0.025, 0.024, 0.023, 0.022, or 0.021 μg/ml), e.g., as determined by ELISA.

34. The antibody molecule of any of the preceding claims, which binds to human FGF23 at an EC₅₀ of between 0.01 μg/ml and 0.04 μg/ml (e.g., between 0.02 μg/ml and 0.04 μg/ml, between 0.02 μg/ml and 0.03 μg/ml, between 0.02 μg/ml and 0.025 μg/ml, or between 0.025 μg/ml and 0.03 μg/ml), e.g., as determined by ELISA.

35. The antibody molecule of any of the preceding claims, which inhibits cell proliferation at an IC₅₀ of less than 10 μg/ml (e.g., less than about 10, 9, 8, 7, 6, 5, 4, 3, 2.8, 2.5, 2, 1.7, 1.6, 1.5, 1.4, 1.3, 1, 0.9, 0.8, 0.7, 0.6, 0.5, 0.4, 0.3, 0.2, or 0.1 μg/ml), e.g., as determined by a cell-based assay, e.g., as described in Example 2.

36. The antibody molecule of any of the preceding claims, which inhibits cell proliferation at an IC₅₀ of between 0.1 μg/ml and 3 μg/ml (e.g., between 0.1 μg/ml and 0.3 μg/ml, between 0.3 μg/ml and 0.6 μg/ml, between 0.6 μg/ml and 1 μg/ml, between 1 μg/ml and 2 μg/ml, or between 2 μg/ml and 3 μg/ml) as determined by a cell-based assay, e.g., as described in Example 2.

37. The antibody molecule of any of the preceding claims, which binds to human FGF23 comprising the amino acid sequence of SEQ ID NO: 82.

38. The antibody molecule of any of the preceding claims, wherein LCDR1, LCDR2, LCDR3, HCDR1 and HCDR2 belong to Chothia CDR canonical classes 2, 1, 3, 1 and 3, respectively.

39. An antibody molecule that competes for binding to FGF23 with an antibody molecule of any of the preceding claims.

40. An antibody molecule that binds to the same or overlapping epitope as the epitope recognized by an antibody molecule of any of the preceding claims.

41. A pharmaceutical composition comprising the isolated antibody molecule of any of the preceding claims and a pharmaceutically acceptable carrier, excipient or stabilizer.

42. An isolated nucleic acid encoding the VH, VL, or both, of the antibody molecule of any of claims 1-40.

43. The isolated nucleic acid of claim 42, wherein the nucleic acid comprises:
(a) the nucleic acid sequence of SEQ ID NO: 111 and/or the nucleic acid sequence of SEQ ID NO: 112;
(b) the nucleic acid sequence of SEQ ID NO: 97 and/or the nucleic acid sequence of SEQ ID NO: 98;
(c) the nucleic acid sequence of SEQ ID NO: 99 and/or the nucleic acid sequence of SEQ ID NO: 100;
(d) the nucleic acid sequence of SEQ ID NO: 101 and/or the nucleic acid sequence of SEQ ID NO: 102;
(e) the nucleic acid sequence of SEQ ID NO: 103 and/or the nucleic acid sequence of SEQ ID NO: 104;
(f) the nucleic acid sequence of SEQ ID NO: 105 and/or the nucleic acid sequence of SEQ ID NO: 106;
(g) the nucleic acid sequence of SEQ ID NO: 107 and/or the nucleic acid sequence of SEQ ID NO: 108;
(h) the nucleic acid sequence of SEQ ID NO: 113 and/or the nucleic acid sequence of SEQ ID NO: 114;
(i) the nucleic acid sequence of SEQ ID NO: 115 and/or the nucleic acid sequence of SEQ ID NO: 116;
(j) the nucleic acid sequence of SEQ ID NO: 117 and/or the nucleic acid sequence of SEQ ID NO: 118;
(k) the nucleic acid sequence of SEQ ID NO: 119 and/or the nucleic acid sequence of SEQ ID NO: 120;
(l) the nucleic acid sequence of SEQ ID NO: 121 and/or the nucleic acid sequence of SEQ ID NO: 122; or
(m) the nucleic acid sequence of SEQ ID NO: 123 and/or the nucleic acid sequence of SEQ ID NO: 124.

44. An expression vector comprising the nucleic acid of claim 42 or 43.

45. A host cell comprising the nucleic acid of claim 42 or 43 or the vector of claim 44.

46. A method of producing an antibody molecule, comprising culturing the host cell of claim 45 under conditions suitable for gene expression.

47. A method of inhibiting FGF23, comprising contacting FGF23 with an antibody molecule of any of claims 1-40, or a pharmaceutical composition of claim 41.

48. The method of claim 47, wherein the contacting step occurs in vitro, ex vivo, or in vivo.

49. A method of treating a disorder, comprising administering to a subject in need thereof an antibody molecule of any of claims 1-40, or a pharmaceutical composition of claim 41, in an amount effective to treat the disorder.

50. The method of claim 49, wherein the disorder is a FGF23-associated disorder, optionally, wherein the FGF23-associated disorder is chosen from X-linked hypophosphatemic rickets (XLH), autosomal recessive hypophosphatemic rickets (ARHR) (e.g., ARHR 1 or ARHR2), autosomal dominant hypophosphatemic rickets (ADHR), osteoglophonic dysplasia, Jansen-type metaphyseal chondrodysplasia, hypophosphatemia with dental abnormality and ectopic calcification, McCune-Albright syndrome, epidermal nevus syndrome (ENS), or tumor-induced osteomalacia (TIO).

51. The method of claim 49 or 50, wherein the antibody molecule is administered to the subject at a dose between 0.1 mg/kg and 50 mg/kg.

52. The method of any of claims 49-51, further comprising administering a second therapeutic agent or modality.

53. The method of claim 52, wherein the second therapeutic agent or modality is administered before, during, or after the antibody molecule is administered.

54. A method of preventing a disorder, comprising administering to a subject in need thereof an antibody molecule of any of claims 1-40, or a pharmaceutical composition of claim 41, in an amount effective to treat the disorder.

55. The method of claim 54, wherein the disorder is a FGF23-associated disorder, optionally, wherein the FGF23-associated disorder is chosen from X-linked hypophosphatemic rickets (XLH), autosomal recessive hypophosphatemic rickets (ARHR) (e.g., ARHR 1 or ARHR2), autosomal dominant hypophosphatemic rickets (ADHR), osteoglophonic dysplasia, Jansen-type metaphyseal chondrodysplasia, hypophosphatemia with dental abnormality and ectopic calcification, McCune-Albright syndrome, epidermal nevus syndrome (ENS), or tumor-induced osteomalacia (TIO).

56. A method of detecting FGF23, comprising (i) contacting a sample or a subject with an antibody molecule of any of claims 1-40 under conditions that allow interaction of the antibody molecule and FGF23 to occur, and (ii) detecting formation of a complex between the antibody molecule and the sample or subject.

57. The method of claim 56, further comprising contacting a reference sample or subject with an antibody molecule of any of claims 1-40 under conditions that allow interaction of the antibody molecule and FGF23 to occur, and (ii) detecting formation of a complex between the antibody molecule and the sample or subject.

58. An antibody molecule of any of claims 1-40, or a pharmaceutical composition of claim 41, for use in treating a disorder in a subject.

59. The antibody molecule, or pharmaceutical composition, for use of claim 58, wherein the disorder is a FGF23-associated disorder, optionally, wherein the FGF23-associated disorder is chosen from X-linked hypophosphatemic rickets (XLH), autosomal recessive hypophosphatemic rickets (ARHR) (e.g., ARHR 1 or ARHR2), autosomal dominant hypophosphatemic rickets (ADHR), osteoglophonic dysplasia, Jansen-type metaphyseal chondrodysplasia, hypophosphatemia with dental abnormality and ectopic calcification, McCune-Albright syndrome, epidermal nevus syndrome (ENS), or tumor-induced osteomalacia (TIO).

60. Use of an antibody molecule of any of claims 1-40, or a pharmaceutical composition of claim 41, in the manufacture of a medicament for treating a disorder in a subject.

61. The use of claim 60, wherein the disorder is a FGF23-associated disorder, optionally, wherein the FGF23-associated disorder is chosen from X-linked hypophosphatemic rickets (XLH), autosomal recessive hypophosphatemic rickets (ARHR) (e.g., ARHR 1 or ARHR2), autosomal dominant hypophosphatemic rickets (ADHR), osteoglophonic dysplasia, Jansen-type metaphyseal chondrodysplasia, hypophosphatemia with dental abnormality and ectopic calcification, McCune-Albright syndrome, epidermal nevus syndrome (ENS), or tumor-induced osteomalacia (TIO).
